(19) United States
(12) Patent Application Publication
(10) Pub. No.: US 2025/0222093 A1
(43) Pub. Date: Jul. 10, 2025

(54) METHODS OF PRODUCING BIOCONJUGATES OF E. COLI O-ANTIGEN POLYSACCHARIDES, COMPOSITIONS THEREOF, AND METHODS OF USE THEREOF

(71) Applicants: Janssen Pharmaceuticals, Inc., Titusville, NJ (US); GlaxoSmithKline Biologicals S.A., Rixensart (BE)

(72) Inventors: Jeroen GEURTSEN, Vleuten (NL); Pieter Jan BURGHOUT, Pijnacker (NL); Eveline Marleen WEERDENBURG, Uithoorn (NL); Jan Theunis POOLMAN, Haarlem (NL); Kellen Cristhina FAE, Oegstgeest (NL); Patricia IBARRA YON, Solothum (CH); Darren Robert ABBANAT, Cornwall, NY (US); Stefan Jochen KEMMLER, Zurich (CH); Michael Thomas KOWARIK, Zurich (CH); Manuela MALLY, Watt (CH); Veronica GAMBILLARA FONCK, Meilen (CH); Martin Edward BRAUN, Cham (CH); Maria Paula CARRANZA SANDMEIER, Rudolfstetten (CH)

(21) Appl. No.: 19/029,679

(22) Filed: Jan. 17, 2025

Related U.S. Application Data
(62) Division of application No. 17/938,454, filed on Oct. 6, 2022, now Pat. No. 12,233,121.
Application No. 17/938,454 is a division of application No. 16/822,403, filed on Mar. 18, 2020, now Pat. No. 11,491,220.
(60) Provisional application No. 62/819,762, filed on Mar. 18, 2019.

(51) Int. Cl. A61K 39/104; A61K 39/00; A61K 39/02; A61K 39/085; A61K 39/09; A61K 39/095; A61K 39/102; A61K 39/108; A61K 47/64

(52) U.S. Cl. CPC A61K 39/104; A61K 39/0258; A61K 39/085; A61K 39/092; A61K 39/095; A61K 39/102; A61K 39/107; A61K 47/646; A61K 2039/6037; A61K 2039/62

(57) ABSTRACT

Methods of producing bioconjugates of O-antigen polysaccharides covalently linked to a carrier protein using recombinant host cells are provided. The recombinant host cells used in the methods described herein encode a particular oligosaccharyl transferase enzyme depending on the O-antigen polysaccharide bioconjugate to be produced. The oligosaccharyl transferase enzymes can be PglB oligosaccharyl transferase or variants thereof. Also provided are compositions containing the bioconjugates, and methods of using the bioconjugates and compositions described herein to vaccinate a subject against extra-intestinal pathogenic E. coli. (ExPEC).

### CROSS REFERENCE TO RELATED APPLICATION

This application is a divisional application of U.S. application Ser. No. 17/938,454 filed Oct. 6, 2022, which is a divisional application of U.S. application Ser. No. 16/822,403 filed Mar. 18, 2020, now U.S. Pat. No. 11,491,220, issued Nov. 8, 2022, which claims priority to U.S. Provisional Application No. 62/819,762 filed on Mar. 18, 2019, each of the disclosures is incorporated herein by reference in its entirety.

### REFERENCE TO SEQUENCE LISTING SUBMITTED ELECTRONICALLY

The contents of the electronic sequence listing (004852-128US4.xml; Size: 156,456 byte; and Date of Creation: Jan. 17, 2025) is herein incorporated by reference in its entirety.

### BACKGROUND OF THE INVENTION

Extraintestinal pathogenic Escherichia coli (ExPEC) strains are normally harmless inhabitants of the human gastrointestinal tract, alongside commensal E. coli strains. ExPEC isolates cannot readily be distinguished from commensal isolates by serotype, although many clonal lineages are dominated by ExPEC, as defined by O-antigen, capsule and flagellar antigen serotypes (abbreviated as O:K:H, for example O25:K1:H4). In contrast to commensal E. coli, ExPEC strains express a broad array of virulence factors enabling them to colonize the gastrointestinal tract, as well as to cause a wide range of extraintestinal infections, which are associated with a significant healthcare cost burden due to hospitalization and death. Neonates, the elderly, and immunocompromised patients are particularly susceptible to ExPEC infection, including invasive ExPEC disease (IED).

ExPEC strains are the most common cause of urinary tract infections (UTI) and important contributors to surgical site infections and neonatal meningitis. The strains are also associated with abdominal and pelvic infections and nosocomial pneumonia, and are occasionally involved in other extraintestinal infections, such as osteomyelitis, cellulitis, and wound infections. All these primary sites of infection can result in ExPEC bacteremia. ExPEC is the most common cause of community-onset bacteremia and a major causative pathogen in nosocomial bacteremia and is found in about 17% to 37% of clinically significant blood isolates. Patients with an ExPEC-positive blood culture typically suffer sepsis syndrome, severe sepsis, or septic shock. Increasing resistance of ExPEC against first-line antibiotics including the cephalosporins, fluoroquinolones, and trimethoprim/sulfamethoxazole has been observed. The emergence and rapid global dissemination of ExPEC sequence type 131 (ST131) is considered a main driver of increased drug resistance, including multi-drug resistance. This clone is found in 12.5% to 30% of all ExPEC clinical isolates, exhibits mostly serotype O25b:H4, and shows high levels of resistance to fluoroquinolones, which is often accompanied by trimethoprim/sulfamethoxazole resistance and extended-spectrum beta-lactamases conferring resistance to cephalosporins.

The O-antigen comprises the immunodominant component of the cell wall lipopolysaccharide (LPS) in Gram-negative bacteria, including E. coli. There are currently >180 serologically unique E. coli O-antigens identified, with the vast majority of ExPEC isolates classified within less than 20 O-antigen serotypes. Full-length E. coli O-antigens are typically comprised of about 10 to 25 repeating sugar units attached to the highly conserved LPS core structure, with each component synthesized separately by enzymes encoded predominantly in the rfb and rfa gene clusters, respectively. Following polymerization of the O-antigen, the O-antigen polysaccharide backbone may be modified, typically through the addition of acetyl or glucose residues. These modifications effectively increase serotype diversity by creating antigenically distinct serotypes that share a common polysaccharide backbone, but differ in side branches. Genes encoding O-antigen modifying enzymes typically reside outside of the rfb cluster on the chromosome, and in some cases, these genes are found within lysogenic bacteriophages.

ExPEC isolates belonging to the O4 serogroup have been commonly identified in contemporary surveillance studies of U.S. and EU blood isolates. The structure of the O4 polysaccharide was determined as -->2) α-L-Rha (1->6) α-D-Glc (1->3) α-L-FucNAc (1->3) (3-D-GlcNAc (1->from an E. coli O4:K52 strain (Jann et al., Carbohydr. Res. (1993) v. 248, pp. 241-250). A distinct form of the O4 polysaccharide structure was determined for O4:K3, O4:K6 and O4:K12 strains, in which the structure above was modified by the addition of an α-D-Glc (1->3) linked to the rhamnose residue of the polysaccharide (Jann et al., 1993, supra), this form of the polysaccharide referred to herein below as ‘glucosylated O4’. The enzymes responsible for the O-antigen modification within E. coli O4 strains were not identified.

Efforts toward the development of a vaccine to prevent ExPEC infections have focused on O-antigen polysaccharide conjugates. A 12-valent O-antigen conjugate vaccine was synthesized through extraction and purification of O-antigen polysaccharide and chemical conjugation to detoxified Pseudomonas aeruginosa exotoxin A and tested for safety and immunogenicity in a Phase 1 clinical study (Cross et al., J. Infect. Dis. (1994) v. 170, pp. 834-40). This candidate vaccine was never licensed for clinical use. A bioconjugation system in E. coli has been developed recently, in which the polysaccharide antigen and the carrier protein are both synthesized in vivo and subsequently conjugated in vivo through the activities of the oligosaccharyl transferase PglB, a Campylobacter jejuni enzyme, expressed in E. coli (Wacker et al., Proc. Nat. Acad. Sci. (2006) v. 103, pp. 7088-93). This N-linked protein glycosylation system is capable of the transfer of diverse polysaccharides to a carrier protein, allowing for straightforward methods to purify the conjugate.

Bioconjugation has been used successfully to produce conjugate polysaccharide for an E. coli four-valent O-antigen candidate vaccine (Poolman and Wacker, J. Infect. Dis. (2016) v. 213(1), pp. 6-13). However, the development of a successful ExPEC vaccine requires coverage of predominant serotypes, and the presence of further O-antigen modifications in subsets of ExPEC isolates presents a further challenge in covering isolates displaying unmodified and modified LPS. Moreover, efficiency of production of the multiple components for more complex vaccine compositions covering multiple serotypes becomes increasingly important, and hence there remains a need for improvements in production of individual bioconjugates of specific O-antigens.

### BRIEF SUMMARY OF THE INVENTION

In view of increasing antibiotic resistance among ExPEC isolates and the presence of further O-antigen modifications among predominant O-serotypes, there is a need for improved prophylactic and therapeutic treatments for these infections. The invention satisfies this need by defining the genetic composition of contemporary clinical isolates, including identifying the genes encoding O-antigen modifying enzymes, thus allowing for the engineering of recombinant host cells capable of synthesizing bioconjugates of the O-antigens including bioconjugates comprising selected O-antigen modifications. In addition, in one aspect of the invention, host cells and methods for improved production of bioconjugates of specific O-antigens by using variants of oligosaccharyl transferase (OST) are provided, based on advantages of use of certain OST variants for bioconjugates of certain E. coli O-antigens in an unpredictable serotype-dependent manner. Use of such OST variants may in certain cases also affect the glycosylation pattern of the bioconjugate, e.g. by increasing the relative number of glycans coupled to the carrier protein as compared to bioconjugates produced using wild-type or other variants of the OST, and hence novel bioconjugates produced by such methods are also provided as an aspect of the invention.

In one aspect, provided is a method of preparing a bioconjugate of an E. coli Ox antigen polysaccharide covalently linked to a carrier protein, the method comprising:
(i) providing a recombinant host cell comprising:
   a. a nucleotide sequence of an rfb gene cluster for the Ox-antigen polysaccharide;
   b. a nucleotide sequence encoding the carrier protein comprising at least one glycosylation site comprising a glycosylation consensus sequence having SEQ ID NO: 1, preferably having SEQ ID NO: 2; and
   c. a nucleotide sequence encoding an oligosaccharyl transferase PglBy; and
(ii) culturing the recombinant host cell under conditions for production of the bioconjugate,
wherein:
when the Ox-antigen is O1A antigen polysaccharide, the PglBy comprises the amino acid mutations of N311V, K482R, D483H, and A669V;
when the Ox-antigen is glucosylated O4 antigen polysaccharide, the PglBy comprises the amino acid mutation N311V or the amino acid mutations Y77H and N311V, and the recombinant host cell further comprises a sequence encoding a glucosyltransferase GtrS having at least 80% identity to SEQ ID NO: 4 and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having at least 80% sequence identity to SEQ ID NOs: 7 and 8 respectively, wherein the translocase is capable of translocating bactoprenol linked glucose and the glycosyltransferase is capable of glucosylating bactoprenol;
when the Ox-antigen is O6A antigen polysaccharide, the PglBy comprises the amino acid mutations of N311V, K482R, D483H, and A669V;
when the Ox-antigen is O8 antigen polysaccharide, the PglBy comprises no amino acid mutations at positions 77, 80, 287, 289, 311, 482, 483 and 669;
when the Ox-antigen is O15 antigen polysaccharide, the PglBy comprises the amino acid mutations of N311V, K482R, D483H, and A669V;
when the Ox-antigen is O16 antigen polysaccharide, the PglBy comprises the amino acid mutations of Y77H, S80R, Q287P, K289R, and N311V;
when the Ox-antigen is O18A antigen polysaccharide, the PglBy comprises no amino acid mutations at positions 77, 80, 287, 289, 311, 482, 483 and 669; and
when the Ox-antigen is O75 antigen polysaccharide, the PglBy comprises the amino acid mutation of N311V,
wherein in each case the amino acid mutations are relative to the wild-type PglB having the amino acid sequence of SEQ ID NO: 6,
wherein the O1A, glucosylated O4, O6A, O8, O15, O16, O18A, and O75 antigen polysaccharides have the structures of Formulas (O1A), (O4-Glc+), (O6A), (O8), (O15), (O16), (O18A), and (O75), respectively, as shown in Table 1, and each n is independently an integer of 1 to 100, preferably 3 to 50, e.g. 5 to 40, e.g. 7 to 25, e.g. 10 to 20.

In one embodiment, the Ox-antigen is O1A antigen polysaccharide, the PglBy comprises the amino acid mutations of N311V, K482R, D483H, and A669V relative to wild-type PglB having the amino acid sequence of SEQ ID NO: 6.

In one embodiment, the Ox-antigen is glucosylated O4 antigen polysaccharide, and the PglBy comprises the amino acid mutation N311V relative to wild-type PglB having the amino acid sequence of SEQ ID NO: 6. In one embodiment, the Ox-antigen is glucosylated O4 antigen polysaccharide, and the PglBy comprises the amino acid mutations Y77H and N311V relative to wild-type PglB having the amino acid sequence of SEQ ID NO: 6. In embodiments wherein the Ox-antigen is glucosylated O4 antigen polysaccharide, the recombinant host cell preferably further comprises a sequence encoding a GtrS having at least 80% identity to SEQ ID NO: 4, and nucleotide sequences encoding a GtrA and a GtrB having at least 80% sequence identity to SEQ ID NOs: 7 and 8 respectively.

In one embodiment, the Ox-antigen is O6A antigen polysaccharide, the PglBy comprises the amino acid mutations of N311V, K482R, D483H, and A669V relative to wild-type PglB having the amino acid sequence of SEQ ID NO: 6.

In one embodiment, the Ox-antigen is O8 antigen polysaccharide, the PglBy comprises no amino acid mutations at positions 77, 80, 287, 289, 311, 482, 483 and 669 relative to wild-type PglB having the amino acid sequence of SEQ ID NO: 6.

In one embodiment, the Ox-antigen is O15 antigen polysaccharide, the PglBy comprises the amino acid mutations of N311V, K482R, D483H, and A669V relative to wild-type PglB having the amino acid sequence of SEQ ID NO: 6.

In one embodiment, the Ox-antigen is O16 antigen polysaccharide, the PglBy comprises the amino acid mutations of Y77H, S80R, Q287P, K289R, and N311V relative to wild-type PglB having the amino acid sequence of SEQ ID NO: 6.

In one embodiment, the Ox-antigen is O18A antigen polysaccharide, the PglBy comprises no amino acid mutations at positions 77, 80, 287, 289, 311, 482, 483 and 669 relative to wild-type PglB having the amino acid sequence of SEQ ID NO: 6, and preferably comprises the amino acid sequence of SEQ ID NO: 6.

In one embodiment, the Ox-antigen is O75 antigen polysaccharide, the PglBy comprises the amino acid mutation of N311V relative to wild-type PglB having the amino acid sequence of SEQ ID NO: 6.

In a particular aspect, provided is a method of preparing a bioconjugate of an E. coli Ox-antigen polysaccharide covalently linked to a carrier protein, the method comprising:
(i) providing a recombinant host cell comprising:
   a. a nucleotide sequence of an rfb gene cluster for the Ox-antigen polysaccharide;
   b. a nucleotide sequence encoding the carrier protein comprising at least one glycosylation site comprising a glycosylation consensus sequence having SEQ ID NO: 1, preferably having SEQ ID NO: 2; and
   c. a nucleotide sequence encoding an oligosaccharyl transferase PglBy; and
(ii) culturing the recombinant host cell under conditions for production of the bioconjugate,
wherein the PglBy comprises the amino acid mutation N311V relative to the wild-type PglB having the amino acid sequence of SEQ ID NO: 6,
wherein the Ox-antigen is O1A antigen polysaccharide, glucosylated O4 antigen polysaccharide, O6A antigen polysaccharide, O15 antigen polysaccharide, O16 antigen polysaccharide, or O75 antigen polysaccharide, and when the Ox-antigen is glucosylated O4 antigen polysaccharide, the recombinant host cell further comprises a sequence encoding a glucosyltransferase GtrS having at least 80% identity to SEQ ID NO: 4 and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having at least 80% sequence identity to SEQ ID NOs: 7 and 8, respectively, wherein the translocase is capable of translocating bactoprenol linked glucose and the glycosyltransferase is capable of glucosylating bactoprenol, and
   wherein the O1A, glucosylated O4, O6A, O15, O16, and O75 antigen polysaccharides have the structures of Formulas (O1A), (O4-Glc+), (O6A), (O15), (O16), and (O75), respectively, as shown in Table 1, and each n is independently an integer of 1 to 100, preferably 3 to 50, e.g. 5 to 40, e.g. 7 to 25, e.g. 10 to 20.

In certain embodiments, the method further comprises isolating the bioconjugate from the recombinant host cell.

In certain embodiments, the carrier protein is selected from the group consisting of detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In certain embodiments, the carrier protein is detoxified exotoxin A of Pseudomonas aeruginosa (EPA). Preferably, the EPA carrier protein comprises 1-10, preferably 2-4, more preferably 4, glycosylation sites. In certain embodiments, each glycosylation site comprises a glycosylation consensus sequence having SEQ ID NO: 2. In a particular embodiment, the EPA carrier protein comprises SEQ ID NO: 3.

In certain embodiments, the recombinant host cell is an E. coli cell, e.g., an E. coli K-12 strain, such as strain W3110.

In another aspect, provided is a bioconjugate produced by a method of preparing a bioconjugate of an Ox antigen polysaccharide covalently linked to a carrier protein as described herein.

In another aspect, provided is a composition comprising such a bioconjugate. In some embodiments, a composition comprises at least 2, preferably at least 3, more preferably at least 5, still more preferably at least 7 of such bioconjugates.

In certain embodiments, a composition according to the invention comprises a bioconjugate of E. coli glucosylated O4 antigen polysaccharide covalently linked to a carrier protein, wherein the glucosylated O4 antigen polysaccharide has the structure of Formula (O4-Glc+) as shown in Table 1, and n is an integer of 1 to 100, preferably 3 to 50, e.g. 5 to 40, e.g. 7 to 25, e.g. 10 to 20. In certain embodiments, a composition according to the invention further comprises at least a bioconjugate of E. coli O25B antigen polysaccharide covalently linked to a carrier protein, wherein the O25B antigen polysaccharide has the structure of Formula (O25B) as shown in Table 1, and n is an integer of 1 to 100, preferably 3 to 50, e.g. 5 to 40, e.g. 7 to 25, e.g. 10 to 20. In certain embodiments, a composition according to the invention further comprises at least a bioconjugate of E. coli O2 antigen polysaccharide covalently linked to a carrier protein, wherein the O2 antigen polysaccharide has the structure of Formula (O2) as shown in Table 1, and n is an integer of 1 to 100, preferably 3 to 50, e.g. 5 to 40, e.g. 7 to 25, e.g. 10 to 20.

In certain embodiments, a composition of the invention comprises: (i) bioconjugate of E. coli O1A antigen polysaccharide covalently coupled to a carrier protein, (ii) bioconjugate of E. coli O2 antigen polysaccharide covalently coupled to a carrier protein, (iii) bioconjugate of E. coli glucosylated O4 antigen polysaccharide covalently coupled to a carrier protein, (iv) bioconjugate of E. coli O6A antigen polysaccharide covalently coupled to a carrier protein, (v) bioconjugate of E. coli O8 antigen polysaccharide covalently coupled to a carrier protein, (vi) bioconjugate of E. coli O15 antigen polysaccharide covalently coupled to a carrier protein, (vii) bioconjugate of E. coli O16 antigen polysaccharide covalently coupled to a carrier protein, (viii) bioconjugate of E. coli O25B antigen polysaccharide covalently coupled to a carrier protein, and (ix) bioconjugate of E. coli O75 antigen polysaccharide covalently coupled to a carrier protein, wherein the O1A, O2, glucosylated O4, O6A, O8, O15, O16, O25B, and O75 antigen polysaccharides have the structures of Formulas (O1A), (O2), (O4-Glc+), (O6A), (O8), (O15), (O16), (O25B), and (O75), respectively, as shown in Table 1, and each n is independently an integer of 1 to 100, preferably 3 to 50, e.g. 5 to 40, e.g. 7 to 25, e.g. 10 to 20. In certain embodiments, such a composition further comprises: (x) bioconjugate of E. coli O18A antigen polysaccharide covalently coupled to a carrier protein, wherein the O18A antigen polysaccharide has the structure of Formula (O18A) as shown in Table 1, and n is an integer of 1 to 100, preferably 3 to 50, e.g. 5 to 40, e.g. 7 to 25, e.g. 10 to 20. In certain embodiments, a composition of the invention is an immunogenic composition.

In other aspects, provided is a method of vaccination a subject against extra-intestinal pathogenic E. coli (ExPEC), comprising administering to the subject such a bioconjugate or composition as described herein. In yet other aspects, provided is such bioconjugate or composition as described herein for use in vaccination against extra-intestinal pathogenic E. coli (ExPEC).

In other aspects, provided are recombinant host cells for preparing a bioconjugate of an E. coli Ox antigen polysaccharide covalently linked to a carrier protein, the recombinant host cell comprising:
(a) a nucleotide sequence of an rfb gene cluster for the Ox-antigen polysaccharide;
(b) a nucleotide sequence encoding the carrier protein comprising at least one glycosylation site comprising a glycosylation consensus sequence having SEQ ID NO: 1, preferably having SEQ ID NO: 2; and
(c) a nucleotide sequence encoding an oligosaccharyl transferase PglBy,
   wherein:
when the Ox-antigen is O1A antigen polysaccharide, the PglBy comprises the amino acid mutations of N311V, K482R, D483H, and A669V;
when the Ox-antigen is glucosylated O4 antigen polysaccharide, the PglBy comprises the amino acid mutation N311V or the amino acid mutations Y77H and N311V, and the recombinant host cell further comprises a sequence encoding a glucosyltransferase GtrS having at least 80% identity to SEQ ID NO: 4 and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having at least 80% sequence identity to SEQ ID NOs: 7 and 8 respectively, wherein the translocase is capable of translocating bactoprenol linked glucose and the glycosyltransferase is capable of glucosylating bactoprenol;
when the Ox-antigen is O6A antigen polysaccharide, the PglBy comprises the amino acid mutations of N311V, K482R, D483H, and A669V;
when the Ox-antigen is O6A antigen polysaccharide, the PglBy comprises the amino acid mutations of N311V, K482R, D483H, and A669V;
when the Ox-antigen is O15 antigen polysaccharide, the PglBy comprises the amino acid mutations of N311V, K482R, D483H, and A669V;
when the Ox-antigen is O16 antigen polysaccharide, the PglBy comprises the amino acid mutations of Y77H, S80R, Q287P, K289R, and N311V;
when the Ox-antigen is O18A antigen polysaccharide, the PglBy comprises no amino acid mutations at positions 77, 80, 287, 289, 311, 482, 483 and 669; and
when the Ox-antigen is O75 antigen polysaccharide, the PglBy comprises the amino acid mutation of N311V,
wherein in each case the amino acid mutations are relative to the wild-type PglB having the amino acid sequence of SEQ ID NO: 6, and
wherein the O1A, glucosylated O4, O6A, O8, O15, O16, O18A, and O75 antigen polysaccharides have the structures of Formulas (O1A), (O4-Glc+), (O6A), (O8), (O15), (O16), (O18A), and (O75), respectively, as shown in Table 1, and each n is independently an integer of 1 to 100, preferably 3 to 50, e.g. 5 to 40, e.g. 7 to 25, e.g. 10 to 20.

In certain embodiments, such host cells are provided wherein the Ox-antigen is O1A antigen polysaccharide, and the PglBy comprises the amino acid mutations of N311V, K482R, D483H, and A669V relative to wild-type PglB having the amino acid sequence of SEQ ID NO: 6.

In certain embodiments, recombinant host cells of the invention are provided wherein the Ox-antigen is glucosylated O4 antigen polysaccharide, and the PglBy comprises the amino acid mutation N311V relative to wild-type PglB having the amino acid sequence of SEQ ID NO: 6. In certain embodiments, recombinant host cells of the invention are provided wherein the Ox-antigen is glucosylated O4 antigen polysaccharide, and the PglBy comprises the amino acid mutations Y77H and N311V relative to wild-type PglB having the amino acid sequence of SEQ ID NO: 6. In certain embodiments wherein the Ox-antigen is glucosylated O4 antigen polysaccharide, the recombinant host cell further comprises a sequence encoding a GtrS having the amino acid sequence of SEQ ID NO: 4, and nucleotide sequences encoding a GtrA and a GtrB having the amino acid sequences of SEQ ID NOs: 7 and 8, respectively.

In certain embodiments, recombinant host cells of the invention are provided wherein the Ox-antigen is O6A antigen polysaccharide, and the PglBy comprises the amino acid mutations of N311V, K482R, D483H, and A669V relative to wild-type PglB having the amino acid sequence of SEQ ID NO: 6.

In certain embodiments, recombinant host cells of the invention are provided wherein the Ox-antigen is O8 antigen polysaccharide, the PglBy comprises no amino acid mutations at positions 77, 80, 287, 289, 311, 482, 483 and 669 relative to wild-type PglB having the amino acid sequence of SEQ ID NO: 6.

In certain embodiments, recombinant host cells of the invention are provided wherein the Ox-antigen is O15 antigen polysaccharide, the PglBy comprises the amino acid mutations of N311V, K482R, D483H, and A669V relative to wild-type PglB having the amino acid sequence of SEQ ID NO: 6.

In certain embodiments, recombinant host cells of the invention are provided wherein the Ox-antigen is O16 antigen polysaccharide, the PglBy comprises the amino acid mutations of Y77H, S80R, Q287P, K289R, and N311V relative to wild-type PglB having the amino acid sequence of SEQ ID NO: 6.

In certain embodiments, recombinant host cells of the invention are provided wherein the Ox-antigen is O18A antigen polysaccharide, the PglBy comprises no amino acid mutations at positions 77, 80, 287, 289, 311, 482, 483 and 669 relative to wild-type PglB having the amino acid sequence of SEQ ID NO: 6.

In certain embodiments, recombinant host cells of the invention are provided wherein the Ox-antigen is O75 antigen polysaccharide, the PglBy comprises the amino acid mutation of N311V relative to wild-type PglB having the amino acid sequence of SEQ ID NO: 6.

In certain embodiments, recombinant host cells of the invention are provided wherein the carrier protein is selected from the group consisting of detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In certain embodiments, recombinant host cells of the invention are provided wherein the carrier protein is detoxified exotoxin A of Pseudomonas aeruginosa (EPA). In certain embodiments thereof, the EPA carrier protein comprises 1-10, preferably 2-4, more preferably 4, of the glycosylation sites. In certain embodiments, each glycosylation site comprises a glycosylation consensus sequence having SEQ ID NO: 2. In certain embodiments, the EPA carrier protein comprises SEQ ID NO: 3.

In certain embodiments, recombinant host cells of the invention are provided wherein the recombinant host cell is an E. coli cell, e.g. an E. coli K-12 strain, such as strain W3110.

In certain embodiments for the host cells and methods for preparing a bioconjugate of an E. coli glucosylated O4 antigen polysaccharide covalently linked to a carrier protein according to the invention, the rfb gene cluster for the E. coli O4 antigen polysaccharide comprises a sequence that encodes the enzymes that create the E. coli O4 antigen polysaccharide (Formula (O4-Glc−) in Table 1) and is at least 80%, e.g. at least 90%, e.g. at least 95%, e.g. at least 98% identical to SEQ ID NO: 9. In certain embodiments the rfb gene cluster comprises SEQ ID NO: 9.

In certain embodiments for the host cells and methods for preparing a bioconjugate of an E. coli glucosylated O4 antigen polysaccharide covalently linked to a carrier protein according to the invention, the glucosyl transferase that is capable of modifying the E. coli O4 antigen polysaccharide to produce the E. coli glucosylated O4 antigen polysaccharide has an amino acid sequence that has at least 90%, preferably at least 95%, preferably at least 98% sequence identity to SEQ ID NO: 4. In certain embodiments, the glucosyl transferase comprises SEQ ID NO: 4.

In certain embodiments for the host cells and methods for preparing a bioconjugate of an E. coli glucosylated O4 antigen polysaccharide covalently linked to a carrier protein according to the invention, the translocase is capable of translocating bactoprenol-linked glucose and has at least 90%, preferably at least 95%, preferably at least 98% sequence identity to SEQ ID NO: 7. In certain embodiments, the translocase comprises SEQ ID NO: 7.

In certain embodiments for the host cells and methods for preparing a bioconjugate of an E. coli glucosylated O4 antigen polysaccharide covalently linked to a carrier protein according to the invention, the glycosyltransferase is capable of glucosylating bactoprenol and has at least 90%, preferably at least 95%, preferably at least 98% sequence identity to SEQ ID NO: 8. In certain embodiments, the glycosyltransferase comprises SEQ ID NO: 8.

### BRIEF DESCRIPTION OF THE FIGURES

The foregoing summary, as well as the following detailed description of the invention, will be better understood when read in conjunction with the appended drawings. It should be understood that the invention is not limited to the precise embodiments shown in the drawings.

In the drawings:

FIG. 1 shows ELISA IgG titers against unmodified (GLC−) or glucose-modified (GLC+) O4 LPS in sera from two rabbits immunized with Glc-modified O4 polysaccharide bioconjugate as described in Example 4; ELISA titers were determined in quadruplicate;

FIG. 2 shows IgG titers in whole cell ELISAs with pooled sera from rabbits immunized with a Glc-modified O4 bioconjugate against E. coli O4 isolates with characterized gtrS status as described in Example 4; the following isolates were gtrS-negative: A2625, stGVXN4988, OC24784, OC24787 and OC24788; the following isolates were gtrS-positive: Y1382, E551, OC24334, stGVXN4983, stGVXN4994 and OC24794; the negative control strain OC9487 (ATCC 35383; serotype O75) was also included;

FIG. 3 shows Western blots of LPS extracted from gtrS-positive and -negative O4 isolates probed with pooled sera from rabbits immunized with modified O4 polysaccharide;

FIGS. 4A and 4B show antibody responses induced by glucosylated O4 (O4-Glc+)-EPA bioconjugates; FIG. 4A shows serum antibody levels measured by ELISA at day 0, 14 and 42 post-immunization; individual titers (log 10 EC50 titer) and GMT±95% CI are shown; the grey dotted line indicates the threshold above which the dilution curves of the samples have a 4PL fitting; FIG. 4B shows the results of the opsonophagocytic (OPK) assay to determine the functionality of the antibodies in serum samples obtained at day 42 post-immunization with glucosylated O4 (O4-Glc+)-EPA bioconjugate (4.0 μg); Wilcoxon rank sum test and Bonferroni correction; *P≤0.05, ***P≤0.0001;

FIG. 5 shows the boost effect of glucosylated O4 (O4 Glc+)-EPA bioconjugate in Sprague Dawley rats immunized at 3 different doses as described in Example 4; serum antibody levels were measured by ELISA at day 0, 14 and 42 post-immunization; individual titers (log 10 EC50 titer) are shown for each animal; the lines between the data points connect IgG titers for each animal in time; the grey dotted line indicates the threshold above which the dilution curves of the samples have a 4PL fitting; statistical analysis was performed with Wilcoxon signed-rank test and Bonferroni correction for multiple comparisons (day 14 vs day 0, P=0.012 for 4.0 μg/dose; day 42 vs day 0, P=0.006 for all doses; day 42 vs day 14, P=0.006 for all doses);

FIG. 6 shows the functionality of antibodies induced by O4-Glc+-EPA bioconjugate; Sprague Dawley rats were immunized intramuscularly 3 times with formulation buffer or O4(Glc+)-EPA bioconjugate at 4.00 μg/dose; functionality of the antibodies was determined by opsonophagocytic killing assay (OPKA) using O4(Glc+) and O4(Glc−) E. coli strains; individual opsonic titers (O1) and GMT±95% CI are shown;

FIG. 7 shows capillary electrophoresis readout of PglB screen visualizing O4-Glc+ bioconjugate production for each tested strain in a blot-like image, using monoclonal antibodies to detect O4-Glc+ bioconjugate in the periplasmic fraction. Mono-glycosylated product approximately 180 kDa, di-glycosylated product approximately 320 kDa and tri-glycosylated product approximately 450 kDa. A) First screening round. Wt PglB in lane 3, N311V-PglB in lanes 2 and 4, empty control strain in lane 1 and other PglB variants in lanes 5 and 6. B) Second screening round. N311V PglB in lane 3, N311V+Y77H PglB in lane 9, empty control strain in lanes 1 and 2, other PglB variants in remaining lanes.

FIG. 8 shows antibody responses induced by ExPEC10V vaccine in New Zealand White rabbits. Animals received 3 intramuscular immunizations with ExPEC10V or saline administered 2 weeks apart. ExPEC10V vaccine was administered at 3 different concentrations (group 1: high dose, group 2: medium dose and group 3: low dose, Table 11) and a control group received only saline (group 4, 0.9% (w/v) sodium chloride solution). Antibody levels were measured by ELISA at day 0 (pre-vaccination) and days 14, 27 and 42 (post-vaccination). Individual titers (EC50 titer) and geometric mean titers (GMT)±95% CI are shown. Wilcoxon Rank Sum test with Bonferroni correction for multiple comparisons. Comparisons ExPEC10V vaccinated animals (group 1, 2 and 3) versus saline control (group 4). *p≤0.05, **p≤0.01; ***p≤0.001; ****p≤0.0001. LOD: limit of detection.

FIG. 9 shows antibody responses induced by ExPEC10V. New Zealand White rabbits received 3 intramuscular immunizations with ExPEC10V (105.6 g total polysaccharide) or 0.9% w/v sodium chloride solution (control). IgG titers were determined by ELISA at day 1 (pre-immunization, n=20/group), day 31 (post-immunization, n=20/group) and day 50 (post-immunization, n=10/group). Plots show individual titers and geometric mean±95% confidence interval for each group. Differences in IgG titers between the ExPEC10V and control group were analyzed using a Tobit model with a likelihood ratio test. P-values≤0.05 were considered significant. *P≤0.05, ****P≤0.0001.

FIG. 10 shows the overall study design for a phase 1/2a clinical trial with ExPEC10V vaccine in humans. FIG. 10A shows the overall study design for Cohort 1, and FIG. 10B shows the overall study design for Cohort 2. See Example 11 for details.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series.

Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The identification of an O-antigen structural modification, namely glucose branching, within the E. coli O4 serotype (Jann et al., 1993) presents a challenge to the discovery and development of a glycoconjugate vaccine targeting bacterial isolates within this serotype. The proportion of clinical contemporary O4 isolates expressing the unmodified (not having a glucose side-branch) and modified (having a glucose side-branch) forms of the O4 O-antigen is unknown. Obtaining information on this characteristic is critical for selecting the relevant antigenic structure. In addition, the extent to which vaccine induced antibodies elicited to one form of the O4 polysaccharide will cross-react with the other form has not been determined. Purification of O-antigen free from lipid A and subsequent chemical conjugation to a carrier protein is a lengthy and laborious process. Additionally, the purification, lipid A detoxification and chemical conjugation processes can result in loss of epitopes, antigen heterogeneity and reduced immunogenicity of the conjugated polysaccharide. Synthesis of glycoconjugates by bioconjugation can overcome these limitations of classical purification and chemical conjugation, but the in vivo synthesis of glucose-branched O4 O-antigen requires the activity of a polysaccharide branching enzyme, which lies outside of the rfb gene cluster. To date, the O-antigen modifying enzyme responsible for glucose-branching in O4 E. coli strains has not been identified. Cloning the O4 rfb gene cluster into the bioconjugation E. coli strain expressing PglB will not be sufficient to synthesize the glucose-branched O4 glycoconjugate, but rather would only produce non-glucose-branched O4 bioconjugates (the structure of the glycan thereof is shown in Formula (O4) in Table 1). As used herein, the terms “glucosylated O4”, “glucose-branched O4”, “O4 Glc+” and “Glc+O4” O-antigen refer to O4 O-antigen with a glucose side-branch, and the structure thereof is shown in formula (O4-Glc+) in Table 1.

Disclosed herein is the gene encoding the O-antigen modifying enzyme responsible for glucose branching of the E. coli O4 antigen polysaccharide. Also disclosed herein are host cells, e.g., recombinantly engineered host cells comprising nucleic acid encoding enzymes capable of producing bioconjugates comprising the glucosylated O4 antigen polysaccharide covalently bound to a carrier protein in vivo. Such host cells can be used to generate bioconjugates comprising the glucosylated O4 antigen linked to a carrier protein, which can be used in, e.g., the formulation of therapeutic and/or prophylactic compositions (e.g., vaccines). Further provided herein are compositions comprising bioconjugates of the glucosylated O4 antigen polysaccharide, alone or in combination with other E. coli antigens (e.g., O1, O2, O6, O8, O15, O16, O18, O25, and/or O75 antigen polysaccharides and subserotypes thereof). The compositions can be used in prophylactic and/or therapeutic methods, e.g., vaccination of hosts against infection with E. coli, and are useful in the generation of antibodies, which can be used, e.g., in therapeutic methods such as for immunization of subjects.

As used here, the terms “O-antigen,” “O-antigen polysaccharide,” “O-antigen saccharide,” and “OPS” refer to the O-antigen of Gram-negative bacteria. Typically, an O-antigen is a polymer of immunogenic repeating polysaccharide units. In a particular embodiment, the terms “O-antigen,” “O-antigen polysaccharide,” and “OPS” refer to the O-antigen of Escherichia coli. Different serotypes of E. coli express different O-antigens. In E. coli, the gene products involved in O-antigen biogenesis are encoded by the rfb gene cluster.

As used herein, “rfb cluster” and “rfb gene cluster” refer to a gene cluster that encodes enzymatic machinery capable of synthesizing an O-antigen backbone structure. The term rfb cluster can apply to any O-antigen biosynthetic cluster, and preferably refers to a gene cluster from the genus Escherichia, particularly E. coli.

As used herein, the term “O1A” refers to the O1A antigen of E. coli (a subserotype of E. coli serotype O1). The term “O2” refers to the O2 antigen of E. coli (E. coli serotype O2). The term “O6A” refers to the O6A antigen of E. coli (a subserotype of E. coli serotype O6). The term “O8” refers to the O8 antigen of E. coli (E. coli serotype O8). The term “O15” refers to the O15 antigen of E. coli (E. coli serotype O15). The term “O16” refers to the O16 antigen of E. coli (E. coli serotype O16). The term “O18A” refers to the O18A antigen of E. coli (a subserotype of E. coli serotype O18). The term “O25B” refers to the O25B antigen from E. coli (a subserotype of E. coli serotype O25). The term “O75” refers to the O75 antigen of E. coli (E. coli serotype O75).

The structures of E. coli O-antigen polysaccharides referred to throughout this application are shown below in Table 1. A single repeating unit for each E. coli O-antigen polysaccharide is shown.

TABLE 1
Structures of E. coli O-antigen Polysaccharides

| E. coli O- | |
|---|---|
| antigen | |
| Polysaccharide | Structure of Repeating Unit¹ |
| Non- | [→2)-α-L-Rhap-(1→6)-α-D-Glcp-(1→3)-α-L-FucpNAc-(1→3)-β-D-GlcpNAc-(1→]ₙ |
| glucosylated | |
| O4 antigen | |
| polysaccharide | |
| (O4-Glc-) | |
| Glucosylated O4 antigen polysaccharide (O4-Glc+) | 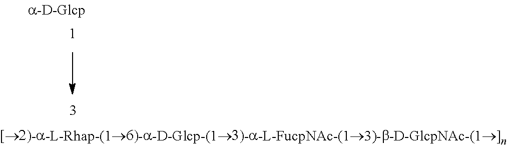 |
| O1A antigen polysaccharide (O1A) | 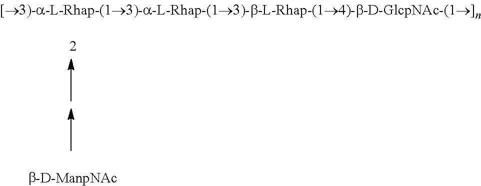 |
| O2 antigen polysaccharide (O2) | 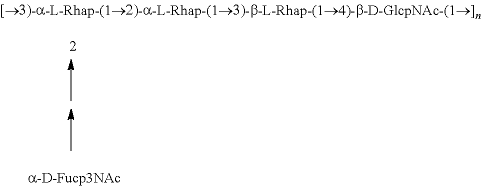 |
| O6A antigen polysaccharide (O6) | 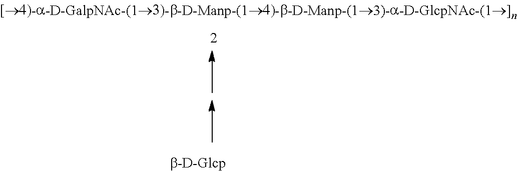 |
| O8 antigen | α-D-Manp3Me-(1→[3)-β-D-Manp-(1→2)-α-D-Manp-(1→2)-α-D-Manp-(1→]n |
| polysaccharide | |
| (O8) | |
| O15 antigen | [→2)-β-D-Galp-(1→3)-α-L-FucpNAc-(1→3)-β-D-GlcpNAc-(1→]ₙ |
| polysaccharide | |
| (O15) | |
| O16 antigen polysaccharide (O16) | 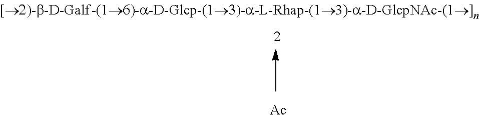 |
| O18A antigen polysaccharide (O18A) | 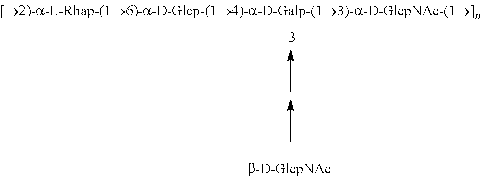 |
| O25B antigen polysaccharide (O25B) | 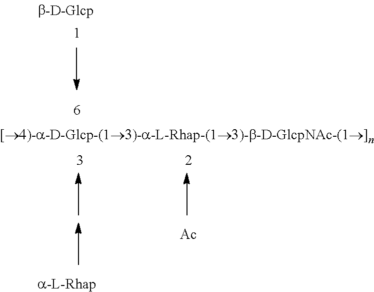 |
| O75 antigen polysaccharide (O75) | 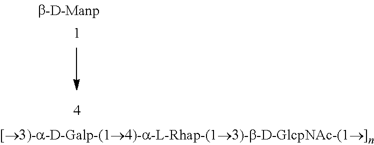 |

¹Each n is independently an integer of 1 to 100, such as 1-50, 1-40, 1-30, 1-20, and 1-10, 3-50, 3-40, e.g. at least 5, such as 5-40, e.g. 7-30, e.g.7 to 25, e.g. 10 to 20, but in some instances can be 1-2.

All monosaccharides described herein have their common meaning known in the art. Monosaccharides can have the D or L configuration. If D or L is not specified, the sugar is understood to have the D configuration. Monosaccharides are typically referred to by abbreviations commonly known and used in the art. For example, Glc refers to glucose; D-Glc refers to D-glucose; and L-Glc refers to L-glucose. Other common abbreviations for monosaccharides include: Rha, rhamnose; GlcNAc, N-acetylglucosamine; GalNAc, N-acetylgalactosamine; Fuc, fucose; Man, mannose; Man3Me, 3-O-methyl-mannose; Gal, galactose; FucNAc, N-acetylfucosamine; and Rib, ribose. The suffix “f” refers to furanose and the suffix “p” refers to pyranose.

The terms “RU,” “repeat unit,” and “repeating unit” as used with respect to an O-antigen refer to the biological repeat unit (BRU) of an O-antigen as it is synthesized in vivo by cellular machinery (e.g., glycosyltransferases). The number of RUs of an O-antigen may vary per serotype, and in embodiments of the invention typically varies from about 1-100 RUs, preferably about 1 to 50 RUs, such as 1-50 RUs, 1-40 RUs, 1-30 RUs, 1-20 RUs, and 1-10 RUs, and more preferably at least 3 RUs, at least 4 RUs, at least 5 RUs, such as 3-50 RUs, preferably 5-40 RUs, e.g. 7-25 RUs, e.g. 10-20 RUs. However, in some instances, the number of RUs of an O-antigen can be 1-2. The structure of each O-antigen that is specifically described herein is shown containing one RU with the variable “n” designating the number of RUs. In each O-antigen polysaccharide in a bioconjugate of the invention, n is independently an integer of 1-100, such as 1-50, 1-40, 1-30, 1-20, 1-10, preferably at least 3, more preferably at least 5, such as 3-50, preferably 5-40 (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40), but in some instances can be 1-2. In some embodiments n is independently an integer of about 7-25, e.g. about 10-20. The values may vary between individual O-antigen polysaccharides in a composition, and are provided here as average values, i.e. if a bioconjugate is described herein as having an n that is independently an integer of 5-40, the composition contains a majority of O-antigen polysaccharides with 5-40 repeat units, but may also contain some O-antigen polysaccharides that have less than 5 repeat units or more than 40 repeat units.

The term “glycoconjugate” refers to a sugar or saccharide antigen (e.g., oligo- and polysaccharide)-protein conjugate linked to another chemical species, including but not limited to proteins, peptides, lipids, etc. Glycoconjugates can be prepared chemically, e.g., by chemical (synthetic) linkage of the protein and sugar or saccharide antigen. The term glycoconjugate also includes bioconjugates.

The term “bioconjugate” refers to a conjugate between a protein (e.g., a carrier protein) and a sugar or saccharide antigen (e.g., oligo- and polysaccharide) prepared in a host cell background, preferably a bacterial host cell, e.g. an E. coli host cell, wherein host cell machinery links the antigen to the protein (e.g., N-links). Preferably, the term “bioconjugate” refers to a conjugate between a protein (e.g., carrier protein) and an O-antigen, preferably an E. coli O-antigen (e.g., O1A, O2, glucosylated O4, O6A, O8, O15, O16, O18A, O25B, O75, etc.) prepared in a host cell background, wherein host cell machinery links the antigen to the protein (e.g., N-links). Because bioconjugates are prepared in host cells by host cell machinery, the antigen and protein are covalently linked via a glycosidic linkage or bond in a bioconjugate. Bioconjugates can be prepared in recombinant host cells engineered to express the cellular machinery needed to synthesize the O-antigen and/or link the O-antigen to the target protein. Bioconjugates, as described herein, have advantageous properties over chemically prepared glycoconjugates where the glycans are purified from bacterial cell walls and subsequently chemically coupled to a carrier protein, e.g., bioconjugates require fewer chemicals in manufacture and are more consistent in terms of the final product generated, and contain less or no free (i.e. unbound to carrier protein) glycan. Thus, in typical embodiments, bioconjugates are preferred over chemically produced glycoconjugates.

The term “about,” when used in conjunction with a number, refers to any number within ±1, ±5 or +10% of the referenced number.

The term “percent (%) sequence identity” or “% identity” describes the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 95%, 97% or 98% identity) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

For example, for amino acid sequences, sequence identity and/or similarity can be determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al, 1984, Nucl. Acid Res. 12:387-395, preferably using the default settings, or by inspection. In certain embodiments, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

Another example of a useful algorithm is the BLAST algorithm, described in: Altschul et al, 1990, J. Mol. Biol. 215:403-410; Altschul et al, 1997, Nucleic Acids Res. 25:3389-3402; and Karin et al, 1993, Proc. Natl. Acad. Sci. U.S.A. 90:5873-5787. A particularly useful BLAST program is the WU-BLAST-2 program which was obtained from Altschul et al, 1996, Methods in Enzymology 266:460-480. WU-BLAST-2 uses several search parameters, most of which are set to the default values.

An additional useful algorithm is gapped BLAST as reported by Altschul et al, 1993, Nucl. Acids Res. 25:3389-3402.

The term “Invasive Extraintestinal pathogenic Escherichia coli (ExPEC) disease (IED)” is defined herein as an acute illness consistent with systemic bacterial infection, which is microbiologically confirmed either by the isolation and identification of E. coli from blood or other normally sterile body sites, or by the isolation and identification of E. coli from urine in a patient with presence of signs and symptoms of invasive disease (systemic inflammatory response syndrome (SIRS), sepsis or septic shock) and no other identifiable source of infection.

Bioconjugates of E. coli Glucosylated O4 Antigen Polysaccharides

In one aspect, provided herein is a bioconjugate of an E. coli glucosylated O4 antigen polysaccharide covalently linked to a carrier protein. As used herein, the term “O4” refers to the O4 antigen from E. coli (E. coli serotype O4). O-antigen structural modification is known to exist within the E. coli O4 serotype. In particular, some O4 serotypes express a modified O-antigen having a branched glucose unit. As used herein, “glucosylated O4 antigen,” “glucosylated O4 antigen polysaccharide, “O4-Glc+ antigen polysaccharide,” and “O4-Glc+ antigen” refer to an O4 antigen (e.g., E. coli O4 antigen) having a glucose branch, in which D-glucose is linked to L-rhamnose in the repeating unit L-Rha→D-Glc→L-FucNAc→D-GlcNAc. In a particular embodiment, an E. coli glucosylated O4 antigen polysaccharide comprises the structure of formula (O4-Glc+), as shown in Table 1, wherein n is an integer of 1 to 100. In preferred embodiments, n is an integer of 3 to 50, e.g. 5 to 40, e.g. 7 to 25, e.g. 10 to 20.

E. coli O4 strains, independent of glucose branching status, carry a substantially identical rfb gene cluster encoding the genes responsible for production of the O4 antigen polysaccharide. However, in vivo synthesis of the modified O4 antigen having glucose branching requires the activity of a polysaccharide branching enzyme, which lies outside of the rfb gene cluster. To the best of the knowledge of the inventors, the identity of the polysaccharide branching enzyme responsible for glucose modification of the O4 antigen has remained unknown to date. Here, the inventors discovered the sequence of the polysaccharide branching enzyme responsible for glucose modification of the O4 antigen. Identification of this enzyme enables production of bioconjugates of the modified O4 antigen polysaccharide having glucose branching. The glucose modified form of the O4 antigen polysaccharide is present in predominant serotypes and can thus be used to provide an improved immune response, e.g for prophylactic or therapeutic use.

In particular, provided herein is the sequence of a gtrS gene encoding a glucosyltransferase enzyme specific for E. coli serotype O4 that glucosylates the O4 antigen. In general, the gtrA, gtrB, and gtrS genes encodes the enzymes responsible for O-antigen glucosylation. While the gtrA and gtrB genes in different serotypes are highly homologous and interchangeable, the gtrS gene encodes a serotype specific O-antigen glucosyl transferase. The gtrS gene of E. coli serotype O4 encodes the GtrS enzyme that modifies the O4 antigen by introducing glucose branching. Characterization of contemporary clinical E. coli isolates of the O4 serotype revealed the presence of gtrS in 78% of tested isolates, indicating that E. coli O4 antigen polysaccharide modified with the addition of a glucose residue is predominant in current infecting isolates.

In one embodiment, provided herein is a nucleic acid of a gtrS gene from E. coli serotype O4 encoding a GtrS glucosyltransferase comprising the amino acid sequence of SEQ ID NO: 4. In another embodiment, a gtrS nucleic acid encodes a GtrS protein from E. coli serotype O4 that is about 80%, 85%, 90%, 95%, 96%, 97%, 98%, 99%, or 100% identical to the amino acid sequence of SEQ ID NO: 4, preferably 98%, 99%, or 100% identical to the amino acid sequence of SEQ ID NO: 4. A GtrS protein that is at least 80% identical to the amino acid sequence of SEQ ID NO: 4 is capable of specifically glucosylating the E. coli O4 antigen polysaccharide to obtain a glucosylated O4 antigen having the structure of Formula (O4-Glc+) as shown in Table 1. One of ordinary skill in the art will be able to make mutated forms of the GtrS protein of SEQ ID NO: 4 having at least 80% sequence identity to SEQ ID NO: 4, and test such sequences for glucosylation activity of the E. coli O4 antigen in view of the present disclosure. Recombinant host cells comprising nucleic acid sequence encoding the glucosyl transferase gtrS gene of E. coli serotype O4, and use of the recombinant host cells in production of the glucose modified O4 antigen polysaccharides and bioconjugates thereof are described in greater detail below.

Sequences for gtrA and gtrB encoded proteins, which function as bactoprenol-linked glucose translocase (GtrA, flips the bactoprenol-linked glucose over the inner membrane to the periplasm) and bactoprenol glucosyl transferase (GtrB, links glucose to bactoprenol), respectively, may comprise amino acid sequences that are at least about 80% identical to SEQ ID NOs: 7 and 8, respectively. In certain embodiments, nucleic acid sequences encoding GtrA and GtrB proteins that are at least about 80%, 85%, 90%, 95%, 96%, 97%, 98%, 99%, or 100% identical to SEQ ID NOs: 7 and 8, respectively, and having bactoprenol-linked glucose translocase and bactoprenol glucosyl transferase activity, respectively, are also present in the host cells of the invention, that further comprise an O4-specific rfb locus, the O4-specific GtrS encoding sequence described above, an oligosaccharyl transferase as described herein, and a sequence encoding a carrier protein having one or more glycosylation consensus sequences as described herein, to produce bioconjugates of E. coli glucosylated O4 serotype (comprising glycan structure of Formula (O4-Glc+) in Table 1).

Bioconjugates of an E. coli glucosylated O4 antigen polysaccharide provided herein are covalently linked to a carrier protein, preferably by a glycosidic linkage. Any carrier protein known to those skilled in the art in view of the present disclosure can be used. Suitable carrier proteins include, but are not limited to, detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae. Bioconjugation with various different carrier proteins containing the required consensus glycosylation sequence has been described, showing that a wide range of proteins can be glycosylated using this technology (see, e.g. WO 06/119987, WO 2015/124769, WO 2015/158403, WO 2015/82571, WO 2017/216286, and WO 2017/67964, together showing a wide variety of carrier proteins that were successfully used in bioconjugation).

In certain embodiments a carrier protein is modified, e.g., modified in such a way that the protein is less toxic and/or more susceptible to glycosylation. In a specific embodiment, the carrier proteins used herein are modified such that the number of glycosylation sites in the carrier proteins is maximized in a manner that allows for lower concentrations of the protein to be administered, e.g., in an immunogenic composition, particularly in its bioconjugate form.

Thus, in certain embodiments, the carrier proteins described herein are modified to include 1, 2, 3, 4, 5, 6, 7, 8, 9, 10 or more glycosylation sites than would normally be associated with the carrier protein (e.g., relative to the number of glycosylation sites associated with the carrier protein in its native/natural, i.e., “wild-type” state). Introduction of glycosylation sites into a carrier protein can be accomplished by insertion of a glycosylation consensus sequence anywhere in the primary structure of the protein by, e.g., adding new amino acids to the primary structure of the protein such that a glycosylation site is added in full or in part, or by mutating existing amino acids in the protein in order to generate a glycosylation site. One of ordinary skill in the art will recognize that the amino acid sequence of a protein can be readily modified using approaches known in the art, e.g., recombinant approaches that include modification of the nucleic acid sequence encoding the protein. In specific embodiments, glycosylation consensus sequences are introduced into specific regions of the carrier protein, e.g., surface structures of the protein, at the N or C termini of the protein, and/or in loops that are stabilized by disulfide bridges at the base of the protein. In some embodiments, a glycosylation consensus sequence can be extended by addition of lysine residues for more efficient glycosylation.

Exemplary examples of glycosylation consensus sequences that can be inserted into or generated in a carrier protein include Asn-X-Ser(Thr), wherein X can be any amino acid except Pro (SEQ ID NO: 1); and Asp(Glu)-X-Asn-Z-Ser(Thr), wherein X and Z are independently selected from any amino acid except Pro (SEQ ID NO: 2).

In some embodiments, the E. coli glucosylated O4 antigen polysaccharide is covalently linked to an asparagine (Asn) residue in the carrier protein (e.g., N-linked), wherein the Asn residue is present in a glycosylation site comprising a glycosylation consensus sequence having SEQ ID NO: 1, more preferably having SEQ ID NO: 2. Typically, a carrier protein comprises 1-10 glycosylation sites, preferably 2 to 4 glycosylation sites, most preferably 4 glycosylation sites, such as 1-10, preferably 2-4, and more preferably 4 glycosylation sites each comprising a glycosylation consensus sequences having the amino acid sequence of SEQ ID NO: 1, and more preferably the amino acid sequence of SEQ ID NO: 2.

In particular embodiments, a carrier protein is a detoxified Exotoxin A of P. aeruginosa. For EPA, various detoxified protein variants have been described in literature and could be used as carrier proteins. For example, detoxification can be achieved by mutating and deleting the catalytically essential residues L552V and ΔE553 according to Lukac et al., 1988, Infect Immun, 56: 3095-3098, and Ho et al., 2006, Hum Vaccin, Hum Vaccin, 2:89-98. As used herein, “EPA” refers to a detoxified Exotoxin A of P. aeruginosa. In those embodiments, wherein the carrier protein is EPA, an E. coli glucosylated O4 antigen polysaccharide can be covalently linked to an Asn residue in a glycosylation site comprising a glycosylation consensus sequence having SEQ ID NO: 1, and preferably covalently linked to an Asn residue in a glycosylation site comprising a glycosylation consensus sequence having SEQ ID NO: 2. Preferably, the EPA carrier protein comprises 1-10 glycosylation sites, preferably 2 to 4 glycosylation sites, most preferably 4 glycosylation sites, such as 1-10, preferably 2-4, and more preferably 4 glycosylation sites each comprising a glycosylation consensus sequence having the amino acid sequence of SEQ ID NO: 1, and more preferably the amino acid sequence of SEQ ID NO: 2.

In some embodiments, the EPA carrier protein comprises four glycosylation sites each comprising a glycosylation consensus sequence, for instance a glycosylation site comprising a glycosylation consensus sequence having SEQ ID NO: 2. As used herein, “EPA-4 carrier protein” and “EPA-4” refer to a detoxified Exotoxin A of P. aeruginosa carrier protein comprising four glycosylation sites each comprising a glycosylation consensus sequences having SEQ ID NO: 2. An exemplary preferred example of an EPA-4 carrier protein is EPA carrier protein comprising the amino acid sequence of SEQ ID NO: 3.

### Compositions

In another aspect, provided herein is a composition comprising a bioconjugate of an E. coli glucosylated O4 antigen polysaccharide covalently linked to a carrier protein. The compositions provided herein can include any bioconjugate of an E. coli glucosylated O4 antigen polysaccharide covalently linked to a carrier protein (e.g., EPA) described herein.

In some embodiments, a composition is an immunogenic composition. As used herein, an “immunogenic composition” refers to a composition that can elicit an immune response in a host or subject to whom the composition is administered. Immunogenic compositions can further comprise a pharmaceutically acceptable carrier. In some embodiments, a composition is a pharmaceutical composition further comprising a pharmaceutically acceptable carrier. As used herein, a “pharmaceutically acceptable carrier” refers to a diluent, adjuvant, excipient or vehicle with which a composition is administered, and that is non-toxic and should not interfere with the efficacy of the active ingredient. For example, saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable pharmaceutically acceptable carriers are described in “Remington's Pharmaceutical Sciences” by E. W. Martin.

In one embodiment, a composition of the invention comprises the bioconjugates of the invention in a Tris-buffered saline (TBS) pH 7.4 (e.g. containing Tris, NaCl and KCl, e.g. at 25 mM, 137 mM and 2.7 mM, respectively). In other embodiments, the compositions of the invention comprise bioconjugates of the invention in about 10 mM KH2PO4/Na2HPO4 buffer at pH of about 7.0, about 5% (w/v) sorbitol, about 10 mM methionine, and about 0.02% (w/v) polysorbate 80. In other embodiments, the compositions of the invention comprise bioconjugates of the invention in about 10 mM KH2PO4/Na2HPO4 buffer at pH of about 7.0, about 8% (w/v) sucrose, about 1 mM EDTA, and about 0.02% (w/v) polysorbate 80 (see e.g. WO 2018/077853 for suitable buffers for bioconjugates of E. coli O-antigens covalently bound to EPA carrier protein).

In some embodiments, the compositions described herein are monovalent formulations, and contain one E. coli O-antigen polysaccharide, e.g., in isolated form or as part of a glycoconjugate or bioconjugate, such as the E. coli glucosylated O4 antigen polysaccharide. Also provided herein are compositions (e.g., pharmaceutical and/or immunogenic compositions) that are multivalent compositions, e.g., bivalent, trivalent, tetravalent, etc. compositions. For example, a multivalent composition comprises more than one antigen, such as an E. coli O-antigen, glycoconjugate, or bioconjugate thereof. In particular embodiments, multivalent compositions provided herein comprise a bioconjugate of an E. coli glucosylated O4 antigen polysaccharide, and at least one additional antigen.

In one embodiment, a composition (e.g., pharmaceutical and/or immunogenic composition) is a monovalent composition comprising a bioconjugate of an E. coli glucosylated O4 antigen polysaccharide covalently linked to a carrier protein as described herein.

In another embodiment, a composition (e.g., pharmaceutical and/or immunogenic composition) is a multivalent composition comprising an E. coli glucosylated O4 antigen polysaccharide covalently linked to a carrier protein as described herein, and at least one additional antigen.

In some embodiments, the additional antigen is antigen saccharide or polysaccharide, more preferably an E. coli O-antigen polysaccharide, such as E. coli O-antigens of one or more of the O1, O2, O6, O8, O15, O16, O18, O25, and O75 serotypes and subserotypes thereof. In some embodiments, each of the additional E. coli O-antigen polysaccharides is a glycoconjugate, meaning that the E. coli O-antigen polysaccharide is covalently linked to another chemical species, e.g., protein, peptide, lipid, etc., most preferably a carrier protein, such as by chemical or enzymatic methods. In preferred embodiments, each of the additional E. coli O-antigen polysaccharides is a bioconjugate in which the O-antigen polysaccharide is covalently linked to, e.g. a carrier protein, via a glycosidic bond enzymatically by host cell machinery. Compositions provided herein in certain embodiments can comprise 1-20 additional glycoconjugates, more preferably bioconjugates of E. coli O-antigen polysaccharides, such as 1, 2, 3, 4, 5, 6, 7, 8, 9, 10, 11, 12, 13, 14, 15, 16, 17, 18, 19 or 20 additional glycoconjugates or preferably bioconjugates of E. coli O-antigen polysaccharides. Other antigens can be included in the compositions provided herein, such as peptide, protein, or lipid antigens, etc.

In some embodiments, a composition (e.g., pharmaceutical and/or immunogenic composition) comprises a bioconjugate of an E. coli glucosylated O4 antigen polysaccharide, and at least one additional antigen polysaccharide selected from the group consisting of E. coli O1A antigen polysaccharide, E. coli O2 antigen polysaccharide, E. coli O6A antigen polysaccharide, E. coli O8 antigen polysaccharide, E. coli O15 antigen polysaccharide, E. coli O16 antigen polysaccharide, E. coli O18A antigen polysaccharide, E. coli O25B antigen polysaccharide, and E. coli O75 antigen polysaccharide. Preferably, each of the additional O-antigen polysaccharides is covalently linked to a carrier protein, and is more preferably a bioconjugate.

In one embodiment, an O1A antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O1A antigen polysaccharide comprises the structure of formula (O1A) as shown in Table 1, wherein n is an integer of 1-100, preferably 3-50, e.g. 5-40, e.g. 7 to 25, e.g. 10 to 20. Preferably, the O1A antigen polysaccharide is part of a bioconjugate and is covalently linked to a carrier protein, e.g., EPA.

In one embodiment, an O2 antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O2 antigen polysaccharide comprises the structure of formula (O2) as shown in Table 1, wherein n is an integer of 1-100, preferably 3-50, e.g. 5-40, e.g. 7 to 25, e.g. 10 to 20. Preferably, the O2 antigen polysaccharide is part of a bioconjugate and is covalently linked to a carrier protein, e.g., EPA.

In one embodiment, an O6A antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O6A antigen polysaccharide comprises the structure of formula (O6A) as shown in Table 1, wherein n is an integer of 1-100, preferably 3-50, e.g. 5-40, e.g. 7 to 25, e.g. 10 to 20. Preferably, the O6A antigen polysaccharide is part of a bioconjugate and is covalently linked to a carrier protein, e.g., EPA.

In one embodiment, an O8 antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O8 antigen polysaccharide comprises the structure of formula (O8) as shown in Table 1, wherein n is an integer of 1-100, preferably 3-50, e.g. 5-40, e.g. 7 to 25, e.g. 10 to 20. Preferably, the O8 antigen polysaccharide is part of a bioconjugate and is covalently linked to a carrier protein, e.g., EPA.

In one embodiment, an O15 antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O15 antigen polysaccharide comprises the structure of formula (O15) as shown in Table 1, wherein n is an integer of 1-100, preferably 3-50, e.g. 5-40, e.g. 7 to 25, e.g. 10 to 20. Preferably, the O15 antigen polysaccharide is part of a bioconjugate and is covalently linked to a carrier protein, e.g., EPA.

In one embodiment, an O16 antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O16 antigen polysaccharide comprises the structure of formula (O16) as shown in Table 1, wherein n is an integer of 1-100, preferably 3-50, e.g. 5-40, e.g. 7 to 25, e.g. 10 to 20. Preferably, the O16 antigen polysaccharide is part of a bioconjugate and is covalently linked to a carrier protein, e.g., EPA.

In one embodiment, an O18A antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O18A antigen polysaccharide comprises the structure of formula (O18A) as shown in Table 1, wherein n is an integer of 1-100, preferably 3-50, e.g. 5-40, e.g. 7 to 25, e.g. 10 to 20. Preferably, the O18A antigen polysaccharide is part of a bioconjugate and is covalently linked to a carrier protein, e.g., EPA.

In one embodiment, an O25B antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O25B antigen polysaccharide comprises the structure of formula (O25B) as shown in Table 1, wherein n is an integer of 1-100, preferably 3-50, e.g. 5-40, e.g. 7 to 25, e.g. 10 to 20. Preferably, the O25B antigen polysaccharide is part of a bioconjugate and is covalently linked to a carrier protein, e.g., EPA.

In one embodiment, an O75 antigen polysaccharide (e.g., in isolated form or as part of a glycoconjugate or bioconjugate) is used in a composition provided herein (e.g., in combination with a glucosylated O4 antigen polysaccharide or bioconjugate thereof). In a specific embodiment, the O75 antigen polysaccharide comprises the structure of formula (O75) as shown in Table 1, wherein n is an integer of 1-100, preferably 3-50, e.g. 5-40, e.g. 7 to 25, e.g. 10 to 20. Preferably, the O75 antigen polysaccharide is part of a bioconjugate and is covalently linked to a carrier protein, e.g., EPA.

In another embodiment, a composition (e.g., a pharmaceutical and/or immunogenic composition) comprises at least the E. coli O1A, O2, glucosylated O4, O6A and O25B antigen polysaccharides, preferably bioconjugates of the O1A, O2, glucosylated O4, O6A and O25B antigen polysaccharides covalently linked to a carrier protein, e.g., EPA (i.e., a pentavalent composition).

In a preferred embodiment, a composition (e.g., a pharmaceutical and/or immunogenic composition) comprises at least the E. coli O1A, O2, glucosylated O4, O6A, O8, O15, O16, O25B and O75 antigen polysaccharides, preferably bioconjugates of the O1A, O2, glucosylated O4, O6A, O8, O15, O16, O25B and O75 antigen polysaccharides covalently linked to a carrier protein, e.g., EPA (i.e., a 9-valent composition).

In another preferred embodiment, a composition (e.g., a pharmaceutical and/or immunogenic composition) comprises at least the E. coli O1A, O2, glucosylated O4, O6A, O8, O15, O16, O18A, O25B and O75 antigen polysaccharides, preferably bioconjugates of the O1A, O2, glucosylated O4, O6A, O8, O15, O16, O18A, O25B and O75 antigen polysaccharides covalently linked to a carrier protein, e.g., EPA (i.e., a 10-valent composition).

Also contemplated herein are compositions which optionally further comprise additional O-antigens (e.g., in isolated form, or as part of a glycoconjugate or bioconjugate) from other E. coli serotypes.

In some embodiments, each of the additional E. coli O1A, O2, O6A, O8, O15, O16, O18A, O25B, and/or O75 antigen polysaccharides is covalently linked to a carrier protein. The O-antigen polysaccharide can be linked to a carrier protein by chemical or other synthetic methods, or the O-antigen polysaccharide can be part of a bioconjugate, and is preferably part of a bioconjugate. Any carrier protein known to those skilled in the art in view of the present disclosure can be used. Suitable carrier proteins include, but are not limited to, detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae. Preferably, the carrier protein is EPA.

In some embodiments, each of the additional E. coli O1A, O2, O6A, O8, O15, O16, O18A, O25B, and/or O75 antigen polysaccharides, particularly when part of a bioconjugate, is covalently linked to an asparagine (Asn) residue in the carrier protein, wherein the Asn residue is present in a glycosylation site comprising a glycosylation consensus sequence Asn-X-Ser(Thr), wherein X can be any amino acid except Pro (SEQ ID NO: 1), preferably wherein the Asn residue is present in a glycosylation site comprising a glycosylation consensus sequence Asp(Glu)-X-Asn-Z-Ser(Thr), wherein X and Z are independently selected from any amino acid except Pro (SEQ ID NO: 2). The carrier protein can comprise 1-10 glycosylation sites, preferably 2 to 4 glycosylation sites, most preferably 4 glycosylation sites, each comprising a glycosylation consensus sequence. In a particular embodiment, the carrier protein is EPA-4 carrier protein, for instance EPA-4 carrier protein comprising the amino acid sequence of SEQ ID NO: 3.

In a particular embodiment, provided herein is a composition (e.g., pharmaceutical and/or immunogenic composition) comprising: (i) a bioconjugate of an E. coli glucosylated O4 antigen polysaccharide covalently linked to a detoxified Exotoxin A of P. aeruginosa carrier protein comprising SEQ ID NO: 3 (EPA-4 carrier protein), wherein the E. coli glucosylated O4 antigen polysaccharide comprises the structure of Formula (O4-Glc+); (ii) a bioconjugate of an E. coli O1A antigen polysaccharide covalently linked to an EPA-4 carrier protein, wherein the E. coli O1A antigen polysaccharide comprises the structure of Formula (O1A); (iii) a bioconjugate of an E. coli O2 antigen polysaccharide covalently linked to an EPA-4 carrier protein, wherein the E. coli O2 antigen polysaccharide comprises the structure of Formula (O2); (iv) a bioconjugate of an E. coli O6A antigen polysaccharide covalently linked to an EPA-4 carrier protein, wherein the E. coli O6A antigen polysaccharide comprises the structure of Formula (O6A); (v) a bioconjugate of an E. coli O8 antigen polysaccharide covalently linked to an EPA-4 carrier protein, wherein the E. coli O8 antigen polysaccharide comprises the structure of Formula (O8); (vi) a bioconjugate of an E. coli O15 antigen polysaccharide covalently linked to an EPA-4 carrier protein, wherein the E. coli O15 antigen polysaccharide comprises the structure of Formula (O15); (vii) a bioconjugate of an E. coli O16 antigen polysaccharide covalently linked to an EPA-4 carrier protein, wherein the E. coli O16 antigen polysaccharide comprises the structure of Formula (O16); (viii) a bioconjugate of an E. coli O25B antigen polysaccharide covalently linked to an EPA-4 carrier protein, wherein the E. coli O25B antigen polysaccharide comprises the structure of Formula (O25B); and (ix) a bioconjugate of an E. coli O75 antigen polysaccharide covalently linked to an EPA-4 carrier protein, wherein the E. coli O75 antigen polysaccharide comprises the structure of Formula (O75), wherein each of the Formulas is provided in Table 1, and for each of the Formulas independently n is an integer of 1 to 100, e.g. 1 to 50, preferably 3 to 50, e.g. 5 to 40.

In a particular embodiment, said composition (e.g. pharmaceutical and/or immunogenic composition) further comprises: (x) a bioconjugate of an E. coli O18A antigen polysaccharide covalently linked to an EPA-4 carrier protein, wherein the E. coli O18A antigen polysaccharide comprises the structure of Formula (O18A) as shown in Table 1, wherein n for this structure is an integer of 1 to 100, e.g. 1 to 50, preferably 3 to 50, e.g. 5 to 40.

In some embodiments, a composition provided herein comprises a bioconjugate of an E. coli glucosylated O4 antigen polysaccharide, and at least a bioconjugate of an E. coli O25B antigen polysaccharide, wherein the bioconjugate of the E. coli O25B antigen polysaccharide is present in the composition at a concentration that is about 1.5 to 6 times, e.g. about 2 to 4 times higher, such as 1.5, 2, 3, 4, 5 or 6 times higher than the concentration of any of the other bioconjugates present in the composition.

In particular embodiments, a composition comprises bioconjugates of E. coli O1A, O2, glucosylated O4, O6A, O8, O15, O16, O25B, and O75 antigen polysaccharides, wherein the bioconjugates of O1A:O2:glucosylated O4:O6A:O8:O15:O16:O25B:O75 are present in a ratio (by weight of O-antigen polysaccharide) of 1:1:1:1:1:1:1:2:1, or 2:1:1:2:1:1:1:4:1.

In particular embodiments, a composition comprises bioconjugates of E. coli O1A, O2, glucosylated O4, O6A, O8, O15, O16, O18A, O25B, and O75 antigen polysaccharides, wherein the bioconjugates of O1A:O2:glucosylated O4:O6A:O8:O15:O16:O18A:O25B:O75 are present in a ratio (by weight of O-antigen polysaccharide) of 1:1:1:1:1:1:1:1:2:1, or 2:1:1:2:1:1:1:1:4:1.

In some embodiments, a composition provided herein comprises a bioconjugate of an E. coli glucosylated O4 antigen polysaccharide, and at least a bioconjugate of an E. coli O25B antigen polysaccharide, wherein the bioconjugate of the E. coli O25B antigen polysaccharide is present in the composition at a concentration of 2 to 50 μg/mL, preferably 8 to 40 μg/mL, more preferably 16-32 μg/mL, such as 16, 18, 20, 22, 24, 26, 28, 30, or 32 μg/mL. In such embodiments, the concentration of the bioconjugate of the E. coli O25B antigen polysaccharide is preferably about 1.5 to 6 times, e.g., about 2 to 4 times higher, such as 1.5, 2, 3, 4, 5, or 6 times higher than the concentration of any of the other bioconjugates present in the composition.

In certain embodiments, the compositions described herein (e.g., pharmaceutical and/or immunogenic compositions) comprise, or are administered in combination with, an adjuvant. The adjuvant for administration in combination with a composition described herein may be administered before (e.g. within 72 hours, 48 hours, 24 hours, 12 hours, 6 hours, 2 hours, 1 hour, 10 minutes), concomitantly with, or after (e.g. within 72 hours, 48 hours, 24 hours, 12 hours, 6 hours, 2 hours, 1 hour, 10 minutes) administration of said composition. As used herein, the term “adjuvant” refers to a compound that when administered in conjunction with or as part of a composition described herein augments, enhances and/or boosts the immune response to an E. coli O-antigen polysaccharide in a bioconjugate, but when the adjuvant compound is administered alone does not generate an immune response to the E. coli O-antigen polysaccharide in the bioconjugate. In some embodiments, the adjuvant enhances an immune response to an E. coli O-antigen polysaccharide in a bioconjugate thereof and does not produce an allergy or other adverse reaction. Adjuvants can enhance an immune response by several mechanisms including, e.g., lymphocyte recruitment, stimulation of B and/or T cells, and stimulation of macrophages.

Examples of suitable adjuvants include, but are not limited to, aluminum salts (alum) (such as aluminum hydroxide, aluminum phosphate, aluminum sulfate and aluminum oxide, including nanoparticles comprising alum or nanoalum formulations), calcium phosphate, monophosphoryl lipid A (MPL) or 3-de-O-acylated monophosphoryl lipid A (3D-MPL) (see e.g., United Kingdom Patent GB2220211, EP0971739, EP 1194166, U.S. Pat. No. 6,491,919), AS01, AS02, AS03 and AS04 (all GlaxoSmithKline; see e.g. EP1126876, U.S. Pat. No. 7,357,936 for AS04, EP0671948, EP0761231, U.S. Pat. No. 5,750,110 for AS02), MF59 (Novartis), imidazopyridine compounds (see WO2007/109812), imidazoquinoxaline compounds (see WO2007/109813), delta-inulin, STING-activating synthetic cyclic-di-nucleotides (e.g. US20150056224), combinations of lecithin and carbomer homopolymers (e.g. U.S. Pat. No. 6,676,958), and saponins, such as QuilA and QS21 (see e.g. Zhu D and W Tuo, 2016, Nat Prod Chem Res 3: el 13 (doi:10.4172/2329-6836.1000e113), Matrix M, Iscoms, Iscomatrix, etc, optionally in combination with QS7 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Further examples of adjuvants are liposomes containing immune stimulants such as MPL and QS21 such as in AS01E and AS01B (e.g. US 2011/0206758). Other examples of adjuvants are CpG (Bioworld Today, Nov. 15, 1998) and imidazoquinolines (such as imiquimod and R848). See, e.g., Reed G, et al., 2013, Nature Med, 19: 1597-1608. In certain embodiments, the adjuvant contains a toll-like receptor 4 (TLR4) agonist. TLR4 agonists are well known in the art, see e.g. Ireton G C and S G Reed, 2013, Expert Rev Vaccines 12: 793-807. In certain embodiments, the adjuvant comprises a TLR4 agonist comprising lipid A, or an analog or derivative thereof, such as MPL, 3D-MPL, RC529 (e.g. EP1385541), PET-lipid A, GLA (glycopyranosyl lipid adjuvant, a synthetic disaccharide glycolipid; e.g. US20100310602, U.S. Pat. No. 8,722,064), SLA (e.g. Carter D et al, 2016, Clin Transl Immunology 5: e108 (doi: 10.1038/cti.2016.63), which describes a structure-function approach to optimize TLR4 ligands for human vaccines), PHAD (phosphorylated hexaacyl disaccharide), 3D-PHAD (the structure of which is the same as that of GLA), 3D-(6-acyl)-PHAD (3D(6A)-PHAD) (PHAD, 3D-PHAD, and 3D(6A)PHAD are synthetic lipid A variants, see e.g. avantilipids.com/divisions/adjuvants, which also provide structures of these molecules), E6020 (CAS Number 287180-63-6), ONO4007, OM-174, and the like.

In certain embodiments, the compositions described herein do not comprise, and are not administered in combination with, an adjuvant.

In certain embodiments, the compositions described herein are formulated to be suitable for the intended route of administration to a subject. For example, the compositions (e.g., pharmaceutical and/or immunogenic) described herein can be formulated for subcutaneous, parenteral, oral, sublingual, buccal, intradermal, transdermal, colorectal, intraperitoneal, rectal administration, intravenous, intranasal, intratracheal, intramuscular, topical, transdermal, or intradermal administration. In a specific embodiment, a composition provided herein (e.g., pharmaceutical and/or immunogenic) is formulated for intramuscular injection.

### Methods of Use

Bioconjugates and compositions provided herein can be used to induce antibodies against an E. coli glucosylated O4 antigen in a subject, and to vaccinate a subject against E. coli in particular extra-intestinal pathogenic E. coli (ExPEC). As used herein, “subject” means any animal, preferably a mammal, to whom will be or has been administered a bioconjugate or composition provided herein. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc. In certain embodiments, a subject is a human. A human subject may be of any age. In certain embodiments, a subject is a human of about two months to about 18 years old, e.g. of 1 year to 18 years old. In certain embodiments, a subject is a human of at least 18 years old. In certain embodiments, a subject is a human of 15 to 50 years old, e.g. 18 to 45 years old, e.g. 20 to 40 years old. In certain embodiments, a subject is a human male. In certain embodiments, a subject is a human female. In certain embodiments, a subject is immunocompromised. In certain embodiments, a subject is a human of at least 50 years, at least 55 years, at least 60 years, at least 65 years old. In certain embodiments, a subject is a human that is not older than 100 years, not older than 95 years, not older than 90 years, not older than 85 years, not older than 80 years, or not older than 75 years. In certain embodiments, a subject is a human of at least 60 years old, and not older than 85 years old. In certain embodiments, a subject is a human in stable health. In certain embodiments, a subject is a human adult of at least 60 and not more than 85 years old in stable health. In certain embodiments, a subject is a human that has a history of a urinary tract infection (UTI, i.e. a bacterial infection in the urethra, bladder, ureters, and/or kidneys), i.e. having had at least one UTI episode in his or her life. In certain embodiments, a subject is a human that has a history of UTI in the past twenty, fifteen, twelve, ten, nine, eight, seven, six, five, four, three, two or one years. In certain embodiments, a subject is a human that has a history of UTI in the past two years. In certain embodiments, a subject is a human subject that has a history of recurrent UTI, i.e. having had at least two UTIs in six months or at least three UTIs in one year. In certain embodiments, a subject is a human subject that has a history of recurrent UTI in the past two years. In certain embodiments, a subject is a human of 60 years or older in stable health. In certain embodiments, a subject is a human of 60 years or older that has a history of UTI in the past two years. In certain embodiments, a subject is a human of at least 60 years and less than 75 years old that has a history of UTI in the past two years. In certain embodiments, a subject is a human subject of 75 years or older that has a history of UTI in the past two years. In certain embodiments, a subject is a patient scheduled for undergoing elective urogenital and/or abdominal procedures or surgeries, e.g. transrectal ultrasound-guided prostate needle biopsy (TRUS-PNB).

In one aspect, provided herein is a method of inducing antibodies against an E. coli glucosylated O4 antigen in a subject, comprising administering to the subject any of the bioconjugates of an E. coli glucosylated O4 antigen covalently linked to a carrier protein described herein, or a composition comprising a bioconjugate of an E. coli glucosylated O4 antigen covalently linked to a protein, alone or further in combination with other E. coli O-antigen polysaccharides or glycoconjugates or bioconjugates thereof.

In certain embodiments, the antibodies induced, elicited or identified against an E. coli glucosylated O4 antigen have opsonophagocytic activity. In particular embodiments, the antibodies induced, elicited or identified are cross-reactive antibodies capable of mediating opsonophagocytic killing of both E. coli glucosylated and non-glucosylated O4 strains.

In certain embodiments, the antibodies induced, elicited or identified identified against an E. coli glucosylated O4 antigen specifically recognize unmodified and glucose modified O4 antigen polysaccharide. In certain embodiments, the antibodies induced, elicited or identified against an E. coli glucosylated O4 antigen specifically recognize E. coli of the O4 serotype. In certain embodiments, the antibodies induced by a bioconjugate of an E. coli glucosylated O4 antigen bind preferentially to glucosylated O4 antigen as compared to non-glucosylated O4 antigen.

Antibodies induced by the bioconjugates and compositions described herein can include immunoglobulin molecules and immunologically active portions of immunoglobulin molecules, i.e., molecules that contain an antigen binding site that specifically binds to an E. coli O-antigen polysaccharide, e.g., glucosylated O4 antigen polysaccharide.

Antibodies induced, elicited or identified using the bioconjugates or compositions provided herein can be used to monitor the efficacy of a therapy and/or disease progression. Any immunoassay system known in the art can be used for this purpose including, but not limited to, competitive and noncompetitive assay systems using techniques such as radioimmunoassays, ELISA (enzyme linked immunosorbent assays), electrochemiluminescence (ECL)-based immunoassays, “sandwich” immunoassays, precipitin reactions, gel diffusion precipitin reactions, immunodiffusion assays, immunoradiometric assays, fluorescent immunoassays, protein A immunoassays and immunoelectrophoresis assays. Several of these assays, e.g. ECL-based immunoassays, can be done in multiplex format, and typically multiplex assay formats are preferred.

Antibodies induced, elicited or identified using a bioconjugate of an E. coli glucosylated O4 antigen polysaccharide can be used to detect E. coli O4 strains, particularly glucosylated O4 strains, for example, from a plurality of E. coli strains and/or to diagnose an infection by an E. coli O4 or glucosylated O4 strain.

In another aspect, provided herein is a method of vaccinating a subject against E. coli (e.g. extra-intestinal pathogenic E. coli, ExPEC), comprising administering to the subject any of the bioconjugates of an E. coli glucosylated O4 antigen covalently linked to a carrier protein described herein, or a composition comprising a bioconjugate of an E. coli glucosylated O4 antigen covalent linked to a carrier protein, alone or further in combination with other E. coli O-antigens or glycoconjugates or bioconjugates thereof. One skilled in the art will understand that the subject will be vaccinated against E. coli strains whose O antigens or glycoconjugates or bioconjugates thereof are present in the composition administered. For example, administration of a composition comprising O1A, O2, glucosylated O4, O6A, and O25B antigen polysaccharides can be used to a vaccinate a subject against E. coli serotypes O1A, O2, O4, O6A, and O25B.

In certain embodiments, vaccination is for preventing an invasive ExPEC disease (IED), e.g., urosepsis, bacteremia, sepsis, etc. In certain embodiments, vaccination is to prevent or reduce the occurrence or severity of urinary tract infections. In certain embodiments, an IED can be hospital-acquired, e.g. in patients undergoing urogenital and/or abdominal procedures or surgeries. In certain embodiments, an IED can be healthcare-associated, e.g. in patients receiving health care for another condition, for instance via central lines, catheters, etc, e.g. in a hospital, ambulatory surgical center end-stage renal disease facility, long-term care facility, etc. In certain embodiments, the IED can be community-acquired, e.g. in a patient that was not recently exposed to healthcare risks.

In another aspect, provided herein is a method of inducing an immune response against E. coli (e.g., ExPEC) in a subject, comprising administering to the subject any of the bioconjugates of an E. coli glucosylated O4 antigen covalently linked to a carrier protein described herein, or a composition comprising a bioconjugate of an E. coli glucosylated O4 antigen covalently linked to a carrier protein, alone or further in combination with other E. coli O-antigens or glycoconjugates or bioconjugates thereof. In one embodiment, the subject has an E. coli (e.g., ExPEC) infection at the time of administration. In a preferred embodiment, the subject does not have an E. coli (e.g., ExPEC) infection at the time of administration.

In certain embodiments, the compositions and bioconjugates described herein can be administered to a subject to induce an immune response that includes the production of antibodies, preferably antibodies having opsonophagocytic activity. Such antibodies can be isolated using techniques known to one of skill in the art (e.g., immunoaffinity chromatography, centrifugation, precipitation, etc.).

The ability of the bioconjugates and compositions described herein to generate an immune response in a subject can be assessed using any approach known to those of skill in the art or described herein. In some embodiments, the ability of a bioconjugate to generate an immune response in a subject can be assessed by immunizing a subject (e.g., a mouse, rat, rabbit, or monkey) or set of subjects with a bioconjugate described herein and immunizing an additional subject (e.g., a mouse, rat, rabbit, or monkey) or set of subjects with a control (PBS). The subjects or set of subjects can subsequently be challenged with ExPEC and the ability of the ExPEC to cause disease (e.g., UTI, bacteremia, or other disease) in the subjects or set of subjects can be determined. Those skilled in the art will recognize that if the subject or set of subjects immunized with the control suffer(s) from disease subsequent to challenge with the ExPEC but the subject or set of subjects immunized with a bioconjugate(s) or composition thereof described herein suffer less from or do not suffer from disease, then the bioconjugate is able to generate an immune response in a subject. The ability of a bioconjugate(s) or composition thereof described herein to induce antiserum that cross-reacts with an O antigen from ExPEC can be tested by, e.g., an immunoassay, such as an ELISA (see e.g., Van den Dobbelsteen et al, 2016, Vaccine 34: 4152-4160), or an ECL-based immunoassay.

For example, the ability of the bioconjugates described herein to generate an immune response in a subject can be assessed using a serum bactericidal assay (SBA) or opsonophagocytic killing assay (OPK assay, or OPKA), which represents an established and accepted method that has been used to obtain approval of glycoconjugate-based vaccines. Such assays are well-known in the art and, briefly, comprise the steps of generating and isolating antibodies against a target of interest (e.g., an O antigen polysaccharide, e.g., E. coli glucosylated O4 antigen polysaccharide) by administering to a subject (e.g., a mouse, rat, rabbit, or monkey) a compound that elicits such antibodies. Subsequently, the bactericidal capacity of the antibodies can be assessed by, e.g., culturing the bacteria in question (e.g., E. coli of the relevant serotype) in the presence of the antibodies and complement and—depending on the assay—neutrophilic cells and assaying the ability of the antibodies to mediate killing and/or neutralization of the bacteria, e.g., using standard microbiological approaches. For an example of OPK assay for E. coli bioconjugate vaccines, see e.g. Abbanat et al, 2017, Clin. Vaccine Immunol. 24: e00123-17. An OPK assay can be performed in monoplex or multiplex format, of which multiplex format (e.g. testing multiple serotypes at the same time) is typically preferred. A multiplex OPK assay is sometimes referred to herein as ‘MOPA’.

In some embodiments, the methods described herein comprise administering an effective amount of bioconjugates of an E. coli glucosylated O4 antigen covalently linked to a carrier protein described herein, or a composition comprising a bioconjugate of an E. coli glucosylated O4 antigen covalently linked to a carrier protein, alone or further in combination with other E. coli O-antigens or glycoconjugates or bioconjugates thereof. In one embodiment, an “effective amount” is an amount that vaccinates a subject against E. coli (e.g., ExPEC). In another embodiment, an “effective amount” is an amount that induces an immune response against E. coli (e.g., ExPEC) in a subject, such as an immune response including the production of antibodies, preferably antibodies having opsonophagocytic activity.

In particular embodiments, wherein a composition provided herein comprises a bioconjugate of an E. coli glucosylated O4 antigen polysaccharide and at least a bioconjugate of an E. coli O25B antigen polysaccharide, an effective amount of the E. coli O25B antigen polysaccharide is about 1.5 to 6 times, e.g. about 2 to 4 times higher, such as 1.5, 2, 3, 4, 5 or 6 times higher than the concentration of any of the other bioconjugates present in the composition. In such embodiments, an effective amount of the E. coli O25B antigen polysaccharide is for instance about 5 to 18 μg per administration, such as 5, 6, 7, 8, 9, 10, 11, 12, 13, 14, 15, 16, 17, 18 μg per administration.

In certain embodiments, a bioconjugate or composition according to the invention is administered to a subject once. In certain embodiments, a bioconjugate or composition according to the invention is administered to a subject more than once, e.g. in a prime-boost regimen. In certain embodiments, the time between two administrations is at least two weeks, at least one month, at least two months, at least three months, at least six months, at least one year, at least two years, at least five years, at least ten years, or at least fifteen years. In humans, a desired immune response can typically be generated by a single administration of a bioconjugate or composition according to the invention. In certain embodiments, a repeat administration after for instance ten years is provided.

### Host Cells

Provided herein are host cells, e.g., prokaryotic host cells, capable of producing E. coli O antigens and bioconjugates comprising such E. coli O antigens. The host cells provided herein preferably are modified to comprise (e.g., through genetic engineering) one or more of the nucleic acids encoding host cell machinery (e.g., glycosyltransferases) used to produce E. coli O-antigen polysaccharides and/or bioconjugates thereof.

Any host cells known to those of skill in the art can be used to produce the E. coli O antigen polysaccharides described herein (e.g., E. coli glucosylated O4 antigen polysaccharide) and bioconjugates comprising the E. coli O antigen polysaccharides described herein (e.g., a bioconjugate of E. coli glucosylated O4 antigen polysaccharide) including archaea, prokaryotic host cells, and eukaryotic host cells. In a preferred embodiment, a host cell is a prokaryotic host cell. Exemplary prokaryotic host cells for use in production of the E. coli O antigen polysaccharides described herein and bioconjugates comprising the E. coli O antigen polysaccharides described herein include, but are not limited to, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species.

In a specific embodiment, the host cell used to produce the E. coli O antigen polysaccharides described herein and bioconjugates comprising the E. coli O antigen polysaccharides described herein is a prokaryotic host cell, and is preferably E. coli.

In certain embodiments, the host cells used to produce the E. coli O antigen polysaccharides and bioconjugates described herein are engineered to comprise heterologous nucleic acids, e.g., heterologous nucleic acids comprising rfb gene clusters of a desired O antigen serotype, heterologous nucleic acids that encode one or more carrier proteins and/or glycosyltransferases. In a specific embodiment, heterologous rfb genes, and/or heterologous nucleic acids that encode proteins involved in glycosylation pathways (e.g., prokaryotic and/or eukaryotic glycosylation pathways) can be introduced into the host cells described herein. Such nucleic acids can encode proteins including, but not limited to, oligosaccharyl transferases and/or glycosyltransferases.

Sequences of various genes and gene clusters encoding glycosyltransferases useful in making recombinant host cells that can, e.g., be used to prepare E. coli O antigen polysaccharides and bioconjugates thereof are described herein. Those skilled in the art will appreciate that due to the degeneracy of the genetic code, a protein having a specific amino acid sequence can be encoded by multiple different nucleic acids. Thus, those skilled in the art will understand that a nucleic acid provided herein can be altered in such a way that its sequence differs from a sequence provided herein, without affecting the amino acid sequence of the protein encoded by the nucleic acid.

Provided herein are host cells (e.g., recombinant host cells) for producing a bioconjugate of an E. coli glucosylated O4 antigen polysaccharide, O1A antigen polysaccharide, O2 antigen polysaccharide, O6A antigen polysaccharide, O8 antigen polysaccharide, O15 antigen polysaccharide, O16 antigen polysaccharide, O18A antigen polysaccharide, O25B antigen polysaccharide, or O75 antigen polysaccharide. The host cells provided herein comprise nucleic acids encoding enzymes (e.g., glycosyltransferases) capable of producing the E. coli 0 antigen polysaccharide. The host cells provided herein can naturally express nucleic acids capable of producing an O antigen of interest, or the host cells can be made to express such nucleic acids. In certain embodiments the nucleic acids are heterologous to the host cells and introduced into the host cells using genetic approaches known in the art. For example, the nucleic acids can be introduced into the host cell by genetic manipulation (e.g., the gene cluster is expressed on a plasmid or plasmids or integrated into the host cell genome (see, e.g., International Patent Application Publications WO 2014/037585, WO 2014/057109, WO 2015/052344).

In one embodiment, provided herein is a host cell (e.g., recombinant host cell) capable of producing a bioconjugate of an E. coli glucosylated O4 antigen polysaccharide covalently linked to a carrier protein. Such a host cell comprises, preferably by engineering a precursor cell, a nucleic acid sequence encoding a gtrS gene, which, to the best of the knowledge of the inventors, was identified herein for the first time as encoding a polysaccharide branching enzyme capable of transferring glucose to the E. coli O4 antigen (i.e., a glucosyltransferase specific to the E. coli O4 antigen polysaccharide), and particularly to L-Rha via an α-1,3-glycosidic linkage. An example of an amino acid sequence of such branching enzyme is provided in SEQ ID NO: 4. Other examples comprise amino acid sequences that are at least 80% identical thereto. Exemplary examples of nucleic acid sequence encoding gtrS genes specific to the E. coli O4 antigen polysaccharide include, but are not limited to, SEQ ID NO: 5, or degenerate nucleic acid sequences thereto that encode SEQ ID NO: 4, or nucleic acid sequences that encode functional O4-specific GtrS enzymes that have at least 80% identity to SEQ ID NO: 4.

In a specific embodiment, a host cell (e.g., recombinant host cell) capable of producing a bioconjugate of an E. coli glucosylated O4 antigen polysaccharide covalently linked to a carrier protein, comprises a nucleotide sequence encoding a glucosyl transferase having at least 80% sequence identity to SEQ ID NO: 4, such as about 80%, 85%, 90%, 91%, 92%, 93%, 94%, 95%, 95%, 97%, 98%, 99%, or 100% sequence identity to SEQ ID NO: 4. In view of the redundancy in the genetic code, one of ordinary skill in the art can make variants of nucleic encoding the amino acid sequences of glucosyl transferases, e.g., using codon optimized sequences, if desired.

In certain embodiments, a host cell (e.g., recombinant host cell) capable of producing a bioconjugate of an E. coli glucosylated O4 antigen polysaccharide covalently linked to a carrier protein, comprising a nucleotide sequence encoding a glucosyl transferase (GtrS) having at least 80% sequence identity to SEQ ID NO: 4, further comprises a nucleotide sequence encoding a bactoprenol-linked glucose translocase (GtrA) having at least 80% sequence identity to SEQ ID NO: 7, and a nucleotide sequence encoding a bactoprenol glucosyl transferase (GtrB) having at least 80% sequence identity to SEQ ID NO: 8. In certain embodiments, said nucleic acid sequences encode GtrA and GtrB proteins that are at least about 80%, 85%, 90%, 95%, 96%, 97%, 98%, 99%, or 100% identical to SEQ ID NOs: 7 and 8, respectively, and have bactoprenol-linked glucose translocase (SEQ ID NO: 7) and bactoprenol glucosyl transferase (SEQ ID NO: 8) activity, respectively. In view of the redundancy in the genetic code, one of ordinary skill in the art can make variants of nucleic encoding the amino acid sequences of bactoprenol-linked glucose translocases and of bactoprenol glucosyl transferases, e.g., using codon optimized sequences, if desired.

A host cell (e.g., recombinant host cell) capable of producing a bioconjugate of an E. coli glucosylated O4 antigen polysaccharide covalently linked to a carrier protein provided herein further comprises a nucleotide sequence of an rfb gene cluster for the E. coli O4 antigen polysaccharide. An example of an rfb gene cluster useful for production of the E. coli O4 antigen polysaccharide is provided herein as SEQ ID NO: 9. Another example can be found in GenBank, locus AY568960. Degenerate nucleic acid sequences encoding the same enzymes as encoded by this sequence, or sequences that encode enzymes that are at least 80% identical, preferably at least 85%, 90%, 91%, 92%, 93%, 94%, 95%, 96%, 97%, 98%, 99% identical, can also be used.

In a specific embodiment, provided herein is a host cell (e.g., a recombinant host cell, preferably a recombinant prokaryotic host cell, preferably a recombinant E. coli host cell) that produces glucosylated O4 antigen polysaccharide, wherein the host cell comprises gtrS, an rfb gene cluster for the E. coli O4 antigen polysaccharide, and nucleic acid encoding a carrier protein. Such host cells can be engineered using recombinant approaches to comprise one or more plasmids comprising the gtrS gene, the rfb gene cluster, and/or nucleic acid encoding a carrier protein, or to comprise some or all of the relevant genes such as gtrS, the rfb cluster and/or the nucleic acid encoding the carrier protein integrated into the host cell genome. In certain embodiments, the genes or gene clusters have been integrated into the genome of the host cell using homologous recombination. An advantage of integration of genes into the genome of the host cell is stability in the absence of antibiotic selection.

In another specific embodiment, provided herein is a host cell (e.g., a recombinant host cell, preferably a recombinant prokaryotic host cell) that produces glucosylated O4 antigen polysaccharide, wherein the host cell comprises GtrS (glucosyltransferase), as well as the enzymes encoded by the O4 rfb cluster. In certain embodiments, some or all of the aforementioned enzymes are heterologous to the host cell.

In other specific embodiments, provided herein is a host cell (e.g. a recombinant host cell, preferably a recombinant prokaryotic host cell) that produces E. coli glucosylated O4 antigen polysaccharide, preferably a bioconjugate of E. coli glucosylated O4 antigen polysaccharide, wherein the host cell further comprises a nucleotide sequence encoding an oligosaccharyl transferase and/or a nucleotide sequence encoding a carrier protein. In one specific embodiment, the oligosaccharyl transferase is heterologous to the host cell. In another specific embodiment, the carrier protein is heterologous to the host cell. Preferably, the host cell comprises a heterologous nucleotide sequence encoding a glucosyl transferase having at least 80% sequence identity to SEQ ID NO: 4. In preferred embodiments, the rfb genes of the O4 cluster are heterologous to the host cell. Preferably the sequence encoding the enzyme that is capable of introducing the branched glucose side chain to the O4 antigen, i.e. the gtrS gene (encoding a glucosyl transferase having at least 80% sequence identity to SEQ ID N0:4) is heterologous to the host cell. A nucleic acid is heterologous to the host cell if the same sequence is not naturally present in said host cell. Heterologous nucleic acid can for instance be introduced in a parent cell by genetic engineering, e.g by transformation (e.g. chemical transformation or electroporation) and/or recombination. In certain embodiments, heterologous nucleic acid such as a desired rfb locus, gtrS coding sequence, carrier protein encoding sequence, and/or glycosyltransferase encoding sequence are integrated into the genome of the host cell, preferably a bacterial host cell, preferably an E. coli host cell. In preferred embodiments, the endogenous rfb locus and if applicable gtrS coding sequence have been inactivated, preferably deleted from the genome of the recombinant host cell as compared to a predecessor thereof, and preferably these are replaced by the desired heterologous rfb locus, and if applicable desired gtrS coding sequence, respectively. In certain embodiments the host cell is a K-12 of E. coli (as a non-limiting example, E. coli strain W3110 is a K-12 strain), or a B strain of E. coli (as a non-limiting example, E. coli strain BL21 is a B strain), or any other well-defined strain of E. coli, e.g. laboratory strains or production strains, in contrast to primary wild-type isolates. In preferred embodiments, the host cell is derived from E. coli that does not express O4 antigen or glucosylated O4 antigen, by introduction into such E. coli of the O4 rfb locus and the gtrS gene encoding a glucosyl transferase having at least 80% sequence identity to SEQ ID N0:4. Advantages of using well-characterized strains, such as E. coli K-12 or E. coli B, as precursors for host cells is the possibility to use a similar production process for different O-antigen bioconjugates, since the characteristics of the production strain are well-defined. Even though bioconjugates of different O-antigens will behave differently and expression processes can be optimized per production strain, at least the basic process for production of O-antigen bioconjugates will be more predictable using such well-defined precursor strains than when unknown strains such as wild-type isolates are used as precursors for production of host strains. This way, experience with production of earlier described E. coli O-antigen bioconjugates such as O1A, O2, O6A and O25B bioconjugates as described in for instance WO 2015/124769 and WO 2017/035181 can be used as basis to design production of other E. coli O-antigen bioconjugates. Unlike gtrS, the gtrA and gtrB genes are not serotype-specific, and in certain embodiments these are homologous to the host cell (e.g. E. coli K12 strain W3110 includes gtrA and gtrB genes that are capable of functioning together with the O4-serotype specific recombinantly introduced gtrS gene encoding a glucosyl transferase of SEQ ID NO: 4 or a glucosyl transferase that is at least 80% identical thereto, replacing the endogenous gtrS gene). In other embodiments, one or both of gtrA and gtrB genes (encoding GtrA and GtrB proteins that are at least about 80% identical to SEQ ID NOs: 7 and 8, respectively, and having bactoprenol-linked glucose translocase and bactoprenol glucosyl transferase activity respectively, are also recombinantly introduced in the host cell, for instance in case the host cell does not have endogenous gtrA and/or gtrB genes.

Also provided herein are host cells (e.g., recombinant host cells) capable of producing a bioconjugate of an E. coli O1A, O2, O6A, O8, O15, O16, O18A, O25B, or O75 antigen polysaccharide covalently linked to a carrier protein. Such host cells (e.g., recombinant host cells) comprise nucleotide sequence of an rfb gene cluster specific to the O-antigen polysaccharide. The rfb gene clusters can be isolated from wild-type E. coli strains, and combined with nucleic acids encoding an oligosaccharyl transferase (e.g., PglB) and carrier protein (e.g., EPA) within one host cell to obtain a recombinant host cell that produces the E. coli O-antigen of interest or bioconjugate thereof. For example, such host cells can be engineered using recombinant approaches to comprise one or more plasmids comprising the rfb gene cluster, oligosaccharyl transferase (e.g., PglB) and carrier protein (e.g., EPA) using bioconjugation technology such as that described in WO 2014/037585, WO 2009/104074, and WO 2009/089396. Preferably the host cells comprise the rfb gene clusters integrated into their genome. The nucleic acids encoding oligosaccharyl transferase, carrier protein, and where applicable gtrS gene, are in certain embodiments also integrated into the genome of the host cell. Heterologous or homologous gtrA and gtrB genes are in certain embodiments also integrated into the genome of the host cell.

Preparation of bioconjugates for O1A, O2, O6A and O25B antigens has been described in detail in WO 2015/124769 and WO 2017/035181. Examplary gene clusters for each E. coli O antigen (rfb loci) have been described in Iguchi A, et al, DNA Research, 2014, 1-7 (doi: 10.1093/dnares/dsu043), and in DebRoy C, et al, PLoS One. 2016, 11(1):e0147434 (doi: 10.1371/journal.pone.0147434; correction in: Plos One. 2016, 11(4):e0154551, doi: 10.1371/journal.pone.0154551). Nucleic acid sequences for the rfb clusters and amino acid sequences for proteins encoded therein can also be found in public databases, such as GenBank. Exemplary sequences for rfb clusters that can be used in production strains for bioconjugates with polysaccharide antigens of the serotypes disclosed herein, are also provided in SEQ ID NOs: 9 and 11-19. Thus, for each of the desired bioconjugates mentioned above, the respective rfb cluster can be introduced into a host cell, to obtain host cells with the specific rfb cluster for the desired O-antigen, as well as containing nucleic acid encoding oligosaccharyl transferase and carrier protein. For reasons indicated above, preferably the host cells are recombinant host cells, and preferably are derived from strains with relatively well-known characteristics, such as E. coli laboratory or production strains, e.g. E. coli K12 or E. coli BL21, etc. Preferably, the rfb clusters are heterologous to the host cell, e.g. introduced into a precursor cell of the host cell, and preferably integrated into the genome thereof. Preferably an original rfb gene cluster, if such was present in a precursor cell, has been replaced by the rfb gene cluster for the O-antigen of interest in the host cell, to enable production of bioconjugate of the O-antigen of interest. Preferably the oligosaccharyl transferase is heterologous to the host cell, and in certain embodiments nucleic acid encoding such oligosaccharyl transferase is integrated into the genome of the host cell.

Any of the host cells provided herein (e.g., recombinant host cells, preferably recombinant prokaryotic host cells) comprise nucleic acids encoding additional enzymes active in the N-glycosylation of proteins, e.g., the host cell provided herein can further comprise a nucleic acid encoding an oligosaccharyl transferase or nucleic acids encoding other glycosyltransferases.

The host cells provided herein comprise a nucleic acid that encodes an oligosaccharyl transferase. Oligosaccharyl transferases transfer lipid-linked oligosaccharides to asparagine residues of nascent polypeptide chains that comprise an N-glycosylation consensus motif. The nucleic acid that encodes an oligosaccharyl transferase can be native to the host cell, or can be introduced into the host cell using genetic approaches. In preferred embodiments, the oligosaccharyl transferase is heterologous to the host cell. E. coli does not naturally comprise an oligosaccharyl transferase, and hence if E. coli is used as a host cell for production of bioconjugates, a heterologous oligosaccharyl transferase is comprised in such host cell, e.g. upon introduction by genetic engineering. The oligosaccharyl transferase can be from any source known in the art in view of the present disclosure.

In certain embodiments, an alternative to an oligosaccharyl transferase with N-glycosyltransferase activity, such as an O-glycosyltransferase, e.g. as a non-limiting example PglL, can be used, in conjunction with its own, different, glycosylation consensus sequence in the carrier protein, as for instance described in WO 2016/82597. Other glycosyltransferases, such as O-glycosyltransferases, can thus also be used as an oligosaccharyl transferase according to the invention.

In certain preferred embodiments, the oligosaccharyl transferase is an oligosaccharyl transferase from Campylobacter. For example, in one embodiment, the oligosaccharyl transferase is an oligosaccharyl transferase from Campylobacter jejuni (i.e., pglB; see, e.g., Wacker et al., 2002, Science 298:1790-1793; see also, e.g., NCBI Gene ID: 3231775, UniProt Accession No. O86154). In another embodiment, the oligosaccharyl transferase is an oligosaccharyl transferase from Campylobacter lari (see, e.g., NCBI Gene ID: 7410986).

In specific embodiments, the oligosaccharyl transferase is PglB oligosaccharyl transferase from Campylobacter jejuni, including the natural (wild-type) protein or any variant thereof, such as those described in International Patent Application Publications WO 2016/107818 and WO 2016/107819. PglB can transfer lipid-linked oligosaccharides to asparagine residues in the consensus sequences SEQ ID NO: 1 and SEQ ID NO: 2. In particular embodiments, the PglB oligosaccharyl transferase comprises SEQ ID NO: 6, or a variant thereof. In certain embodiments one or more endogenous glycosylation consensus sequences in a wild-type PglB have been mutated to avoid PglB autoglycosylation, e.g. SEQ ID NO: 6 comprising the mutation N534Q. Examples of variant PglB oligosaccharyl transferases suitable for use in the recombinant host cells provided herein include the PglB oligosaccharyl transferase of SEQ ID NO: 6 comprising at least one mutation selected from the group consisting of N311V, K482R, D483H, A669V, Y77H, S80R, Q287P, and K289R. In one particular embodiment, a variant PglB oligosaccharyl transferase has SEQ ID NO: 6 comprising the mutation N311V. In another particular embodiment, a variant PglB oligosaccharyl transferase has SEQ ID NO: 6 comprising the mutations Y77H and N311V. In another particular embodiment, a variant PglB oligosaccharyl transferase has SEQ ID NO: 6 comprising the mutations N311V, K482R, D483H, and A669V. In another particular embodiment, a variant PglB oligosaccharyl transferase has SEQ ID NO: 6 comprising the mutations Y77H, S80R, Q287P, K289R, and N311V. It was found and described herein that certain PglB oligosaccharyl transferase variants give surprisingly improved yields in production of E. coli O-antigen bioconjugates of specific serotypes. The improved or optimal PglB variant for a given E. coli O-antigen was not predictable. The invention in certain aspects therefore also provides methods for producing bioconjugates of specific E. coli O-antigens, using specific PglB variants as the oligosaccharyl transferase. Further variants of PglB that are at least 80%, 85%, 90%, 91%, 92%, 93%, 94%, 95%, 96%, 97%, 98%, 99% identical to SEQ ID NO: 6 and still have oligosaccharyl transferase activity, preferably having one or more of the specific amino acids on the indicated positions disclosed in combination herein (e.g. 77Y, 80S, 287Q, 289K, 311N, 482K, 483D, 669A; or 311V; or 311V, 482R, 483H, 669V; or 77H, 80R, 287P, 289R, 311V; or 77H, 311V; etc) can also be used for production of bioconjugates.

In a specific embodiment, a host cell (e.g., recombinant host cell) capable of producing a bioconjugate of an E. coli glucosylated O4 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutation N311V, or more preferably SEQ ID NO: 6 comprising the mutations Y77H and N311V.

In other specific embodiments, a host cell (e.g., recombinant host cell) capable of producing a bioconjugate of an E. coli O1A, O6A, or O15 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutations N311V, K482R, D483H, and A669V.

In a specific embodiment, a host cell (e.g., recombinant host cell) capable of producing a bioconjugate of an E. coli O16 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutations Y77H, S80R, Q287P, K289R, and N311V.

In a specific embodiment, a host cell (e.g., recombinant host cell) capable of producing a bioconjugate of an E. coli O75 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutation N311V.

In a specific embodiment, a host cell (e.g., recombinant host cell) capable of producing a bioconjugate of an E. coli O8, O18A, O25B, or O2 antigen polysaccharide covalently linked to a carrier protein further comprises a nucleotide sequence encoding PglB oligosaccharyl transferase from Campylobacter jejuni having the amino acid sequence of SEQ ID NO: 6, preferably wherein SEQ ID NO: 6 comprises no amino acid mutations at positions 77, 80, 287, 289, 311, 482, 483, and 669.

In some embodiments, any of the host cells provided herein comprise a nucleic acid encoding a carrier protein, e.g., a protein to which the O-antigen polysaccharide(s) produced by the host cell glycosylation machinery can be attached to form a bioconjugate. The host cell can comprise a nucleic acid encoding any carrier protein known to those skilled in the art in view of the present disclosure including, but not limited to, detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In preferred embodiments, a host cell further comprises a nucleic acid encoding detoxified Exotoxin A of P. aeruginosa (EPA). Preferably, the EPA carrier protein comprises 1-10 glycosylation sites, preferably 2 to 4 glycosylation sites, most preferably 4 glycosylation sites, such as 1-10, preferably 2-4, and more preferably 4 glycosylation sites each comprising a glycosylation consensus sequence having the amino acid sequence of SEQ ID NO: 1, and more preferably having the amino acid sequence of SEQ ID NO: 2. In a specific embodiment, a host cell further comprises a nucleic acid encoding EPA-4 carrier protein comprising SEQ ID NO: 3.

In certain embodiments, the carrier proteins used in the generation of the bioconjugates by the host cells described herein comprise a “tag,” i.e., a sequence of amino acids that allows for the isolation and/or identification of the carrier protein. For example, adding a tag to a carrier protein can be useful in the purification of that protein and, hence, the purification of conjugate vaccines comprising the tagged carrier protein. Exemplary tags that can be used herein include, without limitation, histidine (HIS) tags (e.g., hexa-histidine-tag, or 6×His-Tag), FLAG-TAG, and HA tags. In certain embodiments, the tags used herein are removable, e.g., removal by chemical agents or by enzymatic means, once they are no longer needed, e.g., after the protein has been purified. In other embodiments, the carrier protein does not comprise a tag.

In certain embodiments, the carrier proteins described herein comprise a signal sequence that targets the carrier protein to the periplasmic space of the host cell that expresses the carrier protein. In a specific embodiment, the signal sequence is from E. coli DsbA, E. coli outer membrane porin A (OmpA), E. coli maltose binding protein (MalE), Erwinia carotovorans pectate lyase (PelB), FlgI, NikA, or Bacillus sp. endoxylanase (XynA), heat labile E. coli enterotoxin LTIIb, Bacillus endoxylanase XynA, or E. coli flagellin (FlgI). In one embodiment, the signal sequence comprises SEQ ID NO: 10. A signal sequence may be cleaved off after translocation of the protein to the periplasm and may thus no longer be present in the final carrier protein of a bioconjugate.

In certain embodiments, additional modifications can be introduced (e.g., using recombinant techniques) into the host cells described herein. For example, host cell nucleic acids (e.g., genes) that encode proteins that form part of a possibly competing or interfering glycosylation pathway (e.g., compete or interfere with one or more heterologous genes involved in glycosylation that are recombinantly introduced into the host cell) can be deleted or modified in the host cell background (genome) in a manner that makes them inactive/dysfunctional (i.e., the host cell nucleic acids that are deleted/modified do not encode a functional protein). In certain embodiments, when nucleic acids are deleted from the genome of the host cells provided herein, they are replaced by a desirable sequence, e.g., a sequence that is useful for production of an O antigen polysaccharide or bioconjugate thereof.

Exemplary genes or gene clusters that can be deleted in host cells (and, in some cases, replaced with other desired nucleic acid sequences) include genes or gene clusters of host cells involved in glycolipid biosynthesis, such as waaL (see, e.g., Feldman et al., 2005, PNAS USA 102:3016-3021), the lipid A core biosynthesis cluster (waa), galactose cluster (gal), arabinose cluster (ara), colonic acid cluster (wc), capsular polysaccharide cluster, undecaprenol-p biosynthesis genes (e.g. uppS, uppP), und-P recycling genes, metabolic enzymes involved in nucleotide activated sugar biosynthesis, enterobacterial common antigen cluster (eca), and prophage O antigen modification clusters like the gtrABS cluster or regions thereof. In a specific embodiment, the host cells described herein are modified such that they do not produce any 0 antigen polysaccharide other than a desired O antigen polysaccharide, e.g., glucosylated O4 antigen polysaccharide.

In a specific embodiment, the waaL gene is deleted or functionally inactivated from the genome of a host cell (e.g., recombinant host cell) provided herein. The terms “waaL” and “waaL gene” refer to the O-antigen ligase gene encoding a membrane bound enzyme with an active site located in the periplasm. The encoded enzyme transfers undecaprenylphosphate (UPP)-bound O antigen to the lipid A core, forming lipopolysaccharide. Deletion or disruption of the endogenous waaL gene (e.g., AwaaL strains) disrupts transfer of the O-antigen to lipid A, and can instead enhance transfer of the O-antigen to another biomolecule, such as a carrier protein.

In another specific embodiment, one or more of the waaL gene, gtrA gene, gtrB gene, gtrS gene, and the rfb gene cluster is deleted or functionally inactivated from the original genome of a prokaryotic host cell provided herein.

In one embodiment, a host cell used herein is E. coli that produces a bioconjugate of glucosylated O4 antigen polysaccharide, wherein the waaL gene is deleted or functionally inactivated from the genome of the host cell, and a gtrS gene specific to E. coli O4 antigen polysaccharide is inserted. In certain embodiments for production strains for bioconjugates of the glucosylated O4 O-antigen, a gtrS gene encoding a glucosyl transferase having at least 80% sequence identity to SEQ ID NO:4 is inserted in the place of a gtrS gene of the parent strain, so as to replace the gtrS gene in that parent strain with the one that is responsible for glucosylation of the O4 antigen. An example of such a parent strain is E. coli K-12 strain W3110. The gtrA and gtrB genes can be homologous to the parent strain, or alternatively one or both of these genes can be heterologous to the parent strain. Typically, and unlike the gtrS gene, these gtrA and gtrB genes are not specific for the O-antigen structure.

Also provided herein are methods of making recombinant host cells. Recombinant host cells produced by the methods described herein can be used to produce bioconjugates of E. coli O antigens. The methods comprise introducing one or more recombinant nucleic acid molecules into a cell to produce the recombinant host cell. Typically, the recombinant nucleic acid molecules are heterologous. Any method known in the art in view of the present disclosure can be used to introduce recombinant nucleic acid molecules into a host cell. Recombinant nucleic acids can be introduced into the host cells described herein using any methods known to those of ordinary skill in the art, e.g., electroporation, chemical transformation, by heat shock, natural transformation, phage transduction, and conjugation. In specific embodiments, recombinant nucleic acids are introduced into the host cells described herein using a plasmid. For example, the heterologous nucleic acids can be expressed in the host cells by a plasmid (e.g., an expression vector). In another specific embodiment, heterologous nucleic acids are introduced into the host cells described herein using the method of insertion into the genome as for instance described in International Patent Application Publication WO 2014/037585, WO 2014/057109, or WO 2015/052344.

In one embodiment, a method of making a recombinant host cell for producing a bioconjugate of an E. coli glucosylated O4 antigen polysaccharide covalently linked to a carrier protein comprises introducing one or more recombinant nucleic acid molecules into a cell, preferably an E. coli cell, to produce the recombinant host cell. In such embodiments, the recombinant nucleic acid molecules introduced into the cell include (i) a nucleotide sequence of an rfb gene cluster for the E. coli O4 antigen polysaccharide; (ii) a nucleotide sequence encoding a glucosyl transferase having at least 80% sequence identity to SEQ ID NO: 4, wherein the glucosyl transferase is capable of modifying the E. coli O4 antigen polysaccharide to produce the E. coli glucosylated O4 antigen polysaccharide; (iii) a nucleotide sequence encoding a carrier protein; and (iv) a nucleotide sequence encoding an oligosaccharyl transferase capable of covalently linking the E. coli glucosylated O4 antigen polysaccharide to the carrier protein to produce the bioconjugate. In preferred embodiments, the nucleotide sequence encoding a glucosyl transferase having at least 80% sequence identity to SEQ ID NO: 4 replaces the endogenous gtrS gene. Deleting the endogenous gtrS has the advantage that it will not interfere with generation of the glucosylated O4 antigen polysaccharide structure. In certain embodiments, the nucleotide sequence of the rfb gene cluster for the E. coli O4 antigen polysaccharide replaces the endogenous rfb gene cluster of the parent strain that is used to make the recombinant host cell. If the cell does not yet encode gtrA and/or gtrB genes, nucleotide sequences encoding a translocase (gtrA) and a glycosyltransferase (gtrB), having at least 80% identity to SEQ ID NOs: 7 and 8, respectively, can be introduced into the cell. If the cell already encodes gtrA and gtrB genes (such as for instance the case in E. coli K-12 strain W3110), there is no need to introduce or change these genes.

In a specific embodiment, the glucosyl transferase (gtrS specific for adding glucose branch to O4 antigen) has SEQ ID NO: 4.

In a specific embodiment, the oligosaccharyl transferase is PglB from C. jejuni. In one such embodiment, the oligosaccharyl transferase comprises the amino acid sequence of SEQ ID NO: 6. In another such embodiment, the oligosaccharyl transferase comprises the amino acid sequence of SEQ ID NO: 6 comprising the mutation N311V. In another such embodiment, the oligosaccharyl transferase comprises the amino acid sequence of SEQ ID NO: 6 comprising the mutations Y77H and N311V.

In another specific embodiment, the carrier protein comprises at least one glycosylation site comprising a glycosylation consensus sequence having SEQ ID NO: 1, preferably SEQ ID NO: 2. In another specific embodiment, the carrier protein is EPA, preferably EPA-4, such as EPA-4 comprising SEQ ID NO: 3.

E. coli strains that are used routinely in molecular biology as both a tool and a model organism can for instance be used as parents for host cells in certain embodiments according to the invention. Non-limiting examples include E. coli K12 strains (for example, such as W1485, W2637, W3110, MG1655, DH1, DH5α, DH10, etc.), B strains (e.g. BL-21, REL606, etc.), C strains, or W strains. In one particular embodiment, the host strain is derived from parent strain W3110. This strain can for instance be obtained from the E. coli Genetic Stock Center at Yale. For more information on E. coli, see e.g. Ecoliwiki.net.

### Methods of Producing Conjugates and Bioconjugates

Also provided are methods of producing glycoconjugates of the E. coli O antigen polysaccharides described herein. Glycoconjugates, including bioconjugates, can be prepared in vitro or in vivo, e.g., using the recombinant host cells described herein for production.

In some embodiments, glycoconjugates can be prepared by chemical synthesis, i.e., prepared outside of host cells (in vitro). For example, an E. coli O antigen polysaccharide can be conjugated to carrier proteins using methods known to those of ordinary skill in the art, including by means of using activation reactive groups in the polysaccharide/oligosaccharide as well as the carrier protein. See, e.g., Pawlowski et al., 2000, Vaccine 18:1873-1885; and Robbins, et al., 2009, Proc Natl Acad Sci USA 106:7974-7978), the disclosures of which are herein incorporated by reference. Such approaches comprise extraction of antigenic polysaccharides/oligosaccharides from host cells, purifying the polysaccharides/oligosaccharides, chemically activating the polysaccharides/oligosaccharides, and conjugating the polysaccharides/oligosaccharides to a carrier protein.

In some embodiments, the host cells described herein can be used to produce bioconjugates comprising an E. coli O antigen polysaccharide covalently linked to a carrier protein. Methods of producing such bioconjugates using host cells are known in the art. See, e.g., WO 2003/074687 and WO 2006/119987. Such methods comprise culturing any of the recombinant host cells described herein under conditions for production of the bioconjugate. Bioconjugates can be isolated, separated, and/or purified from recombinant host cells using any method known in the art in view of the present disclosure. For example, bioconjugates can be purified by any method known in the art for purification of a protein, for instance, by chromatography (e.g., ion exchange, anionic exchange, affinity, and sizing column chromatography), centrifugation, differential solubility, or by any other standard technique for the purification of proteins. See, e.g., methods described in WO 2009/104074. Further, the bioconjugates can be fused to heterologous polypeptide sequences to facilitate purification. The actual conditions used to purify a particular bioconjugate will depend, in part, on factors such as net charge, hydrophobicity, and/or hydrophilicity of the bioconjugate, and will be apparent to those skilled in the art. Preparation of bioconjugates for O1A, O2, O6A, and O25B, as well as vaccine compositions comprising these, have for instance been described in WO 2015/124769 and in WO 2017/035181.

Also provided are bioconjugates produced by the methods described herein, i.e., using the recombinant host cells described herein.

In some embodiments, a method of preparing a bioconjugate of an E. coli O-antigen polysaccharide covalently linked to a carrier protein comprises: (i) providing a recombinant host cell comprising (a) nucleotide sequence of an rfb gene cluster for the O-antigen polysaccharide; (b) a nucleotide sequence encoding a carrier protein, preferably EPA, comprising at least one glycosylation site comprising a glycosylation consensus sequence having SEQ ID NO: 1, preferably SEQ ID NO: 2, and more preferably comprising four glycosylation sites each comprising a glycosylation consensus sequence having SEQ ID NO: 2; and (c) nucleotide sequence encoding an oligosaccharyl transferase, for instance PglB oligosaccharyl transferase or variant thereof.

In certain embodiments, E. coli O-antigen polysaccharides produced using the recombinant host cells described herein are covalently bound to the carrier protein at a particular polysaccharide to protein ratio by weight (w/w). This ratio of amount of O-antigen polysaccharide by weight covalently bound to the carrier protein by weight is referred to as the “glycan/protein ratio” or “polysaccharide/protein ratio” or “PS/protein ratio”. In some embodiments, the O-antigen polysaccharide is covalently bound to the carrier protein at a polysaccharide to protein (w/w) ratio of about 1:20 to 20:1, preferably 1:10 to 10:1, more preferably 1:3 to 3:1. In certain non-limiting embodiments for bioconjugates described herein, glycan/protein ratio is about 0.1 to 0.5, such as 0.1, 0.15, 0.2, 0.25, 0.3, 0.35, 0.4, 0.45, or 0.5. In such embodiments, the weight ratio of the O-antigen polysaccharide: protein is about 1:10 to 1:2, such as 1:10: 1:9: 1:8, 1:7, 1:6, 1:5, 1:4, 1:3, or 1:2, depending on the particular O-antigen serotype. In certain embodiments the glycan/protein ratio is from about 0.15 to about 0.45. In general, a higher glycan/protein ratio of O-antigen polysaccharide to carrier protein is preferred, because a high amount of carrier protein can lead to immunological interference in some instances. Also, a higher glycan/protein ratio would help getting sufficient O-antigen polysaccharide dosed in the form of bioconjugate, while keeping the amount of carrier protein relatively low, which is especially beneficial for multivalent compositions where multiple serotypes are to be covered by the composition, e.g. compositions comprising bioconjugates from at least 4 different O-antigens, at least 5 different O-antigens, at least 6 different O-antigens, at least 7 different O-antigens, at least 8 different O-antigens, at least 9 different O-antigens, at least 10 different O-antigens, etc.

A glycan/protein ratio of a conjugate according to the invention can be determined by determining the protein amount and the glycan amount. Protein amount can be determined by measurement of UV absorbance at 280 nm (A280). Glycan amount can be determined based on ion chromatography with pulsed amperometric detection (IC-PAD) of a sugar in the repeat unit (e.g. of Man for O8 in Table 1, and of GlcNAc for the other glycans in Table 1), after which the structural information of the repeat unit can be used to calculate the total glycan amount (e.g. the repeat unit of O1A has a molar mass of 845 Da and one mole of such a repeat unit contains one mole of GlcNAc, enabling calculation of the total glycan amount when the amount of GlcNAc has been determined by IC-PAD).

In some embodiments, a bioconjugate of an E. coli O25B antigen polysaccharide covalently linked to a carrier protein produced using a recombinant host cell according to the cells and methods described herein has a certain degree of acetylation at position 2 of the L-Rh sugar. The degree of O-acetylation of O25B antigen polysaccharide in a bioconjugate is preferably at least 30%, preferably at least 50%, such as at least 50%, 55%, 60%, 65%, 70%, 75%, 80%, 85%, 90%, 95%, or 100%.

Similarly, the degree of O-acetylation of an E. coli O16 antigen polysaccharide in a bioconjugate is preferably at least 30%, preferably at least 50%, such as at least 50%, 55%, 60%, 65%, 70%, 75%, 80%, 85%, 90%, 95%, or 100%.

In specific embodiments, a method of preparing a bioconjugate of an O-antigen polysaccharide comprises providing a recombinant host cell comprising nucleic acid sequence encoding a particular oligosaccharyl transferase enzyme, particularly a PglB oligosaccharyl transferase or variant thereof, depending on the O-antigen polysaccharide bioconjugate to be produced. The particular oligosaccharyl transferase enzyme variant may impact the yield of bioconjugate produced by the host cell. Typically, a higher yield is preferred, since the yield will impact the costs for producing a specific bioconjugate, which is especially important for multivalent compositions comprising several different bioconjugates. In some embodiments, the method further comprises isolating the bioconjugate from the recombinant host cell.

In one particular embodiment, when the O-antigen is O1A, O6A, or O15 antigen polysaccharide, the PglB oligosaccharyl transferase comprises the amino acid mutations of N311V, K482R, D483H, and A669V, wherein the amino acid mutations are relative to the wild-type PglB having the amino acid sequence of SEQ ID NO: 6.

In another particular embodiment, when the O-antigen is glucosylated O4 antigen polysaccharide, the PglB oligosaccharyl transferase comprises the amino acid mutation N311V, or the amino acid mutations of Y77H and N311V, wherein the amino acid mutations are relative to the wild-type PglB having the amino acid sequence of SEQ ID NO: 6.

In another particular, embodiment, when the O-antigen is O16 antigen polysaccharide, the PglB oligosaccharyl transferase comprises the amino acid mutations of Y77H, S80R, Q287P, K289R, and N311V, wherein the amino acid mutations are relative to the wild-type PglB having the amino acid sequence of SEQ ID NO: 6.

In another particular embodiment, when the O-antigen is O75 antigen polysaccharide, the PglB oligosaccharyl transferase comprises the amino acid mutation of N311V, wherein the amino acid mutations are relative to the wild-type PglB having the amino acid sequence of SEQ ID NO: 6.

In another particular embodiment, when the O-antigen is O8, O18A, O25B, or O2 antigen polysaccharide, the PglB oligosaccharyl transferase comprises the amino acid sequence of SEQ ID NO: 6, wherein SEQ ID NO: 6 comprises no amino acid mutations at positions 77, 80, 287, 289, 311, 482, 483, and 669. In certain embodiments thereof, the PglB oligosaccharyl transferase comprises the amino acid sequence of SEQ ID NO: 6.

In some embodiments, the carrier protein is selected from the group consisting of detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In certain embodiments, the carrier protein is detoxified exotoxin A of Pseudomonas aeruginosa (EPA). Preferably, the EPA carrier protein comprises 1-10, preferably 2-4, more preferably 4 glycosylation sites. Preferably, each glycosylation site comprises a glycosylation consensus sequence having the amino acid sequence of SEQ ID NO: 2. In a specific embodiment, a host cell comprises a nucleic acid encoding EPA-4 carrier protein comprising SEQ ID NO: 3.

In certain embodiments, the recombinant host cell is an E. coli cell, e.g., an E. coli K-12 strain, such as strain W3110.

Also provided herein are bioconjugates of O-antigen polysaccharides produced using recombinant host cells encoding the oligosaccharyl transferase enzymes per the O-antigen/PglB oligosaccharyl transferase pairings indicated above. Also provided are compositions comprising such bioconjugates. In certain embodiments, a composition comprises at least 2, preferably at least 3, more preferably at least 5, still more preferably at least 7 of such bioconjugates.

In some embodiments, bioconjugates of O-antigen polysaccharides produced by recombinant host cells encoding the oligosaccharyl transferase enzymes per the O-antigen/PglB oligosaccharyl transferase pairings indicated above preferably have one or more of the preferred attributes described herein, e.g., glycan/protein ratio and/or amount or ratio of multi-glycosylated carrier protein.

### EMBODIMENTS

Embodiment 1 is a method of preparing a bioconjugate of an E. coli Ox antigen polysaccharide covalently linked to a carrier protein, the method comprising:
(i) providing a recombinant host cell comprising:
a. a nucleotide sequence of an rfb gene cluster for the Ox-antigen polysaccharide;
b. a nucleotide sequence encoding the carrier protein comprising at least one glycosylation site comprising a glycosylation consensus sequence having SEQ ID NO: 1, preferably having SEQ ID NO: 2; and
c. a nucleotide sequence encoding an oligosaccharyl transferase PglBy; and
(ii) culturing the recombinant host cell under conditions for production of the bioconjugate;
   wherein:
when the Ox-antigen is O1A antigen polysaccharide, the PglBy comprises the amino acid mutations of N311V, K482R, D483H, and A669V;
when the Ox-antigen is glucosylated O4 antigen polysaccharide, the PglBy comprises the amino acid mutation N311V or the amino acid mutations Y77H and N311V, and the recombinant host cell further comprises a sequence encoding a glucosyltransferase GtrS having at least 80% identity to SEQ ID NO: 4 and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having at least 80% sequence identity to SEQ ID NOs: 7 and 8 respectively, wherein the translocase is capable of translocating bactoprenol linked glucose and the glycosyltransferase is capable of glucosylating bactoprenol;
when the Ox-antigen is O6A antigen polysaccharide, the PglBy comprises the amino acid mutations of N311V, K482R, D483H, and A669V;
when the Ox-antigen is O8 antigen polysaccharide, the PglBy comprises no amino acid mutations at positions 77, 80, 287, 289, 311, 482, 483 and 669;
when the Ox-antigen is O15 antigen polysaccharide, the PglBy comprises the amino acid mutations of N311V, K482R, D483H, and A669V;
when the Ox-antigen is O16 antigen polysaccharide, the PglBy comprises the amino acid mutations of Y77H, S80R, Q287P, K289R, and N311V;
when the Ox-antigen is O18A antigen polysaccharide, the PglBy comprises no amino acid mutations at positions 77, 80, 287, 289, 311, 482, 483 and 669; and
when the Ox-antigen is O75 antigen polysaccharide, the PglBy comprises the amino acid mutation of N311V;
wherein in each case the amino acid mutations are relative to the wild-type PglB having the amino acid sequence of SEQ ID NO: 6, and
wherein the O1A, glucosylated O4, O6A, O8, O15, O16, O18A, and O75 antigen polysaccharides have the structures of Formulas (O1A), (O4-Glc+), (O6A), (O8), (O15), (O16), (O18A), and (O75), respectively, as shown in Table 1, and each n is independently an integer of 1 to 100, preferably 3 to 50, e.g. 5 to 40, e.g. 7 to 25, e.g. 10 to 20.

Embodiment 2 is the method of embodiment 1, wherein the Ox-antigen is O1A antigen polysaccharide, and the PglBy comprises the amino acid mutations of N311V, K482R, D483H, and A669V relative to wild-type PglB having the amino acid sequence of SEQ ID NO: 6.

Embodiment 3 is the method of embodiment 1, wherein the Ox-antigen is glucosylated O4 antigen polysaccharide, and the PglBy comprises the amino acid mutation N311V or the amino acid mutations Y77H and N311V relative to wild-type PglB having the amino acid sequence of SEQ ID NO: 6.

Embodiment 4 is the method of embodiment 3, wherein the recombinant host cell further comprises a sequence encoding a GtrS having the amino acid sequence of SEQ ID NO: 4, and nucleotide sequences encoding a GtrA and a GtrB having the amino acid sequences of SEQ ID NOs: 7 and 8, respectively.

Embodiment 5 is the method of embodiment 1, wherein the Ox-antigen is O6A antigen polysaccharide, and the PglBy comprises the amino acid mutations of N311V, K482R, D483H, and A669V relative to wild-type PglB having the amino acid sequence of SEQ ID NO: 6.

Embodiment 6 is the method of embodiment 1, wherein the Ox-antigen is O8 antigen polysaccharide, and the PglBy comprises no amino acid mutations at positions 77, 80, 287, 289, 311, 482, 483 and 669 relative to wild-type PglB having the amino acid sequence of SEQ ID NO: 6.

Embodiment 7 is the method of embodiment 1, wherein the Ox-antigen is O15 antigen polysaccharide, and the PglBy comprises the amino acid mutations of N311V, K482R, D483H, and A669V relative to wild-type PglB having the amino acid sequence of SEQ ID NO: 6.

Embodiment 8 is the method of embodiment 1, wherein the Ox-antigen is O16 antigen polysaccharide, and the PglBy comprises the amino acid mutations of Y77H, S80R, Q287P, K289R, and N311V relative to wild-type PglB having the amino acid sequence of SEQ ID NO: 6.

Embodiment 9 is the method of embodiment 1, wherein the Ox-antigen is O18A antigen polysaccharide, and the PglBy comprises no amino acid mutations at positions 77, 80, 287, 289, 311, 482, 483 and 669 relative to wild-type PglB having the amino acid sequence of SEQ ID NO: 6.

Embodiment 10 is the method of embodiment 1, wherein the Ox-antigen is O75 antigen polysaccharide, and the PglBy comprises the amino acid mutation of N311V relative to wild-type PglB having the amino acid sequence of SEQ ID NO: 6.

Embodiment 11 is a method of preparing a bioconjugate of an E. coli Ox antigen polysaccharide covalently linked to a carrier protein, the method comprising:
(i) providing a recombinant host cell comprising:
(a) a nucleotide sequence of an rfb gene cluster for the Ox-antigen polysaccharide;
(b) a nucleotide sequence encoding the carrier protein comprising at least one glycosylation site comprising a glycosylation consensus sequence having SEQ ID NO: 1, preferably having SEQ ID NO: 2; and
(c) a nucleotide sequence encoding an oligosaccharyl transferase PglBy; and
(ii) culturing the recombinant host cell under conditions for production of the bioconjugate,
wherein the PglBy comprises the amino acid mutation N311V relative to the wild-type PglB having the amino acid sequence of SEQ ID NO: 6,
wherein the Ox-antigen is O1A antigen polysaccharide, glucosylated O4 antigen polysaccharide, O6A antigen polysaccharide, O15 antigen polysaccharide, O16 antigen polysaccharide, or O75 antigen polysaccharide, and when the Ox-antigen is glucosylated O4 antigen polysaccharide, the recombinant host cell further comprises a sequence encoding a glucosyltransferase GtrS having at least 80% identity to SEQ ID NO: 4 and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having at least 80% sequence identity to SEQ ID NOs: 7 and 8, respectively, wherein the translocase is capable of translocating bactoprenol linked glucose and the glycosyltransferase is capable of glucosylating bactoprenol, and
wherein the O1A, glucosylated O4, O6A, O15, O16, and O75 antigen polysaccharides have the structures of Formulas (O1A), (O4-Glc+), (O6A), (O15), (O16), and (O75), respectively, as shown in Table 1, and each n is independently an integer of 1 to 100, preferably 3 to 50, e.g. 5 to 40, e.g. 7 to 25, e.g. 10 to 20.

Embodiment 12 is the method of any one of embodiments ito 11, further comprising isolating the bioconjugate from the recombinant host cell.

Embodiment 13 is the method of any one of embodiments 1 to 12, wherein the carrier protein is selected from the group consisting of detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

Embodiment 14 is the method of embodiment 13, wherein the carrier protein is detoxified exotoxin A of Pseudomonas aeruginosa (EPA).

Embodiment 15 is the method of embodiment 14, wherein the EPA carrier protein comprises 1-10, preferably 2-4, more preferably 4, of the glycosylation sites.

Embodiment 16 is the method of embodiment 15, wherein each glycosylation site comprises a glycosylation consensus sequence having SEQ ID NO: 2.

Embodiment 17 is the method of embodiment 16, wherein the EPA carrier protein comprises SEQ ID NO: 3.

Embodiment 18 is the method of any one of embodiments 1-17, wherein the recombinant host cell is an E. coli cell, e.g. an E. coli K-12 strain, such as strain W3110.

Embodiment 19 is a bioconjugate produced by the method of any one of embodiments 1-18.

Embodiment 20 is a composition comprising a bioconjugate of embodiment 19.

Embodiment 21 is a composition comprising at least 2, preferably at least 3, more preferably at least 5, still more preferably at least 7 bioconjugates of embodiment 19.

Embodiment 22 is a composition of embodiment 20 or 21, comprising a bioconjugate of E. coli glucosylated O4 antigen polysaccharide covalently linked to a carrier protein, wherein the glucosylated O4 antigen polysaccharide has the structure of Formula (O4-Glc+) as shown in Table 1, and n is an integer of 1 to 100, preferably 3 to 50, e.g. 5 to 40, e.g. 7 to 25, e.g. 10 to 20.

Embodiment 23 is a composition of any one of embodiments 20 to 22, further comprising at least a bioconjugate of E. coli O25B antigen polysaccharide covalently linked to a carrier protein, wherein the O25B antigen polysaccharide has the structure of Formula (O25B) as shown in Table 1, and n is an integer of 1 to 100, preferably 3 to 50, e.g. 5 to 40, e.g. 7 to 25, e.g. 10 to 20.

Embodiment 24 is a composition of any one of embodiments 20 to 23, further comprising at least a bioconjugate of E. coli O2 antigen polysaccharide covalently linked to a carrier protein, wherein the O2 antigen polysaccharide has the structure of Formula (O2) as shown in Table 1, and n is an integer of 1 to 100, preferably 3 to 50, e.g. 5 to 40, e.g. 7 to 25, e.g. 10 to 20.

Embodiment 25 is a composition of any one of embodiments 20 to 24, comprising: (i) bioconjugate of E. coli O1A antigen polysaccharide covalently coupled to a carrier protein, (ii) bioconjugate of E. coli O2 antigen polysaccharide covalently coupled to a carrier protein, (iii) bioconjugate of E. coli glucosylated O4 antigen polysaccharide covalently coupled to a carrier protein, (iv) bioconjugate of E. coli O6A antigen polysaccharide covalently coupled to a carrier protein, (v) bioconjugate of E. coli O8 antigen polysaccharide covalently coupled to a carrier protein, (vi) bioconjugate of E. coli O15 antigen polysaccharide covalently coupled to a carrier protein, (vii) bioconjugate of E. coli O16 antigen polysaccharide covalently coupled to a carrier protein, (viii) bioconjugate of E. coli O25B antigen polysaccharide covalently coupled to a carrier protein, and (ix) bioconjugate of E. coli O75 antigen polysaccharide covalently coupled to a carrier protein, wherein the O1A, O2, glucosylated O4, O6A, O8, O15, O16, O25B, and O75 antigen polysaccharides have the structures of Formulas (O1A), (O2), (O4-Glc+), (O6A), (O8), (O15), (O16), (O25B), and (O75), respectively, as shown in Table 1, and each n is independently an integer of 1 to 100, preferably 3 to 50, e.g. 5 to 40, e.g. 7 to 25, e.g. 10 to 20.

Embodiment 26 is a composition of embodiment 25, further comprising: (x) bioconjugate of E. coli O18A antigen polysaccharide covalently coupled to a carrier protein, wherein the O18A antigen polysaccharide has the structure of Formula (O18A) as shown in Table 1, and n is an integer of 1 to 100, preferably 3 to 50, e.g. 5 to 40, e.g. 7 to 25, e.g. 10 to 20.

Embodiment 27 is a composition of any one of embodiments 20 to 26, wherein the composition is an immunogenic composition.

Embodiment 28 is a method of vaccinating a subject against E. coli, in particular extra-intestinal pathogenic E. coli (ExPEC), comprising administering to the subject the bioconjugate of embodiment 19, or the composition or immunogenic composition of any one of embodiments 20 to 27.

Embodiment 29 is the bioconjugate of embodiment 19, or the composition or immunogenic composition of any one of embodiments 20 to 27 for use in vaccination against extra-intestinal pathogenic E. coli (ExPEC).

Embodiment 30 is a recombinant host cell for preparing a bioconjugate of an E. coli Ox antigen polysaccharide covalently linked to a carrier protein, the recombinant host cell comprising:
(a) a nucleotide sequence of an rfb gene cluster for the Ox-antigen polysaccharide;
(b) a nucleotide sequence encoding the carrier protein comprising at least one glycosylation site comprising a glycosylation consensus sequence having SEQ ID NO: 1, preferably having SEQ ID NO: 2; and
(c) a nucleotide sequence encoding an oligosaccharyl transferase PglBy; wherein:
when the Ox-antigen is O1A antigen polysaccharide, the PglBy comprises the amino acid mutations of N311V, K482R, D483H, and A669V;
when the Ox-antigen is glucosylated O4 antigen polysaccharide, the PglBy comprises the amino acid mutation N311V or the amino acid mutations Y77H and N311V, and the recombinant host cell further comprises a sequence encoding a glucosyltransferase GtrS having at least 80% identity to SEQ ID NO: 4 and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having at least 80% sequence identity to SEQ ID NOs: 7 and 8 respectively, wherein the translocase is capable of translocating bactoprenol linked glucose and the glycosyltransferase is capable of glucosylating bactoprenol;
when the Ox-antigen is O6A antigen polysaccharide, the PglBy comprises the amino acid mutations of N311V, K482R, D483H, and A669V;
when the Ox-antigen is O8 antigen polysaccharide, the PglBy comprises no amino acid mutations at positions 77, 80, 287, 289, 311, 482, 483 and 669;
when the Ox-antigen is O15 antigen polysaccharide, the PglBy comprises the amino acid mutations of N311V, K482R, D483H, and A669V;
when the Ox-antigen is O16 antigen polysaccharide, the PglBy comprises the amino acid mutations of Y77H, S80R, Q287P, K289R, and N311V;
when the Ox-antigen is O18A antigen polysaccharide, the PglBy comprises no amino acid mutations at positions 77, 80, 287, 289, 311, 482, 483 and 669; and
when the Ox-antigen is O75 antigen polysaccharide, the PglBy comprises the amino acid mutation of N311V;
wherein in each case the amino acid mutations are relative to the wild-type PglB having the amino acid sequence of SEQ ID NO: 6, and
wherein the O1A, glucosylated O4, O6A, O8, O15, O16, O18A, and O75 antigen polysaccharides have the structures of Formulas (O1A), (O4-Glc+), (O6A), (O8), (O15), (O16), (O18A), and (O75), respectively, as shown in Table 1, and each n is independently an integer of 1 to 100, preferably 3 to 50, e.g. 5 to 40, e.g. 7 to 25, e.g. 10 to 20.

Embodiment 31 is the recombinant host cell of embodiment 30, wherein the Ox-antigen is O1A antigen polysaccharide, and the PglBy comprises the amino acid mutations of N311V, K482R, D483H, and A669V relative to wild-type PglB having the amino acid sequence of SEQ ID NO: 6.

Embodiment 32 is the recombinant host cell of embodiment 30, wherein the Ox-antigen is glucosylated O4 antigen polysaccharide, and the PglBy comprises the amino acid mutation N311V or the amino acid mutations Y77H and N311V relative to wild-type PglB having the amino acid sequence of SEQ ID NO: 6.

Embodiment 33 is the recombinant host cell of embodiment 32, wherein the recombinant host cell further comprises a sequence encoding a GtrS having the amino acid sequence of SEQ ID NO: 4, and nucleotide sequences encoding a GtrA and a GtrB having the amino acid sequences of SEQ ID NOs: 7 and 8, respectively.

Embodiment 34 is the recombinant host cell of embodiment 30, wherein the Ox-antigen is O6A antigen polysaccharide, and the PglBy comprises the amino acid mutations of N311V, K482R, D483H, and A669V relative to wild-type PglB having the amino acid sequence of SEQ ID NO: 6.

Embodiment 35 is the recombinant host cell of embodiment 30, wherein the Ox-antigen is O8 antigen polysaccharide, and the PglBy comprises no amino acid mutations at positions 77, 80, 287, 289, 311, 482, 483 and 669 relative to wild-type PglB having the amino acid sequence of SEQ ID NO: 6.

Embodiment 36 is the recombinant host cell of embodiment 30, wherein the Ox-antigen is O15 antigen polysaccharide, and the PglBy comprises the amino acid mutations of N311V, K482R, D483H, and A669V relative to wild-type PglB having the amino acid sequence of SEQ ID NO: 6.

Embodiment 37 is the recombinant host cell of embodiment 30, wherein the Ox-antigen is O16 antigen polysaccharide, and the PglBy comprises the amino acid mutations of Y77H, S80R, Q287P, K289R, and N311V relative to wild-type PglB having the amino acid sequence of SEQ ID NO: 6.

Embodiment 38 is the recombinant host cell of embodiment 30, wherein the Ox-antigen is O18A antigen polysaccharide, and the PglBy comprises no amino acid mutations at positions 77, 80, 287, 289, 311, 482, 483 and 669 relative to wild-type PglB having the amino acid sequence of SEQ ID NO: 6.

Embodiment 39 is the recombinant host cell of embodiment 30, wherein the Ox-antigen is O75 antigen polysaccharide, and the PglBy comprises the amino acid mutation of N311V relative to wild-type PglB having the amino acid sequence of SEQ ID NO: 6.

Embodiment 40 is the recombinant host cell of any one of embodiments 30 to 39, wherein the carrier protein is selected from the group consisting of detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

Embodiment 41 is the recombinant host cell of any one of embodiments 30-40, wherein the carrier protein is detoxified exotoxin A of Pseudomonas aeruginosa (EPA).

Embodiment 42 is the recombinant host cell of embodiment 41, wherein the EPA carrier protein comprises 1-10, preferably 2-4, more preferably 4, of the glycosylation sites.

Embodiment 43 is the recombinant host cell of embodiment 42, wherein each glycosylation site comprises a glycosylation consensus sequence having SEQ ID NO: 2.

Embodiment 44 is the recombinant host cell of embodiment 43, wherein the EPA carrier protein comprises SEQ ID NO: 3.

Embodiment 45 is the recombinant host cell of any one of embodiments 30 to 44, wherein the recombinant host cell is an E. coli cell, e.g. an E. coli K-12 strain, such as strain W3110.

Embodiment 46 is a bioconjugate according to embodiment 19, wherein the bioconjugate is a bioconjugate of E. coli glucosylated O4 antigen polysaccharide covalently linked to a carrier protein.

Embodiment 47 is a bioconjugate according to embodiment 46, wherein the carrier protein is an EPA carrier protein comprising SEQ ID NO: 3.

Embodiment 48 is a bioconjugate according to embodiment 46 or 47, wherein the glucosylated O4 antigen polysaccharide has the structures of Formula (O4-Glc+) as shown in Table 1, and n is an integer of 5 to 40.

Embodiment 49 is a composition comprising a bioconjugate according to any one of embodiments 46-48.

Embodiment 50 is a composition according to embodiment 49, further comprising one or more conjugates each comprising an E. coli antigen polysaccharide covalently coupled to a carrier protein.

Embodiment 51 is a composition according to embodiment 50, wherein the one or more conjugates comprise E. coli antigen polysaccharide of one or more of the following E. coli serotypes: O1A, O2, O6A, O8, O15, O16, O18A, O25B, and O75, wherein the O1A, O2, O6A, O8, O15, O16, O25B, and O75 antigen polysaccharides have the structures of Formulas (O1A), (O2), (O6A), (O8), (O15), (O16), (O18A), (O25B), and (O75), respectively, as shown in Table 1, and each n is independently an integer of 1 to 100, preferably 3 to 50, e.g. 5 to 40, e.g. 7 to 25, e.g. 10 to 20.

Embodiment 52 is a composition according to embodiment 51, comprising conjugates of E. coli serotypes: O1A, O2, O6A, O8, O15, O16, O18A, O25B, and O75.

Embodiment 53 is a composition according to embodiment 52, wherein each of the conjugates is a bioconjugate.

### EXAMPLES

The following examples of the invention are to further illustrate the nature of the invention. It should be understood that the following examples do not limit the invention and the scope of the invention is to be determined by the appended claims.

### Example 1: Epidemiological Data of E. coli Infections

To determine the O-serotype distribution of bacteremia-causing E. coli, global surveillance studies were performed. Between 2011 and 2017, more than 3200 E. coli bloodstream isolates were collected from patients≥60 years of age hospitalized in countries within North America, Europe, the Asia-Pacific region, and South America. Each strain was analyzed for O antigen serotype using classical agglutination techniques and sequence-based O-genotyping. See Table 2.

Isolated human blood samples were analyzed to determine the identity of pathogens therein and their antibiotic resistance patterns. E. coli isolates were obtained from the samples following the analysis. E. coli identity was verified by MALDI-TOF MS. Further analysis on the E. coli isolates was performed using an antisera-based agglutination assay to determine their O-antigen serotype (DebRoy et al. (2011) Animal health research reviews/Conference of Research Workers in Animal Diseases 12, 169-185). Isolates un-typeable by the agglutination method, were further analyzed by whole-genome sequencing followed by O-genotyping based on O-serotype specific wzy and wzx gene sequences.

TABLE 2
distribution of the most common bacteremia-associated E. coli
O-serotypes from a collection of 3217 blood isolates collected
globally between 2011 and 2017, based on O-serotyping by agglutination
plus O-genotyping of isolates un-typeable by agglutination. Subjects
were hospitalized in the following countries: USA, Canada, Argentina,
Brazil, UK, Germany, Spain, Italy, The Netherlands, France, Japan,
Thailand, South Korea and Australia.

| O-serotype | Prevalence n (%) | |
|---|---|---|
| O25 | 737 | (22.9%) |
| O2 | 268 | (8.3%) |
| O6 | 261 | (8.1%) |
| O1 | 255 | (7.9%) |
| O75 | 145 | (4.5%) |
| O15 | 110 | (3.4%) |
| O8 | 104 | (3.2%) |
| O16 | 103 | (3.2%) |
| O4 | 96 | (3.0%) |
| O18 | 91 | (2.8%) |

Stratification of on geographical location in the global set of bacteremia-associated E. coli showed a prevalence of the top 10 O-serotypes independent of location, suggesting these to be the predominant O-serotypes globally associated with bacteremia-causing E. coli.

In the global set of bacteremia-associated multi-drug resistant E. coli isolates (n=345), i.e. those strains that are resistant to at least three classes of clinically relevant antimicrobial drugs, the prevalence of the top 10 O-serotypes is 75.4%.

All information from epidemiology analysis taken together, the 10 predominant O-serotypes could cover an estimated 60-80% of E. coli-associated bacteremia infections, assuming coverage of subportions of the un-typeable strains.

A multivalent vaccine covering a significant proportion of bacteremia-causing E. coli serotypes would be very useful. The O-serotypes of Table 2 would thus be good candidates for an O-antigen based multivalent vaccine. Such a vaccine could beneficially be prepared using bioconjugation technology.

One of the serotypes in the top-10 (Table 2) is O4. It would thus be beneficial to prepare a bioconjugate vaccine that includes O-antigen polysaccharide of E. coli serotype O4 coupled to a carrier protein.

### Example 2: Characterization of Contemporary O4 Clinical Isolates for Genes Encoding O-antigen Modifying Enzymes

Two variants of E. coli O4 antigen polysaccharide have been described (see, e.g. Jann B, et al., 1993, Carbohydr. Res. 248: 241-250), one having an unbranched structure (structure shown as (O4-Glc−) in Table 1) and another variant substituted with an additional glucose side-branch (structure shown as (O4-Glc+) in Table 1). The proportion in which these two variants are found in contemporary clinical isolates was not known. Although both variants react with O4 antisera, it was also not known whether immunological differences between these variants exist. Moreover, an enzyme responsible for attaching the glucose side-branch to generate the (O4-Glc+) antigen polysaccharide was hitherto not identified, and a putative coding sequence thereof is likely residing outside the O4 rfb gene cluster.

A set of 32 agglutination-confirmed E. coli O4 clinical isolates originally isolated during the period of 2011-2012 from subjects in the United States and the European Union were subjected to whole genome sequence analysis. Extracted rfb gene cluster sequences from the 32 sequenced O4 isolates were aligned with those of the reference strain and compared at the nucleotide level. Except for some naturally occurring single nucleotide polymorphisms, the characterized isolates all displayed an rfb cluster that was identical to the O4 reference strain, indicating that E. coli O4 strains, independent of their Glc-branching status, carry an identical rfb gene cluster. Thus, to generate the E. coli O4-Glc+ antigen polysaccharide, a gene with unknown sequence that encodes an E. coli O4-specific branching enzyme and that must reside somewhere outside of the E. coli O4 rfb gene cluster is likely needed. The sequence of this unknown gene needs to be identified and employed if one wants to produce bioconjugates with the E. coli O4-Glc+ antigen polysaccharides in a strain that would otherwise only produce bioconjugates with E. coli O4-Glc− antigen polysaccharides.

The whole-genome sequence data were then analyzed for the presence of genes outside of the rfb gene cluster that may encode O-antigen modifying enzymes. Homologs of gtrAB in Shigella flexneri were first identified in E. coli O4. An open reading frame downstream of gtrAB in E. coli was then putatively identified as the E. coli O4-specific gene gtrS, that could encode the putative E. coli O4 specific branching enzyme GtrS responsible for adding a glucose branch to the E. coli O4 antigen.

The amino acid sequence of the O4 specific GtrS enzyme is provided as SEQ ID NO: 4. An exemplary nucleic acid sequence encoding this protein is provided as SEQ ID NO: 5.

Of the characterized E. coli O4 isolates, approximately 80% were found to carry the here identified gtrS gene (26 out of 32). Prevalence of the E. coli O4-specific gtrS sequence was also determined by PCR using sequence specific primers in an independent set of 20 agglutination-confirmed E. coli O4 clinical isolates isolated during the period of 2014-2016 from subjects in the United States and the European Union. This analysis demonstrated that 17 out of 20 isolates carried the O4 gtrS sequence, which corresponds to a prevalence of 85%.

### Example 3: Cloning of O4 gtrS into E. coli W3110, Production and Structural Confirmation of Glc-Modified O4 Bioconjugates

To test whether bioconjugates comprising O4-antigen polysaccharide modified with a branching glucose could be prepared, E. coli O4-antigen EPA bioconjugate production strains with the putative branching enzyme were constructed. For this, the endogenous O16-gtrS gene was substituted by the putative O4-gtrS gene (SEQ ID NO: 5, see Example 2) and the O16 rfb cluster was replaced with the O4 rfb cluster in E. coli strain W3110 ΔwzzE-wecG ΔwaaL ΔwbbI-J-K by homologous recombination. Alternatively, in some strains, the O4 rfb cluster was encoded on a plasmid.

Subsequently, plasmids encoding a detoxified exotoxin A of Pseudomonas aeruginosa (EPA) carrier protein (a variant either having 2 or 4 consensus glycosylation sites, referred to as ‘EPA-2’ and ‘EPA-4’, respectively), and oligosaccharyl transferase PglB were introduced into the strains. O4-EPA bioconjugates modified with Glc were produced by growing the E. coli production strains in bioreactor cultures, and induction of PglB and EPA expression by IPTG and arabinose, respectively. The O4-EPA bioconjugates were extracted from the biomass periplasmic extract.

To confirm the detailed polysaccharide composition and linkage of the O4-EPA bioconjugates, multiple NMR experiments were performed on the bioconjugates having EPA-4 carrier protein (data not shown). The assignments obtained agreed with literature published (Jansson, P. E., et al., 1984, Carbohydr. Res. 134(2): 283-291; Jann B, et al., 1993, Carbohydr. Res. 248: 241-250). The 1D spectrum recorded at 313K showed a large HOD signal and small sharp signals from the O4 pentasaccharide RU with five anomeric, two NAc and two H6 signals (Rha and FucNAc).

The 1D proton assignments were confirmed by use of 2D proton-proton and proton-carbon correlation NMR experiments. First, 2D TOCSY (120 ms) experiments demonstrated the expected cross peaks from H1 and H6 (for Rha and FucNAc) for the O4 pentasaccharide RU and small peaks from the terminal RU and EPA. In the methyl region, TOCSY showed cross peaks from H6 to H1 for α-Rha and H6 to H5 for α-FucNAc for the O4 RU. Other peaks observed were from EPA amino acids and terminal Rha (tRha). Second, a carbon NMR spectrum contained well-dispersed and diagnostic single peaks for the O4 RU. The carbons were profiled indirectly through the attached protons by use of the HSQC experiment. The HSQC-DEPT experiment gave inverted peaks for CH2 groups. The HSQC gave cross peaks for the O4 pentasaccharide RU [5 anomeric, ring, two N-acetyl and two methyl (Rha & FucNAc)] groups as well as EPA amino acids in characteristic regions. Each of the proton/carbon pairs for the O4 could be assigned based on the proton assignments and literature.

The structural characterization experiments thus confirmed that Glc-branched O4 bioconjugates (comprising polysaccharide antigen structures as indicated by Formula (O4-Glc+) in Table 1) could be produced, using the putative E. coli O4-gtrS gene identified in Example 2.

### Example 4: Immunogenicity of a Glc-Branched O4 Bioconjugate in Rabbits

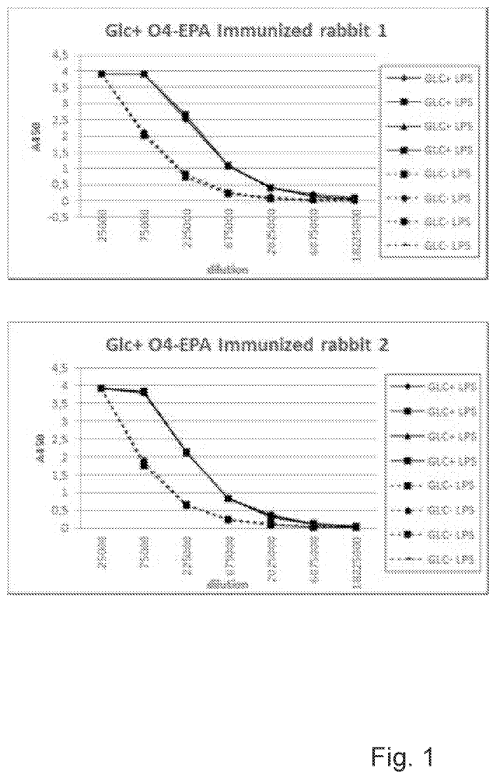

Glc-modified O4 bioconjugates (i.e. having glycans with the structure of Formula (O4-Glc+) as shown in Table 1) were used for rabbit immunization by applying a speedy-rabbit protocol (Eurogentec). Sera from immunized rabbits were analyzed by ELISA for anti-O4 IgG titers against purified O4 lipopolysaccharide (LPS) with (Glc+; i.e. containing glucosylated O4 polysaccharide) or without Glc-branching (Glc−; i.e. containing non-glucosylated O4 polysaccharide). Immunization with the bioconjugate resulted in high IgG titers in both rabbits (FIG. 1). In both cases, antibody titers induced by the O4 bioconjugate were higher against Glc+ LPS as compared to Glc− LPS.

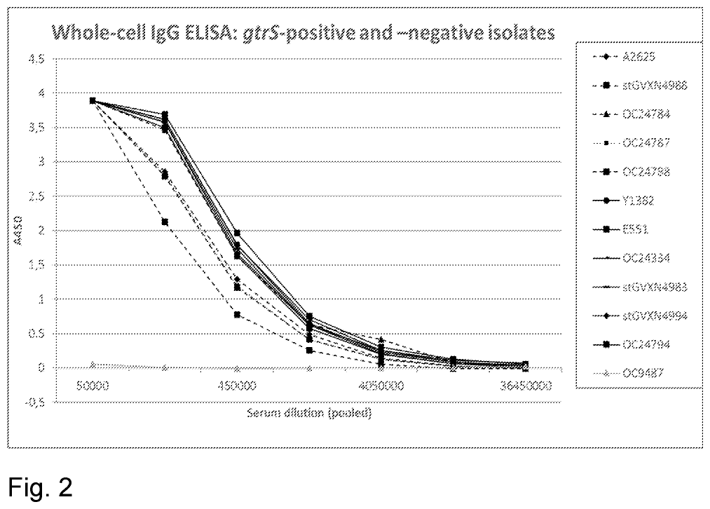

Sera were also pooled and used in whole cell ELISA studies with test sets of E. coli O4 isolates with characterized gtrS status. Five gtrS-negative (no Glc-branching) and six gtrS-positive (Glc-branching) E. coli O4 isolates and a negative control strain were tested. Pooled sera from rabbits immunized with a Glc-modified O4 bioconjugate contained high titers of IgG specifically recognizing the tested O4 isolates (FIG. 2). In concordance with the LPS ELISA, all tested O4 isolates were recognized by the immune sera. The gtrS-positive isolates displayed an overall higher binding than the gtrS-negative isolates (FIG. 2). In particular, the following isolates were gtrS-positive: Y1382, E551, OC24334, stGVXN4983, stGVXN4994 and OC24794, and the following isolates were gtrS-negative: A2625, stGVXN4988, OC24784, OC24787, and OC24788. Immune sera did not bind the negative control strain of a non-related O-serotype, E. coli OC9487 (ATCC 35383).

The profiles of LPS extracted from the test set of gtrS-positive and -negative isolates in silver-stained polyacrylamide gels did not reveal marked differences between isolates expressing unmodified and modified forms of the O4 antigen confirming that the observed differences are not explained by quantitative differences in LPS expression levels (data not shown).

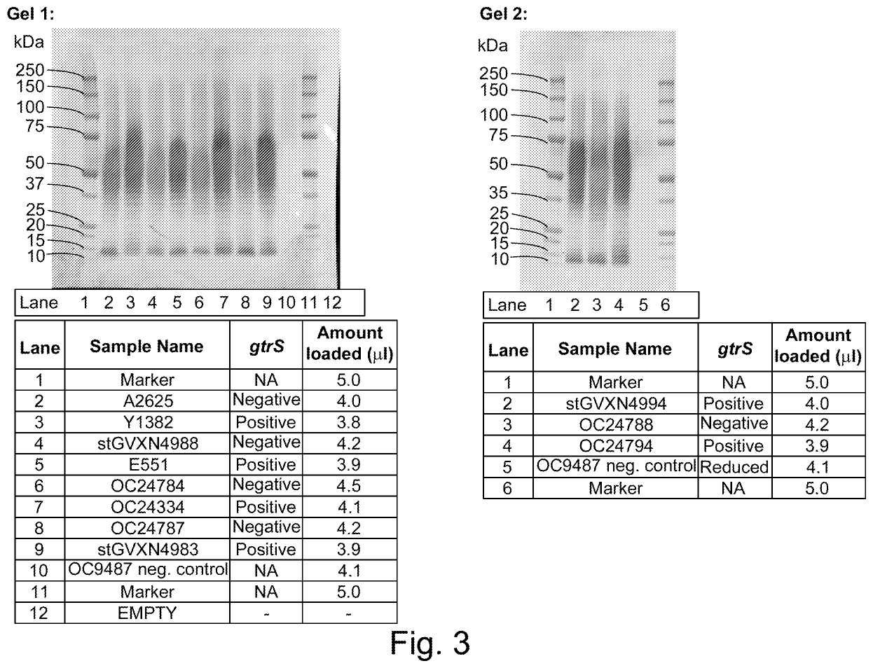

Western blots of extracted LPS using pooled immune sera were performed to assess recognition of O4 O-antigen by IgGs elicited in response to immunization with a Glc-modified O4 bioconjugate. Binding of both modified and unmodified O4 LPS by IgGs from modified O4 immunized rabbits was observed and included specific recognition of LPS bands spanning a wide range of sizes, including high molecular weight LPS bands (FIG. 3).

In the further experiments below, when reference is made to ‘O4’ bioconjugate or production strains or ‘EcoO4’, the bioconjugate or production strain of Glc-branched O4 (having glycan structure (O4-Glc+) in Table 1) is meant, unless specifically indicated otherwise (the terms ‘O4’ and ‘O4-Glc+’ are thus used interchangeably for bioconjugates or production strains in those experiments).

### Example 5: Immunogenicity of a Glc-Branched O4 Bioconjugate in Rats

Sprague Dawley rats were immunized intramuscularly 3 times with formulation buffer or (O4-Glc+)-EPA bioconjugate (i.e. bioconjugate of glucosylated O4 antigen polysaccharide covalently coupled to EPA carrier protein; carrier protein was EPA-2 as described in Example 3 above) at 3 different doses (0.04 μg, 0.40 μg or 4.0 μg). Serum antibody levels were measured by ELISA at day 0, 14 and 42 post-immunization.

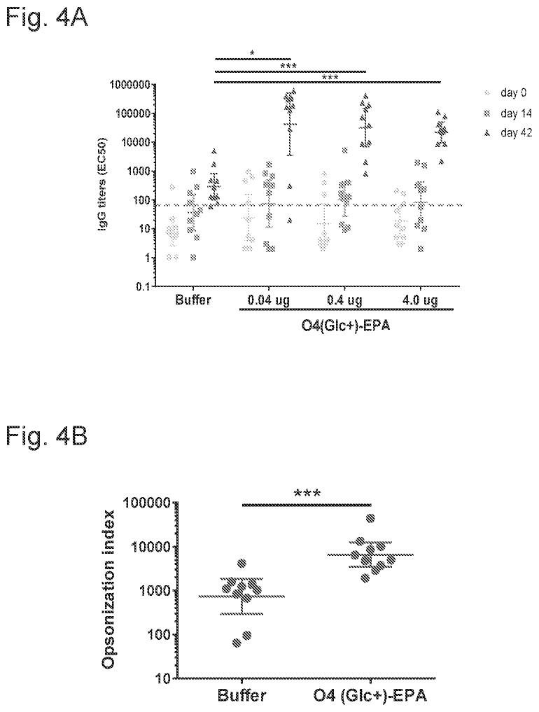

Immunization with 0.04 μg, 0.40 μg and 4.00 μg of (O4-Glc+)-EPA bioconjugate induced significant increase in the levels of IgG antibodies at day 42 post-immunization when compared to formulation buffer (FIG. 4A). The antibodies induced by (O4-Glc+)-conjugate were functional, i.e., capable of mediating killing of (O4-Glc+) E. coli strain (FIG. 4B).

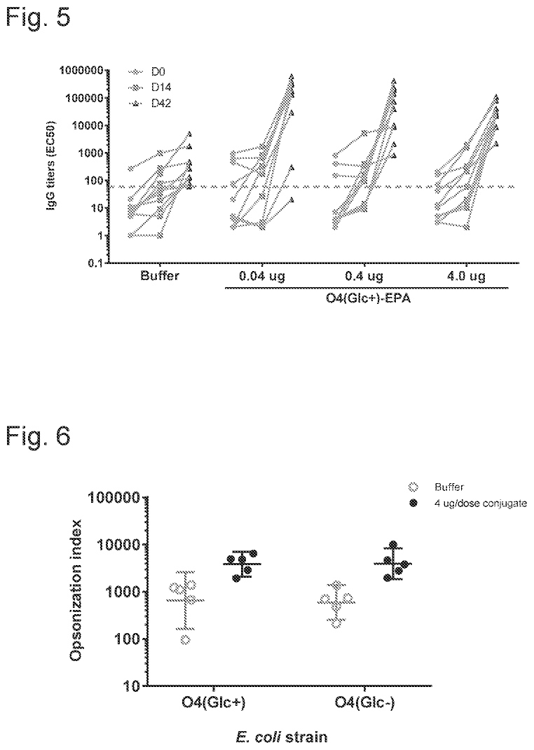

Antibody levels induced by 0.04 μg, 0.40 μg and 4.0 μg of (O4-Glc+)-EPA bioconjugate were significantly increased at day 42 as compared to those detected at baseline (day 42 vs day 0, P=0.006 for all doses) and at day 14 post-immunization (day 42 vs day 14, P=0.006 for all doses) (FIG. 5). In the group that received 4.0 μg of bioconjugate, titers were also significantly increased at day 14 compared to day 0, indicating that a single dose of 4.0 μg of (O4-Glc+)-EPA bioconjugate induces significant increase in IgG titers (day 14 vs day 0, P=0.012). The significant increase in IgG titers observed between day 14 and 42, for all three concentrations of bioconjugate tested showed that a third dose of (O4-Glc+)-EPA bioconjugate is able to boost antibody responses (FIG. 5).

Functionality of antibodies induced by O4-Glc+-EPA conjugate in the rats immunized intramuscularly 3 times with formulation buffer or the bioconjugate at 4.00 μg/dose was determined by opsonophagocytic killing assay (OPKA) using O4(Glu+) and O4(Glu−) E. coli strains. The antibodies induced by (O4-Glc+)-EPA bioconjugate were functional, i.e., capable of mediating killing of an (O4-Glc+) E. coli strain (FIG. 4B, FIG. 6). Notably, antibodies induced by (O4-Glc+)-EPA bioconjugate were capable of mediating killing of both (O4-Glc+) and (O4-Glc−, i.e. having glycans with structure of Formula (O4-Glc−) in Table 1, i.e. O4 polysaccharide without Glc-branching) E. coli strains (FIG. 6).

In conclusion, antibodies induced by O4-Glc+-EPA bioconjugate are cross-reactive and capable of mediating killing of E. coli O4 strains with and without glucose branching.

### Example 6: Production Strains for E. coli O-Antigen Bioconjugates and Resulting Bioconjugate Products

In addition to (O4-Glc+)-EPA bioconjugates prepared as described above, nine (9) other bioconjugates were produced. In particular, the additionally produced bioconjugates included E. coli O1A-EPA bioconjugate, O2-EPA bioconjugate, O6A-EPA bioconjugate, O8-EPA bioconjugate, O15-EPA bioconjugate, O16-EPA bioconjugate, O18A-EPA bioconjugate, O25B-EPA bioconjugate, and O75-EPA bioconjugate. The chemical structures of the glycans of these conjugates can be seen in the respective Formulas in Table 1. A composition comprising the 10 bioconjugates is referred to herein as ‘ExPEC10V’. A composition comprising the O1A-EPA, O2-EPA, O6A-EPA and O25B-EPA bioconjugates is referred to as ‘ExPEC4V’ (and was previously described in for instance WO 2015/124769 and WO 2017/035181).

Escherichia coli W3110 Parental Strain

The non-pathogenic E. coli K12 strain W3110 was used as the parental strain for the construction of all ten production strains. The E. coli K12 strain W3110 was obtained from the Coli Genetic Stock Center (Yale University, New Haven (CT), USA, product number CGSC #4474). Its relevant genotype was previously described (E. coli W3110, F-, lambda-, IN(rrnD-rrnE)1, rph-1) and its genomic sequence was previously published (Hayashi K, et al., 2006, Mol. Syst. Biol. 2006.0007 (doi:10.1038/msb4100049). The E. coli W3110 strain was genetically modified to enable production of each of the E. coli O-antigen bioconjugates (Table 3).

### Bioconjugate Production Strains

The “ExPEC4V” and “ExPEC10V” compositions both comprise the O2-EPA and O25B-EPA bioconjugates from the same production strains. The “ExPEC4V” composition comprises the O1A-EPA bioconjugate from the stGVXN4411 or stLMTB10217 production strains, while the “ExPEC10V” composition comprises the O1A-EPA bioconjugate from the stLMTB10217 production strain. The “ExPEC4V” composition comprises the O6A-EPA bioconjugate from the stGVXN4112 production strain, while the “ExPEC10V” composition comprises the O6A-EPA bioconjugate from the stLMTB10923 production strain. Furthermore, the “ExPEC10V” composition comprises the O4-EPA (i.e. (O4-Glc+)-EPA), O8-EPA, O15-EPA, O16-EPA, O18A-EPA, and O75-EPA bioconjugates from production strains that are not used for “ExPEC4V”. Different production strains could vary in the plasmids for expression of the EPA carrier protein and/or the oligosaccharyl transferase PglB, as indicated below. An overview of several production strains is given in Table 3 below.

TABLE 3
Overview of genetic engineering of E. coli production strains for
O-antigen bioconjugates for ExPEC4V and ExPEC10V vaccine compositions

| | | Genomic mutations | | | Plasmids | |
|---|---|---|---|---|---|---|
| Serotype | Strain name | rfb gene cluster | waaL | gtrABS | pglB | epa |
| O1A | stGVXN4411 | Δrfb::O1A rfb | ΔwaaL | — | pGVXN970 | PGVXN1076 |
| (ExPEC4V) | | upecGVXN_032 | | | | |
| O1A | stLMTB10217 | Δrfb::O1A rfb | ΔwaaL | — | pGVXN1221 | PGVXN1076 |
| (ExPEC4V; | | upecGVXN_032 | | | | |
| ExPEC10V) | | | | | | |
| O2 | stGVXN4906 | Δrfb::O2 rfb | ΔwaaL | — | pGVXN971 | pGVXN1076 |
| | | upecGVXN_116 | | | | |
| O4 | BVEC-L-00684 | Δrfb::O4 rfb | ΔwaaL | ΔgtrS::gtrS | pGVXN1217 | pGVXN1076 |
| | | CCUG11450 | | O4 | | |
| O6A | stGVXN4112 | Δrfb::O6A rfb | ΔwaaL | — | pGVXN114 | pGVXN659 |
| (ExPEC4V) | | CCUG11309 | | | | |
| O6A | stLMTB10923 | Δrfb::O6A rfb | ΔwaaL | — | pGVXN1221 | pGVXN1076 |
| (ExPEC10V) | | CCUG11309 | | | | |
| O8 | stLMTB11734 | Δrfb::O8 rfb | ΔwaaL | ΔgtrABS | pGVXN970 | pGVXN1076 |
| | | E2420 | | | | |
| O15 | stLMTB11738 | Δrfb::O15 rfb | ΔwaaL | ΔgtrABS | pGVXN1221 | pGVXN1076 |
| | | OC24891 | | | | |
| O16 | stLMTB11739 | Δrfb::O16 rfb | ΔwaaL | ΔgtrABS | pGVXN2381 | pGVXN1076 |
| | | OC24208 | | | | |
| O18A | BVEC-L-00559 | Δrfb::O18A rfb | ΔwaaL | ΔgtrABS | pGVXN970 | pGVXN1076 |
| | | OC24255 | | | | |
| O25B | stGVXN4459 | Δrfb::O25B rfb | ΔwaaL | ΔgtrABS | pGVXN970 | pGVXN1076 |
| | | upecGVXN_138 | | | | |
| O75 | stLMTB11737 | Δrfb::O75 rfb | ΔwaaL | ΔgtrABS | pGVXN1217 | pGVXN1076 |
| | | CCUG31 | | | | |

### O-Antigen Biosynthesis (Rfb) Gene Cluster

In all E. coli O-antigen production strains, the naturally occurring E. coli W3110 genomic O16::IS5-antigen biosynthesis (rfb) gene cluster was replaced by the selected O-antigen-specific biosynthesis clusters from E. coli strains of the selected serotype, encoding for the serotype-specific O-antigen structures (see Table 1 for these O-antigen structures). The ten donor rfb clusters were selected or confirmed after whole-genome analysis of E. coli blood isolates. Replacement of the W3110 O16::IS5 rfb gene cluster, which is defective in O-antigen biosynthesis, has been achieved in a single homologous recombination event. In case of the O16 and O18A rfb gene clusters, the donor DNA recombined via the flanking gnd and rmlCA genes, while the rfb gene cluster for the other strains recombined via the flanking gnd and galF genes. Sequences of the rfb clusters in the production strains are provided in SEQ ID NOs: 9 and 11-19.

O-Antigen Ligase (waaL) Gene

All E. coli O-antigen production strains carry an artificially introduced deletion of the E. coli W3110 genomic O-antigen ligase encoded by the waaL gene. In the ΔwaaL strains the transfer of the O-antigen to lipid A is disrupted, which instead directs transfer of the O-antigen to the carrier protein to increase product yield.

O-Antigen Glucosylation (gtrABS) Genes

In the E. coli O8, O15, O16, O18A, O25B, and O75 production strains the E. coli W3110 genomic gtrABS genes, which are responsible for O16 O-antigen glucosylation, have been deleted. While the gtrA and gtrB genes in different serotypes are highly homologous and interchangeable, the gtrS gene encodes a serotype-specific O-antigen glycosyl transferase. In E. coli W3110 GtrS can transfer a glucose (Glc) residue to the GlcNAc sugar in the α-1-Rha-(1→3)-d-GlcNAc motif of the E. coli O16 O-antigen. In the E. coli O1A, O2 and O6A production strains no deletion or replacement of the gtrABS gene has occurred. These O-antigens miss the α-1-Rha-(1→3)-d-GlcNAc motif that is the natural substrate for E. coli O16 gtrS. In the E. coli O4 production strain, the W3110 gtrS gene has been replaced with the E. coli O4 gtrS gene to accommodate proper glucosylation of the E. coli O4 O-antigen.

### Oligosaccharyl Transferase PglB

All E. coli O-antigen production strains expressed a variant of the C. jejuni glycosyl transferase PglB, which can transfer the O-antigen onto an amino acid consensus sequence on a carrier protein by N-glycosylation. PglB has broad substrate recognition, but due to low product yields several production strains were prepared expressing a PglB variant having modified substrate specificities, which resulted in improved product yield (see e.g. WO 2016/107818, WO 2016/107819). The pglB gene was placed behind an Isopropyl β-d-1-thiogalactopyranoside (IPTG) inducible promoter on a plasmid. Table 4 below lists the PglB variants encoded by the plasmids used for production of the E. coli O-antigen production strains for the bioconjugates for the ExPEC4V and ExPEC10V compositions described above. Further plasmids with variation in vector backbone, antibiotic resistance marker, and/or alternative PglB variants have also been tested successfully for bioconjugate production.

TABLE 4
PglB and EPA plasmids used in E. coli O-antigen Production Strains

| Plasmid | | |
|---|---|---|
| name | Gene | Description1 |
| pGVXN114 | pglB | C. jejuni codon usage; SpR |
| pGVXN970 | pglB | E. coli codon usage optimized; SpR |
| pGVXN971 | pglBN534Q | E. coli codon usage optimized; The |
| | | natural glycosylation site of PglB was |
| | | inactivated; SpR |
| pGVXN1217 | pglBN311V | E. coli codon usage optimized; Substrate |
| | | optimized PglB; SpR |
| pGVXN1221 | pglBN311V, | E. coli codon usage optimized; Substrate |
| | K482R, D483H, | optimized PglB; SpR |
| | A669V | |
| pGVXN2381 | pglBY77H, S80R, | E. coli codon usage optimized; Substrate |
| | Q287P, K289R, | optimized PglB; SpR |
| | N311V | |
| pGVXN659 | EPA-4 | EPA with four bioconjugation sites; |
| | | AmpR |
| pGVXN1076 | EPA-4 | EPA with four bioconjugation sites; |
| | | KanR |

1SpR, spectinomycin resistant; AmpR, ampicillin resistant; KanR, kanamycin resistant

### Carrier Protein (EPA)

All E. coli O-antigen production strains expressed a genetically detoxified P. aeruginosa ADP-ribosyltransferase toxoid (EPA) as a carrier protein for the O-antigen. The EPA toxoid differs from wild-type EPA toxin in two residues: Leu552 was changed to Val and Glu553 (in the catalytic domain) was deleted. Glu553 deletions were reported to significantly reduce toxicity. In addition to the detoxification mutation, four (EPA-4) consensus N-glycosylation site motifs were introduced. The epa gene was placed behind a 1-Arabinose (Ara) inducible promoter on a plasmid (Table 4). Table 4 is limited to the plasmids used in production strains for bioconjugates used in the “ExPEC4V” and “ExPEC10V” compositions described above. Plasmids with variation in vector backbone, antibiotic resistance marker, and/or EPA variants, e.g. varying in the number of consensus N-glycosylation site motifs (e.g. having two such motifs, EPA-2), have also been tested successfully for bioconjugate production.

### Example 7: Optimizing the Oligosaccharyl Transferase for Generation of Bioconjugates with Glucosylated O4 (O4-Glc+) Antigen

Yield optimization for bioconjugate production can be achieved by modification of the C. jejuni oligosaccharyl transferase PglB, which can lead to a more efficient or higher degree of N-glycosylation of the O-antigen of interest to the EPA carrier protein. In an E. coli strain for production of bioconjugate with glucosylated O4 (O4-Glc+) O-antigen polysaccharide, such optimization strategy was applied and resulted in an (O4-Glc+)-specific optimized PglB variant improving bioconjugate product yield.

In this approach, an O4-Glc+O-antigen polysaccharide producing strain containing an EPA-expression plasmid was transformed with a variety of different PglB expression plasmids, each of which contained different amino acid substitutions in the PglB protein, altering substrate specificity. Bioconjugate production level and profile of each strain was assessed at shake-flask level in osmotic shock experiments, and readout was performed by capillary electrophoresis immunoassays on the periplasmic extract using O4-Glc+-specific monoclonal antibodies.

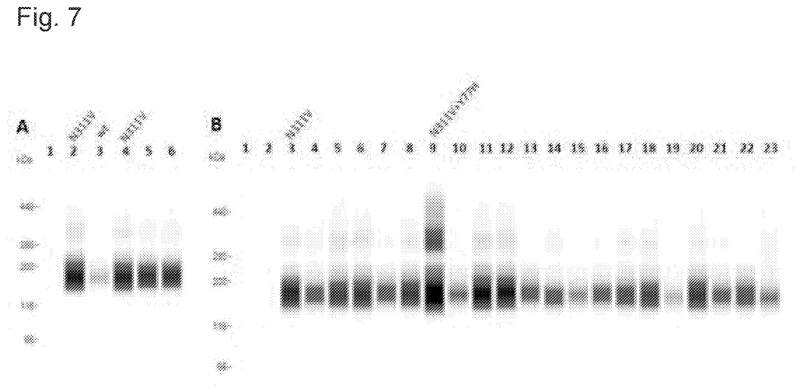

One of the tested PglB variants containing an N311V amino acid substitution was found to improve product yield of glucosylated O4 bioconjugates significantly (FIG. 7A).

In a further improvement where the N311V PglB-variant was further modified, an Y77H amino acid substitution further enhanced O4-Glc+-specific product yield and showed an increased degree of di- and tri-glycosylated product compared to the N311V PglB-variant, where other modifications were found to be neutral or had a negative effect on product yield (FIG. 7B). Plasmid pLMTB4008 (SpR) encodes E. coli codon usage optimized, (O4-Glc+)-substrate optimized, PglB variant with mutations Y77H and N311V.

The PglB variant with optimized substrate specificity for O4-Glc+O-antigen polysaccharide, containing N311V and Y77H amino acid substitutions relative to wild-type (wt) C. jejuni glycosyl transferase PglB, was found to double bioconjugate yield compared to the first round optimized PglB-N311V variant.

Similarly using screens, the most optimal yielding PglB variants were also determined for E. coli O-antigen bioconjugate production of the of the other nine serotypes in the ExPEC10V composition.

For bioconjugates having the O1A, O6A, or O15 antigen polysaccharide, PglB with amino acid mutations N311V, K482R, D483H, and A669V was found to give the highest yields.

For bioconjugates having the O2, O8, O18A, or O25B antigen polysaccharide, wild-type PglB (i.e. not having amino acid mutations at positions 77, 80, 287, 289, 311, 482, 483 and 669) was found to give the highest yields.

For bioconjugates having the O16 antigen polysaccharide, PglB with amino acid mutations Y77H, S80R, Q287P, K289R, and N311V was found to give the highest yields.

For bioconjugates having the O75 antigen polysaccharide, PglB with amino acid mutation N311V was found to give the highest yields.

It can be seen from these results that the optimal PglB variant is different for different O-antigens, and that the optimal PglB variant for producing a bioconjugate with a given O-antigen polysaccharide is unpredictable.

### Example 8: Bioconjugates of O-Antigens from 10 E. coli Serotypes and their Quality Attributes

O-glycan residues of the target O-antigens are structurally diverse and have variable repeating units. The specificity and affinity of the glycosyl transferase PglB is linked to the glycan structure. Thus, making a bioconjugate that has the desired quality attributes, e.g., purity, glycan/protein ratio, etc., is a challenging, non-straightforward, task. The right combination of PglB and EPA carrier protein determines the yield and may influence glycosylation efficiency. By optimizing the PglB and carrier proteins, bioconjugates having the desired quality attributes were produced. It may be also important to maintain a lower threshold value of total carrier protein, particularly when one or more O-antigen bioconjugates are combined together and administered in a single composition or vaccine, because very high amounts of carrier protein may lead to immunological interference. In order to avoid such a phenomenon, conjugates having a higher glycan/protein ratio are preferred. Hence, for ExPEC10V vaccine, bioconjugates with at least comparable (to the previously described ExPEC4V vaccine that has been subject to clinical trials) glycosylation ratio were developed.

The bioconjugates were each produced by culturing the respective host cells (Example 6, Table 3) in bioreactors (10 L and/or 200 L volumes) and expression of the bioconjugates, following methods previously described. Each drug substance was manufactured batch-wise by bacterial fed-batch fermentation to generate biomass containing the expressed bioconjugates of the corresponding polysaccharide serotype. Cells were cultured and induced with IPTG and arabinose. The bioconjugates were isolated from the periplasm of the cells in the bioreactor cultures by osmotic shock followed by chromatographic purification. This process was performed for each of the 10 bioconjugates.

The E. coli O-antigen bioconjugates thus prepared that are drug substances (DSs) for ExPEC10V and ExPEC4V showed comparable critical quality attributes: (1) process-related purity (measured by RP-HPLC) was higher than 95%, (2) polysaccharide/protein ratio ranged between about 0.1-0.5, mostly between 0.15 and 0.45, (3) bacterial endotoxin (Ph. Eur. 2.2.3) was less than 0.5 EU/μg polysaccharide. The average length of the individual polysaccharide chains was typically between about 10-20 repeating units (measured using high resolution SDS-PAGE).

The structures of the polysaccharide repeat units were confirmed (by NMR and MS/MS of the conjugates, intact or trypsin-digested) to be the ones shown in the Formulas for the corresponding serotypes in Table 1, for all ten bioconjugates that are DSs for the ExPEC10V composition described above.

The O18 serotype had the lowest yields of bioconjugate production amongst the ten serotypes of which bioconjugates were made for the ExPEC10V composition.

ExPEC10V drug product (DP) comprises a mixture of the ten monovalent DSs described above.

### Example 9: Toxicology of ExPEC10V Vaccine

A single-dose pilot toxicity and local tolerance study (non-GLP) with ExPEC10V was conducted in female NZW rabbits. One group (n=2) received an intramuscular (IM) injection (on Day 0) of the control (saline), and a second group (n=4) received an IM injection of ExPEC10V at 105.6 μg total polysaccharide (PS)/dose (9.6:9.6:9.6:9.6:9.6:9.6:9.6:9.6:19.2:9.6 μg PS per dose, for respectively O-serotypes O1A, O2, O4, O6A, O8, O15, O16, O18A, O25B and O75) using a dosing volume of 0.6 mL (176 μg PS/mL). Necropsy was performed on Day 2.

There were no mortalities observed. In addition, there were no vaccine-related effects noted for clinical observations (including injection site effects using Draize scoring), body weight, food consumption, and body temperature. Histopathologically, there were no vaccine-related changes observed at the administration site or draining (iliac) lymph node. A minimal increase in germinal center formation in the spleen was observed in one out of four treated animals (Day 2), and was considered a normal, immunological response to the injected vaccine. Overall, the administration of a single IM dose of ExPEC10V to female rabbits was well-tolerated.

### Example 10: Immunogenicity of ExPEC10V Blended Formulation in Rabbits

An ExPEC4V vaccine (comprising bioconjugates of E. coli O1A, O2, O6A, and O25B serotypes) has previously been shown to be immunogenic for these four serotypes in rats, rabbits, and humans (see e.g. WO 2015/124769; WO 2017/035181; Huttner et al, 2017, Lancet Infect Dis, http://dx.doi.org/10.1016/S1473-3099(17)30108-1; R W Frenck Jr, et al, abstract 5587, ASM Microbe 2018). The novel bioconjugates of the invention having the E. coli glucosylated O4 serotype were shown to be immunogenic in Examples 4 and 5 above. Immunogenicity of the bioconjugates of E. coli serotypes O8, O15, O16, O18A, and O75 (all having EPA-2 as carrier protein in this experiment) when separately administered (monovalent) to rats confirmed that also each of these bioconjugates was immunogenic, since ELISA data indicated that each of these bioconjugates could elicit high levels of E. coli O-antigen specific antibodies (not shown).

Immunogenicity of the 10-valent vaccine that contained a mixture of the 10 bioconjugates as described above was also tested. New Zealand White (NZW) rabbits (female, 12-16 weeks old) received 3 intramuscular immunizations with ExPEC10V or saline administered 2 weeks apart (Table 5; administration at days 0, 14, and 27). The 10 polysaccharides that are part of the ExPEC10V vaccine used in these experiments were conjugated to the carrier protein EPA containing 4 sites of glycosylation (EPA-4). The vaccine was formulated in 3 different doses: Group 1 (‘high dose’): 8 ug/dose of O1A, O2, O6A, O4, O8, O15, O16, O18 and O75 and 16 ug/dose of O25B; Group 2 (‘medium dose’): 4 ug/dose of O2, O4, O8, O15, O16, O18 and O75, 8 ug/dose of O1A and O6A and 16 ug/dose of O25B; Group 3 (‘low dose’): 0.4 ug/dose of O2, O4, O8, O15, O16, O18 and O75, 0.8 ug/dose of O1A and O6A and 1.6 ug/dose of O25B. Animals from the control group (Group 4) received only saline (0.9% (w/v) sodium chloride solution) (Table 5).

Antibody responses were evaluated at day 0 (pre-immunization) and days 14, 27 and 42 post-immunization. Serum antibody levels induced by each of the bioconjugates included in the vaccine and the carrier protein EPA were measured by ELISA (total IgG), using type-specific LPS as coating material. The antibody titers were reported as EC50 values that correspond to the half maximal effective concentration based on duplicates of 12-step titration curves plotted in a 4-parameter logistic nonlinear regression model. Functional activity was determined by OPK.

TABLE 5
Description of experimental groups.

| Experimental | Dosing (μg/PS) | Sample |
|---|---|---|
| groups | O1A:O2:O6A:O25B:O4:O8:O15:O16:O18A:O75 | size |
| Group 1 | 8:8:8:16:8:8:8:8:8:8 | 7 |
| (high dose) | | |
| Group 2 | 8:4:8:16:4:4:4:4:4:4 | 7 |
| (medium | | |
| dose) | | |
| Group 3 | 0.8:0.4:0.8:1.6:0.4:0.4:0.4:0.4:0.4:0.4 | 7 |
| (low dose) | | |
| Group 4 | 0.9% (w/v) sodium chloride solution | 7 |
| (control) | | |

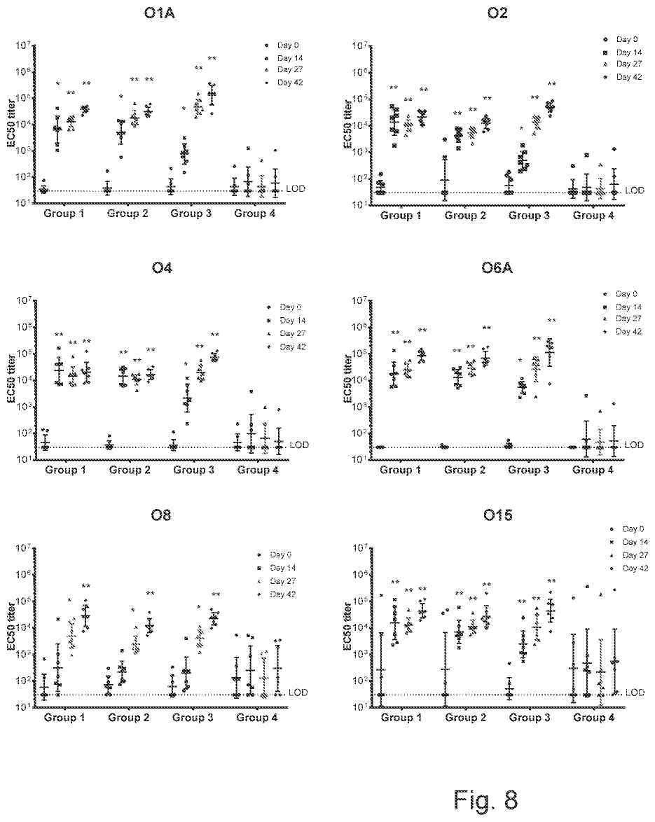

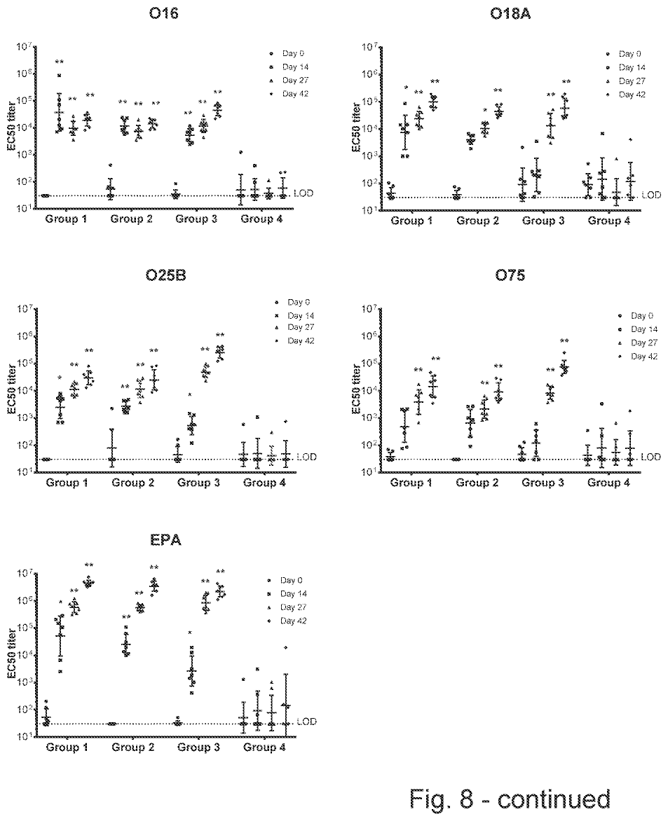

Results are shown in FIG. 8 and summarized in Table 6.

TABLE 6
Summary of E. coli O-antigen specific antibody responses induced by ExPEC10V in NZW rabbits.

| ExPEC10V | | | | | | | | | | |
|---|---|---|---|---|---|---|---|---|---|---|
| dose | O1A | O2 | O6A | O25B | O4 | O8 | O15^{#} | O16 | O18A | O75 |
| | Antibody responses day 14 post-vaccination | | | | | | | | | |
| High | * | ** | ** | * | ** | ns | ** | ** | * | ns |
| Mid | * | ** | ** | ** | ** | ns | ** | ** | ns | ns |
| Low | * | * | * | * | * | ns | ** | ** | ns | ns |
| | Antibody responses day 27 post-vaccination | | | | | | | | | |
| High | ** | ** | ** | ** | ** | * | ** | ** | ** | ** |
| Mid | ** | ** | ** | ** | ** | * | ** | ** | * | ** |
| Low | ** | ** | ** | ** | ** | * | ** | ** | ** | ** |
| | Antibody responses day 42 post-vaccination | | | | | | | | | |
| High | ** | ** | ** | ** | ** | ** | ** | ** | ** | ** |
| Mid | ** | ** | ** | ** | ** | ** | ** | ** | ** | ** |
| Low | ** | ** | ** | ** | ** | ** | ** | ** | ** | ** |

Serotype-specific antibody responses in which p values were statistically significant are shown by asterisks.
Serotype-specific antibody responses in which p values were not statistically significant are designated as ns.
Wilcoxon Rank Sum test with Bonferroni correction for multiple comparisons.
Comparisons ExPEC10V vaccinated animals (Group 1, 2 and 3) versus saline control (Group 4).
*p ≤ 0.05,
**p ≤ 0.01.
^{#}P values were statistically significant after excluding an outlier animal from the control group (sensitivity analysis).

The high dose of ExPEC10V (Group 1) induced significantly higher IgG antibody levels at all time-points investigated (Days 14, 27 and 42 post-immunization) when compared to saline control for O1A, O2, O4, O6A, O16, O18A and O25B (FIG. 8, Table 6). Significantly higher antibody titers induced by O8 and O75 conjugates when compared to saline control were observed at Days 27 and 42 post-immunization (FIG. 8, Table 6).

The medium dose of ExPEC10V (Group 2) and the low dose (Group 3) induced significantly higher antibody levels at all time-points investigated (Days 14, 27 and 42 post-immunization) when compared to saline control for O1A, O2, O4, O6A, O16 and O25B (FIG. 8, Table 6). Significantly higher antibody titers induced by O8, O18A and O75 conjugates when compared to saline control were observed at Days 27 and 42 post-immunization suggesting that the boost dose in rabbits increases the response to these O-serotypes (FIG. 8, Table 6).

For O15 conjugates, sensitivity analysis omitting an outlier animal from the control group showed that all three doses of ExPEC10V vaccine induced a significant increase in antibody responses when compared to saline control at Days 14, 27 and 42 post-immunization (FIG. 8, Table 6).

Antibodies induced by the carrier protein EPA were significantly higher than EPA antibody titers in the saline-treated (control) group for the three doses of ExPEC10V tested (high, medium and low) at all time points investigated (Days 14, 27 and 42) (FIG. 8).

Between dose comparisons (not shown) showed that at Day 14 post-vaccination, the high dose of ExPEC10V induced significantly higher antibody responses when compared to the low dose for most of the conjugates tested (O1A, O2, O4, O6A, O15, O16, O18A and O25B). The medium dose of ExPEC10V also induced significantly higher antibody responses compared to the low dose for O1A, O2, O4, O18A, O25B and O75. For O8 conjugate, all three formulations of ExPEC10V induced similar levels of antibodies at Day 14 post-vaccination.

The low dose of ExPEC10V induced a significant increase in antibody responses at Day 42 post vaccination (after a prime and two boost doses) when compared to the high and medium doses of ExPEC10V for O1A, O2, O4, O16, O25B and O75 conjugates. These findings are in line with other experiences with conjugate vaccines, where for instance no clear relationship between dose and the magnitude of the antibody response to primary vaccination was observed in infants vaccinated with pneumococcal conjugate vaccine (Poolman J T, et al. Expert Rev Vaccines. 2013, 12(12):1379-94).

There were no significant differences between the three doses of ExPEC10V tested at Day 42 post-vaccination for O6A, O8 and O15 conjugates. For the O18A conjugate, the high dose of ExPEC10V induced a significantly higher antibody response when compared to the medium dose at Day 42 post-vaccination.

For the carrier protein (EPA), the high and medium dose of ExPEC10V induced significantly higher antibody responses when compared to the low dose at day 14 post-vaccination. The high dose of the vaccine also induced significantly higher antibody responses when compared to the low dose at day 42 post-vaccination.

In conclusion, the three formulations of ExPEC10V (high, medium and low), administered via intramuscular injection on Days 0, 14, 27 are immunogenic in rabbits.

So far, functional antibodies capable of killing E. coli strains induced by this vaccine in rabbits were shown for serotypes O1A, O2, O4, O6A, O15, O16 and O25B.

In a further experiment, a GMP batch of the ExPEC10V vaccine (see Example 8 above for production) was prepared and injected into NZW rabbits as part of a toxicology study (Table 7). In this study, NZW rabbits (males and females) received 3 intramuscular injections (0.6 mL) of the ExPEC10V vaccine (day 1, 15 and 29) and a control group received 0.9% (w/v) sodium chloride solution (saline). Each dose of the vaccine contained 9.6 g polysaccharide (PS) for serotypes O1A, O2, O4, O6A, O8, O15, O16, O18A and O75 and 19.2 g PS for serotypes O25B, corresponding to 105.6 μg total PS (176 μg total PS/mL) and 382.8 μg of total EPA (638 μg EPA/mL). IgG titers against O-antigens and carrier protein (EPA) were determined from samples collected during the pre-treatment period (day 1) and days 31 and 50 post-immunization.

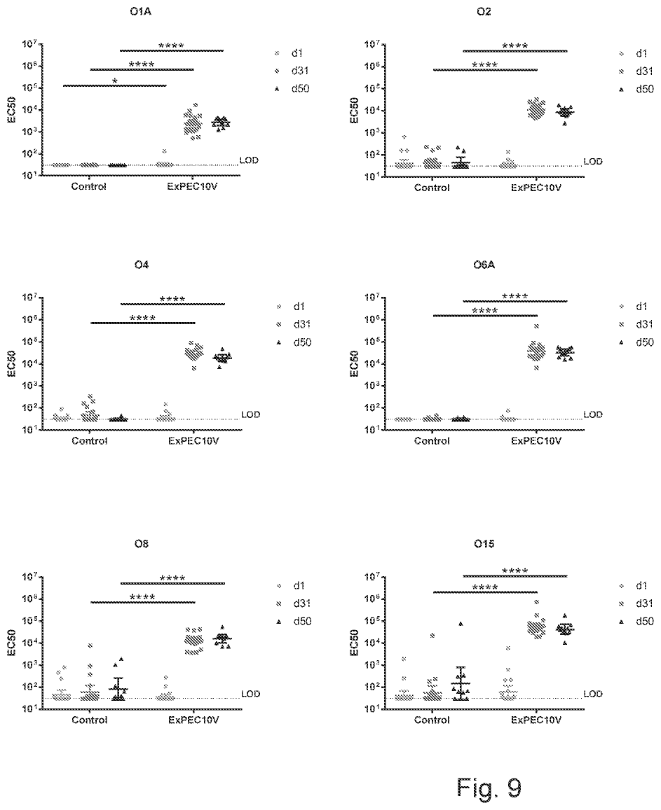

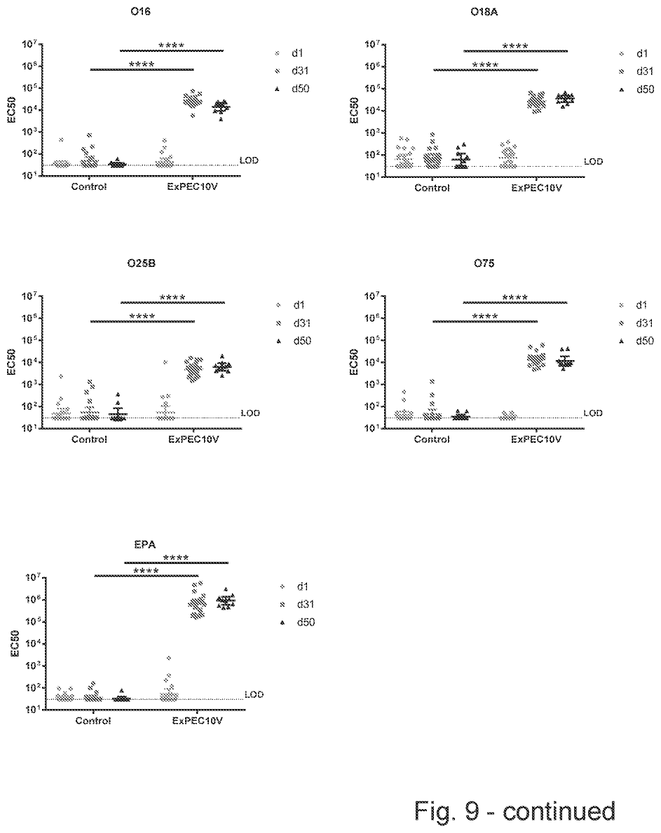

A significant increase in antibody responses against all O-antigens and the carrier protein EPA were observed at day 31 and 50 post-vaccination in the group that received ExPEC10V when compared to the control group that received only saline (FIG. 9, Table 8). For O1A serotype, a significantly higher antibody response was also observed at day 1 (baseline) when vaccinated animals were compared with the controls. These results suggest that some animals were pre-exposed to E. coli or have antibodies that cross-react with O1A-LPS.

TABLE 7
Experimental groups and ExPEC10V dose used in NZW rabbits.

| | | | Dosing | Main (day 31) | Recovery (day 50) |
|---|---|---|---|---|---|
| Groups | Treatment | Dose | days | (males/females) | (males/females) |
| 1 | control | 0 | 1, 15, 29 | 10 | 10 |
| 2 | ExPEC10V | 105.6 μg PS* | 1, 15, 29 | 10 | 10 |

*Each dose (0.6 mL dosing volume) contains 9.6:9.6:9.6:9.6:9.6:9.6:9.6:9.6:19.2:9.6 μg polysaccharide (PS) for serotypes O1A, O2, O4, O6A, O8, O15, O16, O18A, O25B, O75, respectively (176 μg total PS/mL). Each dose contains 382.8 μg EPA protein (638 μg EPA/mL).

TABLE 8
Immunogenicity of ExPEC10V in NZW rabbits as part to a toxicology study.

| Treatment | Antibody responses day 14 post-vaccination | | | | | | | | | |
|---|---|---|---|---|---|---|---|---|---|---|
| ExPEC10V | O1A | O2 | O6A | O25B | O4 | O8 | O15 | O16 | O18A | O75 |
| Day 31 | **** | **** | **** | **** | **** | **** | **** | **** | **** | **** |
| Day 50 | **** | **** | **** | **** | **** | **** | **** | **** | **** | **** |

Antibody responses induced by ExPEC10V.
Serotypes in which a significant increase in antibody responses was observed in the vaccine group compared to control are shown by asterisks. Tobit model with a likelihood ratio test.
****P ≤ 0.0001.

### Example 11: Phase 1/2a Trial with the ExPEC10V Vaccine in Humans

At present, there is no vaccine available to prevent IED. The serotypes comprising the ExPEC10V vaccine (O1A, O2, O4, O6A, O8, O15, O16, O18A, O25B and O75) were selected to address invasive disease caused by the majority of clinically relevant ExPEC strains that also represent the majority of ExPEC isolates causing antimicrobial resistant IED, including ST131. The selected serotypes are representative for the ten prevalent ExPEC O-serotypes causing bloodstream infections in the older population and responsible for approximately 70% of bloodstream infections caused by ExPEC.

Since the mechanism of action of conjugate vaccines in the prevention of invasive disease is not expected to be affected by antibiotic resistance mechanisms, it is believed that ExPEC10V vaccine provides protection against IED caused by drug-resistant- and drug-susceptible O1A, O2, O4, O6A, O8, O15, O16, O18A, O25B and O75 serotypes.

There is preceding clinical experience with ExPEC4V, an earlier vaccine candidate which comprised a subset of four of the E. coli O-antigen conjugates (O1A, O2, O6A and O25B) also found in ExPEC10V. Based on the results from four clinical studies (two completed phase 1 studies, one completed phase 2 study and an ongoing phase 2 study), ExPEC4V was well-tolerated by the study participants and no vaccine-related safety signals were observed at doses up to 16 μg polysaccharide (PS) per serotype (O1A, O2, O6A and O25B). Most adverse events (AEs) were Grade 1 and 2, very few Grade 3 AEs were reported. Late-onset solicited local AEs (AEs which start after Day 5 post-vaccination) were observed mainly with the higher doses of ExPEC4V. In each study, the ExPEC4V vaccine was shown to be immunogenic, demonstrating a dose-dependent vaccine immune response, and O-antigen specific Immunoglobulin G (IgG) titer increases, as measured by enzyme-linked immunosorbent assay (ELISA). Functional activity of the antibodies was demonstrated with an ExPEC4V-optimized opsonophagocytic killing assay (OPKA). Co-analysis of ELISA and OPKA test results showed correlation between the assay responses (Pearson correlation coefficients≥0.61 and >0.48 for Day 30 and Day 360, respectively in a Phase 2 clinical trial [study 4V-BAC2001]), substantiating the use of ELISA as a primary measure of ExPEC4V antibody titers and to predict functional antibody activity. Analysis of the immunogenicity data has demonstrated the durability of the immune response through three years after vaccination with ExPEC4V. It has now also been observed that sera from humans vaccinated with ExPEC4V and that had high titers of serotype-specific opsonophagocytic antibodies, when passively transferred into mice that were subsequently intraperitoneally challenged with E. coli strains of O25B or O2 serotype, were able to mediate protection in vivo (not shown). Hence, ExPEC4V-specific opsonophagocytic human antibodies mediate bacterial killing in vivo, which is in line with other conjugate vaccines in which the proposed mechanism of protection is by induction of opsonophagocytic antibodies that mediate bacterial killing.

ExPEC10V includes a total often serotypes and increases coverage from about 50% (ExPEC4V) to approximately 70% of bloodstream infections caused by ExPEC in adults aged 60 years and older. Based on the clinical experience with ExPEC4V, and on the pre-clinical data for ExPEC10V as discussed in the examples above, it is expected that administration of ExPEC10V will induce immune responses to E. coli serotypes O1A, O2, O4, O6A, O8, O15, O16, O18A, O25B and O75 also in humans.

A randomized, observer-blind, first-in-human phase 1/2a study to evaluate the safety, reactogenicity, and immunogenicity of three different doses of the ExPEC10V vaccine is conducted in humans aged 60 to 85 years in stable health (study 10V-BAC1001). The study design includes 2 cohorts: A total of 1,004 participants are enrolled in the study with 404 participants (100 participants/ExPEC10V dose) aged ≥60 to ≤85 years in stable health in Cohort 1 and an additional of 600 participants aged ≥60 years in stable health with a history of UTI in the past 5 years in Cohort 2.

ExPEC10V is a 10-valent vaccine candidate in development for the prevention of invasive extraintestinal pathogenic Escherichia coli (ExPEC) disease (IED) in adults 60 years of age and older. ExPEC10V consists of the O-antigen polysaccharides (PSs) of the ExPEC serotypes O1A, O2, O4, O6A, O8, O15, O16, O18A, O25B and O75 separately bioconjugated to the carrier protein, a genetically detoxified form of exotoxin A (EPA) derived from Pseudomonas aeruginosa, and its production has been described above. The O4 PS is the glucosylated form, having the structure of Formula (O4-Glc+) in Table 1.

### Objectives and Endpoints

COHORT 1—Phase 1/2a observer-blind period with open-label long-term follow-up period (N=404):

| | |
|---|---|
| Objectives | Endpoints |

Primary

| | |
|---|---|
| To evaluate the safety and reactogenicity of | Solicited local and systemic adverse events |
| different doses of ExPEC10V in | (AEs) collected for 14 days post- |
| participants ≥60 to ≤85 years of age | vaccination (from Day 1 to Day 15) |
| | Unsolicited AEs collected from the |
| | administration of the study vaccine until |
| | 29 days post-vaccination (from Day 1 to |
| | Day 30) |
| | Serious adverse events (SAEs) collected |
| | from the administration of the study |
| | vaccine until Day 181 |
| To evaluate the dose-dependent | Antibody titers for ExPEC10V, as |
| immunogenicity of ExPEC10V on Day 15 in | determined by multiplex |
| participants ≥60 to ≤85 years of age | electrochemiluminescent (ECL)-based |
| | immunoassay and multiplex |
| | opsonophagocytic assay (MOPA) on |
| | Day 15 |

Secondary

| | |
|---|---|
| To evaluate the correlation between multiplex | Antibody titers for ExPEC10V, as |
| ECL-based immunoassay (total antibody) and | determined by multiplex ECL-based |
| MOPA (functional antibody) serum titers on | immunoassay and MOPA on Day 15 |
| Day 15 | |
| To evaluate the dose-dependent | Antibody titers for ExPEC10V, as |
| immunogenicity of ExPEC10V on Days 30 | determined by multiplex ECL-based |
| and 181 in participants ≥60 to ≤85 years of age | immunoassay and MOPA on Days 30 and |
| | 181 |
| To evaluate, in the long-term follow-up | SAEs related to the study vaccine or study |
| (LTFU) period, the safety of the ExPEC10V | procedures collected from Day 182 until the |
| dose selected for further clinical development | end of the study |
| based on the Day 30 primary analysis in | |
| participants ≥60 to ≤85 years of age | |
| To evaluate, in the LTFU period, the | Antibody titers for ExPEC10V, as |
| immunogenicity of the ExPEC10V dose | determined by multiplex ECL-based |
| selected for further clinical development | immunoassay and MOPA at Year 1 |
| based on the Day 30 primary analysis | (Day 366), Year 2 (Day 731) and Year 3 |
| | (Day 1096) |

COHORT 2—Double-Blind Period with Double-Blind Long-Term Follow-Up Period (N=600):

| | |
|---|---|
| Objectives | Endpoints |

Primary

| | |
|---|---|
| To evaluate the safety and reactogenicity of | Solicited local and systemic AEs collected |
| the selected dose of ExPEC10V in | for 14 days post-vaccination (from Day 1 to |
| participants ≥60 years of age with a history | Day 15) |
| of UTI in the past 5 years | Unsolicited AEs collected from the |
| | administration of the study vaccine until |
| | 29 days post-vaccination (from Day 1 to |
| | Day 30) |
| | SAEs collected from the administration of |
| | the study vaccine until Day 181 |
| To evaluate the immunogenicity of the | Antibody titers for ExPEC10V, as |
| selected dose of ExPEC10V on Day 30 in | determined by multiplex ECL-based |
| participants ≥60 years of age with a history of | immunoassay and MOPA on Day 30 |
| UTI in the past 5 years | |

Secondary

| | |
|---|---|
| To evaluate the correlation between multiplex | Antibody titers for ExPEC10V, as |
| ECL-based immunoassay (total antibody) and | determined by multiplex ECL-based |
| MOPA (functional antibody) serum titers on | immunoassay and MOPA on Day 30 |
| Day 30 in participants ≥60 years of age with a | |
| history of UTI in the past 5 years | |
| To evaluate the immunogenicity of the | Antibody titers for ExPEC10V, as |
| selected dose of ExPEC10V on Days 15 and | determined by multiplex ECL-based |
| 181 in participants ≥60 years of age with a | immunoassay and MOPA on Days 15 and |
| history of UTI in the past 5 years | 181 |
| To evaluate, in the LTFU period, the safety of | SAEs related to the study vaccine or study |
| the selected dose of ExPEC10V in | procedures collected from Day 182 until the |
| participants ≥60 years of age with a history | end of the study |
| of UTI in the past 5 years | |
| To evaluate, in the LTFU period, the | Antibody titers for ExPEC10V, as |
| immunogenicity of the selected dose of | determined by multiplex ECL-based |
| ExPEC10V in participants ≥60 years of age | immunoassay and MOPA at Year 1 |
| with a history of UTI in the past 5 years | (Day 366), Year 2 (Day 731), and Year 3 |
| | (Day 1096) |

Exploratory

| | |
|---|---|
| To evaluate the effect of ExPEC10V on the | Metagenomics of stool samples from a |
| intestinal (stool) microbiome by metagenomic | selected subset1 of participants to evaluate |
| analyses | the effect of ExPEC10V on: |
| | Prevalence of pathogens |
| | (eg, Clostridium difficile) in the |
| | intestinal flora |
| | Prevalence of ExPEC10V serotypes in |
| | the intestinal flora |

### Overall Design

This is a randomized, multicenter, interventional study including two cohorts.

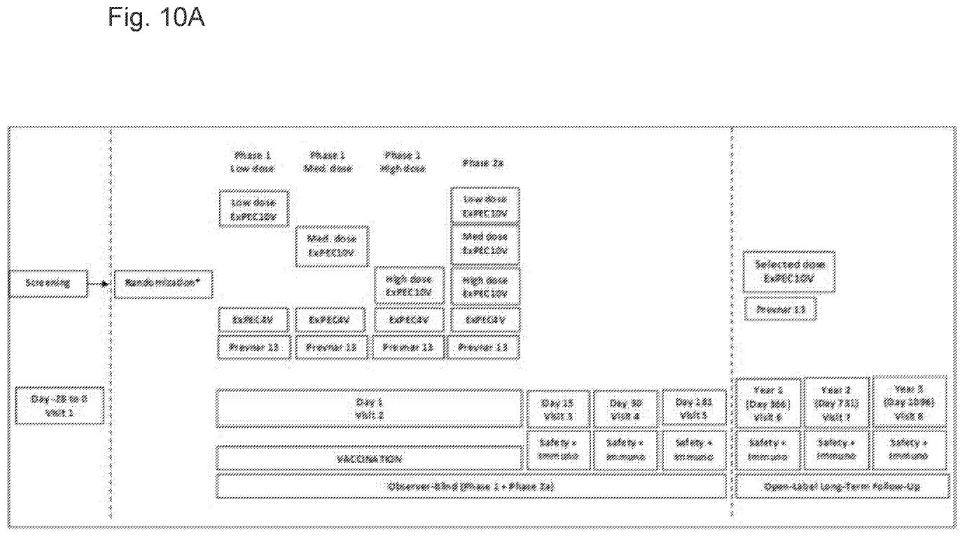

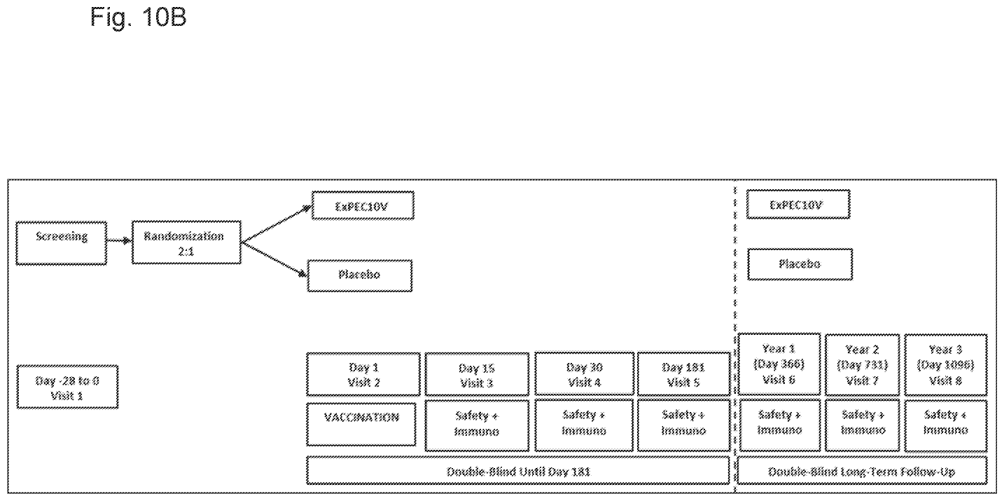

For Cohort 1, the study has an observer-blind, active-controlled design, and a total of 404 adult participants aged ≥60 to ≤85 years in stable health with or without a history of UTI are included. The study design for Cohort 1 is comprised of three periods: a maximum of 28-day screening period, an observer-blinded 181-day follow-up period with vaccination on Day 1 and an open-label LTFU period which lasts from Day 182 until 3 years (Day 1096) post-vaccination (FIG. 10A). Only participants from the ExPEC10V selected dose group (approximately 100 participants) and participants from the Prevnar 13 group progress to the LTFU period. The end of Cohort 1 is the last participant's Year 3 visit (Day 1096).

For Cohort 2, the study has a double-blind, placebo-controlled design, and a total of 600 adult participants aged ≥60 years in stable health with a history of UTI in the past 5 years is included. Enrollment commences after completion of the Phase 1/2a primary analysis and ExPEC10V dose selection from Cohort 1. The study design for Cohort 2 is comprised of three periods: a maximum 28-day screening period, a double-blind 181-day follow-up period with vaccination on Day 1, and a double-blind LTFU period which lasts from Day 182 until 3 years (Day 1096) post-vaccination. All participants in Cohort 2 progress to the LTFU period. The end of study is the last participant's Year 3 visit (Day 1096) in Cohort 2.

### Cohort 1: Phase 1

In Phase 1 of Cohort 1, a total of 84 participants are enrolled in a staggered approach following stepwise dose-escalating procedures with safety evaluations in place before progressing from one step to the next. An internal Data Review Committee (DRC) is commissioned for this study to review the physical examination data (baseline as well as targeted), baseline demographic data and the 14-day post-vaccination safety data (including solicited local and systemic AEs, unsolicited AEs, SAEs, clinical laboratory data and vital signs) of these 84 Phase 1 participants. In this phase of the study, participants were enrolled and randomized in six steps:

Step 1: Four sentinel participants were enrolled and randomized; two participants in the ExPEC10V low dose group, and one participant each in the ExPEC4V and Prevnar 13 groups.
Step 2: Twenty-four participants were enrolled and randomized; 18 participants in the ExPEC10V low dose group, and three participants each in the ExPEC4V and Prevnar 13 groups.
Step 3: Four sentinel participants were enrolled and randomized; two participants in the ExPEC10V medium dose group, and one participant each in the ExPEC4V and Prevnar 13 groups.
Step 4: Twenty-four participants were enrolled and randomized; 18 participants in the ExPEC10V medium dose group, and three participants each in the ExPEC4V and Prevnar 13 groups.
Step 5: Four sentinel participants were enrolled and randomized; two participants in the ExPEC10V high dose group, and one participant each in the ExPEC4V and Prevnar 13 groups.
Step 6: Twenty-four participants were enrolled and randomized; 18 participants in the ExPEC10V high dose group, and three participants each in the ExPEC4V and Prevnar 13 groups.

All participants received a single intramuscular (IM) injection of either ExPEC10V (1 of 3 doses), ExPEC4V or Prevnar 13 on Day 1 per the assigned study vaccination groups. The four sentinel participants at each of Steps 1, 3 and 5 were contacted by telephone 24 hours post-vaccination to collect safety information. The blinded 24-hour post-vaccination safety data in each group of four sentinel participants were reviewed by the principal investigator (PI), study responsible physician (SRP) and sponsor medical lead (SML). Randomization of additional participants for the next step was halted until this Day 2 sentinel safety evaluation was completed.

In the absence of any clinically significant findings, an additional 24 participants (for Steps 2, 4, and 6) were enrolled and randomized to one of three study vaccination groups to receive a single IM injection of either ExPEC10V (1 of 3 doses), ExPEC4V or Prevnar 13 on Day 1.

After vaccination of an additional 24 participants at each dose level (low dose in Step 2, medium dose in Step 4, and high dose in Step 6), 14-day post-vaccination safety data of all 28 (4+24) participants at each dose level was reviewed by the DRC before progressing to the next dose level or Phase 2a.

### Cohort 1: Phase 2a

Based on acceptable safety and reactogenicity (in the absence of any safety concerns or any events meeting a specific study pausing rule) as determined by DRC after the review of 14-day post-vaccination safety data for the initial 84 participants, the remaining 320 participants from Cohort 1 are randomized and dosed in Phase 2a of the study. These additional 320 participants were enrolled and randomized in parall in a ratio of 2:2:2:1:1 to one of the five study vaccination groups to receive a single IM injection of either ExPEC10V (1 of 3 doses), ExPEC4V or Prevnar 13 on Day 1 (Table 11).

In addition to performing the 14-day safety review for the initial 84 participants, the DRC also evaluates safety data of Cohort 1 over the course of the study and review any events that meet a specific study vaccination pausing rule or any other safety issue that may arise.

For Cohort 1, the primary analysis occurs when all participants have completed the Day 30 visit (Visit 4) or have discontinued earlier. The final analysis occurs when all participants have completed the Day 181 visit or have discontinued earlier. For participants progressing to the open-label long-term follow-up (LTFU) period (ExPEC10V selected dose group and Prevnar 13 group), yearly follow-up analyses include safety and immunogenicity data (multiplex ECL-based immunoassay and MOPA) collected up to the time of the visit at Year 1 (Day 366), Year 2 (Day 731) and Year 3 (Day 1096) after vaccination.

### Cohort 2

In Cohort 2, the safety, reactogenicity, and immunogenicity of the selected dose of ExPEC10V (based on the primary analysis results of Cohort 1) is evaluated in participants aged ≥60 years in stable health with a history of UTI in the past 5 years. For Cohort 2, the study has a double-blind, placebo-controlled design, and a total of 600 participants are enrolled and randomized in parallel in a 2:1 ratio (400 participants in the ExPEC10V group and 200 in the placebo group).

All participants receive a single IM injection of either the selected dose of ExPEC10V or placebo on Day 1 per the assigned study vaccination groups.

For Cohort 2, the primary analysis includes safety and immunogenicity data and occurs when all participants have completed the Day 30 visit (Visit 4) or have discontinued earlier. The final analysis occurs when all participants have completed the Day 181 visit or have discontinued earlier. For all participants, yearly follow-up analyses include safety and immunogenicity data (multiplex ECL-based immunoassay and MOPA) collected up to the time of the visit at Year 1 (Day 366), Year 2 (Day 731), and Year 3 (Day 1096) after vaccination.

A stool sample analysis is performed in a selected subset of participants to evaluate the effect of ExPEC10V on the prevalence of pathogens (eg, Clostridium difficile) and ExPEC10V serotypes in the intestinal flora using metagenomics.

### Number of Participants

A total of 1004 participants is enrolled in the study; 404 participants in Cohort 1 and 600 participants in Cohort 2.

### Intervention Groups

### Description of Interventions

ExPEC10V: E. coli bioconjugate vaccine in phosphate buffered solution containing O-antigen PS of ExPEC serotypes O1A, O2, O4, O6A, O8, O15, O16, O18A, O25B and O75 separately bioconjugated to the EPA carrier protein. Single 0.5 mL IM (deltoid) injection of one of the three doses of ExPEC10V on Day 1.

ExPEC4V: E. coli bioconjugate vaccine in saline buffer solution containing O-antigen PS of ExPEC serotypes O1A, O2, O6A, O25B (4:4:4:8 μg PS/ExPEC serotypes) separately bioconjugated to the EPA carrier protein. Single 0.5 mL IM (deltoid) injection of ExPEC4V on Day 1.

Prevnar 13: Sterile suspension of saccharides of the capsular antigens of Streptococcus pneumoniae serotypes 1, 3, 4, 5, 6A, 6B, 7F, 9V, 14, 18C, 19A, 19F, and 23F, individually linked to non-toxic Diphtheria CRM197 protein. Single 0.5 mL IM (deltoid) injection on Day 1, supplied in a single-dose prefilled syringe.

Placebo: normal saline. Single 0.5 mL IM (deltoid) injection of placebo on Day 1.

The ExPEC study intervention materials are described in Table 9.

TABLE 9
BAC1001MV ExPEC Study Vaccines

| | O1A | O2 | O4 | O6A | O8 | O15 | O16 | O18A | O25B | O75 | EPA | PS (Total) |
|---|---|---|---|---|---|---|---|---|---|---|---|---|
| Study Arm | (μg) | (μg) | (μg) | (μg) | (μg) | (μg) | (μg) | (μg) | (μg) | (μg) | (μg) | (μg) |
| Low dose | 4 | 4 | 4 | 4 | 4 | 4 | 4 | 4 | 8 | 4 | 160 | 44 |
| ExPEC10V | | | | | | | | | | | | |
| Medium | 8 | 4 | 4 | 8 | 4 | 4 | 4 | 4 | 16 | 4 | 221 | 60 |
| dose | | | | | | | | | | | | |
| ExPEC10V | | | | | | | | | | | | |
| High dose | 8 | 8 | 8 | 8 | 8 | 8 | 8 | 8 | 16 | 8 | 320 | 88 |
| ExPEC10V | | | | | | | | | | | | |
| ExPEC4V | 4 | 4 | — | 4 | — | — | — | — | 8 | — | 72 | 20 |

EPA = a genetically detoxified form of exotoxin A derived from Pseudomonas aeruginosa;
PS = polysaccharide ExPEC4V consists of the O-antigen polysaccharides (PSs) of the ExPEC serotypes O1A, O2, O6A, and O25B separately bioconjugated to the EPA carrier protein.
ExPEC10V consists of the O-antigen polysaccharides (PSs) of the ExPEC serotypes O1A, O2, O4, O6A, O8, O15, O16, O18A, O25B and O75 separately bioconjugated to the EPA carrier protein.
Dose is based on PS only.
The EPA (μg) are measured values.

ExPEC10V is composed of 10 monovalent drug substances (DSs). For this clinical study, 2 different concentrations (medium and high) of drug product (DP) are produced (Table 10). A third (low) concentration is obtained in the clinic by diluting the high concentration 1:1 with dilution buffer, which is the same as the formulation buffer. Each DP is formulated in Sodium/Potassium phosphate buffer at pH 7.0 (0.02% [w/w] Polysorbate 80, 5% [w/w]sorbitol, 10 mM methionine).

TABLE 10
Composition of ExPEC10V vaccine for phase 1/2a clinical study

| | Amount (μg/mL)a | | | |
|---|---|---|---|---|
| Ingredient | Low | Medium | | High |
| Activea | Concentration b | Concentration | | Concentration |
| O-antigen | | | | |
| polysaccharide | | | | |
| EcoO1A | 8 | 16 | | 16 |
| EcoO2 | 8 | 8 | | 16 |
| EcoO4 | 8 | 8 | | 16 |
| EcoO6A | 8 | 16 | | 16 |
| EcoO8 | 8 | 8 | | 16 |
| EcoO15 | 8 | 8 | | 16 |
| EcoO16 | 8 | 8 | | 16 |
| EcoO18A | 8 | 8 | | 16 |
| EcoO25B | 16 | 32 | | 32 |
| EcoO75 | 8 | 8 | | 16 |
| Carrier protein | | | | |
| EPA | 320 | 441 | | 640 |
| Excipients | | | | |
| KH2PO4 | | 6.19 | mM | |
| Na2HPO4 | | 3.81 | mM | |
| Sorbitol | | 5% | (w/w) | |
| Methionine | | 10 | mM | |
| Polysorbate 80 | | 0.02% | (w/w) | |

EPA = genetically detoxified P. aeruginosa exotoxin A used as carrier protein
aThe active ingredient is a biologically synthesized conjugate composed of the PS antigen and a carrier protein (EPA); the dose is calculated on the PS moiety only.
b The “low concentration” is obtained in the clinic by diluting the “high concentration” 1:1 with dilution buffer

### Safety Evaluations

Key safety assessments include solicited local and systemic AEs, unsolicited AEs, SAEs, physical examinations, vital sign measurements, and clinical laboratory tests.

### Immunogenicity Evaluations

Key immunogenicity assessments of collected sera include the assessment of ExPEC10V and ExPEC4V serotype-specific total IgG antibody levels elicited by the vaccine as measured by a multiplex ECL-based immunoassay, and ExPEC10V and ExPEC4V serotype-specific functional antibodies as measured by an opsonophagocytic killing assay (OPKA) in multiplex format (MOPA). Immunogenicity assessments of pneumococcal antibody titers elicited by Prevnar 13 are not performed.

The levels of serum antibodies induced by ExPEC10V are measured by a multiplex electrochemiluminescent (ECL)-based immunoassay. This assay combines high binding carbon electrodes in a multi-spot 96-well format microplate that is coated with different E. coli O-LPS antigens or the carrier protein EPA. The levels of antigen-specific antibodies present in serum samples are detected using a secondary antibody (anti-human IgG) labeled with SULFO-TAG. The SULFO-TAG emits light in the presence of electrical stimulation at an intensity that increases proportionally to the amount of bound IgG antibodies. This assay was qualified according to International Conference on Harmonisation (ICH) recommendations.

The levels of functional antibodies induced by ExPEC10V are measured by a multiplex opsonophagocytic assay (MOPA). Briefly, heat-inactivated serum samples are serially diluted and incubated with different E. coli strains that are specifically resistant to different types of antibiotics. After that, human complement and phagocytic cells (HL60) are added to the reaction and, after a second incubation period, an aliquot of the reaction mix is transferred to different PVDF hydrophilic membrane filter plates containing media supplemented with specific antibiotic that selectively allow growth of a strain that is resistant to that particular antibiotic. After overnight grown, the colony forming units (CFUs) are counted to determine the number of surviving bacteria. This assay was qualified according to ICH recommendations.

For ExPEC10V serotype antibodies as measured by multiplex ECL-based immunoassay and MOPA, and EPA as measured by multiplex ECL-based immunoassay only, the following measures of immunogenicity are evaluated and tabulated by the study vaccination groups, for all immunogenicity time points:
proportion of participants with a ≥2-fold and >4-fold increase in serum antibody titers from Day 1 (pre-vaccination)
geometric mean titer (GMT)
GMR: fold change from baseline, calculated from the post-baseline/baseline value.
   For the LTFU period, descriptive summaries of immunogenicity are provided for each serotype.

Dose selection for later phases considers the totality of the evidence available at the time of the primary analysis of Cohort 1 (Day 30 results).

TABLE 11
Cohort 1: Vaccination Schedule

| | | Phase 1 | | | | | | | |
|---|---|---|---|---|---|---|---|---|---|
| | | | | Step 3 | Step 4 | | | Phase 2a | |
| | | Step 1 | Step 2 | Sentinel | Additional | Step 5 | Step 6 | Step 7 | |
| Study | | Sentinel | Additional | participants | participants | Sentinel | Additional | Additional | |
| Vaccination | Vaccination | participants | participants | (Medium | (Medium | participants | participants | Phase 2a | |
| Group | on Day 1 | (Low dose) | (Low dose) | dose) | dose) | (High dose) | (High dose) | Participants | Total |
| G1 | Low dose | 2 | 18 | | | | | 80 | 100 |
| | ExPEC10V* | | | | | | | | |
| G2 | Medium dose | | | 2 | 18 | | | 80 | 100 |
| | ExPEC10V* | | | | | | | | |
| G3 | High dose | | | | | 2 | 18 | 80 | 100 |
| | ExPEC10V* | | | | | | | | |
| G4 | ExPEC4V** | 1 | 3 | 1 | 3 | 1 | 3 | 40 | 52 |
| G5 | Prevnar 13*** | 1 | 3 | 1 | 3 | 1 | 3 | 40 | 52 |
| Total | | 4 | 24 | 4 | 24 | 4 | 24 | 320 | 404 |

*ExPEC10V consists of the O-antigen polysaccharides (PSs) of the ExPEC serotypes O1A, O2, O4, O6A, O8, O15, O16, O18A, O25B and O75 separately bioconjugated to the carrier protein, a genetically detoxified form of exotoxin A (EPA) derived from Pseudomonas aeruginosa.
**ExPEC4V consists of the O-antigen polysaccharides (PSs) of the ExPEC serotypes O1A, O2, O6A, and O25B separately bioconjugated to the carrier protein, a genetically detoxified form of exotoxin A (EPA) derived from Pseudomonas aeruginosa.
***Prevnar 13, Pneumococcal 13-valent conjugate vaccine (Diphtheria CRM197 protein) is a sterile suspension of saccharides of the capsular antigens of Streptococcus pneumoniae serotypes 1, 3, 4, 5, 6A, 6B, 7F, 9V, 14, 18C, 19A, 19F, and 23F, individually linked to non-toxic Diphtheria CRM197 protein.

TABLE 11
Cohort 2: Vaccination Schedule

| Study Vaccination Group | Vaccination on Day 1 | Total |
|---|---|---|
| G6 | ExPEC10Va | 400 |
| G7 | Placebo | 200 |
| Total | | 600 |

aExPEC10V consists of the O-antigen polysaccharides (PSs) of the ExPEC serotypes O1A, O2, O4, O6A, O8, O15, O16, O18A, O25B, and O75 separately bioconjugated to the carrier protein, a genetically detoxified form of exotoxin A (EPA) derived from Pseudomonas aeruginosa.

The randomization ratio for the participants enrolled in Cohort 2 of the study is 2:1 (ExPEC10V:Placebo). The ExPEC10V dose used in Cohort 2 is based on the primary analysis (Day 30) results of Cohort 1.

### Status

Enrollment and vaccination of Cohort 1 of the study described above was completed. The study is ongoing in a blinded manner. Based on ongoing review of the safety data, no major safety issues were identified, and the ExPEC10V vaccine has an acceptable safety profile.

The analysis of the immunogenicity of the Cohort 1 clinical samples is ongoing in a blinded fashion. The ECL data were 100% Acceptance Quality Limits (AQL) checked and uploaded for data management. Analysis of the MOPA samples is ongoing. Data unblinding and statistical analysis is performed by using a clinical research organization (CRO).

The Cohort 2 vaccinations are started once the ExPEC10V dose for that Cohort has been identified based on the finalized primary analysis of the Day 30 results from Cohort 1.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

SEQUENCES

SEQ ID NO: 1 (Glycosvlation consensus sequence)
Asn-X-Ser(Thr), wherein X can be any amino acid except Pro
SEQ ID NO: 2 (Optimized glycosylation consensus sequence)
Asp(Glu)-X-Asn-Z-Ser(Thr), wherein X and Z are independently selected from any amino acid except Pro
SEQ ID NO: 3 (EPA carrier protein comprising 4 glycosylation consensus sequences (EPA-4))
G SGGGDQNATG SGGGKLAEEA FDLWNECAKA CVLDLKDGVR SSRMSVDPAI ADTNGQGVLH YSMVLEGGND
ALKLAIDNAL SITSDGLTIR LEGGVEPNKP VRYSYTRQAR GSWSLNWLVP IGHEKPSNIK VFIHELNAGN
QLSHMSPIYT IEMGDELLAK LARDATFFVR AHESNEMQPT LAISHAGVSV VMAQAQPRRE KRWSEWASGK
VLCLLDPLDG VYNYLAQQRC NLDDTWEGKI YRVLAGNPAK HDLDIKDNNN STPTVISHRL HFPEGGSLAA
LTAHQACHLP LEAFTRHRQP RGWEQLEQCG YPVQRLVALY LAARLSWNQV DQVIRNALAS PGSGGDLGEA
IREQPEQARL ALTLAAAESE RFVRQGTGND EAGAASADVV SLTCPVAKDQ NRTKGECAGP ADSGDALLER
NYPTGAEFLG DGGDVSFSTR GTQNWTVERL LQAHRQLEER GYVFVGYHGT FLEAAQSIVF GGVRARSQDL
DAIWRGFYIA GDPALAYGYA QDQEPDARGR IRNGALLRVY VPRWSLPGFY RTGLTLAAPE AAGEVERLIG
HPLPLRLDAI TGPEEEGGRV TILGWPLAER TVVIPSAIPT DPRNVGGDLD PSSIPDKEQA ISALPDYASQ
PGKPPREDLK LGSGGGDQNA T
SEQ ID NO: 4 (O4 GtrS amino acid sequence)
MNNLIMNNWCKLSIFIIAFILLWLRRPDILTNAQFWAEDSVFWYKDAYENGFLSSLTTPRNGYFQTVSTFI
VGLTALLNPDYAPFVSNFFGIMIRSVIIWFLFTERFNFLTLTTRIFLSIYFLCMPGLDEVHANITNAHWYL
SLYVSMILIARNPSSKSWRFHDIFFILLSGLSGPFIIFILAASCFKFINNCKDHISVRSFINFYLRQPYAL
MIVCALIQGTSIILTFNGTRSSAPLGFSFDVISSIISSNIFLFTFVPWDIAKAGWDNLLLSYFLSVSILSC
AAFVFVKGTWRMKVFATLPLLIIIFSMAKPQLTDSAPQLPTLINGQGSRYFVNIHIAIFSLLCVYLLECVR
GKVATLFSKIYLTILLFVMGCLNFVITPLPNMNWREGATLINNAKTGDVISIQVLPPGLTLELRKK
SEQ ID NO: 5 (Example O4 gtrS nucleic acid sequence)
ATGAATAATTTAATTATGAATAACTGGTGTAAATTATCTATATTTATTATTGCATTTATTTTGCTATGGCT
TAGAAGGCCGGATATACTCACAAACGCACAATTTTGGGCAGAAGATTCCGTTTTCTGGTATAAGGACGCCT
ATGAGAACGGATTCTTAAGTTCACTAACAACGCCTAGGAATGGGTATTTCCAGACTGTTTCTACATTTATA
GTTGGTCTGACTGCTTTATTAAATCCAGATTATGCACCTTTTGTTTCTAATTTTTTTGGCATAATGATTCG
CTCAGTAATTATATGGTTTTTATTTACAGAAAGATTCAACTTCCTCACATTGACTACTAGGATTTTCTTAT
CTATTTATTTTCTATGCATGCCTGGATTGGATGAAGTTCATGCAAATATAACAAATGCACATTGGTATTTG
TCATTATATGTATCAATGATCCTGATAGCTCGCAATCCAAGTTCAAAATCATGGAGGTTTCATGATATATT
CTTTATCTTGCTATCCGGGCTCAGTGGCCCATTTATAATTTTCATTTTAGCAGCTTCATGCTTTAAATTTA
TAAATAATTGTAAAGATCATATTAGTGTAAGATCTTTCATAAATTTCTACTTGCGTCAGCCATACGCATTA
ATGATTGTTTGCGCTTTAATTCAAGGAACTTCTATAATTCTAACTTTCAATGGCACACGTTCCTCAGCACC
GCTAGGATTCAGTTTTGATGTGATTTCGTCTATTATATCATCGAATATTTTTTTATTTACATTTGTCCCAT
GGGATATTGCAAAGGCTGGGTGGGATAATTTACTGTTATCTTATTTTTTGTCTGTTTCGATTTTGTCGTGT
GCGGCCTTTGTTTTTGTTAAAGGTACGTGGCGAATGAAAGTATTTGCAACTTTACCATTGCTAATTATAAT
ATTTTCAATGGCAAAACCACAATTGACAGACTCGGCACCTCAATTGCCAACACTTATTAATGGGCAAGGTT
CAAGATACTTCGTAAATATACATATTGCGATATTCTCTTTGCTATGTGTTTACTTACTTGAGTGCGTCAGG
GGGAAAGTGGCAACTTTATTTTCCAAAATATACTTAACAATTTTGCTATTCGTGATGGGATGTTTGAATTT
TGTTATCACCCCACTCCCAAACATGAACTGGAGGGAAGGTGCTACTTTGATTAATAATGCAAAAACTGGTG
ATGTCATTTCGATTCAAGTGCTACCACCTGGCCTAACACTTGAACTAAGGAAAAAATAA
SEQ ID NO: 6 (Example PglB sequence (‘wild-type’))
MLKKEYLKNPYLVLFAMIILAYVFSVFCRFYWVWWASEFNEYFFNNQLMIISNDGYAFAEGARDMIAGFHQ
PNDLSYYGSSLSALTYWLYKITPFSFESIILYMSTFLSSLVVIPTILLANEYKRPLMGFVAALLASIANSY
YNRTMSGYYDTDMLVIVLPMFILFFMVRMILKKDFFSLIALPLFIGIYLWWYPSSYTLNVALIGLFLIYTL
IFHRKEKIFYIAVILSSLTLSNIAWFYQSAIIVILFALFALEQKRLNFMIIGILGSATLIFLILSGGVDPI
LYQLKFYIFRSDESANLTQGFMYFNVNQTIQEVENVDLSEFMRRISGSEIVFLFSLFGFVWLLRKHKSMIM
ALPILVLGFLALKGGLRFTIYSVPVMALGFGFLLSEFKAIMVKKYSQLTSNVCIVFATILTLAPVFIHIYN
YKAPTVFSQNEASLLNQLKNIANREDYVVTWWDYGYPVRYYSDVKTLVDGGKHLGKDNFFPSFALSKDEQA
AANMARLSVEYTEKSFYAPQNDILKTDILQAMMKDYNQSNVDLFLASLSKPDFKIDTPKTRDIYLYMPARM
SLIFSTVASFSFINLDTGVLDKPFTFSTAYPLDVKNGEIYLSNGVVLSDDFRSFKIGDNVVSVNSIVEINS
IKQGEYKITPIDDKAQFYIFYLKDSAIPYAQFILMDKTMFNSAYVQMFFLGNYDKNLFDLVINSRDAKVFK
LKI
SEQ ID NO: 7 (example gtrA amino acid sequence; E. coli W3110 yfdG. GenBank: BAA16209.1)
MLKLFAKYTSIGVLNTLIHWVVFGVCIYVAHTNQALANFAGFVVAVSFSFFANAKFTFKASTTTMRYMLYV
GFMGTLSATVGWAADRCALPPMITLVTFSAISLVCGFVYSKFIVFRDAK
SEQ ID NO: 8 (example gtrB amino acid sequence - E. coli W3110 yfdH. GenBank: BAA16210.1)
MKISLVVPVFNEEEAIPIFYKTVREFEELKSYEVEIVFINDGSKDATESIINALAVSDPLVVPLSFTRNFG
KEPALFAGLDHATGDAIIPIDVDLQDPIEVIPHLIEKWQAGADMVLAKRSDRSTDGRLKRKTAEWFYKLHN
KISNPKIEENVGDFRLMSRDVVENIKLMPERNLFMKGILSWVGGKTDIVEYVRAERIAGDTKFNGWKLWNL
ALEGITSFSTFPLRIWTYIGLVVASVAFIYGAWMILDTIIFGNAVRGYPSLLVSILFLGGIQMIGIGVLGE
YIGRTYIETKKRPKYIIKRVKK
SEQ ID NO: 9 (example O4 rfb locus nucleotide sequence - O4-EPA production strain BVEC-L-
00684f)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGTGAAAATACTT
GTTACTGGTGGCGCAGGATTTATTGGTTCAGCTGTAGTTCGTCACATTATAAATAATACGCAGGATAGTGT
TGTTAATGTCGATAAATTAACGTACGCCGGAAACCGGGAATCACTTGCTGATGTTTCTGATTCTGAACGCT
ATGTTTTTGAACATGCGGATATTTGCGATGCACCTGCAATGGCACGGATTTTTGCTCAGCATCAGCCGGAT
GCAGTGATGCACCTGGCTGCTGAAAGCCATGTTGACCGTTCAATTACAGGCCCTGCGGCATTTATTGAAAC
CAATATTGTTGGTACTTATGTCCTTTTGGAAGCCGCTCGCAATTACTGGTCTGCTCTTGATAGCGACAAGA
AAAATAGCTTCCGTTTTCATCATATTTCTACTGACGAAGTATATGGTGATTTGCCTCATCCTGACGAGGTA
AATAATACAGAAGAATTACCCTTATTTACTGAGACAACAGCTTACGCGCCAAGCAGCCCTTATTCCGCATC
CAAAGCATCCAGCGATCATTTAGTCCGCGCGTGGAAACGTACCTATGGTTTACCGACCATTGTGACTAATT
GCTCTAACAATTATGGTCCTTATCATTTCCCGGAAAAATTGATTCCATTGGTTATTCTCAATGCTCTGGAA
GGTAAAGCATTACCTATTTATGGTAAAGGGGATCAAATTCGCGACTGGCTGTATGTTGAAGATCATGCGCG
TGCGTTATATACCGTCGTAACCGAAGGTAAAGCGGGTGAAACTTATAACATTGGTGGGCACAACGAAAAGA
AAAACATAGATGTAGTGCTCACTATTTGTGATTTGCTGGATGAGATTGTACCGAAAGAGAAATCTTATCGT
GAGCAAATCACTTATGTTGCCGATCGTCCGGGACACGATCGCCGTTATGCGATTGATGCTGAGAATATTGG
TCGCGAATTGGGATGGAAACCACAGGAAACGTTTGAGAGCGGGATTCGGAAGACAGTGGAATGGTATCTGT
CCAATACAAAATGGGTTGATAATGTGAAAAGTGGTGCCTATCAATCGTGGATTGAAGAGAACTATGAGGGC
CGCCAGTAATGAATATCCTCCTTTTTGGCAAAACAGGGCAGGTAGGTTGGGAACTACAGCGTGCTCTGGCA
CCTCTGGGTAACTTGATTGCTCTTGATGTTCATTCCACTGATTATTGTGGCGATTTCAGTAACCCCGAAGG
TGTGGCTGAAACCGTCAAAAAAATTCGCCCAGATGTTATTGTTAATGCTGCTGCTCATACCGCGGTAGATA
AGGCTGAGTCAGAACCAGAATTTGCACAATTACTCAATGCGACCAGCGTTGAAGCAATTGCAAAAGCGGCT
AATGAAGTTGGGGCTTGGGTAATTCATTACTCAACTGACTACGTCTTCCCTGGAAATGGCGACATGCCATG
GCTCGAGACTGATGTAACCGCTCCGCTCAATGTTTATGGCAAAACCAAATTGGCTGGAGAAAGAGCATTAC
AAGAACATTGCGCAAAGCATCTTATTTTCCGTACCAGCTGGGTATATGCAGGTAAAGGAAATAACTTTGCC
AAAACAATGTTACGTCTGGCAAAAGAGCGCGAAGAACTGGCTGTGATAAACGATCAGTTTGGCGCACCAAC
AGGTGCTGAATTGCTGGCTGATTGCACCGCTCATGCCATTCGCGTGGCATTAAAAAAACCAGAAGTTGCTG
GCTTGTACCATCTGGTAGCAAATGGCACAACAACCTGGCACGATTACGCCGCGCTAGTATTCGAAGAAGCC
CGTAAAGCAGGGATTGACCTTGCACTTAACAAACTCAACGCCGTACCAACAACGGCTTATCCTACTCCAGC
CCGCCGTCCTCATAATTCTCGCCTCAATACCGAAAAGTTTCAGCAGAACTTTGCGCTTGTCTTGCCTGACT
GGCAGGTGGGCGTGAAACGTATGCTCAACGAATTATTTACGACTACGGCAATTTAACAAATTTTTGCATCT
CGCTCATGATGCCAGAGCGGGATGAATTAAAAGGAATGGTGAAATGAAAACGCGTAAAGGTATTATTCTGG
CTGGTGGTTCCGGCACTCGTCTTTATCCTGTGACGATGGCAGTGAGTAAACAACTGCTGCCGATTTATGAT
AAGCCGATGATTTATTATCCGCTTTCAACGCTTATGTTAGCGGGTATTCGCGATATTCTTATTATCAGTAC
GCCACAGGATACACCGCGTTTCCAACAATTGTTGGGGGACGGGAGTCAGTGGGGGCTTAATCTACAGTATA
AAGTACAACCGAGTCCGGATGGCCTGGCGCAAGCGTTTATTATTGGTGAAGACTTTATTGGTGGTGATGAT
TGTGCACTCGTACTTGGCGATAATATCTTCTATGGACACGACTTGCCGAAATTAATGGAAGCTGCTGTTAA
CAAAGAAATCGGTGCAACGGTATTTGCTTATCACGTCAATGATCCTGAACGTTATGGTGTCGTGGAGTTTG
ATAATAACGGTACTGCAATTAGCCTGGAAGAAAAACCGCTGGAACCAAAAAGTAACTATGCGGTTACTGGG
CTTTATTTCTATGACAATGATGTTGTAGAAATGGCGAAAAACCTTAAGCCTTCTGCCCGTGGCGAACTGGA
AATTACCGATATTAACCGTATTTATATGGAGCAGGGACGTTTGTCTGTCGCTATGATGGGGCGTGGTTATG
CCTGGTTGGATACTGGTACACATCAAAGTCTTATTGAAGCAAGTAACTTCATTGCCACCATTGAAGAGCGT
CAGGGATTAAAGGTATCTTGCCCGGAAGAGATTGCTTACCGTAAAGGGTTTATTGATGCTGAGCAGGTGAA
AGTATTAGCCGAACCGCTGAAGAAAAATGATTATGGTCAGTATCTGCTAAAAATGATTAAAGGTTATTAAT
AAAATGAACGTAATTAAAACTGAAATTCCTGATGTGCTGATTTTTGAACCAAAAGTTTTTGGTGATGAACG
TGGCTTCTTTTTTGAGAGTTTTAACCAGAAAGTATTTGAAGAAGCTGTAGGACGGAAGGTTGAATTTGTTC
AGGATAACCATTCTAAGTCTAAAATAAATGTATTGCGTGGGATGCATTATCAAACACAAAATACTCAAGGA
AAACTGGTTCGGGTAATTTCTGGTTCAGTATATGATGTTGCCGTAGATTTAAGAGAAAAATCAAAGACATT
TGGCAAATGGGTGGGTGTAGAATTATCTGGGAATAATAAAAGACAATTGTGGATCCCCGAAGGTTTTGCCC
ATGGTTTTTATGTGTTGGAGGAGAATACCGAATTTGTTTATAAATGTACCGATACTTATAACCCTGCTCAT
GAACACACATTGCTATGGAATGATCCAACTATCAATATAAGTTGGCCAATCATACAAAACTGCAAGCCAAT
TATTTCTGAAAAAGATGCTAATGGACATCTTTTTTCACATAAAACCTATTTCTGAAATGCAATATTATGAG
TTTAATTAGAAACAGTTTCTATAATATTGCTGGTTTTGCTGTGCCGACATTAGTTGCAGTCCCTGCTTTGG
GGATTCTTGCCAGGCTGCTTGGACCGGAGAATTTTGGACTTTTCACACTAGCATTCGCTTTGATAGGATAT
GCAAGTATTTTCGACGCCGGGATTAGTCGAGCTGTAATCAGAGAAATCGCTCTTTATCGAGAAAGTGAAAA
AGAGCAAATACAAATTATTTCGACAGCAAGTGTAATCGTACTATTCTTAGGGGTGGTTGCAGCTTTGTTAC
TTTATTTTAGTAGTAATAAAGTTGTTGAGTTATTGAATGTTAGTTCCGTTTATATTGAAACAGCAGTGCGT
GCATTCTCTGTTATTTCATTTATAATACCTGTGTATCTGATTAACCAGATTTGGCTTGGTTATCTGGAAGG
GCTAGAAAAATTTGCAAATATAAATGTTCAGAGAATGATTTCTAGCACAAGCTTGGCTATATTACCAGTGA
TATTTTGTTATTACAATCCCTCGTTGCTTTATGCTATGTATGGGTTGGTGGTTGGGCGTGTGATTTCATTT
TTGATTAGCGCAATAATTTGTCGAGATATTATTCTTAAAAGTAAACTTTACTTTAATGTGGCAACTTGCAA
TCGTCTTATCTCTTTTGGTGGATGGATAACAGTTAGTAATATCATAAGCCCAATCATGGCATATTTCGACC
GCTTTATCATCTCTCATATTATGGGGGCTTCGAGAATTGCATTTTATACAGCGCCCTCAGAGGGTGTATCA
AGGTTAATTAATATCCCATATGCTTTGGCAAGAGCTCTATTTCCTAAATTGGCATATAGCAATAATGATGA
TGAACGAAAAAAATTACAACTACAGAGCTACGCAATTATAAGCATTGTATGTCTACCCATAGTTGTTATTG
GTGTCATTTTTGCCTCATTCATAATGACAACATGGATGGGACCTGATTATGCCTTAGAAGCAGCAACTATC
ATGAAAATACTTCTTGCTGGTTTTTTCTTTAACTCTTTAGCGCAAATACCTTATGCATACTTGCAATCTAT
CGGAAAGTCAAAAATTACCGCATTTGTGCATCTCATAGAACTTGCGCCATACTTATTATTATTGTATTACT
TCACAATGCATTTCGGCATAATTGGCACGGCAATCGCTTGGTCACTTAGAACATTTTGTGATTTTGTTATA
CTACTTTCGATATCGAGAAGAAAATGATTGCGGTTGATATTGCGCTTGCAACCTACAATGGTGCTAATTTT
ATTCGGCAACAGATTGAATCTATCCAGAAACAAACTTATAGAAATTGGCGTCTTATAATAAGTGATGATAA
CTCGAGTGATGATACTGTTGATATTATTAAGGATATGATGTCTAACGACAGTCGTATCTATTTGGTAGGAA
ATAAAAGACAAGGAGGGGTTATTCAGAACTTTAATTATGCTCTTTCACAAACTACATCTGAAATTGTGTTA
CTATGTGACCAGGATGACATTTGGCCGGAGGAGCGTCTGGAAATTCTTATAGATAAATTTAAGGCCTTGCA
GCGTAATGATTTTGTTCCGGCAATGATGTTTACTGATTTGAAATTAGTAGACGAAAATAATTGTTTGATTG
CAGAAAGTTTTTATCGAACGAATAATATTAATCCACAAGATAATCTGAAAAATAATAATCTTCTCTGGCGT
TCAACGGTATATGGCTGTACTTGCATCATGAATAAGAAACTTGTTGATATTGCATTGCCTATACCTACATA
TGCACATATGCATGATCAATGGTTGGCATTATTAGCGAAGCAATATGGTAACATTTTTTATTTCGACTATG
CGTCTGTTCGTTATAGGCAACATTCTACAAATGTTGTTGGTGGTAGAAATAAAACGCCATTTCAAAAATTT
AATTCCATACAAAAAAACCTAAAAAGGATTAATTTGCTAGTGGATAGAACTGTTGCTTTAATTAAATCAAA
TAACGATTTCTATCCAGGGAATAAAATGGAAAATAAAATTGATTACTTAAAATTTGGAGTGAATGAAGTAT
TACCTTATCTTTTTAAAGGAAACAAGAAAGTTTTTTCACTTTGTGTATTAATTAGTTTGGCATTACAAAAA
TGATATATTTATTATTTTTTTTTGCACTGTTTATGATCTGTACGTTTTTAACACACAGGCGACAGGCATTA
TATGTTGTATCTGCGTTAGTATTTCTTTTTTTGGCTTTAACCTATCCATCAGGAGGGGACTGGATAGGTTA
TTTTCTCCATTATGACTGCATGGTTAATGAGCAGTGTAATAATGGTTTTATAATGTTTGAACCTGGATATG
AATTAATTGTTTCCTTATTTGGATATTTGGGATTTCAGACAATTATTATTTTTATAGCCGCTGTAAATGTA
ATTCTAATATTAAATTTTGCAAAGCATTTTGAAAACGGAAGTTTTGTTATTGTTGCGATAATGTGCATGTT
CCTTTGGAGTGTTTATGTTGAGGCGATTAGACAGGCTCTGGCCTTATCTATAGTTATATTTGGGATTCATT
CTCTTTTTTTGGGTAGAAAAAGGAAATTTATAACATTAGTATTATTTGCGTCAACTTTCCATATAACTGCT
TTGATTTGTTTTCTTCTAATGACTCCTCTATTTTCAAAGAAATTAAGCAAGATAATAAGTTATAGCCTATT
AATTTTCAGTAGCTTCTTTTTCGCTTTTTCTGAAACCATATTAAGTGCACTCCTTGCAATTTTGCCAGAAG
GATCCATTGCCAGTGAAAAATTAAGTTTTTACTTAGCAACCGAGCAATACAGGCCACAGTTATCTATTGGG
AGTGGCACTATTCTTGACATTATACTTATTTTTCTGATATGTGTAAGTTTTAAACGAATAAAGAAATATAT
GCTCGCTAATTATAATGCTGCAAATGAGATATTGCTTATTGGTTGCTGTCTTTATATTTCTTTCGGTATTT
TTATCGGGAAAATGATGCCAGTTATGACTCGCATTGGTTGGTATGGTTTTCCATTTGTTATAGTACTTCTT
TATATTAACTTGGGTTATTCAGAATATTTTAAGAGGTATATAAATAAAAGAGGGTGTGGGTATAGCAAATT
ATTAATTGCTTTTTATTTTTTGCTACAAATTTTGCGACCATTAACATATGATTATAGCTATTATAATATAA
TGCACCAGGATACTTTGCTGAATAGGTTTGATGCATTAGATGATGCATCATTAAGACAATCAGCGAAGAGA
AAATGTTTCGATTTGGGAAAGATAGGATATGGTTTCTTATGTAGTATATAATATCCTGCATTCATTCGGAT
AATTTCCTATGGAAGTGTCCTTTGCTCTGTCTGTCCTCATTTGTTGAAATTTTATGTTAATAAGAAGCTTT
AGATAACCACTTAGGAACTGTATGTTTGATCTGTCCAAAAATTATATTATTGTAAGTGCGACGGCGCTGGC
TTCCGGAGGTGCATTAACTATATTAAAGCAATTTATAAAACATGCATCACAAAATTCAAATGACTATATTA
TGTTTGTATCTGCGGGATTGGAGTTGCCGGTCTGTGATAACATCATTTACATAGAAAACACACCAAAAGGA
TGGTTGAAAAGAATATATTGGGATTGGTTCGGTTGTCGGAAGTTTATCTCGGAACATAAGATTAACGTTAA
GAAAGTAATTTCTCTACAAAATTCCAGTTTGAATGTTCCTTACGAACAGATTATTTACTTGCACCAGCCAA
TTCCTTTTAGTAAAGTTGATTCTTTTTTAAAAAATATCACATCCGATAACGTAAAGCTTTTTTTATATAAA
AAGTTTTATTCCTATTTTATATTTAAATATGTGAATGCCAATACAACCATCGTAGTGCAAACGAATTGGAT
GAAAAAAGGAGTGCTGGAGCAATGTGATAAAATTAGTACCGAAAGGGTCCTTGTTATAAAACCTGATATCA
AAGCATTTAATAATACTAATTTTGATGTAGATATGGATGTATCTGCAAAAACACTCTTATATCCAGCGACA
CCACTTACCTATAAAAATCATTTGGTCATTCTGAAGGCGTTGGTTATTTTAAAGAAAAAGTATTTTATAGA
TGATCTGAAATTCCAAGTGACTTTTGAAAAGAATAGGTACAAAAATTTTGATAAGTTTGTGCAATTAAATA
ACTTAAGCAAAAACGTTGATTATCTCGGCGTTCTTTCATACTCGAACTTGCAAAAAAAATATATGGCGGCA
TCTTTAATCGTTTTTCCTAGCTATATCGAATCATATGGGTTACCACTCATCGAAGCTGCTAGTTTAGGAAA
AAAAATCATTAGTAGTGATCTTCCTTATGCCCGGGATGTTTTAAAGGATTATAGCGGCGTAGATTTTGTAA
TTTACAATAATGAAGATGGCTGGGCTAAGGCGTTGTTTAATGTTTTAAATGGCAATTCGAAGCTCAATTTT
AGGCCTTATGAAAAAGATAGTCGTTCATCTTGGCCACAGTTCTTCTCTATTTTGAAATAAGGTGTATTATG
TTTAATGGTAAAATATTGTTAATTACTGGTGGTACGGGGTCTTTCGGTAATGCTGTTCTAAGACGTTTTCT
TGACACTGATATCAAAGAAATACGTATTTTTTCCCGGGATGAAAAAAAACAAGATGACATGAGGAAAAAAT
ATAATAATCCGAAACTTAAGTTCTATATAGGTGATGTTCGCGACTATTCGAGTATCCTCAATGCTTCTCGA
GGTGTTGATTTTATTTATCATGCTGCAGCTCTGAAGCAAGTACCTTCCTGCGAATTCCACCCAATGGAAGC
TGTAAAAACGAATGTTTTAGGTACGGAAAACGTACTGGAAGCGGCAATAGCTAATGGAGTTAGGCGAATTG
TATGTTTGAGTACAGATAAAGCTGTATATCCTATCAATGCAATGGGTATTTCCAAAGCGATGATGGAAAAA
GTAATGGTAGCAAAATCGCGCAATGTTGACTGCTCTAAAACGGTTATTTGCGGTACACGTTATGGCAATGT
AATGGCATCTCGTGGTTCAGTTATCCCATTATTTGTCGATCTGATTAAATCAGGTAGACCAATGACGATAA
CAGACCCTAATATGACTCGTTTCATGATGACTCTCGAAGACGCTGTTGATTTGGTTCTTTACGCATTTGAA
CATGGCAATAATGGTGATATTTTTGTCCAAAAGGCACCTGCGGCTACCATCGAAACGTTGGCTATTGCACT
CAAAGAATTACTTAATGTAAACCAACACCCTGTAAATATAATCGGCACCCGACACGGGGAAAAACTGTACG
AAGCGTTATTGAGCCGAGAGGAAATGATTGCAGCGGAGGATATGGGTGATTATTATCGTGTTCCACCAGAT
CTCCGCGATTTGAACTATGGAAAATATGTGGAACATGGTGACCGTCGTATCTCGGAAGTGGAAGATTATAA
CTCTCATAATACTGATAGGTTAGATGTTGAGGGAATGAAAAAATTACTGCTAAAACTTCCTTTTATCCGGG
CACTTCGGTCTGGTGAAGATTATGAGTTGGATTCATAATATGAAAATTTTAGTTACTGGCGCTGCAGGGTT
TATCGGTCGAAATTTGGTATTCCGGCTTAAGGAAGCTGGATATAACGAACTCATTACGATAGATCGTAACT
CTTCTTTGGCGGATTTAGAGCAGGGACTTAAGCAGGCAGATTTTATTTTTCACCTTGCTGGGGTAAATCGT
CCCGTGAAGGAGTGTGAATTTGAAGAGGGAAATAGTAATCTAACTCAACAGATTGTTGATATCCTGAAAAA
AAACAATAAAAATACTCCTATCATGCTGAGTTCTTCCATCCAGGCTGAATGTGATAACGCTTATGGAAAGA
GTAAAGCAGCTGCGGAAAAAATCATTCAGCAGTATGGGGAAACGACAAACGCTAAATATTATATTTATCGC
TTGCCGAATGTATTCGGTAAGTGGTGTCGACCAAATTATAACTCCTTTATAGCAACTTTCTGCCATCGCAT
TGCAAATGATGAAGCTATTACAATTAATGATCCTTCAGCAGTTGTAAATCTGGTGTATATAGATGACTTTT
GTTCTGACATATTAAAGCTATTAGAAGGAGCGAACGAAACTGGTTACAGGACATTTGGTCCAATTTATTCT
GTTACTGTTGGTGAAGTGGCACAATTAATTTACCGGTTTAAAGAAAGTCGCCAAACATTAATCACCGAAGA
TGTAGGTAATGGATTTACACGTGCATTGTACTCAACATGGTTAAGTTACCTGTCTCCTGAACAGTTTGCGT
ATACGGTTCCTTCTTATAGTGATGACAGAGGGGTATTCTGTGAAGTATTGAAAACGAAAAACGCGGGCCAG
TTTTCGTTCTTTACTGCGCATCCAGGAATTACTCGGGGTGGTCATTATCATCATTCCAAAAATGAGAAATT
TATTGTCATCCGAGGAAGTGCTTGTTTCAAATTTGAAAATATTGTCACGAGTGAACGATATGAACTTAATG
TTTCCTCTGATGATTTTAAAATTGTTGAAACAGTTCCGGGATGGACGCATAACATTACTAATAATGGCTCG
GATGAGCTAGTTGTTATGCTTTGGGCAAATGAAATATTTAATCGTTCTGAACCAGATACTATAGCGAGAGT
TTTATCGTGAAAAAATTGAAAGTCATGTCGGTTGTTGGGACTCGTCCAGAAATTATTCGACTCTCGCGTGT
CCTTGCAAAATTAGATGAATATTGTGACCACCTTATTGTTCATACCGGGCAAAACTACGATTATGAACTGA
ATGAAGTTTTTTTCAAAGATTTGGGTGTTCGCAAACCTGATTATTTTCTTAATGCCGCAGGTAAAAATGCA
GCAGAGACTATTGGACAAGTTATCATTAAAGTTGATGAGGTCCTTGAACAGGAAAAACCAGAAGCCATGTT
AGTACTTGGCGATACTAACTCCTGTATTTCAGCAATACCAGCAAAGCGTCGAAGAATTCCGATCTTCCATA
TGGAGGCTGGGAATCGTTGTTTTGACCAACGCGTACCGGAAGAAACTAACAGAAAAATAGTTGATCATACC
GCTGATATCAATATGACATATAGTGATATCGCGCGTGAATATCTTCTGGCTGAAGGTGTACCAGCCGATAG
AATTATTAAAACCGGTAGCCCAATGTTTGAAGTACTCACTCATTATATGCCGCAGATTGATGGTTCCGATG
TACTTTCTCGCCTGAATTTAACACCTGGGAATTTCTTTGTGGTAAGTGCCCACAGAGAAGAAAATGTTGAT
ACCCCTAAACAACTTGTGAAACTGGCGAATATACTTAATACCGTGGCTGAAAAATATGATGTCCCGGTAGT
TGTTTCTACTCATCCTCGCACTCGTAACCGCATCAACGAAAACGGTATTCAATTCCATAAAAATATCTTGC
TTCTTAAGCCATTAGGATTTCACGATTACAACCATCTGCAAAAAAATGCACGTGCTGTTTTATCGGATAGT
GGGACTATTACAGAAGAGTCCTCCATTATGAACTTCCCTGCACTCAATATACGAGAAGCGCACGAACGCCC
GGAAGGCTTCGAAGAAGGGGCAGTAATGATGGTCGGTCTTGAATCTGATCGCGTTTTACAGGCATTAGAAA
TTATTGCAACACAGCCTCGTGGAGAAGTACGCTTACTTCGTCAGGTTAGTGACTATAGCATGCCAAATGTT
TCAGATAAAGTTCTGCGTATTATCCATTCATATACTGACTACGTTAAACGGGTTGTCTGGAAGCAATACTA
ATGAAACTTGCATTAATCATTGATGATTATTTGCCCCATAGCACACGCGTTGGGGCTAAAATGTTTCATGA
GTTAGGCCTTGAATTACTGAGCAGAGGCCATGATGTAACTGTAATTACGCCTGACATCTCATTACAAGCAA
TTTATTCTATTAGTATGATTGATGGTATAAAGGTTTGGCGTTTCAAAAGTGGACCTTTAAAGGATGTAGGT
AAGGCTAAACGTGCCATAAATGAAACTCTTTTATCTTTTCGCGCATGGCGCGCATTTAAGCACCTCATTCA
ACATGATACATTTGATGGTATCGTTTATTATTCCCCCTCTATTTTTTGGGGCGACTTGGTTAAAAAAATAA
AACAACGATGCCAGTGCCCAAGCTATCTGATCCTAAGGGATATGTTTCCACAGTGGGTCATTGATGCAGGT
ATGTTGAAAGCCGGTTCACCAATTGAAAAATATTTTAGGTATTTTGAAAAAAAGTCATATCAGCAGGCTGG
CCGGATAGGGGTAATGTCTGATAAGAATCTTGAGATATTTCGCCAGACCAATAAAGGTTATCCGTGTGAAG
TTTTACGTAATTGGGCCTCAATGACTCCTGTGTCTGCCAGCGATGATTATCATTCACTTCGTCAAAAATAC
GATCTAAAAGATAAAGTCATTTTTTTCTATGGCGGTAATATTGGGCATGCTCAGGATATGGCAAACTTAAT
GCGCCTTGCGCGTAATATGATGCGTTATCATGATGCTCATTTCCTGTTTATAGGGCAGGGTGATGAAGTTG
AGCTGATAAAATCTCTTGCTGCAGAATGGAATTTAACTAATTTCACTCATCTACCTTCAGTGAACCAGGAA
GAGTTTAAATTAATTTTATCTGAAGTTGATGTCGGCCTGTTCTCCCTTTCATCTCGCCATTCTTCACATAA
TTTCCCCGGAAAATTACTAGGGTATATGGTTCAATCAATCCCGATCCTTGGGAGTGTGAATGGCGGCAATG
ATTTAATGGATGTAATTAATAAGCACAGAGCCGGTTTCATTCATGTTAATGGTGAAGATGATAAACTGTTT
GAATCTGCACAATTGCTTCTTAGTGATTCAGTTTTAAGAAAACAGCTAGGTCAGAACGCTAATGTGTTGTT
AAAGTCTCAATTTTCGGTTGAATCGGCGGCACATACTATCGAAGTCCGACTGGAGGCTGGAGAATGCGTTT
AGTTGATGACAATATTCTGGATGAACTTTTTCGCACTGCAGCAAATTCTGAACGTTTGCGCGCTCATTATT
TATTGCACGCATCTCATCAGGAGAAGGTTCAACGTTTACTTATTGCATTTGTACGCGACAGCTATGTTGAA
CCCCATTGGCATGAGTTACCGCATCAGTGGGAAATGTTTGTCGTCATGCAAGGGCAATTAGAAGTTTGTTT
GTATGAGCAAAATGGTGAGATCCAAAAACAGTTTGTTGTTGGAGACGGTACGGGAATAAGCGTCGTGGAAT
TTTCCCCAGGAGATATACATAGTGTCAAATGCCTGTCACCAAAAGCCCTTATGTTGGAGATAAAGGAGGGG
CCATTTGACCCACTCAAAGCTAAGGCTTTTTCTAAGTGGTTATAGGGCGATACACCACCGTTTATTCTTCT
ATCTTATTCTATACATGCTGGGTTACCATCTTAGCTTCTTCAAGCCGCGCAACCCCGCGGTGACCACCCCT
GACAGGAGTAGCTAGCATTTGACCACCCCTGACAGGATTAGCTAGCATATGAGCTCGAGGATATCTACTGT
GGGTACCCGGGATCCGTGTAGGCTGGAGCTGCTTCGAAGTTCCTATACTTTCTAGAGAATAGGAACTTCGG
AATAGGAACTAAGGAGGATATTCATAT
SEQ ID NO: 10 (example signal sequence for EPA carrier protein)
MKKIWLALAGLVLAPSASA
SEQ ID NO: 11 (example O1A rfb locus nucleotide sequence - O1A-EPA production strain
stGVXN4411 and stLMTB10217)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGCTAGCGTGAAG
ATACTTGTTACTAGGGGCGCAGGATTTATTGGTTCTGCTGTAGTTCGTCACATTATAAATAATACGCAGGA
TAGTGTTGTTAATGTCGATAAATTAACGTACGCCGGAAACCTGGAATCACTTGCTGATGTTTCTGACTCTG
AACGCTATGTTTTTGAACATGCGGATATTTGCGATGCTGCTGCAATGGCGCGGATTTTTGCTCAGCATCAG
CCGGATGCAGTGATGCACCTGGCTGCTGAAAGCCATGTGGATCGTTCAATTACAGGCCCTGCGGCATTTAT
TGAAACCAATATTGTTGGTACTTATGTCCTTTTGGAAGCGGCTCGCAATTACTGGTCTGCTCTTGATGGCG
ACAAGAAAAATAGCTTCCGTTTTCATCATATTTCTACTGACGAAGTCTATGGTGATTTGCCTCATCCTGAC
GAAGTAAATAATAAAGAACAATTACCCCTCTTTACTGAGACGACAGCTTACGCGCCTAGTAGTCCTTATTC
CGCATCAAAAGCATCCAGCGATCATTTAGTCCGCGCGTGGAAACGTACCTATGGTTTACCGACTATTGTGA
CTAACTGTTCGAATAACTACGGTCCTTATCACTTTCCGGAAAAATTGATTCCACTAGTAATTCTTAATGCT
CTGGAAGGTAAGGCATTACCTATTTATGGCAAAGGGGATCAAATTCGTGACTGGCTGTATGTTGAAGATCA
TGCGCGTGCGTTATATACCGTAGTTACTGAAGGTCAAGCGGGTGAAACCTATAACATTGGCGGACACAACG
AAAAGAAAAACATCGATGTTGTGCTGACTATTTGTGATTTGTTGGACGAGATAGTCCCGAAAGAGAAATCT
TATCGTGAGCAAATTACTTATGTTGCTGATCGCCCAGGGCATGATCGCCGTTATGCGATTGATGCTGAGAA
GATTGGTCGCGAATTGGGATGGAAACCACAGGAAACGTTTGAGAGTGGGATTCGTAAAACGGTGGAATGGT
ATTTGGCTAATGCAAAATGGGTTGATAATGTGAAAAGTGGTGCCTATCAATCGTGGATTGAACAGAACTAT
GAGGGCCGCCAGTAATGAATATCCTCCTTTTTGGCAAAACAGGGCAGGTAGGTTGGGAACTACAGCGTGCT
CTGGCACCTCTGGGTAATTTGATTGCTCTTGATGTTCACTCCACTGATTACTGTGGTGATTTTAGTAACCC
TGAAGGTGTGGCTGAAACAGTCAAAAGAATTCGACCTGATGTTATTGTTAATGCTGCGGCTCACACCGCAG
TAGATAAGGCTGAGTCAGAACCCGAATTTGCACAATTACTCAATGCGACTAGCGTTGAATCAATTGCAAAA
GCGGCAAATGAAGTTGGGGCTTGGGTAATTCATTACTCAACTGACTACGTATTCCCTGGAAATGGCGACAC
GCCATGGCTGGAGATGGATGCAACCGCACCGCTAAATGTTTACGGTGAAACCAAGTTAGCTGGAGAAAAAG
CATTACAAGAGCATTGTGCGAAGCACCTAATTTTCCGTACCAGCTGGGTCTATGCAGGTAAAGGAAATAAT
TTCGCCAAAACGATGTTGCGTCTGGCAAAAGAGCGTGAAGAACTAGCCGTTATTAATGATCAGTTTGGTGC
GCCAACAGGTGCTGAACTGCTGGCTGATTGTACGGCACATGCCATTCGTGTCGCACTGAATAAACCGGATG
TCGCAGGCTTGTACCATTTGGTAGCCAGTGGTACCACAACCTGGTACGATTATGCTGCGCTGGTTTTTGAA
GAGGCGCGCAATGCAGGCATTCCTCTTGCACTCAACAAGCTCAACGCAGTACCAACAACTGCCTATCCTAC
ACCAGCTCGTCGTCCACATAACTCTCGCCTTAATACAGAAAAATTTCAGCAGAATTTTGCGCTTGTATTGC
CTGACTGGCAGGTTGGTGTGAAACGCATGCTCAACGAATTATTTACGACTACAGCAATTTAATAGTTTTTG
CATCTTGTTCGTGATGGTGGAGCAAGATGAATTAAAAGGAATGATGAAATGAAAACGCGTAAAGGTATTAT
TTTAGCGGGTGGTTCTGGTACTCGTCTTTATCCTGTGACTATGGTCGTCAGTAAACAGCTATTACCTATAT
ATGATAAACCGATGATCTATTATCCGCTTTCTACACTGATGTTAGCGGGTATTCGCGATATTCTGATTATT
AGTACGCCACAGGATACTCCTCGTTTTCAACAACTGCTGGGTGACGGTAGCCAGTGGGGCCTGAATCTTCA
GTACAAAGTGCAACCGAGTCCGGATGGTCTTGCGCAGGCATTTATTATCGGTGAAGAGTTTATTGGTGGTG
ATGATTGTGCTTTGGTACTTGGTGATAATATCTTCTACGGTCACGACCTGCCTAAGTTAATGGATGCCGCT
GTTAACAAAGAAAGTGGTGCAACGGTATTTGCCTATCACGTTAATGATCCTGAACGCTATGGTGTCGTTGA
GTTTGATAAAAACGGTACGGCGATCAGCCTGGAAGAAAAACCGCTACAACCAAAAAGTAATTATGCGGTAA
CCGGGCTTTATTTTTATGATAACGACGTTGTCGAAATGGCGAAAAATCTTAAGCCTTCTGCCCGCGGTGAA
CTGGAAATTACCGATATTAACCGTATCTATATGGAACAAGGGCGTTTATCTGTTGCCATGATGGGGCGTGG
TTATGCGTGGTTAGACACGGGGACACATCAGAGCCTGATTGAGGCAAGCAACTTTATTGCAACAATTGAAG
AGCGTCAGGGGCTGAAAGTTTCCTGCCCGGAAGAAATTGCTTACCGTAAAGGGTTTGTTGATGCTGAGCAG
GTGAAAGTATTAGCTGAACCTCTGAAAAAAAATGCTTATGGTCAGTATCTGCTGAAAATGATTAAAGGTTA
TTAATAAAATGAACGTAATTAAAACAGAAATTCCTGATGTACTGATTTTTGAACCGAAAGTTTTTGGTGAT
GAGCGTGGTTTCTTTTTTGAGAGCTTTAACCAGAAGGTTTTTGAGGAAGCTGTAGGCCGCAAAGTTGAATT
TGTTCAGGATAACCATTCGAAGTCTAGTAAAGGTGTTTTACGCGGGCTGCATTATCAGTTGGAACCTTATG
CACAAGGAAAATTGGTGCGTTGCGTTGTCGGTGAAGTTTTTGACGTAGCTGTTGATATTCGTAAATCGTCA
TCGACTTTTGGCAAATGGGTTGGGGTGAATTTATCTGCTGAGAATAAGCGGCAATTGTGGATTCCTGAGGG
ATTTGCACATGGTTTTTTAGTGCTGAGTGAGACGGCGGAGTTTTTGTATAAGACGACAAATTATTATCATC
CTCAGAGTGATAGAGGAATAAAATGGGATGATCCAAGCATCAATATTTCATGGCCAGTCGATTCACAAGTG
CTGCTATCAGCTAAAGATAATAAGCATCCTCCATTAACAAAGATTGAAATGTATAGTTAAGATCACGATAA
ATCTTGGAAGGGTTGCAAAATTGAATAAAATAGTGAGCAAAAGTGAAATAAGGAACGTAATCCACAATGCT
GGCTATATGATGATTACTCAGATAGCTTTATATGTTGCACCATTATTTATACTGAGTTATCTGTTAAAAAC
ACTGGGGGTTGCACAGTTTGGTAATTATGCCTTAATACTATCAATCGTTGCATATTTACAGATTATAACGG
ATTATGGTTTTTCTTTTAGTGCAAGTCGTGCGATCTCACAGAATAGAGAGGACAAAGAATATATATCAAAA
ATTTATCTGTCAACTATGACTATCAAGTTGGCGATATGCGCTTTCTTATTCTTATTGCTCATGCTATTTTT
AAATCTTTTGCCTGTGCAAGCTGAATTAAAACAAGGAATATTATATGGATATCTTCTTGTAATAGGAAATA
CTTTCCAACCACAATGGTTTTTCCAAGGTATCGAAAAATTAAAAATCATAGCCCTTTCTAATGTTATATCA
AGATGCGCCGCGTGTTTACTTGTATTTATCTATGTGAGGAATAGCGAGGATTTACAAAAAGCACTTTTAGT
ACAGTCACTTCCATTAGTAATTTCTGCGATTGGATTAAATATATTTATATTGAAATATATCAATATTATTT
TTCCGGAAAAAAAATTATTTAAGGTAATTTTAAAAGAAGGTAAGGATTTTTTTCTTGCATCACTTTATTCT
GTTATTCTCAATAATAGTGGCATTTTTCTATTAGGGATTTTTACTAATCCTGTTATTGTTGGTGTATATGC
CGCCGCTGAAAAGATAGTCAAGGCCGTATTGTCGCTATTTACACCACTGACGCAAGCTATATATCCTTATA
ATTGTCGTAAGTTTTCACTATCCGTATTTGACGGCATTGAGGCAGCAAAAAAAACTGGTATACCAATTATA
ATTTTAGCATTTATAGCTGCTGTTATCGTTGCAATTACCTTACCTGTTGCAATCGACTATCTTAATTTTCC
AAAAGAAACAATTTTTGTAGGTCAAATATTAAGTGCATGGATCTTTTTTGGTGTTCTTAATAATGTATTCG
GCATTCAGATATTGAGTGCATCAGGAAGAAGTAAAATATATAGTAGGATGGTATTCGTATCAGCGCTTATA
ACATTACTTTTGATTACTCTATTATTGCAGTTTTGTAACGCCACTGGAGTGGCATGTGCAATATTATTGGG
TGAAATGTTCTTATCAATATTGTTACTTAAGCGATATAAAAAAATAATTTAAGGAATAGTTATGAAGAAGT
TATTATTAGTGTTCGGTACTAGGCCTGAAGCAATAAAGATGGCCTCTATCATTGAATTATTAAAAAAAGAT
TGTAGATTCGAATATAAAATATGTGTGACAGGCCAACATAAAGAGATGCTTGATCAAGTTATGCAAGTATT
TGATGTTAAACCTGATTATAATTTACGGATTATGCAGCCTGGGCAAACATTAGTATCTATAGCAACAAATA
TACTCTCACGGTTAAGTGAAGTTTTAATTATAGAAAAGCCAGATATTATACTTGTGCATGGGGATACAACG
ACTACCCTTGCTGCTACTTTAGCTGGGTATTACCACCAAATAAAAGTTTGTCATGTGGAAGCAGGATTAAG
AACAGGGGATATTTACTCTCCTTGGCCTGAAGAGGGCAATCGTAAAGTTACAGGGGCATTAGCATGTATTC
ATTTCGCCCCAACAGAGAGATCAAAAGATAATCTCCTGAGGGAGGGGGTCAAAGTAAATAATATATTTGTA
ACGGGTAATACCGTCATCGACTCTTTATTTATTGCAAAAGATATCATAGATAATGACCCTAATATAAAGAA
CGCTTTACATAATAAATTTAATTTTCTTGATAAAAGCCGACGAGTAGTACTTATAACAGGTCATCGAAGAG
AAAATTTCGGGAAAGGTTTTGAAGATATATGCTTTGCAATAAAGGAATTAGCTTTCATTTATCCTAATGTA
GATTTTATTTATCCGGTGCATCTTAATCCCAATGTAATGGAACCAGTACATCGTATATTAGATAATATATG
TAATATTTACCTTATTGAGCCCTTGGATTATTTGCCTTTTGTTTATTTAATGAATGAGTCATATTTAATAT
TGACTGATTCAGGGGGGATACAAGAAGAAGCGCCTTCGTTAGGTAAACCGGTTTTGGTTATGCGTGATACT
ACTGAACGCCCTGAGGCGGTTGAGGCTGGTACTGTTGTATTAGTGGGGACTTCTAAGATAAAAATAGTAAA
TAAAGTAACGGAGCTATTAAACAATGCTGATATCTACAATGCTATGTCTCTGTTACATAATCCATATGGCG
ATGGAACAGCTGCTCAAAAAATTCTTAATGTGCTCGCCCAAGAGCTAATTTAATTTAAGCTAAAAATATGT
TATTAATTATTGCTGATTATCCAAACGAAATGAATATGCGCGAGGGAGCTATGCAACGAATAGATGCGATA
GACTCTCTCATTCGAGATCGCAAGCGAGTGTATTTGAATATTTCATTCAAAAAGCATCTAGTTCGCTCAAA
TAGTTCCTTTAATAATGTTATAGTTGAAAATCTAAATGCAATTATTCACAGAAACATCATAAAACAGTACA
TGCAAAAATCAACAACTATATATGTTCATTCTGTTTATAATTTATTAAAGGTTATAACGCTCATTGATCTA
AAAAAAACAATTCTTGATATACATGGTGTTGTACCGGAAGAACTTTTGGCAGATAATAAAAAATTACTTAG
TAAAGTATATAACATGGTGGAAAAAAAAGGTGTCCTTGGATGCAAAAAATTAATACACGTCAGTACAGAAA
TGCAAAAACACTATGAAGCAAAATATGGAGTAAACTTGGCTGAAAGGTCAATAGTGCTCCCGATTTTTGAA
TATAAAAATATAACCCAATCGCAAAACAAATGGACAGAAAATAAAATACGAAGTATCTATCTTGGAGGATT
ACAAACATGGCAAAATATTGATAAAATGATTCAAGTTTGTGATGACACAGTGATAAACAATGAAGCAGGTA
AGTATGAATTCAACTTTTTCATCCCACAGAGTAACTTGGAAGGGTTTATAGATAAATATTCGTTAAAATTA
CATAATATCAATGCTAATGCATCTACGCTATCACGTGATGAAGTAATTCCCTTTCTAAAAGAATGTCATAT
TGGTTTTGTATTGCGCGATGATATAATAGTAAACAGAGTTGCGTGCCCTACAAAATTGGTTGAATATTTAG
AGTGTGGTGTCGTTCCAGTTGTGCTCTCCCCACTTATAGGTGATTTTTATTCGATGGGATATCAATACATT
ACTACAGAGGAAATGGCTAACAGAAGTATAAGTTTGTTGGATCTTGAAAAAATGGCTGCACATAATTTACA
AATTTTGACTTCTTATCAGAAGAGAACCTACAAGGCACAGAAAGAACTTATTGCTCAACTGTGCTGAATTT
TTTACATATATAAAATTATGTAAGCATATCGCGGGTCAGGTAATTGTATGCGTATCAAATATAAAGATAAC
GGTTATATATTATGTTTTCTATTATGTTTCATTTTGAGCTACTTAGTTTTACTCAAATCTGACTACTTTCC
TGCTGATTTTCTGCCATATACAGAAATATACGATGGGACATACGGAGAAATCAATAATATTGAGCCTGCCT
TTTTATATTTAACACGGTTGTTTCATTATTTAAATTTCCCCTATATATTTTTTGCAATGTTAGTTTGTGCC
TTATGTTTAAGTTGGAAAATAAAATATGCAAGAAAAATAATTAAAGATAGTTATATATATTTGTTCTTGTA
TGTATATGTATCATTTTATGTGTTTTTGCATGAAATGACTCAATTGCGCATAGCAATTGCAGTCACTATGT
GCTATGTGTCGGTTTATTATTACTTTTATAAAAATTGTATTAAACATGCACTGCCATGGATGGTGTTGGCT
ATTTTGTTTCATTACAGCGCCTTGCTTTTATTTATGTCATTATTTATATACAGTTATAGGAGGTTATTAAT
AGTAATTATAGGGTTTGTAATATGTATGAGCTTTTTAAACGTGTATGCAGATACAATTGCACTATATTTGC
CAAATGAAAAAATAGTAAATTATTTATATAGTATTTCATCATCATTAGACAATAGAAATGATTTGGCAATA
TTCAACCTGAATAATATAATATTTTTATCAATATTTATTTTGATCTTTTATCTTAGCCGATATATAAAATT
AAATGATAATGAGGCGAAGTTTATTAAGTATGTGCAATGTTCAGGAATATTAGCCTTTTGTATTTTCTTTC
TGGCTAGTGGAGTCCCGGTCATTGCTTATCGAACTGCAGAGTTGCTGCGAATATTTTATCCGATGGCTTTA
GTATTAATCCTTTCGCATATAAAAAATAATAATATGCGTTATTTTATTGCAGTCATTATAGTTATCCTTTC
AGGCTTAATGTTGTTTATAACACTAAGGGCTGTATCAATAGTTGGTCAAGGATTATAAAATGAATGTTGCT
ATTTTGTTGTCTACGTATAATGGCGAAAAATATTTAGAGGAACAACTGGATTCATTGCTGCTTCAAAGTTA
TCAGGATTTTGTAGTGTATATCCGTGATGACGGATCATCTGATAGAACTGTAAATATAATAAACCAATACG
TAATGAAAGATAACAGATTTATTAACGTGGGTAATTCAGAAAATCTTGGTTGTGCTGCTTCGTTTATTAAT
TTATTAAGAAATGCTTCAGCCGATATTTATATGTTTTGTGACCAAGATGATTATTGGCTTCCGAATAAATT
ACAGCGTGCTGTGGATTATTTTTCGGCTATTGATCCTTTACAACCTACCTTGTATCATTGCGATCTAAGCG
TTGTTGATGAAAAACTTAATATTATACAAAATTCATTTTTGCAGCATCAGAAAATGTCAGCGTATGATTCA
ATGAGAAAAAATAATCTTTTCATACAAAATTTTGTTGTTGGTTGTTCATGTGCTGTTAATGCTTCACTTGC
GGAATTTGTTCTTTCGCGAATTGGAGAGCAGCATGTAAAAATGATAGCTATGCATGACTGGTGGTTAGCCG
TGACTGCAAAACTTTTTGGTCGAATCCATTTTGATAATACTCAAACGATTCTTTATCGACAACATCAGGGC
AATGTATTAGGTGCAAAATCATCAGGTATGATGCGTTTTATTCGATTAGGATTAAATGGGCAAGGGATTTC
GCGAGTAGTATCTTTTAGAAAAAAAGTTTGTGCGCAAAATAAGCTTCTTTTAGATGTCTATGATAAAGATT
TAAATCTTGAGCAAAAAAAATCTATCAGGCTTGTAATTGAGGGCCTTAAAGAGAACTCTTCAATTGCTGAC
CTTTTAAAATGTTTCTATCATGGTAGCTATATGCAAGGTTTTAAACGTAATCTTGCCTTAATATATTCAGT
TCTTTACACAAAAAAAAGAAGATAGTGTATCCTTATGAAAAAAATTGCTATTATCGGTACTGTTGGCATAC
CAGCATCATATGGCGGATTTGAAACATTAGTTGAAAATTTAACAAGATACAATTCCTCGGGAGTTGAATAT
AATGTTTTTTGTTCATCGTTTCACTACAAATCCCACCAAAAAAAACATAATGGGGCCCGTTTAATTTATAT
TCCGCTTAAAGCCAATGGATGGCAGAGCATTGCGTATGACATAATTTCGTTAGCATATTCTATTTTTTTGA
AGCCTGATGTGATTCTGATTTTAGGGGTTTCTGGTTGTTCATTTTTGCCTTTCTTCAAACTCTTAACACGC
GCTAAGTTTATTACTAATATTGATGGCCTGGAATGGCGAAGAGATAAATGGAATTCAAAAGTGAAACGTTT
CTTAAAATTTTCAGAAAAAATCGCAGTTCAATATTCGGATGTCGTTATTACGGATAATGAGGCAATTTCTG
AGTACGTTTTTAACGAGTATAATAAAGATAGCCGAGTTATTGCCTATGGAGGGGATCATGCATGGTTAAAT
ACTGAGGATGTATTTACAACAAGAAATTATAAAAGCGATTACTACCTTTCTGTATGTCGTATCGAACCCGA
AAACAATGTAGAATTAATTTTAAAAACATTTTCAAAGCTAAAATATAAAATAAAATTTATTGGAAATTGGA
ATGGCAGCGAGTTTGGAAAGAAACTTAGGCTGCATTATTCTAACTATCCAAATATTGAAATGATTGATCCG
ATTTATGATCTTCAACAATTATTTCACTTACGAAATAATTGCATAGGATATATACATGGTCATTCGGCTGG
AGGAACAAACCCTTCTTTAGTCGAGGCAATGCATTTTAGTAAACCTATATTTGCATATGATTGTAAGTTTA
ATAGGTACACTACTGAAAATGAAGCATGTTATTTTTCTAATGAATCTGACCTCGCAGAGAAAATCATAATG
CATTGTGAGCTATCATTAGGTGTCTCTGGCACGAAAATGAAAGAAATTGCTAACCAGAAATACACTTGGAG
ACGAATAGCAGAAATGTATGAGGATTGCTATTAACTCTGTTAAACTTCAAATCTTTTACAATATATGGCAT
GACTATAAGCGCATTAATTGTTTTTCAAGCCGCTCTCGCGGTGACCACCCCCTGACAGGGGATCCGTGTAG
GCTGGAGCTGCTTCGAAGTTCCTATACTTTCTAGAGAATAGGAACTTCGGAATAGGAACTAAGGAGGATAT
TCATATGGATAAAGCCGTAAGCATATAAGCATGGATAAGCTATTTATACTTTAATAAGTACTTTGTATACT
TATTTGCGAACATTCCAGGCCGCGAGCATTCAGCGCGGTGATCACACCTGACAGGAGTATGTAATGTCCAA
GCAACAGATCGGCGTAGTCGGTATGGCAGTGATGGGACGCAACCTTGCGCTCAACATCGAAAGCCGTGGTT
ATACCGTCTCTATTTTCAACCGTTCCCGTGAGAAGACGGAAGAAGTGATTGCCGAAAATCCAGGCAAGAAA
CTGGTTCCTTACTATACGGTGAAAGAGTTTGTCGAATCTCTGGAAACGCCTCGTCGCATCCTGTTAATGGT
GAAAGCAGGTGCAGGCACGGATGCTGCTATTGATTCCCTCAAACCATATCTCGATAAAGGAGACATCATCA
TTGATGGTGGTAACACCTTCTTCCAGGACACTATTCGTCGTAATCGTGAGCTTTCAGCAGAGGGCTTTAAC
TTCATCGGTACCGGTGTTTCTGGCGGTGAAGAGGGGGCGCTGAAAGGTCCTTCTATTATGCCTGGTGGCCA
GAAAGAAGCCTATGAATTGGTAGCACCGATCCTGACCAAAATCGCCGCCGTAGCTGAAGACGGTGAACCAT
GCGTTACCTATATTGGTGCCGATGGCGCAGGTCACTATGTGAAGATGGTTCACAACGGTATTGAATACGGC
GATATGCAGCTGATTGCTGAAGCCTATTCTCTGCTTAAAGGTGGCCTGAACCTCACCAACGAAGAACTGGC
GCAGACCTTTACCGAGTGGAATAACGGTGAACTGAGCAGTTACCTGATCGACATCACCAAAGATATCTTCA
CCAAAAAAGATGAAGACGGTAACTACCTGGTTGATGTGATCCTGGATGAAGCGGCTAACAAAGGTACCGGT
AAATGGACCAGCCAGAGCGCGCTGGATCTCGGCGAACCGCTGTCGCTGATTACCGAGTCTGTGTTTGCACG
TTATATCTCTTCTCTGAAAGATCAGCGTGTTGCCGCATCTAAAGTTCTCTCTGGTCCGCAAGCACAGCCAG
CAGGCGACAAGGCTGAGTTCATCGAAAAAGTTCGTCGTGCGCTGTATCTGGGCAAAATCGTTTCTTACGCC
CAGGGCTTCTCTCAGCTGCGTGCTGCGTCTGAAGAGTACAACTGGGATCTGAACTACGGCGAAATCGCGAA
GATTTTCCGTGCTGGCTGCATCATCCGTGCGCAGTTCCTGCAGAAAATCACCGATGCTTATGCCGAAAATC
CACAGATCGCTAACCTGTTGCTGGCTCCGTACTTCAAGCAAATTGCCGATGACTACCAGCAGGCGCTGCGT
GATGTCGTTGCTTATGCAGTACAGAACGGTATTCCGGTTCCGACCTTCTCCGCAGCGGTTGCCTATTACGA
CAGCTACCGTGCTGCTGTTCTGCCTGCGAACCTGATCCAGGCACAGCGTGACTATTTTGGTGCGCATACTT
ATAAGCGTATCGATAAAGAAGGTGTGTTCCATACCGAATGGCTGGATTAA
SEQ ID NO: 12 (example O2 rfb locus nucleotide sequence - O2-EPA production strain stGVXN4906)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGTGAAAATACTT
GTTACTGGTGGCGCAGGATTTATTGGTTCAGCTGTAGTTCGTCACATTATAAATAATACGCAGGATAGTGT
TGTTAATGTCGATAAATTAACGTACGCCGGAAACCGGGAATCACTTGCTGATGTTTCTGATTCTGAACGCT
ATGTTTTTGAACATGCGGATATTTGCGATGCACCTGCAATGGCACGGATTTTTGCTCAGCATCAGCCGGAT
GCAGTGATGCACCTGGCTGCTGAAAGCCATGTTGACCGTTCAATTACAGGCCCTGCGGCATTTATTGAAAC
CAATATTGTTGGTACTTATGTCCTTTTGGAAGCCGCTCGCAATTACTGGTCTGCTCTTGATAGCGACAAGA
AAAATAGCTTCCGTTTTCATCATATTTCTACTGACGAAGTCTATGGTGATTTGCCTCATCCAGATGAAGTA
AATAATACAGAAGAATTACCCTTATTTACTGAGACGACAGCTTACGCGCCAAGCAGCCCTTATTCCGCATC
CAAAGCATCCAGCGATCATTTAGTCCGCGCATGGAAACGTACGTATGGTTTACCGACCATTGTGACTAATT
GCTCGAACAACTATGGTCCGTATCACTTCCCGGAAAAGCTTATTCCATTGGTTATTCTTAATGCACTGGAA
GGTAAGGCATTACCTATTTATGGCAAAGGGGATCAAATTCGCGACTGGTTGTATGTAGAGGATCATGCTCG
TGCGTTATATACCGTCGTAACCGAAGGTAAAGCGGGTGAAACTTATAACATTGGCGGACACAACGAAAAGA
AAAACATCGATGTTGTGCTGACTATTTGTGATTTGTTGGATGAGATTGTACCGAAAGAGAAATCTTATCGT
GAGCAAATTACTTATGTTGCTGATCGCCCAGGGCATGATCGCCGTTATGCAATTGATGCCGATAAAATTAG
CCGCGAATTGGGCTGGAAACCACAGGAAACGTTTGAGAGCGGGATTCGCAAAACGGTGGAATGGTATCTGG
CTAATACAAATTGGGTTGAGAATGTGAAAAGCGGTGCTTATCAGTCATGGATCGAACAAAACTATGAGGGC
CGTCAGTAATGAATATCCTGCTTTTCGGCAAAACAGGGCAGGTGGGTTGGGAACTGCAGCGTGCTCTGGCG
CCGCTGGGTAATCTGATCGCTCTTGATGTTCACTCCACTAATTATTGTGGAGATTTCAGCAACCCCGAAGG
TGTGGCAGAAACCGTCAAAAAAATTCGTCCTGACGTTATTGTTAATGCTGCTGCTCACACTGCAGTAGATA
AAGCAGAATCAGAACCGGATTTCGCACAATTACTTAACGCGACAAGCGTCGAAGCGATTGCAAAAGCTGCT
AATGAAGTCGGGGCCTGGGTTATACACTACTCTACTGATTATGTTTTCCCAGGCAGTGGTGACGCGCCATG
GCTGGAAACGGATGCAACAGCACCGCTAAATGTTTACGGTGAAACAAAATTAGCTGGGGAAAAGGCATTAC
AAGAACATTGCGCAAAGCATCTTATTTTCCGTACCAGCTGGGTATACGCTGGTAAAGGAAATAACTTTGCT
AAAACGATGTTGCGTTTGGCAAAAGAACGCGAAGAACTGGCTGTGATAAACGATCAGTTTGGCGCACCAAC
AGGTGCTGAATTGCTGGCTGATTGCACCGCTCATGCCATTCGCGTGGCATTAAAAAAACCAGAAGTCGCTG
GCTTGTACCATCTGGTAGCAAGTGGCACAACAACCTGGCACGATTATGCTGCGCTGGTTTTTGAAGAGGCG
CGCAAAGCAGGGATTAATCTTGCACTTAACAAACTTAACGCCGTGCCAACAACGGCCTATCCCACACCAGC
CCGTCGACCCCATAACTCTCGCCTCAATACAGAAAAGTTTCAGCAGAACTTTGCGCTTGTCTTGCCTGACT
GGCAGGTGGGCGTGAAACGTATGCTCAACGAATTATTTACGACTACGGCAATTTAACAAATTTTTGCATCT
CGCTCATGATGCCAGAGCGGGATGAATTAAAAGGAATGGTGAAATGAAAACGCGTAAAGGTATTATTCTGG
CTGGTGGTTCCGGCACTCGTCTTTATCCTGTGACGATGGCAGTGAGTAAACAATTGCTGCCGATTTATGAT
AAGCCGATGATTTATTATCCGCTTTCAACGCTTATGTTAGCGGGTATTCGCGATATTCTTATTATTAGTAC
GCCACAGGATACACCGCGTTTCCAACAATTATTGGGGGACGGGAGCCAGTGGGGTCTTAATCTACAGTATA
AAGTACAACCGAGTCCGGATGGCCTGGCGCAAGCGTTTATTATTGGCGAAGACTTTATTGGTGGTGATGAT
TGTGCACTCGTACTTGGCGATAATATCTTCTATGGACACGACTTGCCGAAATTGATGGAAGCTGCTGTTAA
CAAAGAAAGCGGTGCAACGGTATTTGCTTATCACGTTAATGATCCTGAACGCTATGGTGTCGTGGAGTTTG
ATAATAACGGTACGGCAATTAGCCTGGAAGAAAAACCGCTGGAGCCAAAAAGCAACTATGCGGTTACTGGG
CTTTATTTCTATGACAATGACGTTGTGGAAATGGCTAAAAACCTTAAGCCTTCTGCCCGTGGCGAACTGGA
AATTACCGATATTAACCGTATTTATATGGAACAAGGACGTTTGTCTGTAGCCATGATGGGGCGTGGCTATG
CATGGTTGGATACAGGGACGCATCAAAGCCTTATTGAAGCAAGTAACTTCATTGCAACAATTGAAGAGCGT
CAGGGATTAAAGGTATCTTGCCCGGAAGAGATTGCTTACCGTAAAGGGTTTATTGATGCCGAGCAGGTGAA
AGTATTAGCCGAACCGCTTATCAAGAATCAATATGGTCAATATTTGCTGAAAATGATCAGCGAATAGTATA
TGGGAACTCAATGATGGATATTAAATTAATCTCTTTGCAAAAACATGGGGATGAGCGCGGTGCATTAATTG
CTCTTGAAGAGCAACGAAATATACCTTTCGAAGTCAAAAGAATATATTACATACTTGAGACTCTTAATGGA
GTAAGACGCGGATTTCATGCGCACAAGGTTACTCGTCAGTTAGCTATTGTAGTCAAGGGAGCTTGTAAATT
TCATCTGGATAATGGTAAAGAAACAAAGCAGGTGGAACTTAATGATCCAACAATTGCGTTGCTGATAGAAC
CCTATATATGGCATGAAATGTATGATTTTAGTGATGATTGTGTGCTGCTTGTAATTGCGGATGATTTCTAT
AAAGAGTCTGATTATATCCGCAATTATGATGATTTTATTAGAAGAGTAAATTCAATTGAGAATTCATAAGC
TAAGTGACGTCCAGACAACATCAATTGGTGATGGAACAACTATCTGGCAGTTTGTTGTGATACTAAAAGGT
GCTGTAATTGGTAATAATTGCAACATCTGTGCAAATACCTTAATTGAAAATAACGTTGTAATTGGTAACAA
TGTCACAGTCAAAAGCGGTGTGTATATTTGGGATGGCGTTAAAATAGAGGATAATGTTTTTATTGGTCCTT
GTGTAGCATTTACAAATGATAAGTATCCTCGCTCTAAAGTCTATCCTGATGAATTTTTGCAAACAATAATA
CGCAAAGGAGCATCAATAGGTGCTAACGCAACCATCCTGCCAGGAATTGAAATTGGTGAAAAAGCAATCGT
TGGTGCGGGGAGTGTTGTAACCAAAAATGTACCGCCATGCGCAATAGTAGTAGGTAATCCAGCTCGATTTA
TTAAATGGGTAGAGGATAATGAATAAAATTGATTTTTTAGATCTTTTTGCAATTAACCAGCGACAGCACAA
AGAATTAGTCTCTGCGTTTAGTAGGGTGCTAGATTCTGGTTGGTATATCATGGGCGAAGAACTTGAGCAGT
TCGAGAAAGAGTTCGCAGAATACTGTGGAGTTAAGTATTGCATTGGTGTAGCAAATGGCCTTGATGCGTTG
ATACTAGTATTGAGGGCATGGAAAGAACTTGGCTATCTTGAAGACGGTGACGAGGTATTAGTACCGGCAAA
TACATATATTGCTTCTATTCTTGCTATAACAGAGAACAAACTTGTTCCTGTTCTTGTTGAACCAGATATAG
AAACTTATAATATTAATCCTGCTTTAATTGAAAATTACATTACGGAAAAAACTAAAGCAATATTACCGGTT
CACTTATATGGTCTATTGTGCAATATGCCAGAAATTAGTGCAATCGCCAGAAAATATAATCTGTTGATTCT
TGAAGATTGTGCACAAGCACATGGTGCAATACGTGATGGTCGCAAAGCTGGAGCTTGGGGGGATGCTGCAG
GATTTAGTTTTTATCCAGGAAAAAACCTTGGAGCTTTGGGGGATGCGGGAGCTGTTACTACAAATAATGCA
GAATTATCCTCAACTATAAAAGCTTTGCGAAATTATGGGTCACATAAGAAATATGAAAATATTTATCAGGG
ATTGAATAGTCGATTGGATGAACTGCAAGCAGCCTTATTGCGTGTAAAAATCCATACATTACCGGAAGATA
CTGCGATTCGGCAAAGGATTGCTGAAAAATATATTCGTGAAATAAAAAACCCTGCGATTACGTTACCAGTG
TACGAAGGCCAAGGTGCGCATGTTTGGCATTTATTTGTAGTAAGAATCGCTAATCGTGAAAAATTCCAGTC
ATACTTATTAGAGAAGGGTATCAAAACCTTAATTCACTATCCATTACCACCCCATAAGCAGCAAGCATATC
AAAATATGTCTAGCCTTAGCCTTCCAATTACTGAGCAAATTCATGATGAAGTCATTTCTTTACCTATAAGT
CCGGTAATGAGTGAAGATGATGTCAATTATGTAATCAAAATGGTCAATGATTACAAGTAATGAAAAAATTT
CTTCAGGTAACTATATTATCCGCTATCTATACATTCATTAAAATGATTGCGGGTTTTATCATCGGTAAGGT
AGTAGCAATTTATACAGGGCCATCAGGGGTAGCAATGCTTGGCCAAGTGCAAAGTTTAATCACAATAGTTG
CAGGTACTACCTCTGCACCTGTAAGCACAGGCCTTGTTCGATATACTGCGGAAAATTGGCAAGAAGGACAA
GAAGCATGCGCGCCATGGTGGCGCGCATGCTTAAGGGTTACTCTGTTTTTATTCTTGCTTATTATTCCCGT
TGTTATTATATTGTCGAAAAATATTAGTGAGTTACTTTTTAGCGATGGACAATACACATGGTTAATCATTT
TCGCATGTTGTATATTGCCATTCTCCATTATAAATACATTGATCGCTTCAGTTTTAAATGGTCAACAATTT
TATAAGCAATATATATTGGTTGGGATGTTTTCTGTATTCATTTCTACTATGTTTATGATTTTGTTGATTGT
AGCTTATAATCTTAAAGGTGCATTGATTGCCACAGCTATAAATAGTGCTATTGCTGGTCTTGTATTGGTTT
TATTTTGTCTCAATAAATCTTGGTTTAGATTTAAATATTGGTGGGGTAAAACGGATAAAGACAAAATTATA
AAAATTATTCATTATACTCTGATGGCTCTGGTTTCTGTTATCTCCATGCCTACAGCATTGATGTGTATTAG
AAAAATATTGATTGCTAAAACTGGTTGGGAGGATGCAGGGCAATGGCAGGCCGTATGGAAGATATCTGAGG
TTTATCTTGGTGTTGTGACAATTGCTTTGTCAACATATTTCTTACCAAGATTGACAATTATAAAAACAAGT
TTCCTTATAAAAAAAGAAGTAAATAGTACTATATTATACATAATATCTATTACTTCATTCATGGCGTTGAG
TATCTATTTATTCCGCGATTTGGTAATAACAGTTTTATTTACTGAACAGTTTCGCTCAGCTCGTGAATTAT
TTTTATTACAACTTATAGGGGATGTAATAAAAATTGCTGGGTTTCTTTATGCATACCCTCTTCAAAGTCAG
GGGCATACTAAACTATTCATCAGTTCAGAAGTGATTTTTTCTATGCTCTTTATCATTACCACCTATATTTT
TGTTGTAAATTATGGAGTACATGGTGCTAACATAAGTTATGTCATTACATATAGTTTATATTTTGTGTTTG
CATTTGTGTTTACTAATTTTATTAATGTTAGAAGAAATAATTAAAAACAGAGGTTGAATTTTGAAAATAAT
TATACCTGTCTTAGGATTTGGCAGGGCTGGTGGTGAAAGAGTTCTTTCTAAGCTGGCAACTGAATTGATGA
ATTATGGACATGATGTAAGTTTTGTTGTTCCAGATAATAGAACTAATCCATATTATGCTACCACAGCAAAA
ATTGTCACGAGTAAATCTAGTCAAAACCGTGTAAAAATATTGAGAATCATTAAAAATTACTATAATCTGTG
GCGTAAATGCATAGAGTTAAATCCTGATGCTGTAGTTGCTAGTTTTCATTTGACTGCCTATCTTGTCGCAT
TATTACCAATCACCCGTCGTAAGAAATATTATTATATTCAGGCGTATGAAGTTAATTTTTTTGATAATATA
ATATGGAAATTAATAGCGGGTTTAACATATTATTTACCGCTTAAAAAAATACTAAATAGTCCTAATTTGCT
TCCTCATAAACATGATGATTTTATAGGAGTAGTTCCTGCAGGAGTAGATTTAAACGTTTTCTATCCGAAAC
CATCAAATAGGTTATTAAATGGTCACACATCAATAGGGATTATTGGTAGAAAAGAGAAGCACAAAGGAACT
AGCGAAATTATTTCAGTATTGTGTTCACTGGAAAATAAAGCTGGAATTATAATCAATATTGCGATCTATCT
TGAAGAAGTTGATAAGCAGCGTTTAATCGCTGCCGGGTTTCAGGTTAATTTTTTTCCGATTACTTCTGATT
TAGAATTGGCATCCTTTTATCGAAGCAATGACATCATGATTGCTGTTGGGTTAATTGAAGATGGCGCTTTC
CATTATCCTTGTGCTGAATCAATGGCTTGTGGTTGTCTTGTTATTTCAAATTATGCGCCACTTACTGAAAC
TAACAGTGTACTTAAATTAGTCAAGTTTGATGCTTGCAAACTTGGTGAAGCAATTAATCTTTGTCTCAATC
TTGACCTAGAAGAAAAAAGCAAAGAAATCCAATCTAATATTTCTGTGTTGAATAAATATGACTGGAAAATT
GTTGGTGAAACTTTCAATAGTTTATTGTTAGATGCAAATAAATAGTATACGTTGATGGGGAAAATATGAAT
ATTGTTAAAACTGATATTCCAGATCTGATCGTTCTTGAACCAAAAGTGTTTAGTGATGAACGCGGCTTTTT
TATGGAGAGTTATAATCAGATTGAATTTGAGAAGGCAATAGGAAGGCACGTAAATTTTGTTCAGGATAATC
ATTCAAAATCTAGTAAAGGCGTACTACGTGGGTTGCATTATCAATTAGCACCGTATGCACAGGCTAAATTA
GTTCGATGTGTTGTAGGTCAGGTATTTGATGTTGCTGTTGATCTTAGAAAAAATTCACCAACGTTCAAAAA
ATGGTTTGGAATAACCCTTTCCGCAGAAAATAAACGACAATTATGGATACCCGAAGGATTTGCTCATGGTT
TCTTGGTGACCAGTGATGAAGCTGAGTTCATTTATAAGACAACTAACTACTATGCTCCTGGTCATCAGCAA
GCAATTATTTACAATGATCCTATTTTAAACATCGATTGGCCTTTCTGCAGTAGTGCTCTGTCATTATCACA
AAAAGATCAAGAAGCAAAATTATTTTCAGAATTATTGGACAGTGAACTGTTCTAATAAAGTGTGCCACCTT
ATCCGTCTGAAGGATAGGTGGTTGCTTATATTTTTTTGAGTATGTTTGTATAATGACAGAAAATAGTCCGA
AATATAAACACGATAAAAGCTTAATAAGTTTTATCTACTTATTTTTTATATTTACACTTATTGTAGGCTTT
ATTATCGCAAATACCCAGTTTTTGGGGCGAAGTAGAGACTATGATAATTATATACAGATCTTTTCTGGTAA
AGAAGGGGAGGGGGTTCTTGAATTATTTTATCGCGGATTGATGTTAATAACGACCAGCTATGAAACTATCA
TTTTTATAATTTTAACATGTTCTTTTTTTATAAAGGCAAGGTTTCTCGCTAACTATTCGCGTAATTTTTCA
GGCTTGACCTTATTCTTTATTTATTATGCAAGCGTTGCACTTTGGGTTTTAGATTATACTCAATTCAGAAA
TGGTCTATGTATTTCCATTTTAATGTTTTCCGTATACTATTTATTTATAAATAAACCGACTTATTTTTATT
TCTCGGTATTATGTGCAATTGCAACTCATTGGTCTGCTTTGCCTTTTTTGCTTTTATATCCTTTTGTCTAT
TCAACAAAAATAAGACGCCTTGGTTATTTTTGTTTCAGTATTCTTGTTTTGATTGCGATCTCAGGAGAAGG
AAAAGAGATCATATCTTTTATAAGAAATTTTGGAGTGGGACAAAAAATAGGAAATGAAGCTGGTGTAAATT
TAATAAATTCATTATCCCTTACCGCTATTTCCTGGTTTATTATTAGTTACATATCAAGCATTGGAAATGAA
AGGAGAAATTTAAGGCTTTTCTTTTGTTATGGTGTCATGCAATACGTGACTTTTAGCCTTTTCTCTCTACC
TGTTATGGCTTTCCGTATTTTGGAAATGTATTTTTTCCTTATGCTAACCATTGGGGTGTTTATTAAGCAAA
AAAAGAATTATTATTTTATTTTTTGCAAAGTGTTAATTTTATTGTATCTAACATACTATTATCATATGGTC
TTTGGAGTGATTAATGTGTAAGGCTAAGGTGTTGGCTATAATTGTTACTTACAACCCGGAAATTATTCGAT
TGACGGAATGTATTAACTCTTTAGCCCCACAAGTTGAGAGAATAATTCTTGTAGATAATGGCTCAAATAAT
AGTGATTTGATAAAAAATATCAGTATTAATAACCTTGAAATTATTTTACTTTCGGAAAACAAAGGCATTGC
ATTTGCTCAGAACCATGGTGTTAAGAAGGGCCTGGAAGCAAAAGAGTTTGACTATTTATTTTTCTCAGATC
AGGATACTTGCTTTCCTAGCGATGTTATTGAAAAACTTAAGAGTACATTTACGAAAAATAATAAAAAAGGT
AAAAATGTTGCTTGTGCTTCTCCTTTTTTTAAAGACCATCGTTCAAATTATATGCATCCGTCAGTCAGCCT
AAATATTTTTACGAGTACAAAAGTTATATGTAGTGAAGTAGACGATGATCTTTATCCCTCGCATGTTATTG
CTTCTGGGATGTTAATGTCTCGTGAAGCATGGCGCGTCGTCGGACCATTTTGTGAAAAACTCTTTATAGAC
TGGGTTGATACAGAATGGTGTTGGCGTGCATTAGCTAATAATATGATTATTGTTCAGACACCATCAGTCAT
CATTTCTCATGAACTTGGGTATGGGCAGAAAATTTTTGCTGGTCGATCTGTTACAATACATAATTCTTTCA
GAAATTTTTATAAAATACGCAATGCAATATACTTAATGCTGCATTCAAATTATAGCTTCAAGTATCGTTAT
CATGCTTTTTTTCATGCGACAAAGAATGTTGTATTTGAAATTTTATATTCGAAAGAAAAATTAAATTCACT
GAAGGTTTGTTTTAAAGCTGTACGTGATGGTATGTTCAATAATTTTTAATACGAAAATAGTTAGGCTCAAG
GTGTTTAAATGGAAGAAAATAATATGAAGACGGTCGCTGTAGTTGGCACAGTGGGTGTTCCTGCTTGTTAT
GGTGGGTTCGAATCACTTGTTCAGAATCTAATTGATTATCAATCTGATGGTATACAATATCAGATATTTTG
CTCTTCAAAAAAATATGATAAAAAATTTAAAAATTATAAAAATGCAGAATTAATCTATTTGCCGATAAATG
CCAATGGCGTCTCTAGCATAATTTATGATATTATGTGTTTAATTATTTGTTTATTCAAAAGGCCAGATGTT
GTTTTAATATTGGGGGTGTCTGGTTGTTTATTTCTACCAATTTATAAACTATTTTCAAAATCAAAGATTAT
TGTCAATATTGATGGGCTTGAATGGCGTAGAAATAAATGGGGAACGTTTGCTAAGAAATTTCTTAAAATAT
CTGAGGCGATATCTATTAGAATAGCTGATATTATCATTTCAGATAATCAAGCAATAGCTGATTATGTGGAA
AATAAGTACAAGAAAAAAAGTGTAGTTATAGCTTATGGCGGAGATCATGCCACTAATCTTAGTACACCGAT
AGACAATGATCAAAAAAAAGAAGGTTATTATTTGGGGCTTTGTAGGATAGAGCCTGAGAATAATATAGAAA
TGATTCTGAATGCCTTCATTAATACAGATAAAAAAATTAAATTTATGGGTAATTGGGATAACAGCGAGTAT
GGACGCCAGCTAAAAAAATATTATTCAAACTATCCAAATATCACCCTACTAGAACCTAACTATAATATTGA
AGAGCTTTATAAACTAAGAAAAAATTGTCTTGCATACATTCATGGACACTCGGCTGGTGGAACAAACCCTT
CTTTAGTTGAAGCGATGCATTTTAATATTCCTATTTTTGCTTTCGATTGTGACTTTAATCGTTACACAACT
AACAATTTAGCTCATTACTTTAATGATTCTGAACAACTTAGCTTATTAGCAGAAAGTTTGTCTTTTGGAAA
TCTTAAATGTCGAGTATTAGATTTAAAAAATTATGCTGAAGATATGTATAACTGGAGGCATATAGCTGCTA
TGTATGAATCTATTTATTAAACGCATTAACAATAATATAATTGACCTTATATAGCAGGGAAAGATCACGTA
ACGCTGCGGCGCGCCGATCCCCATATGAATATCCTCCTTAGTTCCTATTCCGAAGTTCCTATTCTTTCTAG
AGAATAGGAACTTCGGAATAGGAACTAAGGAGGATATTCATATGGATAAAGCCGTAAGCATATAAGCATGG
ATAAGCTATTTATACTTTAATAAGTACTTTGTATACTTATTTGCGAACATTCCAGGCCGCGAGCATTCAGC
GCGGTGATCACACCTGACAGGAGTATGTAATGTCCAAGCAACAGATCGGCGTAGTCGGTATGGCAGTGATG
GGACGCAACCTTGCGCTCAACATCGAAAGCCGTGGTTATACCGTCTCTATTTTCAACCGTTCCCGTGAGAA
GACGGAAGAAGTGATTGCCGAAAATCCAGGCAAGAAACTGGTTCCTTACTATACGGTGAAAGAGTTTGTCG
AATCTCTGGAAACGCCTCGTCGCATCCTGTTAATGGTGAAAGCAGGTGCAGGCACGGATGCTGCTATTGAT
TCCCTCAAACCATATCTCGATAAAGGAGACATCATCATTGATGGTGGTAACACCTTCTTCCAGGACACTAT
TCGTCGTAATCGTGAGCTTTCAGCAGAGGGCTTTAACTTCATCGGTACCGGTGTTTCTGGCGGTGAAGAGG
GGGCGCTGAAAGGTCCTTCTATTATGCCTGGTGGCCAGAAAGAAGCCTATGAATTGGTAGCACCGATCCTG
ACCAAAATCGCCGCCGTAGCTGAAGACGGTGAACCATGCGTTACCTATATTGGTGCCGATGGCGCAGGTCA
CTATGTGAAGATGGTTCACAACGGTATTGAATACGGCGATATGCAGCTGATTGCTGAAGCCTATTCTCTGC
TTAAAGGTGGCCTGAACCTCACCAACGAAGAACTGGCGCAGACCTTTACCGAGTGGAATAACGGTGAACTG
AGCAGTTACCTGATCGACATCACCAAAGATATCTTCACCAAAAAAGATGAAGACGGTAACTACCTGGTTGA
TGTGATCCTGGATGAAGCGGCTAACAAAGGTACCGGTAAATGGACCAGCCAGAGCGCGCTGGATCTCGGCG
AACCGCTGTCGCTGATTACCGAGTCTGTGTTTGCACGTTATATCTCTTCTCTGAAAGATCAGCGTGTTGCC
GCATCTAAAGTTCTCTCTGGTCCGCAAGCACAGCCAGCAGGCGACAAGGCTGAGTTCATCGAAAAAGTTCG
TCGTGCGCTGTATCTGGGCAAAATCGTTTCTTACGCCCAGGGCTTCTCTCAGCTGCGTGCTGCGTCTGAAG
AGTACAACTGGGATCTGAACTACGGCGAAATCGCGAAGATTTTCCGTGCTGGCTGCATCATCCGTGCGCAG
TTCCTGCAGAAAATCACCGATGCTTATGCCGAAAATCCACAGATCGCTAACCTGTTGCTGGCTCCGTACTT
CAAGCAAATTGCCGATGACTACCAGCAGGCGCTGCGTGATGTCGTTGCTTATGCAGTACAGAACGGTATTC
CGGTTCCGACCTTCTCCGCAGCGGTTGCCTATTACGACAGCTACCGTGCTGCTGTTCTGCCTGCGAACCTG
ATCCAGGCACAGCGTGACTATTTTGGTGCGCATACTTATAAGCGTATCGATAAAGAAGGTGTGTTCCATAC
CGAATGGCTGGATTAA
SEQ ID NO: 13 (example O6A rfb locus nucleotide sequence - O6A-EPA production strain
stGVXN4112 and stLMTB10923)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATTTGCCCGCC
GGGCGTGACAATTATGAACGTGCGTCAGGGCGAACCTTTAGGTTTGGGCCACTCCATTTTATGTGCACGAC
CTGCCATTGGTGACAATCCATTTGTCGTGGTGCTGCCAGACGTTGTGATCGACGACGCCAGCGCCGACCCG
CTGCGCTACAACCTTGCTGCCATGATTGCGCGCTTCAACGAAACGGGCCGCAGCCAGGTGCTGGCAAAACG
TATGCCGGGTGACCTCTCTGAATACTCTGTCATCCAGACCAAAGAGCCGCTGGACCGCGAAGGTAAAGTCA
GCCGCATTGTTGAATTCATCGAAAAACCGGATCAGCCGCAGACGCTGGACTCAGACATCATGGCCGTTGGT
CGCTATGTGCTTTCTGCCGATATTTGGCCGGAACTTGAACGCACTCAGCCTGGTGCATGGGGGCGTATTCA
GCTGACTGATGCCATTGCCGAACTGGCGAAAAAACAGTCCGTTGATGCCATGCTGATGACCGGCGACAGCT
ACGACTGCGGTAAAAAAATGGGTTATATGCAAGCGTTCGTGAAGTATGGACTACGCAACCTCAAAGAAGGG
GCGAAGTTCCGTAAAGGGATTGAGAAGCTGTTAAGCGAATAATGAAAATCTGACCGGATGTAACGGTTGAT
AAGAAAATTATAACGGCAGTGAAGATTAGCGGCGAAAGTAATTTGTTGCGAATTTTCCTGCCGTTGTTTTA
TATAAACAATCAGAATAACAACGACTTAGCAATAGGATTTTCGTCAAAGTTTTCCAGGATTTTCCTTGTTT
CCAGAGCGGATTGGTAAGACAATTAGCATTTGAATTTTACGGGTTTAGCGCGAGTGGGTAACGCTCGTCAC
ATCGTAGACATGCATGCAGTGCTCTGGTAGCTGTAAAGCCAGGGGCGGTAGCGTGCTGAAATTATAAAGTC
ATTCTTATAGAACATCGCATTTCAATAATATAATTACACCTAAATGAATAGGATACAACGTGTGCACAATT
ATTTAAGGCTTAAAGATAAAATAAAAAACGTATTTTTAGGGTTGTATATATTGCAGTTATTTAATTATATC
GCGCCATTGGTAATTATCCCTATCCTGATAAAATATATTGGGTTGGGGGAATATGGGGAATTGGTCTATAT
TACATCTATTTATCAAATAGTGGCTTTGATTATTGATTTTGGCTTTACTTACACAGGACCTGTGGTTGCTG
CGAGACATAGATGTGAGACCCAAAATTTACAGCGCTATTACTCAATAGTTGTTCTTTTAAAATCATTGCTT
TTTATAATTGCATTAACATGTGTATTTTTATTGTGCAGATTAAATATAGTCCACTTGTCATTTTTTGGGTT
TTTGTCAATTTTTCTATGCACTATTGGTAATATATTATCGCCCAATTGGTTTTTGCAGGGGATTGGTGATT
TTAAAAAACTTTCATACTCACAAGTAATAGTGAGAATAACATTGTTTATCATACTTCTTGTTTATGTCTGT
AGTGGCGGAGATAATGTTTTTATCCTAAGTTTTTTGCAAAATGCAACATTACTCATATGCTGTATATACTT
ATGGCCAAATATTCATATTAGCCATGTTGTTCATCTTAAACCTAATGAATGCATTGTGGAATTTAAGAAGG
CAGGAAATGTTTTTATTGGCGTAATAGGTACGATTGGTTACAATGGTCTAATTCCTGTGTTAATTGGAAAC
CTTTGCGGTAATACGAGTCTTGGTGTTTTTTCAATCGTTCAAAAAATGACAACAGCATGTCAAAGTCTAAT
TAATCCAATATCACAGTATATGTTATCTCAAGTTTCAGAAATTAAACCTCAAGATAAACTGTTTTATTATA
GAATTAAAAAAAGTTTTTTTGTGCATTTAACAATTAGCATAATTGCATGTTTATGTTATATGGGGTTAGGG
CAATATGTGGCGACTTTTATAGGTAAAGTTGACGTTTCATTTGTTATTATTTTATTTGCGTCAATAATTAC
CATTTTTTCATCTTTAAATAATGTCCTTGGTATACAGTTTCTTATACCGACAGATAATGTAAAAATACTAC
GAAGTATAAATGTTATGGCGGGAATTATTGTTGTTAGTTTGTCCTGGCTGTTAATATCACGCTTTGACATT
CTGGGGGGGGTTTTATTAAACCTAATTGGTGAGTTTCTTGTATTCAGTATGCTAGCTTTTATTGCCCATCG
AAAGTGGGGAGCGAGAGTATAATGAAAGTGAAGGCGGTTCCTGCTATTACATTCTATTTAAGTTTAATGCT
GACAATTTTAGTGTTACTGTTTGGTAATGAACCAAATAAATCACAATATATCCTTGTTATAGCAACGATAA
CAGTTTTTTATATCGCATATATCACTAATAAAATAACTTCTCCGGCCAGCCTTCTCGTTATATCATCTTTT
GTGTTTTTAGGTTGTCGCCCTTTATTATCTTTGTTTGCAAACTATGATTATAGGATTGCCGATTGGTTTAT
TGAAGGATATATGGATGACGATGTGATTTTGGCTAACTATGCTATAACACTAATGTATTATGGTTATACAT
TGGGACTAATTCTATGCAAAAATACTGAAAAATTTTATCCGCATGGTCCTTATCCTGAAAAACAATTGCTA
AAAATAAAGTTTCTTTTGACTTTATTTTTTCTGGGTTCGATAGGTATGGTTGTAAAAGGGATATTCTTTTT
TAACTTTATAGAATCTAATAGTTATGTTGATATTTATCAATCAAATATAACAACGCCAATAGGTTATGATT
TTCTATCTTATTTATTTTATTGTTCTTTTTTCCTTATATGTGCGTTTCATATACAGTTCAGAACAAATAAA
AAATTTCTTTTTATTGCGATATGCATTGCTGCATTTAGCACCTTGAAGGGTAGTCGTAGTGAAGCTATAAC
GTTTCTTTTAACGGTTACATGTATATATTTTAATGAAGTAAAGACAAGAAACTTACGTCTGCTGATTACAA
TGATTTTTGTTTTTAGCGTCATTTTTGTGATTAGTGAATTTATCTCAATGTGGCGCACTGGAGGGAGTTTT
TTTCAATTAATGCAGGGTAATAATCCTGTTATAAACTTTGTATACGGCATGGGAGTATCATATCTTTCCAT
TTATCAATCAGTAAAACTACAACTATTGTCAGGGGGATATAATGTTACCTATCTATTCAGCCAGTTAATAA
TAACTTGCTCGTCAATATTTAATGTCAAATTGAGCTTGCCGGAAATAAGCTATAGCCATTTGGCCTCATAC
ACAGCAAACCCAGAACTATATAATCTTGGGTTCGGACTTGGGGGGAGTTATTTAGCAGAATCGTTTTTAGC
ATTTGGTCTGATTGGATGTTTCATTATACCCTTTTTACTTTTACTTAATTTAAATGTATTGGAAAAATATA
CAAAAAACAAACCAATTATATATTTTGTTTATTATAGTGTGTTGCCACCTATATTATTCACACCAAGAGAG
ACTTTGTTCTATTTCTTCCCCTATCTTGTCAAAAGTATATTTGTTGCTTTTTTAGTTACATTATACATCCA
GTATAAAAAGGATTGACCAAAATGTCAGAAAAAAATGTCAGCATAATAATCCCAAGTTATAACAGGGCTCA
TATTCTTAAGGAGGTCATACCAAGTTATTTTCAGGATGAGACTTTAGAGGTTATAGTTATCAATGATGGAT
CAACAGATAATACAAATAGTGTATTAGCTGAACTGAAGGAAAAATATTCTCAGTTAGTTATTTTAGAAAAT
GAAACGAATAAAAAACAGATGTATTCTAAAAACCGAGGGATTGAAATAGCCAAAGGGAAATATATTTTTTT
TGGTGATGATGACTCTTACCTCTTACCCGGTGTTATATCTCGGTTATTGGCTACAAAATATGAGACAGGCG
CTGATGTAATCGGCGCAAGAATACTTTATATGAATAATAACGAGAAAACAATTGAAGATTGCATAAATCGA
CATAAAAAAGAGGGGCGTTTTGTTAGTGATCTAAATAGATTGGATTTTAGTTATACATGTGATTTGGACCA
TCCGATTGAATGTTTTTATGCACAGCCTTTTGTTCTAGCTGAAAGGGAACTAATATCGAAATATCGATTTG
ATATATCTTATACGGGAAACTGCTATCGTGAGGAAACTGATTTCATGCTATCTCTATTTATTAAAAATAAA
AAATTTATATATGATTCAAAGGCTTTGTTAATAAATTTACCTCCAAGAAAAGCGACGGGAGGGGCAAGAAC
AGCTAATCGATTAAAATATCATTACGAAAGTTGCATAAATAATTATAGATTTTTAAAAAAATATAATGATA
ATTTGAATCTTCTTTCAGGACAAAAGCATGCTATATTTTACCGACAGTGTCAATTCGTTCTGCTAAAAATG
AAGTCGTTTATCGGGAAGTTTTTAAAATGATTATATATATCGCCGCGTATAATGGTTCAGGAGGGCAAGGT
GGGGTGGAAAGGGTTGTTGCCCAACAATGTAACATTCTTAAAAATTTGGGGGTTAAAGTCATTATACTTGA
TAAAACATACTTCAAAATTTCTAACAAAATTCGTAACAAAAAAATACAAGTAGCACTTTATCCAATATTAG
TTTCTCTTTATTTAACCTTACAAAAATTACGTGGCGTGACGTTTAAAGTTATTGCACATGGCTATTGTTCT
CCTTTTTATAGGAATGACATCTTAATAGCTCATGGCAATATGAAATGTTATTTTCAAACAGTCATGAATAA
AAAACCTAATCGGTTGTCTGGCAGTGGTCTTTTATCTTTCTATGAGCGTTGGGCTGGAGCATTTTCAAAAA
ATATCTGGGCTGTTTCAAATAAGGTTAAAAGTGAATGGAATGAGCTTTACAATATTAATTCACATAAAATC
AAAGTTGTTCGAAATTTTATAAATCTTGCACAATTTGATTACACTGATGTTAATGAAGCAGAATATGTGAC
ATTTGTCGGGCGATTGGAAAAAGGAAAAGGAATAGATGATCTGTATTACATATGTAAAAATCTGCCAGATA
CTTCCTTCCATTTAGTTTCAAGTATTCCCGCCCCACAAAATTTTGCTTCGCTAAATAATGTTCTGACCAGC
ATTGCTGTCCCCTATGCGAAAATGCCAGAAATATTTAAGAAATCCAGAGTACTTATTTTACCGTCCTATTA
TGAAGGATATGAGCTGGTTACTATTGAAGCGCTATGCTGTGGTTGCCCTGTGATAGGCTATAATGTTGGTG
CAATTAGAGAGTTGTATGCAGAAAGTTTTCCTGGCGTATTTATTGCCAATAATAAAGAAGATTTAGCACAA
GTAGCCTACAAATTAATTAGTCTTGATAATGAAAAATATTATCATTTGAGACAAACTATTTATAGCAAGCG
TGAGCTTTTTTCTGAAGAGAGATATGCGGAAATTTTAACGGCGGCATTTAATGAAAAAAAATAAGAAACTC
TGTCTCATTTCAATTAACTCATATAATGAACTTACCGGAGGAGGAGTATATTTACGTACGCTTGTTAGTTT
TCTACAAAAACAGAATGTTAATTTAACACTTATTGATAAAAAATCCTCAGGTAAACTATTCGAAGACAATA
CTTTTCAACATATATCATTTATTAAAGGTAAACGTCAGGATATAATATCCAGGCTTTTTTTTATACCATCA
TTTTATGTCCCTTATATTTTCTCAATAATTAAAATTTTACGGAAGCAAGATATTCTTGCTTTTCACAACTC
TCGGCTTGGATTGTTATGTCTGCTTTTTAGAATACTCATGCCCCACAAAAAGATCATATTGTTTACGGATA
ACTTCGAATATGACTTAATAAGACAAAAAGATAAAAACATAACTACTTTTATTGAAAAATTAATTGTTTAT
CTCAATGAATTTATCGGGCTTAAGAATTCAGATTTAGTTAGCTATATTACCCGGCAAGATAAAAATGCAAT
GGATAAATTTTATGGGATTAAAAAAAGCAGAAATTTAATTCTCCCTGTGATATTTAGTAGAGAAAAACCAA
CTGATGTATTGTCAGCTCACTTTATTAATGAGTATAATCGATTGAATAATGATAATAGGAAAAAAGTAGTA
TTTACTGCATCTTTTGATTTTTTTCCAAATATAGATGCTGCCAACTATGTTTTAAATGCAGCAAAGTCTAA
TAATGATTATTGCTATATTTTGGCAGGTAGGAAAAGTACTACTTTGAATCTTCCTGATTTGGATAATTTAT
TTTTTTTCGATAATCTATCTAATAGTGAAATGTCATATTTATTATCTGCTTGTGATGTTTTTTATTCTCCT
ATAGTTTTAGGAAGTGGAATGAAAACAAAAATTGCAGAAGCACTATCATATGGATTATATATTTATGCGAC
AGAGCATTCCTTAATCGGCTATGATGAAATTATACACAATAAGGAGTGTGTTAAAAAAATCTCACATTTGG
ATGAGGAATTTCCTAAAGATTTCAAGATGAAAAGTATCAATAAACAGCTAATAATGTCTTATCAGCAAAAA
TATTATTCACATTATCGGTTTAATGGCCATGAACTTGATATAATAAATTTTGACGATTAGTTAGTGGAGAT
ATAATATGAACATATTAGTAACTGGTGGTGCTGGATATATCGGATCTCATACGGCTATTGAATTACTGAAT
GCAGGTCATGAGATTATCGTTCTGGACAATTTCAGTAATGCTTCATACAAGTGTATCGAAAAAATAAAAGA
AATTACTCGACGTGATTTTATAACAATTACTGGAGATGCTGGGTGTAGGAAGACACTCTCCGCTATTTTCG
AGAAACACGCCATAGATATAGTTATTCATTTTGCTGGCTTTAAATCTGTTTCAGAGTCTAAAAGTGAACCC
TTAAAGTATTACCAGAATAATGTTGGAGTGACCATTACTTTATTACAGGTAATGGAAGAGTACAGAATTAA
AAAATTTATCTTTAGTTCATCTGCGACAGTCTATGGTGAACCAGAGATAATTCCAATTCCAGAAACAGCTA
AAATTGGAGGAACTACGAATCCATATGGCACATCGAAGTATTTTGTTGAAAAAATTCTAGAGGATGTTAGT
TCCACGGGAAAACTGGATATAATTTGCTTGAGATATTTTAATCCTGTCGGTGCTCATTCTAGTGGTAAAAT
AGGTGAGGCTCCATCTGGTATCCCTAATAATCTTGTTCCTTATTTATTGGATGTTGCGAGTGGTAAACGTG
ATAAATTATTTATTTATGGCAATGATTACCCTACTAATGATGGAACAGGTGTAAGGGATTTTATTCATGTT
GTTGACTTAGCGAAAGGTCATTTGGCTGCAATGAATTATTTAAGTATCAATTCGGGATATAATATCTTTAA
TCTTGGTACAGGAAAAGGTTATTCGGTACTTGAATTAATCACTACATTTGAAAAATTAACAAACATTAAGG
TCAATAAATCTTTTATAGAGAGAAGGGCAGGGGATGTTGCGTCTTGTTGGGCTGATGCAGATAAAGCTAAT
TCTTTATTGGACTGGCAAGCCGAACAAACTCTAGAACAGATGTTATTGGACTCGTGGCGTTGGAAAAAAAA
TTATCCAGACGGATTCTGAATATAAAAGGTTTCAGTTTTATGAATCAATCAGAGCAGAGAAAAAAAATACT
GGTTCTTACACCTCGCTTTCCCTACCCTGTCATTGGAGGGGATAGATTAAGAGTCTATATGTTATGTAAAG
AACTTTCCAAAAAATATGATCTTATTCTTCTGAGCTTATGTGATCAACCACTAGAACTTGAAATAAATATA
AATGACTCGGTCTTCAAAGAAATTCATCGTGTCTATCTACCAAAATATAAATCATATTATAATGTATTAAA
AGCTTTGGTTACGCAAAAACCGTTGCAAATTGCTTATTATCAATCGGACACATTTAAGAATAAATACAATA
AATTAATTAAACAATGCGATGCAGTATTTTGTCATCTGATAAGAGTTGCTGATTATGTTAAGGATACAGAC
AAGTTCAAAATTCTTGATATGACAGATGCAATATCTTTGAATTACAGTCGCGTTAAAAAATTAGCAAGTAA
AAAAAGTTTGCGTGCAATTATTTATTCTCTGGAACAAAAAAGATTAGAATCATATGAACGTTCTGTGGCGA
ATCTTTTTGATTTGACCACTTTTATTTCATCCGTAGACCGTGACTATCTCTACCCTAATCTGGGCAGTAAT
ATCCATATAGTCAATAATGGGGTTGATACATCAGCCTTGAGATATATAAAAAGAGAAATAAAAATCGATAA
GCCTGTGGAACTTATATTTATCGGAAATATGTATTCTTTACAAAATATGGATGCTGCAAAACATTTTGCTA
AGAATATTTTACCTTGCTTGTATGATGAGTTTAATATTATTTTTAAAGTGATTGGTAAGATCTCAGAAACT
AATAAAAATATATTAAATTCATTTAAAAATACAATTGCTTTAGGTACTGTTGATGATATCAATTCTTCCGC
TTCTACAGGGCATATAGGTATATGTCCTGTTCGTCTTGGAGCAGGCGTACAAAATAAAATTCTTGAATACA
TGGCTTTAGGTTTACCATGTATTACATCTAGCATTGGTTATGAAGGTATTAATGCAAAATCAGGTAGCGAA
ATTTTTGTTGCAGATACAGTAGAGCAATATAAAAACGTACTAAGAGAAATAATTTACGATTATAATCGTTA
TACTGAAGTGGCTGAAAATGCCCGTAGTTTTGTAGAAAATAATTTTTCTTGGGAATCAAAAGTTGCCAATT
TAATGAATACATTAGATGAGAAATTATATGAACAATAATAAAATTATTACACCTATCATTATGGCTGGTGG
TTCAGGCAGTCGGTTGTGGCCACTATCAAGAATTCTCTATCCGAAACAATTTCTTAGCCTAATCGGTAGTC
ATACCATGCTTCAAACAACGGCTAATCGTCTGGATGGTTTGGATTGTACCAACCCTTATGTCATTTGTAAT
GAACAATACCGCTTTATAGTTGCTGAACAGCTTAGAAAAATCGATAGATTGACTTCAAAGAATATCATCCT
TGAGCCTGTTGGGCGTAACACTGCCCCTGCAATTGCATTAGCGGCGTTGCTGATGTCTAAGTCTGATAAAA
GTGCAGATGATCTTATGCTCGTACTGGCTGCAGATCACGTTATACACGATGAAGAAAAATTTTGTAACGCT
GTTAGATCGGCAATTCCATACGCTGCTGATGGGAAATTGGTAACATTTGGTATAATTCCAGACAAAGCAGA
AACTGGTTATGGTTATATACATCGAGGACAATATATTAATCAGGAAGATTCGGATGCATTTATAGTGTCAT
CATTTGTTGAAAAGCCAAATCATGAGACAGCCACTAAATATCTTGCTTCCGGTGAGTATTATTGGAATAGC
GGTATGTTTTTGTTTAGTGCAAATCGTTATATAGAGGAACTTAAACAATTTCGGCCTGATATTTTATCCGC
TTGTGAAAAAGCAATTGCTTCAGCGAACTTTGACCTTGATTTTGTGCGTTTAGATGAAAGTTCTTTCTCTA
AGTGCCCTGAAGAATCAATTGATTACGCTGTAATGGAAAAAACAAAAGACGCAATTGTTATTCCAATGGAT
GCTGGCTGGAGTGATGTCGGTTCATGGTCTTCTCTTTGGGAAATTAATGATAAAGACTCAGACGGCAACGT
AATAGTTGGGGATATTTTCTCTCATGAAACAAAGAATTCTTTCATATATGCCGAATCGGGAATTGTTGCTA
CAGTTGGAGTGGAAAATTTAGTTGTTGTCCAAACAAAGGATGCTGTTCTTGTCTCAGAGAGAAATAAAGTT
CAGGATGTAAAGAAAATAGTAGAACAAATTAAAAATTCAGGTCGTAGCGAGCATTATGTTCATCGCGAAGT
ATATCGTCCTTGGGGTAAATATGATTCCATTGACACAGGGGAGCGTTATCAGGTCAAACGTATAACAGTAA
ATCCTGGTGAAGGACTTTCTTTACAAATGCACCATCATAGGGCAGAACATTGGATCATAGTTTCTGGAACT
GCAAGGGTGACTATAGGTTCTGAAACTAAGATTCTTAGCGAAAATGAATCTGTTTACATACCTCTTGGTGT
AATACACTGCTTGGAAAATCCAGGGAAAATTCCTCTTGATTTAATTGAAGTTCGTTCTGGATCTTATTTAG
AAGAAGACGATGTTATCCGTTTTCAGGACCGATATGGTCGTAGCTAAATTTTTGATAATGTAACGTTAGTA
GAAGAGCGCTAATATTTTTAGTTAATCTGTAATAAGTATTATTTGTTTAAGGTATATCATGTCGAGTTTAC
CCTGCTTTAAAGCCTATGATATTCGCGGGAAATTAGGCGAAGAACTGAATGAAGATATTGCCTGGCGCATT
GGTCGCGCTTATGGCGAATTTCTCAAACCGAAAACCATTGTGTTAGGCGGTGACGTCCGACTCACCAGCGA
AACCTTAAAACTGGCGCTGGCGAAGGGGTTACAGGATGCGGGCGTCGATGTGCTGGATATTGGCATGTCCG
GCACCGAAGAGATCTATTTCGCCACGTTCCATCTCGGCGTGGATGGCGGCATCGAAGTTACCGCCAGCCAT
AACCCGATGGATTACAACGGCATGAAACTGGTGCGCGAAGGGGCTCGCCCGATCAGCGGTGATACCGGACT
GCGCGACATCCAGCGTCTGGCAGAAGCCAACGACTTTCCTCCCGTTGATGAAACCAAACGCGGTCGCTATC
AGCAAATCAATCTGCGTGACGCTTACGTTGATCACCTGTTCGGTTATATCAACGTCAAAAACCTCACGCCG
CTCAAGCTGGTGATTAACTCCGGGAACGGCGCGGCGGGTCCGGTGGTGGACGCCATTGAAGCCCGCTTTAA
AGCCCTCGGCGCACCCGTGGAATTAATCAAAGTGCACAACACGCCGGACGGCAATTTCCCCAACGGTATTC
CTAACCCGCTACTGCCGGAATGTCGCGACGACACCCGCAATGCGGTCATCAAACACGGCGCGGATATGGGC
ATTGCCTTTGATGGCGATTTTGACCGCTGTTTCCTGTTTGACGAAAAAGGGCAGTTTATTGAGGGCTACTA
CATTGTCGGCCTGCTGGCAGAAGCGTTCCTCGAAAAAAATCCCGGCGCGAAGATCATCCACGATCCACGTC
TCTCCTGGAACACCGTTGATGTGGTGACTGCCGCAGGCGGCACCCCGGTAATGTCGAAAACCGGACACGCC
TTTATTAAAGAACGTATGCGCAAGGAAGACGCTATCTACGGTGGCGAAATGAGCGCCCACCATTACTTCCG
TGATTTCGCTTACTGCGACAGCGGCATGATCCCGTGGCTGCTGGTCGCCGAACTGGTGTGCCTGAAAGGAA
AAACGCTGGGCGAACTGGTGCGCGACCGGATGGCAGCGTTTCCGGCAAGCGGTGAGATCAACAGCAAACTG
GCACACCCCGTTGAGGCGATTAACCGCGTGGAACAGCACTTTAGCCGCGAGGCGCTGGCGGTGGATCGCAC
CGATGGCATCAGCATGACCTTTGCCGACTGGCGCTTTAACCTGCGCTCCTCTAACACCGAACCGGTGGTGC
GGTTGAATGTGGAATCGCGCGGCGATGTACCGCTGATGGAAGAAAAGACAAAACTTATCCTTGAGTTACTG
AACAAGTAATTCAGTAATTTCATATAAATGGGTTTTAAAAAACGGAAAAGATGAGATATCCGGTGTGGTAT
ATCCAAGGTAATGCTATTCAGTATCTCTATGAGTGAGTTAACATCTATACCACATTTAAGCCGCACACTTC
GGGATCCCCATATGAATATCCTCCTTAGTTCCTATTCCGAAGTTCCTATTCTTTCTAGAGAATAGGAACTT
CGGAATAGGAACTAAGGAGGATATTCATATGGATAAAGCCGTAAGCATATAAGCATGGATAAGCTATTTAT
ACTTTAATAAGTACTTTGTATACTTATTTGCGAACATTCCAGGCCGCGAGCATTCAGCGCGGTGATCACAC
CTGACAGGAGTATGTAATGTCCAAGCAACAGATCGGCGTAGTCGGTATGGCAGTGATGGGACGCAACCTTG
CGCTCAACATCGAAAGCCGTGGTTATACCGTCTCTATTTTCAACCGTTCCCGTGAGAAGACGGAAGAAGTG
ATTGCCGAAAATCCAGGCAAGAAACTGGTTCCTTACTATACGGTGAAAGAGTTTGTCGAATCTCTGGAAAC
GCCTCGTCGCATCCTGTTAATGGTGAAAGCAGGTGCAGGCACGGATGCTGCTATTGATTCCCTCAAACCAT
ATCTCGATAAAGGAGACATCATCATTGATGGTGGTAACACCTTCTTCCAGGACACTATTCGTCGTAATCGT
GAGCTTTCAGCAGAGGGCTTTAACTTCATCGGTACCGGTGTTTCTGGCGGTGAAGAGGGGGCGCTGAAAGG
TCCTTCTATTATGCCTGGTGGCCAGAAAGAAGCCTATGAATTGGTAGCACCGATCCTGACCAAAATCGCCG
CCGTAGCTGAAGACGGTGAACCATGCGTTACCTATATTGGTGCCGATGGCGCAGGTCACTATGTGAAGATG
GTTCACAACGGTATTGAATACGGCGATATGCAGCTGATTGCTGAAGCCTATTCTCTGCTTAAAGGTGGCCT
GAACCTCACCAACGAAGAACTGGCGCAGACCTTTACCGAGTGGAATAACGGTGAACTGAGCAGTTACCTGA
TCGACATCACCAAAGATATCTTCACCAAAAAAGATGAAGACGGTAACTACCTGGTTGATGTGATCCTGGAT
GAAGCGGCTAACAAAGGTACCGGTAAATGGACCAGCCAGAGCGCGCTGGATCTCGGCGAACCGCTGTCGCT
GATTACCGAGTCTGTGTTTGCACGTTATATCTCTTCTCTGAAAGATCAGCGTGTTGCCGCATCTAAAGTTC
TCTCTGGTCCGCAAGCACAGCCAGCAGGCGACAAGGCTGAGTTCATCGAAAAAGTTCGTCGTGCGCTGTAT
CTGGGCAAAATCGTTTCTTACGCCCAGGGCTTCTCTCAGCTGCGTGCTGCGTCTGAAGAGTACAACTGGGA
TCTGAACTACGGCGAAATCGCGAAGATTTTCCGTGCTGGCTGCATCATCCGTGCGCAGTTCCTGCAGAAAA
TCACCGATGCTTATGCCGAAAATCCACAGATCGCTAACCTGTTGCTGGCTCCGTACTTCAAGCAAATTGCC
GATGACTACCAGCAGGCGCTGCGTGATGTCGTTGCTTATGCAGTACAGAACGGTATTCCGGTTCCGACCTT
CTCCGCAGCGGTTGCCTATTACGACAGCTACCGTGCTGCTGTTCTGCCTGCGAACCTGATCCAGGCACAGC
GTGACTATTTTGGTGCGCATACTTATAAGCGTATCGATAAAGAAGGTGTGTTCCATACCGAATGGCTGGAT
TAA
SEQ ID NO: 14 (example O8 rfb locus nucleotide sequence - O8-EPA production strain
stLMTB11734)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGCTAGCGATCGC
TTAAGATCTAGGATTTCATTATGTTACTTCCTGTAATTATGGCTGGTGGTACCGGCAGTCGTCTCTGGCCG
ATGTCACGCGAGCTTTATCCGAAACAGTTCCTCCGCCTGTTCGGGCAGAACTCCATGCTGCAGGAAACCAT
CACCCGACTCTCGGGCCTTGAAATCCATGAACCGATGGTCATCTGTAACGAAGAGCACCGCTTCCTGGTGG
CTGAACAGCTACGCCAGCTCAATAAGCTGTCGAATAATATTATTCTTGAGCCGGTCGGGCGCAACACCGCC
CCGGCCATCGCCCTGGCAGCCCTTCAGGCCACCCGCGACGGCGACGACCCGCTGATGCTGGTTCTCGCCGC
TGACCATATCATCAATAACCAGTCGGCCTTCCACGACGCCATCCGGGTCGCCGAGCAGTATGCTGATGAAG
GTCATCTGGTCACCTTCGGTATCGTGCCGAATGCCCCGGAAACTGGCTACGGTTACATTCAGCGCGGCGTG
GCGCTCACCGATAGTGCCCATTCCGCGTACCAGGTGGCCCGCTTTGTGGAGAAGCCGGATCGCGAGCGCGC
CGAGGCTTACCTCGCCTCCGGGGAGTACTACTGGAACAGCGGCATGTTTATGTTCCGCGCCAAGAAATACC
TCATCGAGCTGGCCAAATACCGTCCGGATATCCTGGAAGCCTGCCAGGCTGCGGTGAATGCCGCCGATAAT
GGCAGCGATTTCATCAATATCCCGCATGATATTTTCTGCGAGTGCCCGGATGAGTCCGTGGACTATGCCGT
TATGGAGAAAACCGCCGATGCGGTGGTGGTCGGTCTCGATGCTGACTGGAGCGACGTCGGCTCCTGGTCCG
CACTATGGGAGGTCAGCCCGAAAGACGAGCAGGGCAATGTCCTCAGCGGTGACGCGTGGGTACACAACAGC
GAAAACTGCTACATCAACAGCGACGAGAAGCTAGTGGCGGCCATTGGCGTAGAGAATCTGGTGATTGTCAG
CACTAAGGACGCCGTGCTGGTGATGAATCGCGAGCGTTCCCAGGACGTGAAGAAGGCGGTCGAGTTCCTCA
AGCAGAACCAGCGCAGCGAGTACAAGCGCCACCGTGAGATTTACCGCCCCTGGGGCCGTTGCGACGTAGTG
GTCCAGACCCCGCGCTTCAACGTCAACCGCATCACGGTGAAACCAGGCGGTGCCTTCTCGATGCAGATGCA
CCACCATCGCGCCGAGCATTGGGTTATTCTCGCCGGCACCGGTCAGGTGACTGTCAACGGTAAGCAGTTCC
TGTTGTCCGAGAACCAGTCCACCTTTATTCCGATTGGCGCCGAGCACTGCCTGGAAAACCCTGGCTGTATT
CCGCTGGAAGTGCTGGAGATCCAGTCGGGGGCGTACCTTGGCGAGGACGACATTATTCGTATTAAAGACCA
GTATGGTCGTTGCTAATTATTTTCGGGACAAGACGCAGAATGACACAGTTAACTTGTTTTAAAGCTTATGA
CATCCGTGGTGAACTGGGTGAGGAACTGAACGAGGACATCGCCTACCGTATCGGTCGCGCCTACGGCGAAT
TTCTGAAACCCGGGAAGATAGTGGTGGGGGGCGATGTGCGCCTCACAAGCGAGTCGCTGAAGCTGGCGCTG
GCCCGCGGGTTAATGGACGCCGGTACCGACGTGCTGGACATCGGCCTGAGCGGTACCGAAGAGATTTACTT
TGCCACCTTCCACCTTGGGGTAGATGGTGGCATCGAGGTGACCGCGAGCCACAATCCTATGAACTACAACG
GCATGAAGCTGGTGCGCGAGAATGCGAAGCCCATCAGCGGCGACACCGGCCTGCGGGATATCCAGCGCCTG
GCGGAGGAAAACCAGTTCCCGCCAGTGGACCCGGCGCGTCGCGGGACCCTGAGCAAGATATCGGTACTGAA
GGAGTATGTTGACCATCTGATGAGCTACGTGGACTTCTCGAACTTCACCCGTCCACTGAAGTTGGTGGTGA
ACTCCGGAAACGGGGCTGCGGGGCACGTGATTGATGAGGTGGAGAAACGCTTCGCGGCGGCTGGGGTGCCG
GTAACCTTTATCAAGGTGCATCACCAGCCGGATGGCCATTTCCCTAACGGTATCCCGAATCCGCTGCTGCC
GGAGTGCCGCCAGGATACCGCCGACGCGGTGCGCGAGCATCAGGCCGACATGGGGATTGCCTTTGACGGCG
ACTTCGATCGCTGCTTCCTGTTCGATGACGAAGCTTCGTTTATCGAGGGGTATTACATTGTCGGCCTGCTG
GCTGAGGCGTTCCTGCAGAAGCAGCCGGGAGCGAAAATCATTCACGACCCGCGCTTGACGTGGAACACGGT
AGACATCGTGACCCGCAACGGCGGCCAGCCGGTGATGTCGAAGACGGGGCATGCGTTCATCAAGGAGCGGA
TGCGTCAGGAAGACGCTATCTACGGCGGGGAGATGAGTGCGCACCATTACTTCCGCGATTTCGCCTACTGC
GATAGCGGGATGATCCCGTGGCTGCTGGTGGCGGAGCTGCTGTGTCTGAAGAACAGCTCGCTGAAATCGCT
GGTGGCGGACCGCCAGAAGGCGTTCCCTGCGTCGGGAGAGATCAACCGCAAGCTAAGTAATGCTGCTGAGG
CGATCGCCCGCATCCGGGCGCAGTATGAGCCGGCGGCTGCACACATCGACACAACGGACGGGATCAGTATT
GAATACCCTGAATGGCGCTTTAACCTGCGCACGTCTAACACCGAGCCGGTGGTGCGTCTGAACGTTGAGTC
CAGAGCTGATGTGGCGCTTATGAATGAAAAAACGACCGAGCTGTTACACCTGTTAAGCGGGGAATAAGGTG
AGAGATTTACTAACGACGATTTATCGTTATCGGGGATTTATCTGGAGCAGTGTTAAACGTGATTTTCAGGC
ACGCTATCAAACTAGTATGCTGGGCGCACTATGGCTCGTTTTACAACCGCTCTCTATGATTCTGGTCTATA
CCCTGGTTTTTTCCGAGGTGATGAAGGCAAGAATGCCCGATAATACCGGGTCGTTTGCCTATAGTATTTAT
CTCTGTTCCGGGGTACTGACCTGGGGATTATTTACTGAGATGCTGGATAAAGGTCAGAGCGTATTTATTAA
CAATGCTAATCTGATCAAGAAACTCAGTTTTCCGAAAATCTGTCTGCCGATCATCGTGACGTTATCGGCGG
TGCTAAATTTCGCGATTATTTTCAGTCTGTTTCTAATTTTTATCATTGTCACCGGTAACTTCCCCGGCTGG
CTCTTTCTCTCGGTGATACCGGTCCTGCTTTTGCAGATCCTGTTTGCCGGTGGGCTGGGGATGATCCTTGG
TGTCATGAACGTCTTTTTCAGGGATGTGGGGCAACTGGTTGGCGTTGCGCTGCAATTCTGGTTTTGGTTCA
CACCCATTGTTTATGTACTGAATTCATTACCTGCATGGGCAAAAAATCTGATGATGTATAACCCGATGACT
CGGATCATGCAATCTTATCAGTCCATCTTCGCCTATCATCTGGCCCCCAACTGGTATTCGCTATGGCCAGT
ATTGGCTCTCGCCATTATTTTCTGCGTCATCGGTTTCAGGATGTTCCGCAAGCATGCGGCGGATATGGTGG
ATGAATTATAATGAGTTATATCAGAGTAAATAATGTCGGTAAGGCGTATCGCCAGTATCACTCAAAGACCG
GGAGACTGATCGAATGGTTATCCCCTCTGAATACCAAACGCCATAATTTGAAATGGATCCTCCGCGATATT
AATTTCGAAGTCGCTCCGGGCGAGGCTGTCGGTATTATCGGTATCAACGGTGCAGGCAAGAGTACCCTGCT
TAAACTCATAACCGGGACGTCCAGGCCGACGACTGGAGAAATTGAAATCTCCGGACGTGTCGCTGCATTAC
TCGAATTGGGGATGGGGTTTCATTCTGATTTCACTGGTCGGCAGAATGTTTATATGTCTGGGCAACTGTTG
GGGTTATCGTCAGAGAAAATAACTGAACTGATGCCGCAAATTGAAGAGTTTGCTGAGATTGGGGACTATAT
CGATCAACCTGTGCGCGTCTACTCCAGTGGGATGCAAGTTCGATTAGCTTTTAGTGTAGCGACGGCTATCC
GTCCTGATGTGCTAATTATCGATGAGGCATTATCTGTTGGGGATGCATATTTCCAGCATAAAAGCTTTGAG
CGTATTCGAAAATTTCGTCAGGAAGGGACCACGCTGTTGCTGGTATCCCATGATAAACAAGCGATCCAAAG
CATTTGCGACCGGGCCATTTTATTGAATAAAGGCCAAATTGAAATGGAAGGTGAACCTGAAGCAGTGATGG
ATTATTACAATGCTCTTCTGGCCGATAAACAAAATCAGTCCATTAAACAAGTTGAGCATAATGGTAAAACG
CAAACTGTTTCAGGCACTGGTGAGGTGACTATCTCTGAGGTTCATCTTCTCGATGAACAGGGCAATGTGAC
TGAATTTGTTTCGGTAGGGCATCGTGTCAGCTTGCAGGTCAACGTTGAGGTCAAGGACGATATTCCTGAGC
TTGTTGTCGGATATATGATTAAGGATCGACTTGGGCAGCCGATTTTCGGGACCAATACGTACCATCTCAAT
CAGACACTCACCTCCCTGAAAAAAGGAGAAAAGCGTTCGTTCTTATTTTCTTTCGATGCGAGATTGGGGGT
TGGCTCCTATTCTGTCGCTGTCGCGTTGCATACTTCCAGTACGCACCTCGGCAAAAACTATGAATGGCGCG
ATCTGGCCGTGGTATTCAACGTCGTTAACACGGAACAACAAGAGTTTGTCGGCGTGTCCTGGTTGCCGCCT
GAACTGGAGATTTCTTAATGGGTTCGTCGTTTTATCGTTCATTTGAAGAACGACACAGAGGTTCGGTTGAA
GAAATCAAGCGCCGCTTGAGTTTTTATTTACCTTTTCTTGCAGGTCTGAAGGACATTTATCCTGATGGCGT
GATTGCGGATATTGGTTGCGGACGTGGCGAATGGTTGGAGATCCTGACTGAAAATGGCATTGCGAACATCG
GCGTCGATCTCGATGATGGCATGCTGGCGCGCGCCAGGGAGGCCGGACTGAATGTGCAGAAAATGGATTGT
CTGCAGTTTTTGCAAAGTCAGGCGGATCAGAGCCTGATAGCGTTGACCGGTTTTCATATTGCTGAGCATTT
GCCGTTTGAGGTCCTGCAGCAACTCGCCATGCATACCCTACGGGTGCTGAAACCAGGTGGTTTGCTGATCC
TCGAAACGCCGAACCCGGAGAATGTAAGCGTCGGCACCTGTTCATTTTATATGGATCCAACGCATAATCAT
CCTCTGCCACCGCCACTGCTTGAGTTTTTACCTATTCATTATGGTTTTACCCGAGCAATTACCGTTCGTCT
GCAGGAAAAAGAGGTTCTTCAATCTCCGGATGCAGCCGTTAATTTGGTCGATGTACTCAAAGGGGTGAGCC
CCGACTACAGCATCATTGCTCAGAAAGCAGCGCCAACAGATATTCTTGAACGCTTTGACACCCTGTTTACC
CAGCAGTACGGTCTGACGCTGGATGCTCTGAGCAACCGTTACGATGCGATTTTGCGCCAACAGTTTTCGTC
CGTTGTCTCACGGCTGGAGACGTTGAACCAAACCTATATGCAACAGATAAGCCAAATGTCAGAGACTATTC
AGACGTTGCAAGGTGAGGTTGACGATCTGAGTCATGTCATCGATCAGAACCATCAGCTTCATCAGCAAATG
GCGGATTTACATAACAGTCGTTCATGGCGTATTACTCAACCACTACGCTGGTTGTCTTTGCAACGTCAATT
ATTACGTCAGGAAGGGGCTAAAGTGCGAGCCCGTAGGGCTGGGAAAAAAATATTGCGCAAAGGGATGGCGC
TCTCGCTGGTCTTTTTCCATCGTTACCCTAAGTCTAAGGTTTATCTGTTTAAGGTTCTGAGAAAAACTGGC
TGCTATACATTGCTACAACGTTTGTTCCAACGCGTAATGCTGGTGCAATCTGACACGATGATGATGCAGTC
CAGAAGATATGATGTGGGTACTGAAGAAATGACAAGTCGCGCGATGAGTATTTATAACGAATTAAAAAATA
AAAATACGGAGAAATAACGATGCGTATTGTCATAGATTTACAAGGCGCACAGACGGAAAGCCGCTTTCGTG
GCATCGGTCGTTATAGTATCGCAATCGCCAGAGGCATAATCAGAAATAACAGCCGGCATGAGATTTTCATC
GCGCTATCCGCCATGCTGGATGAGTCGATTGCAAATATTAAGGCGCAATTTGCCGATCTCCTGCCGGCAGA
AAATATAGTCGTATGGCATGCCGTAGGCCCTGTTCGTGCGATGGACCAAGGTAATGAATGGCGTCGGGAGA
GCGCAGAACTGATTCGGGAAGCGTTTCTTGAATCATTGTGTCCAGATGTCGTTTTCATTACGAGTTTGTTT
GAAGGTCATGTCGACGATGCGGCTACATCGGTACACAAATTTAGTCGTCAGTATAAAGTAGCCGTACTGCA
CCACGATCTTATCCCCCTCGTGCAGGCGGAAACCTATCTGCAGGACGATGTATACAAACCCTACTATTTAC
AGAAAGTTGAGTGGTTAAAAAACGCTGACCTTTTGTTGACTAACTCTGCTTATACCGCACAGGAAGCGATC
GAGCATCTGCATTTACAGGGCGATCATGTGCAGAATATTGCAGCCGCAGTCGATTCTCAGTTTTGTATGGC
GGAGGTGGCAGCGAGCGAAAAAGAGACCGTCCTTGGCCATTACGGTATTCAGCGCGAGTTCATGTTGTATG
CGCCCGGAGGATTTGACTCAAGGAAAAACTTTAAACGGTTGATTGAGGCCTATGCCGGGCTCAGTGATGCC
TTACGTCGCAGTCATCAACTGGTCATCGTCAGTAAGCTTTCCATCGGTGATCGTCAGTATCTGGAATCCCT
TGCGTCAGGTAATGGTTTACAGCAGGGCGAACTGGTACTCACTGGTTATGTGCCGGAAGATGAGCTGATCC
AGCTCTATCGCCTATGTAAGCTGTTCATCTTTGCTTCACTACATGAAGGTTTTGGGTTGCCGGTTCTGGAA
GCAATGTCGTGCGGTGCGCCGGTGATTGGCTCAAATGTCACCAGTATTCCTGAAGTCATCGGTAATCCTGA
GGCATTATTCGACCCGTATTCTGTCTCTTCCATGAGGGATAAGATCGCGCAATGTTTGACTGATGATACCT
TCCTCGCGCGTCTGAAAGAAATGGCGCAGCAGCAAGCGCGTAATTTCTCTTGGGATAAAGCTGCGGTGACT
GCTCTGGAAGCTTTCGAAAAGATCGCGGTAGAAGACACCGGTACTGCGCAGGTTTTGCCTGAAGCTTTGAT
TCAGAAGATCCTTGCTATCTCACAAGGGCAGCCAGATGACCGCGATCTGCGCTTGTGCGCAACGGCCATTG
ATTACAATCTGAAAACGGCAGAACTTTATCAAATCGACGATAAATCGCTGAACTGGCGTGTGGAAGGCCCA
TTCGATAGCTCATATAGTCTGGCGTTGGTCAACCGCGAATTTGCCCGGGCACTCTCAGCCGATGGTGTAGA
GGTTTTATTGCATTCCACTGAAGGACCAGGTGATTTTGCCCCAGATGCCTCGTTTATGGCACAGTCGGAAA
ATAGTGATCTTCTGGCATTTTATAATCAATGTCAGACCCGCAAGAGTAACGAAAAGATAGATATTATTAGC
AGAAATATCTATCCACCGCGGGTTACCAAAATGGATGCCAAAGTAAAATTCCTTCATTGTTATGCTTGGGA
AGAAACGGGCTTTCCGCAACCGTGGATCAATGAATTTAATCGGGAACTTGACGGAGTGCTGTGTACTTCGG
AACATGTTCGTAAAATACTGATTGATAACGGACTGAATGTGCCCGCATTTGTTGTTGGCAATGGCTGTGAC
CATTGGCTCAATATCCCAGCCGAGACGACAAAAGATGTGGATCACGGAACATTCCGTTTCCTGCACGTCTC
TTCTTGTTTCCCACGCAAAGGGATACAGGCAATGCTTCAGGCTTGGGGGAAGGCGTTCACTCGTCGTGACA
ATGTTATCTTAATCATTAAGACTTTTAACAATCCGCACAATGAAATTGACGCATGGCTGGCTCAGGCCCAG
GCTCAATTCATAGACTATCCCAAAGTTGAAGTGATCAAAGAGGATATGTCAGCCACCGAGCTTAAAGGGCT
TTATGAAAGCTGTGATGTTTTGGTTGCTCCAGGTTGCGCTGAAGGCTTTGGTTTACCTATTGCTGAAGCAA
TGCTGAGTGGGCTACCGGCTATCGTCACCAATTGGAGCGGGCAACTTGATTTTGTTAATTCACAAAATTCA
TGGCTGGTTGACTATCAGTTCACTCGGGTAAAAACGCACTTTGGTCTGTTTTCCTCAGCCTGGGCCAGTGT
GGATATTGACAACTTAACAGATGCATTAAAAGCGGCAGCCTCAACCGATAAATCAGTGCTGCGTGACATGG
CCAATGCTGGTCGCGAGCTTCTTCTGCAGCAGTTTACCTGGAAAGCGGTGGCTGATCGTTCTTGCCAGGCG
GTCAAGACTCTGCGTGCGCATATTGATATTGCACAGCATCGGGCGCGCATTGGCTGGGTGACGACCTGGAA
CACGAAATGTGGGATCGCAACCTATTCCCAGCATCTGGTGGAAAGCGCACCTCATGGCGCGGATGTTGTTT
TTGCTCCCCAGGTCAGCGCTGGCGATCTTGTGTGTGCAGACGAAGAGTTTGTACTTCGCAACTGGATTGTA
GGTAAAGAGAGCAACTATCTGGAAAACCTCCAGCCACACATTGATGCTCTGAGACTCGATGTCATTGTGAT
CCAATTCAACTATGGATTCTTTAATCATCGAGAACTGTCGGCGTTTATTCGTCGCCAGCATGACGCCGGTC
GTTCAGTTGTTATGACGATGCACTCAACTGTGGATCCGCTGGAAAAAGAGCCGAGCTGGAATTTCCGTCTT
GCTGAAATGAAAGAGGCGCTGGCACTTTGCGACCGGTTGTTGGTGCATTCGATTGCCGATATGAACCGCCT
TAAAGATTTAGGCTTAACTGCGAATGTTGCTTTATTCCCGCACGGTGTTATCAACTACTCCGCAGCGAGCG
TCACACGTCAACAGCAGTCTTTACCGCTAATTGCGAGCTATGGCTTCTGCTTACCGCATAAGGGCCTGATG
GAACTAGTAGAATCCGTCCATAGACTCAAGCAAGCCGGTAAACCGGTTCGTTTACGACTGGTGAACGCAGA
GTATCCTGTTGGGGAGTCACGCGATCTGGTGGCAGAGCTTAAAGCTGCTGCTCAGCGGTTAGGTGTTACCG
ATCTGATTGAGATGCATAATGATTTCCTACCTGATGCGGAGAGTCTGCGGTTGCTTTCAGAAGCCGATCTT
CTGATTTTTGCTTATCAGAATACTGGGGAGTCTGCTAGCGGGGCGGTACGTTATGGTATGGCGACTCAAAA
ACCTGTTGCGGTAACGCCCCTGGCGATATTTGATGATTTGGACGATGCCGTCTTTAAATTTGATGGATGCA
GCGTCGATGATATCAGTCAGGGGATTGACCGGATCCTGAATTCCATCCGTGAACAGAACTCTTGGGCAACC
AGGACTCAACAACGTGCCGATGCATGGCGGGAACAACATGATTATCAAGCTGTTTCACGCCGTCTGGTTAA
TATGTGTCAAGGCTTAGCTAAAGCTAAATATTTTAAATAAAAATATCTCTCTTGTATTTTTTGCCTTTGAA
TACAAGAGGGGTTAGATAATGTGTCATTTATTATGAAAATTATTTTTGCTACTGAGCCAATTAAATACCCA
TTAACGGGCATCGGTCGGTATTCCCTGGAGCTGGTTAAGCGGCTGGCGGTCGCCCGCGAAATTGAAGAATT
AAAGCTATTTCACGGTGCGTCGTTTATAGAACAGATCCCTTTGGTGGAGAATAAAAGCGATACCAAAGCCA
GCAATCATGGTCGTCTGTCGGCGTTTCTACGCCGACAGACGCTGTTGATTGAGGCTTATCGCTTGCTGCAT
CCGCGGCGCCAGGCGTGGGCATTGCGCGACTATAAGGATTATATCTACCATGGCCCCAATTTTTATCTGCC
GCATAAACTGGAACGCGCCGTGACCACGTTTCATGACATATCCATTTTTACCTGCCCGGAATATCATCCAA
AAGATCGGGTTCGCTATATGGAGAAGTCCCTGCATGAGAGTCTGGATTCGGCAAAGCTGATCCTGACCGTT
TCTGATTTCTCGCGCAGTGAAATTATCCGCTTGTTCAACTATCCGGCGGAGCGGATCGTAACCACCAAGCT
AGCCTGCAGCAGTGACTATATCCCACGCAGCCCGGCAGAGTGTCTGCCGGTACTGCAGAAATATCAGCTGG
CGTGGCAGGCCTACGCGCTATATATCGGCACTATGGAGCCACGTAAAAATATCCGAGGCCTGCTGCATGCC
TATCAGCTGCTACCGATGGAGATCCGCATGCGCTATCCGCTAATCCTTAGCGGCTATCGCGGCTGGGAAGA
CGATGTGCTGTGGCAGTTAGTCGAGCGCGGTACTCGGGAAGGCTGGATCCGTTACCTCGGATATGTTCCGG
ATGAAGACCTGCCGTATCTGTACGCAGCGGCCAGAGTCTTTGTTTATCCCTCCTTCTACGAGGGATTCGGT
TTACCTATTCTTGAAGCGATGTCTTGCGGTGTGCCGGTAGTATGCTCCAATGTCACCTCTTTGCCTGAGGT
TGTTGGCGATGCCGGCCTCGTTGCCGATCCTAATGATATAGACGCGATTAGCGCGCAAATTTTGCAGAGCC
TGCAAGATGATAGCTGGCGGGAAATCGCCACCGCGCGCGGTCTTGCTCAGGCGAAACAGTTTTCGTGGGAG
AACTGTGCGACACAGACCATTAACGCCTATAAATTACTCTAAGGGTGTCAGTTGAGAGTTCTACACGTCTA
TAAGACTTACTATCCCGATACCTACGGCGGTATTGAGCAGGTCATTTATCAGCTAAGTCAGGGCTGCGCCC
GCCGGGGAATCGCAGCCGATGTTTTCACTTTTAGCCCGGACAAAGATACAGGTCCTGTCGCTTACGAAGAT
CATCGGGTCATTTATAATAAACAGCTTTTTGAAATTGCCTCCACGCCGTTTTCGCTGAAAGCGTTAAAGCG
TTTTAAGCTGATTAAAGATGACTACGATATCATCAACTACCATTTTCCGTTTCCCTTTATGGATATGCTGC
ATCTTTCGGCGCGGCCTGACGCCAGGACTGTGGTGACCTATCACTCTGATATAGTGAAACAAAAACGGTTA
ATGAAGCTGTACCAGCCGCTGCAGGAGCGATTTCTCAGCGGCGTAGATTGCATCGTTGCCTCGTCGCCCAA
TTACGTGGCTTCCAGCCAGACCCTGAAAAAATATCTGGATAAAACGGTGGTGATCCCGTTTGGTCTGGAGC
AGCAGGACGTGCAGCACGATCCGCAGAGGGTCGCGCACTGGCGGGAAACTGTCGGCGATAAGTTCTTTCTC
TTCGTCGGCACTTTCCGCTACTACAAAGGGCTGCATATTCTGATGGATGCCGCTGAGCGTAGCCGACTGCC
AGTGGTGGTTGTAGGGGGCGGGCCGCTGGAATCGGAAGTGCGGCGTGAAGCGCAGCAGCGCGGGCTGAGCA
ATGTGATGTTTACCGGCATGCTCAACGACGAAGATAAGTACATTCTCTTCCAGCTCTGCCGGGGCGTGGTA
TTCCCCTCGCATCTGCGCTCTGAGGCGTTTGGCATTACGTTATTGGAAGGCGCACGCTTTGCAAGGCCGCT
GATCTCTTGCGAGATCGGTACAGGTACCTCTTTCATTAACCAGGACAAAGTGAGTGGTTGCGTGATTCCGC
CGAATGATAGCCAGGCGCTGGTGGAGGCGATGAATGAGCTCTGGAATAACGAGGAAACCTCCAACCGCTAT
GGCGAAAACTCGCGTCGTCGTTTTGAAGAGATGTTTACTGCCGACCATATGATTGACGCCTATGTCAATCT
CTACACTACATTGCTGGAAAGCAAATCCTGAGCGGCCGCGAGCTCGTCGACTCGAGGATCCGTGTAGGCTG
GAGCTGCTTCGAAGTTCCTATACTTTCTAGAGAATAGGAACTTCGGAATAGGAACTAAGGAGGATATTCAT
ATGGATAAAGCCGTAAGCATATAAGCATGGATAAGCTATTTATACTTTAATAAGTACTTTGTATACTTATT
TGCGAACATTCCAGGCCGCGAGCATTCAGCGCGGTGATCACACCTGACAGGAGTATGTAATGTCCAAGCAA
CAGATCGGCGTAGTCGGTATGGCAGTGATGGGACGCAACCTTGCGCTCAACATCGAAAGCCGTGGTTATAC
CGTCTCTATTTTCAACCGTTCCCGTGAGAAGACGGAAGAAGTGATTGCCGAAAATCCAGGCAAGAAACTGG
TTCCTTACTATACGGTGAAAGAGTTTGTCGAATCTCTGGAAACGCCTCGTCGCATCCTGTTAATGGTGAAA
GCAGGTGCAGGCACGGATGCTGCTATTGATTCCCTCAAACCATATCTCGATAAAGGAGACATCATCATTGA
TGGTGGTAACACCTTCTTCCAGGACACTATTCGTCGTAATCGTGAGCTTTCAGCAGAGGGCTTTAACTTCA
TCGGTACCGGTGTTTCTGGCGGTGAAGAGGGGGCGCTGAAAGGTCCTTCTATTATGCCTGGTGGCCAGAAA
GAAGCCTATGAATTGGTAGCACCGATCCTGACCAAAATCGCCGCCGTAGCTGAAGACGGTGAACCATGCGT
TACCTATATTGGTGCCGATGGCGCAGGTCACTATGTGAAGATGGTTCACAACGGTATTGAATACGGCGATA
TGCAGCTGATTGCTGAAGCCTATTCTCTGCTTAAAGGTGGCCTGAACCTCACCAACGAAGAACTGGCGCAG
ACCTTTACCGAGTGGAATAACGGTGAACTGAGCAGTTACCTGATCGACATCACCAAAGATATCTTCACCAA
AAAAGATGAAGACGGTAACTACCTGGTTGATGTGATCCTGGATGAAGCGGCTAACAAAGGTACCGGTAAAT
GGACCAGCCAGAGCGCGCTGGATCTCGGCGAACCGCTGTCGCTGATTACCGAGTCTGTGTTTGCACGTTAT
ATCTCTTCTCTGAAAGATCAGCGTGTTGCCGCATCTAAAGTTCTCTCTGGTCCGCAAGCACAGCCAGCAGG
CGACAAGGCTGAGTTCATCGAAAAAGTTCGTCGTGCGCTGTATCTGGGCAAAATCGTTTCTTACGCCCAGG
GCTTCTCTCAGCTGCGTGCTGCGTCTGAAGAGTACAACTGGGATCTGAACTACGGCGAAATCGCGAAGATT
TTCCGTGCTGGCTGCATCATCCGTGCGCAGTTCCTGCAGAAAATCACCGATGCTTATGCCGAAAATCCACA
GATCGCTAACCTGTTGCTGGCTCCGTACTTCAAGCAAATTGCCGATGACTACCAGCAGGCGCTGCGTGATG
TCGTTGCTTATGCAGTACAGAACGGTATTCCGGTTCCGACCTTCTCCGCAGCGGTTGCCTATTACGACAGC
TACCGTGCTGCTGTTCTGCCTGCGAACCTGATCCAGGCACAGCGTGACTATTTTGGTGCGCATACTTATAA
GCGTATTGATAAAGAAGGTGTGTTCCATACCGAATGGCTGGATTAA
SEQ ID NO: 15 (example O15 rfb locus nucleotide sequence - O15-EPA production strain
stLMTB11738)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGCTAGCATGAGC
AAAACTAAACTAAATGTTCTTTACCTTGCAATAAGTCAGGGTGCCAATTACCTACTGCCATTATTAATTTT
TCCTTATCTTGTTAGAGTCATTGGTGTATCGAATTTTGGTGATCTGAGTTTTTCATTGATAACTATACAAG
TGTTGTTAATGGTTGTTGAATATGGTTTTGGATATAGTGGGACAAGAGAAATAGCACTAAATAACGATAAA
AAATACCATTCTGAATTTTTTTGCGGTGTGGTGCTTGCTCGTTTTATATTAATGCTAATTGCAGCTATAAT
ACTCATAATACTCTGTTTTTTTTATGTTTTTAACGACGTTAAGTCTTTGTTATGTGTTGGTTTTCTGTCCG
TAATTGCAGGTGTTTTCAATCCAAATTGGTTTTTGCAAGGTAAGGAAATGATGAGTGTGATGGCTGTGCTG
TCACTATTTTCACGAGGCATAGCAGTCGTTGCAGTTTATCTAATTATAAAACCCGCAACGCCGATGTACAT
CAGTGCCTTATTATTGAGCATGCCATATATTTTGTATTCATTCTGTGGCGTTGCCTACTTACTTATTATCA
AGGAGATTTTTTTATGTAGGCCACCGATAAAGAAAATTCAAGTAATTTTAAAAAATGGATTTCATTTTTTT
TGTTCAACACTTGCGACTAGTGCATACACAATGTTGACCCCTCTTGTATTGGGTGGCGTATCTGGAAAGTT
TGATGTAGGCATCTTTAACTCAGCTAACATGATCAAACAAGGTTTGGCTGGACTTGCATCACCATTAGTCC
AAGCTTTTTATCCAAGAATTAACATTTTGCAAAGAGAGAATCCATATATTGCAAACTTAAAATCTAGAATG
ATTCTTAAATACTTGCTTGTTTTTTACATGGCTTTAGCAATACCATTTTTACTTTTTGCCAACCAATTATC
ATTATTAATATTCGGCATGAAAGGTGAAGTAATTGCAGGTGCAATGCAATTAATGACATTGCTTCCTATAT
TCATAGGTTTTAATACAGTTGTCGGGTTACTTGTATTAGTACCTAATGGGATGCAAAAACAGTATTTCAAA
TCTATTTTCCTAGGAACTATTACTTGTTTAAGCATAGTTTATCCAGCATGTAAATATTATGGAGCAACGGG
TGCGATTGTGAGTCTTATTGTAGCTGAAATTTTCGTTGGCATGGGAATGCTTAAACAATTCATTAAAGTAA
ATAAAACCGTATGTAGGCCTCATAAATTATGAATATCTCGGTAATAATATCTGTTTGGAAACGCCCAGTTC
AATTAGAATTGATTCTCTCTGAGCTCGATTCTCAGGCTAAAGACAATAGTCTACACCTAGAAGTAATTGTT
TCCGATAGTCATAGTGGTAAAGAAATTGATGATGTAGTTGCTGATAATATTCATAAAAAGAAAAATATTAA
TATTATCCATCAACATACTAAAAATATACTCTCCGCTAAGCGCAATTTCGGAGCATCCCTAGCCCATGGGG
ATTATTTAATATTTCTTGATGATGATTGTATACCCGCAAGTGGATATATATCATCGTTGCTGAACTATTTA
AAAAAAATGAATAGTAAAAGCGTTTTATGTGGGGAAGTTAGATTCGAAAATGAACTCATTGAGACCAGCAA
TTACTATCGCTACAGGAACTCTTTACACCCTAAGTTTAGTGATAGTCCTGATATCTCTATGAATGCCTGGA
CTTTTGTCGCAATGAATTGTGTTCTTGATAGAAAGGCATTTTCATCAGGTATAGTTTCATATAATGAAAAT
TTTATTGGTTATGGTTGTGAAGATCATGAGTTTGGGTGGCAACTTGAAAAAAATGACTTCAAAATTATTTT
TGCTGATTTTAAAATATTACATCACGAATACAGTGGCGATATAGAAGGATATACAAAAAAAATTCGTGCTA
CAGCACGTGATGGTATGAATGTATTAAGCAAAGTAAGGCCTGAAATGTTTTCTACTAATAAAAAATTATTC
CTAGTTGAGAAAATATTTAGTAAACACAAAACGTTTAGTAAAATATGCCAATCAATATTTTTCAATAAATT
TATTTTTAAAAAAATAATACAATTTTTAAAAAAAACAGATGCAAATAAAAAACTCTATTTCCCAATTCTTT
ACAGATATGTGTTGATTTCGGCATATATACATGGTATTGGAGAGCGTGGCACCTCAAAAACAGATGATTTG
CTTAAGAACTGGTATATATAGATGATGCTATCTTCATTTATTAAGACATTTGTATGGAAGGTAAAAAACAA
TGAAGTATAATGCATTGATGGCTTTTTTATTATTTTTTGTTGTTTTTTTTAGATTGTCGCTGATAATACCT
TTCTTATATTTGGCATTTATTCCTGCATTTTTTGGTATTATGTATTTAGTGCGTAATTTTATGATTACTAT
GGGCAATGGATTGGTATCTATAGATCGTAAAAATTTGTTGCTGTTATCTATATTCATAATTATTTTTTTAT
TTTGTTTGGTTTTCGATTTGTTTCAAAAAAGCCATTCTTTTCAAAGTTATTTTACCGTTAGATTATTTATG
TTGTTTTTATTTTCATTTGTTCCTGCGTATTATTTAGTAAATAGATTCATAAAGGGTGACTTGAAATTAAT
GGAGCGAATATTAGTGTATTCTCTCTGGGTTCAAATAGTTATTTTTTTTGGTATGTATATAAGTCCAGAGT
TAAAAAGATTGTTATATACTTTCTTTGGTATGTCTGACTCTGTTAATCTTTGGGAACAAAATGCTAAAGTA
AGAGGATTTGGGTTGTCGGGTGAAATAAATTTCATGACACCATTTTTGATGATCTATATGTCATTTTTTAT
GATGAAAAGGCGTTATGCTTTAATTACTTTAATTTGTCTGACTCAAATCGTAAATTCTAACATGGCTGTGA
TTGCAGCCATTATTGGTATCGGTTGCTCTAGACTTAATATTAATATAAAAATTGCAACAGTATTGATTTTG
GGAGTTTTAGTTTATAGCTTAGGAGCGGTGTTCTTTCCTCGATTTTATGATGAGTTCGTTTCTGGAGATGG
CACAAGAACTCTGGATATCTTATTACAGCAACATGTGTTTGTTGTAGGTAATTTAGATTTTTTTAATATTA
TATTTGGATTACAGCAAAACATATCTTCATCAATCCCCGATATTAAACAAAGTTCGGATATGGGCTGGGTT
ATACTGTTTAATTACGGTGGGTTAACATTTATTACACTCTTTTTATTTTTAATCTTTACTATTTCTATTGC
GACATTTGGAATGACATATCAAGCAATTATATGGATGTTAATTGGGATAATTTTCAATACCAAAGGTTTAG
TTTTAGGATCTAACGGCTATTTCTTTCTATCTTTTATATATATGTTTTTGAATAGAGTAACACTTAGTGGA
CAGAGTTCAATTACTAATAAGTTAGGTCAAGTAAGTAAATAGCTTCCAGAGTATATTTGTCAATGATTTGA
GGTTCGGTTATTATGTTTTCATCTAAAACACTGTTAATTACTGGTGGTACTGGCTCTTTCGGGAATGCTGT
ATTAAATAGATTTCTTGATACAGATATTGCAGAAATCCGTATATTTAGTCGTGATGAAAAAAAACAAGATG
ATATGCGGAAAAAATACAATAATCAAAAATTAAAGTTCTATATTGGTGATGTCAGAGATTACCGTAGTATT
TTGAATGCGACTCGCGGTGTTGATTTTATATATCATGCAGCGGCACTTAAGCAAGTTCCATCATGTGAATT
TCATCCTATGGAAGCCGTTAAAACTAATATCCTTGGTACGGAAAATGTTCTTGAAGCAGCTATAGCGAATG
AAGTGAAGAGGGTTGTATGCCTAAGTACTGATAAAGCTGTATACCCGATTAACGCAATGGGTATTTCAAAA
GCTATGATGGAAAAGGTCATGGTCGCGAAATCCCGTAATGTTGATCGCAATAAAACAGTAATATGTGGTAC
CCGTTATGGGAATGTTATGGCATCTCGCGGTTCAGTTATTCCATTATTTGTTGATCTTATTAGAGCGGGCA
AGCCACTCACAATAACTGATCCTAATATGACCCGCTTTATGATGACTCTTGAGGATGCGGTAGATTTAGTT
CTTTATGCGTTTGAACATGGTAATAATGGTGATATCTTTGTGCAAAAAGCACCTGCAGCAACTATTGACAC
ATTAGCTATTGCTTTAAAGGAATTACTAAATGTTCCTGACCATCCGGTAAATGTCATTGGAACGCGTCATG
GCGAGAAATTATATGAAGCTCTACTTAGTCGTGAGGAAATGATCGCTGCTATAGATATGGGCGATTATTAC
CGTGTCCCGCCAGATCTTCGTGACCTTAATTATGGCAAATATGTTGAGCAAGGTGATAGCCGAATATCTGA
AATAGAAGATTATAACTCTCATAATACTCAACGGTTAGATGTTGAAGGCATGAAAGAGCTCTTGCTAAAAT
TAGCCTTTATTCGAGCAATTCGTGCTGGTGAAAAATATAATCTGGATTCATGATATGAAAATATTAGTTAC
TGGTGCAAATGGTTTTATTGGTCGTAATTTATGTTTGAGGCTTGAGGAACTTGGTTATAAAGATCTTATTA
GAATTGATCGAGAATCAACGAAGCAAGATCTTGAACAAGGCTTACAGGATGCCGATTTTATTTATCACTTA
GCTGGTATCAATAGACCTAAGACTGATGATGAGTTTATTTCTGGAAACAGTGATTTAACAAAGCATATAGT
TGAGTATCTCCTTTCTATTGGTAAGAATACACCAATTATGCTAAGTTCTTCGATACAAGCTGAACTTAATA
ATGCTTATGGGGTTAGCAAAGCTGTAGCTGAAAGCTATGTCGAAAAATATGCTGCTGCTAGTGGTTCTTCG
TATTATATTTTCAGATATCCAAACGTTTTTGGTAAATGGTGTAAGCCAAACTATAATTCTTTTATAGCAAC
TTTTTGCTACAATATTTCCAATGATATTGAGATTACTATCAATGATGCAGCAGCGCCAGTCAATCTGGTCT
ATATTGATGATGTTTGTACTGATGCTATAGCTCTTCTCTCTGGGACGGTTGAAAGTGGATATAAAGTTGTT
GCACCAATTTATTCAACAACAGTTGGTGAAGTTGCAGAATTAATTTATAGCTTCAAAAATAGCCGTTCCAC
CCTGATCACAGAGGCTGTCGGGGCGGGATTTACCCGTGCATTGTATTCTACATGGCTGAGTTATTTACCAG
CAGAGAAGTTTGCGTACAAGGTACCTTTTTATGGGGATGCCCGCGGAGTCTTTTGTGAGATGTTGAAAACG
CCTTCAGCGGGGCAGTTTTCATTTTTTACTGCTCACCCTGGTATTACGCGTGGCGGACATTACCATCACAG
TAAAAATGAGAAGTTTTTGGTCATTCGAGGTCAGGCATGCTTTAAATTTGAACATGTGATTACCGGTGAGC
GATATGAACTGAAAGTTTCATCGGGTGAGTTTAAGATTGTTGAAACAGTTCCTGGTTGGACACATGACATT
ACAAATATTGGAACTGATGAATTAATAGTCATGCTCTGGGCAAATGAAATTTTCAACCGTGATGAGCCCGA
TACTATTGCGAGACCTCTATAATGAAAAAATTAAAAGTTATGTCTGTTGTTGGAACCCGTCCTGAGATTAT
CCGTTTGTCGAGGGTTCTTGCTAAGTTTGATGAATACTGCGAGCATATTATTGTCCATACTGGTCAAAATT
ATGATTACGAATTAAATGAAGTGTTCTTCAATGACTTGGGTGTTCGAAAACCTGATTATTTTTTAAATGCA
GCGGGTAAAAATGCGGCGGAAACCATTGGTCAGGTTATTATTAAGGTAGATGAAGTATTAGAAATCGAAAA
ACCTGAAGCAATACTGGTATTGGGCGATACGAATTCATGTATTTCTGCCATTCCGGCCAAACGCCGTAAAG
TGCCTATATTTCATATGGAAGCAGGTAACCGTTGTTTCGATCAACGCGTGCCTGAAGAAACCAACAGACGT
ATTGTTGACCATACGGCTGATATCAATATGACCTACAGTGATATTGCTCGTGAATATCTCTTGGCTGAAGG
TATCCCAGCTGATCGGATCATAAAAACTGGTAGCCCTATGTTTGAGGTTCTTTCATATTATATGCCCCAAA
TTGATGGTTCAGATGTGCTATCGCGTTTGAATCTACAGTCTGGTGAGTTTTTTGTAGTAAGTGCGCATCGT
GAAGAGAATGTTGATTCTCCAAAACAGCTCGTAAAGCTTGCGAACATTCTAAATACTGTTGCTGAAAAATA
TAATCTTCCAGTTATTGTCTCCACACACCCAAGGACACGTAACCGAATCCGTGAGCAAGGAATTGAATTTC
ATTCAAATATAAATCTACTGAAACCATTGGGTTTCCATGATTATAACCACTTGCAGAAGAACTCACGAGCT
GTGCTTTCAGATAGCGGTACTATCACTGAAGAGTCATCCATCATGAATTTCCCAGCGGTAAACATCCGGGA
AGCGCATGAGCGTCCGGAAGGCTTTGAGGAAGCATCCGTCATGATGGTGGGGTTAGAGTGTGAACGCGTAT
TACAAGCGCTGGATATTCTGGCAACACAACCGCGAGGTGAAGTCCGTCTTTTACGTCAGGTTAGTGATTAC
AGCATGCCAAATGTGTCGGATAAAGTTGTCAGAATTGTTCACTCTTACACAGATTATGTTAAGAGAGTCGT
CTGGAAAGAATATTGATGAAACTTGCTTTAATCATAGATGATTACCTGCCCAACAGTACTCGTGTTGGTGC
AAAAATGTTTCATGAACTTGCTCAAGAATTTATCCAGCGTGGGCACGATGTTACGGTAATTACTCCTGGTA
CGGGCATGCAAGAAGAGATTTCTTTTGATACCTTTCAGGGGGTAAAAACATGGCGTTTTAAAAGCGGGCCG
CTCAAGGATGTAAGTAAAATTCAGCGAGCGGTCAATGAAACGCTTTTGTCCTATCGGGCGTGGAAAGCCAT
CAAAAAATGGGTAAAAAAAGAGACCTTTGAGGGGGTGATTTATTATTCACCTTCCATATTCTGGGGGCCTT
TAGTTAAAAAAATTAAAGCTCGTTGCCAATGTCCTGCTTATCTTATTTTAAGAGATATGTTTCCACAATGG
GTAATTGATGCAGGAATGCTTAATGCTGGTTCCCCAATAGAACGCTACTTTCGTCTTTTTGAAAAAATATC
TTATCGTCAGGCAAATCGTATTGGACTTATGTCTGATAAGAATCTTGATGTTTTTCGGAAAGATAATAAAG
GCTATCCGTGCGAAGTTTTGCGTAATTGGGCATCCCTAACACCAACGATCATACCCAAGGATTATATACCA
CTACGTAAGCGACTTGGCCTAGAGGATAAAACCATTTTCTTCTATGGTGGAAACATAGGTCATGCACAGGA
CATGACAAACTTGATGCGACTTGTGAGAAACATGGCAGCATATCCTCAAGCTCATTTCCTATTTATTGGCC
AGGGGGATGAAGTTGAATTAATTAATTCATTAGCATCTGAGTGGGCATTGACGAATTTCACCTATTTGCCC
TCGGTTAACCAAGATGAATTTAAGTTCATTTTGTCGGAAATGGATATCGGCTTGTTTTCTCTTTCCGCTAG
ACACTCTTCCCATAATTTTCCTGGTAAGTTATTAGGCTATATGGTTCAGTCGCTACCTATTTTAGGTAGCG
TAAATGCCGGAAATGATTTGCTCGACATTGTCAATCAAAATAATGCGGGATTAATCCATGTCAATGGTGAG
GACGATAAATTATGTCAATCTGCGCTATTAATGTTGCATGATATTGATGTGCGCCGGCAACTTGGTTCGGG
GGCGAATATATTGTTGAAAGAACAATTCTCCGTTGAGTCTGCGGCACAGACGATAGAAATGAGGTTGGAGG
CATGCAATGCGATTAATTGATAATGACCAACTCGACGAATTATATGATCAAGCCGGGCAATCGGAACGTTT
ACGTTCCCACCTTATGATGCACGGCTCGCATCAAGAAAAGGTACAGCGTTTACTTATTGCATTAGTAAAGG
GCAGCTATGTTGAACCGCATTATCACGAACTTCCTCATCAGTGGGAAATGTTCATTGTTATGGAGGGGCAA
CTTCAGGTTTGTTTGTATGGTAGAAATGGTGAGGTTATAAAGCAATTTATAGCAGGAGATAATACTGGAAT
GAGCATTGTGGAGTTTTCTCCGGGCGATATACACAGTGTCGAATGCCTATCTCCGCGTGCTCTTATGGTGG
AAGTTAAGGAGGGGCCATTTGACCCTTCTTTTGCAAAATCGTTCGTGTGAGCGGCCGCGAGCTCGTCGACT
CGAGGATCCGTGTAGGCTGGAGCTGCTTCGAAGTTCCTATACTTTCTAGAGAATAGGAACTTCGGAATAGG
AACTAAGGAGGATATTCATATGGATAAAGCCGTAAGCATATAAGCATGGATAAGCTATTTATACTTTAATA
AGTACTTTGTATACTTATTTGCGAACATTCCAGGCCGCGAGCATTCAGCGCGGTGATCACACCTGACAGGA
GTATGTAATGTCCAAGCAACAGATCGGCGTAGTCGGTATGGCAGTGATGGGACGCAACCTTGCGCTCAACA
TCGAAAGCCGTGGTTATACCGTCTCTATTTTCAACCGTTCCCGTGAGAAGACGGAAGAAGTGATTGCCGAA
AATCCAGGCAAGAAACTGGTTCCTTACTATACGGTGAAAGAGTTTGTCGAATCTCTGGAAACGCCTCGTCG
CATCCTGTTAATGGTGAAAGCAGGTGCAGGCACGGATGCTGCTATTGATTCCCTCAAACCATATCTCGATA
AAGGAGACATCATCATTGATGGTGGTAACACCTTCTTCCAGGACACTATTCGTCGTAATCGTGAGCTTTCA
GCAGAGGGCTTTAACTTCATCGGTACCGGTGTTTCTGGCGGTGAAGAGGGGGCGCTGAAAGGTCCTTCTAT
TATGCCTGGTGGCCAGAAAGAAGCCTATGAATTGGTAGCACCGATCCTGACCAAAATCGCCGCCGTAGCTG
AAGACGGTGAACCATGCGTTACCTATATTGGTGCCGATGGCGCAGGTCACTATGTGAAGATGGTTCACAAC
GGTATTGAATACGGCGATATGCAGCTGATTGCTGAAGCCTATTCTCTGCTTAAAGGTGGCCTGAACCTCAC
CAACGAAGAACTGGCGCAGACCTTTACCGAGTGGAATAACGGTGAACTGAGCAGTTACCTGATCGACATCA
CCAAAGATATCTTCACCAAAAAAGATGAAGACGGTAACTACCTGGTTGATGTGATCCTGGATGAAGCGGCT
AACAAAGGTACCGGTAAATGGACCAGCCAGAGCGCGCTGGATCTCGGCGAACCGCTGTCGCTGATTACCGA
GTCTGTGTTTGCACGTTATATCTCTTCTCTGAAAGATCAGCGTGTTGCCGCATCTAAAGTTCTCTCTGGTC
CGCAAGCACAGCCAGCAGGCGACAAGGCTGAGTTCATCGAAAAAGTTCGTCGTGCGCTGTATCTGGGCAAA
ATCGTTTCTTACGCCCAGGGCTTCTCTCAGCTGCGTGCTGCGTCTGAAGAGTACAACTGGGATCTGAACTA
CGGCGAAATCGCGAAGATTTTCCGTGCTGGCTGCATCATCCGTGCGCAGTTCCTGCAGAAAATCACCGATG
CTTATGCCGAAAATCCACAGATCGCTAACCTGTTGCTGGCTCCGTACTTCAAGCAAATTGCCGATGACTAC
CAGCAGGCGCTGCGTGATGTCGTTGCTTATGCAGTACAGAACGGTATTCCGGTTCCGACCTTCTCCGCAGC
GGTTGCCTATTACGACAGCTACCGTGCTGCTGTTCTGCCTGCGAACCTGATCCAGGCACAGCGTGACTATT
TTGGTGCGCATACTTATAAGCGTATTGATAAAGAAGGTGTGTTCCATACCGAATGGCTGGATTAA
SEQ ID NO: 16 (example O16 rfb locus nucleotide sequence - O16-EPA production strain
stLMTB11739)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGTGAAAATACTT
GTTACTGGTGGCGCAGGATTTATTGGTTCAGCTGTAGTTCGTCACATTATAAATAATACGCAGGATAGTGT
TGTTAATGTCGATAAATTAACGTACGCCGGAAACCGGGAATCACTTGCTGATGTTTCTGATTCTGAACGCT
ATGTTTTTGAACATGCGGATATTTGCGATGCACCTGCAATGGCACGGATTTTTGCTCAGCATCAGCCGGAT
GCAGTGATGCACCTGGCTGCTGAAAGCCATGTTGACCGTTCAATTACAGGCCCTGCGGCATTTATTGAAAC
CAATATTGTTGGTACTTATGTCCTTTTGGAAGCCGCTCGCAATTACTGGTCTGCTCTTGATAGCGACAAGA
AAAATAGCTTCCGTTTTCATCATATTTCTACTGACGAAGTCTATGGTGATTTGCCTCATCCAGATGAAGTA
AATAATACAGAAGAATTACCCTTATTTACTGAGACGACAGCTTACGCGCCAAGCAGCCCTTATTCCGCATC
CAAAGCATCCAGCGATCATTTAGTCCGCGCGTGGAAACGTACATATGGTTTACCGACAATTGTGACTAATT
GCTCGAACAACTATGGTCCTTATCATTTCCCGGAAAAGCTTATTCCACTGGTTATTCTTAATGCACTGGAA
GGTAAGGCATTACCTATTTATGGCAAAGGAGATCAGATCCGCGACTGGTTGTATGTTGAAGATCATGCGCG
TGCGTTATATACCGTCGTAACCGAAGGTAAAGCGGGTGAAACTTATAACATTGGTGGGCACAACGAAAAGA
AAAACATCGATGTAGTGCTCACTATTTGTGATTTGCTGGATGAGATTGTACCGAAAGAGAAATCTTATCGT
GAGCAAATCACTTATGTTGCTGATCGTCCGGGACACGATCGCCGCTATGCTATTGATGCTGAGAAGATTGG
TCGCGCATTGGGATGGAAACCACAGGAAACGTTTGAGAGCGGGATTCGTAAAACGGTGGAATGGTACCTGT
CCAATACAAAATGGGTTGATAATGTGAAAAGTGGTGCCTATCAATCGTGGATTGAACAGAACTATGAGGGC
CGCCAGTAATGAATATCCTCCTTTTTGGCAAAACAGGGCAGGTAGGTTGGGAACTACAGCGTGCTCTGGCA
CCTTTGGGTAATTTGATTGCTTTTGATGTTCACTCTACTGATTATTGCGGTGATTTTAGTAATCCTGAAGG
TGTAGCTGAAACCGTAAGAAGCATTCGGCCGGATATTATTGTCAATGCAGCCGCTCACACCGCAGTAGACA
AAGCAGAATCAGAACCGGAGTTTGCACAATTAATTAACGCAACAAGTGTCGAAGCGATTGCGAAAGCAGCA
AATGAAGTTGGAGCCTGGGTTATCCATTACTCGACTGATTACGTCTTCCCTGGAAATGGCGATATGCCATG
GCTGGAGACGGATGCAACCGCACCACTAAATGTTTACGGTGAAACCAAGTTAGCCGGAGAAAAAGCGTTAC
AGGAATATTGCGCGAAGCATCTTATTTTCCGGACCAGCTGGGTCTATGCAGGAAAAGGAAATAACTTCGCC
AAAACGATGTTACGTCTGGCAAAAGAGCGTGAAGAATTAGCGGTTATTAACGATCAGTTTGGTGCGCCAAC
AGGTGCTGAACTGCTGGCTGATTGTACAGCACATGCCATTCGTGTCGCACTGAATAAACCGGATGTCGCAG
GCTTGTACCATTTGGTAGCCAGTGGTACCACAACCTGGTACGATTATGCTGCGCTGGTTTTTGAAGAGGCG
CGCAAAGCAGGCATTCCCCTTGCACTCAACAAGCTCAACGCAGTACCAACAACAGCCTATCCTACACCAGC
TCGTCGTCCACATAACTCTCGCCTTAATACAGAAAAATTTCAGCAGAACTTTGCGCTTGTCTTGCCTGACT
GGCAGGTTGGCGTGAAACGAATGCTCAATGAATTATTTACGACTACAGCAATTTAATAGTTTTTGCATCTT
GTTCGTGATGGTGGAGCAAGATGAATTAAAAGGAATGATGAAATGAAAATGCGTAAAGGTATTATTTTAGC
GGGTGGTTCTGGTACACGTCTTTATCCTGTGACTATGGCTGTCAGTAAACAGCTATTACCTATTTATGATA
AACCGATGATCTATTACCCGCTCTCTACACTGATGTTGGCGGGTATTCGCGATATTTTGATTATCAGTACA
CCTCAGGATACTCCTCGTTTTCAACAATTGCTGGGTGACGGTAGCCAGTGGGGCCTGAATCTTCAGTACAA
AGTGCAACCTAGCCCAGATGGCCTCGCGCAGGCATTTATCATCGGTGAAGAGTTTATTGGTGGTGATGATT
GTGCTTTGGTTCTTGGTGATAATATCTTTTACGGTCACGATCTGCCGAAGCTAATGGAGGCCGCTGTTAAC
AAAGAAAGTGGTGCAACGGTATTTGCCTATCACGTTAATGATCCAGAACGCTATGGTGTCGTTGAGTTTGA
TAAAAACGGTACGGCAATCAGTCTGGAAGAAAAACCGTTAGAACCAAAGAGTAATTACGCCGTTACAGGTC
TGTACTTTTATGATAACGACGTGGTTCAGATGGCGAAAAACTTGAAGCCGTCTGCACGTGGTGAGTTAGAA
ATTACAGATATTAACCGTATTTATCTTGAGCAGGGACGTCTGTCTGTCGCGATGATGGGGCGTGGCTACGC
GTGGCTGGACACGGGGACTCATCAGAGTCTGATAGAAGCAAGTAATTTTATTGCGACAATTGAAGAGCGCC
AGGGATTGAAGGTTTCCTGTCCTGAAGAGATTGCATTTCGTAAAGGTTTTATTGATGTTGAGCAAGTAAGA
AAATTAGCTGTACCACTAATAAAGAATAATTATGGGCAGTATCTTTATAAAATGACGAAGGATTCAAATTA
ATGAATGTGATTAGAACTGAAATTGAAGATGTGCTAATTCTGGAGCCAAGAGTATTTGGTGATGATAGAGG
TTTCTTTTATGAGAGCTTTAATCAATCAGCATTTGAACATATTCTAGGCTATCCGGTCAGCTTTGTTCAAG
ACAATCACTCACGTTCATCAAAAAATGTACTCAGAGGCCTTCACTTTCAACGCGGCGAGTACGCACAAGAT
AAACTTGTACGCTGCACTCATGGAGCAGTTTTTGATGTTGCTGTTGATATTCGACCCAATTCGGTATCCTT
TGGTAAATGGGTTGGTGTTCTGCTTTCAGCTGATAATAAGCAGCAGTTGTGGATACCAAAAGGGTTTGCTC
ATGGCTTTTTGGTTCTGTCTGATATCGCTGAATTTCAATATAAAACTACAAACTATTATCATCCTGAAAGC
GATTGTGGAATATGTTGGAATGATGAACGCATTGCAATTGATTGGCCCCAAACATCAGGGTTAATCCTTTC
GCCAAAAGATGAAAGGCTCTTTACGTTAGATGAGCTTATCAGATTAAAATTAATTGCATGAATACGAATAA
ATTATCTTTAAGAAGAAACGTTATATATCTGGCTGTCGTTCAAGGTAGCAATTATCTTTTACCATTGCTTA
CATTTCCATATCTTGTAAGAACACTTGGTCCTGAAAATTTCGGTATATTCGGTTTTTGCCAAGCGACTATG
CTATATATGATAATGTTTGTTGAATATGGTTTCAATCTCACAGCAACTCAGAGTATTGCCAAAGCAGCAGA
TAGTAAAGATAAAGTAACGTCTATTTTTTGGGCGGTGATATTTTCAAAAATAGTTCTTATCGTCATTACAT
TGATTTTCTTAACGTCGATGACCTTGCTTGTTCCTGAATATAACAAGCATGCCGTAATTATATGGTCGTTT
GTTCCTGCATTAGTCGGGAATTTAATCTACCCTATCTGGCTGTTTCAGGGAAAAGAAAAAATGAAATGGCT
GACTTTAAGTAGTATTTTATCCCGCTTGGCTATTATCCCTCTAACATTTATTTTTGTGAACACAAAGTCAG
ATATAGCAATTGCCGGTTTTATTCAGTCAAGTGCAAATCTGGTTGCTGGAATTATTGCACTAGCTATCGTT
GTTCATGAAGGTTGGATTGGTAAAGTTACGCTATCATTACATAATGTGCGTCGATCTTTAGCAGACGGTTT
TCATGTTTTTATTTCCACATCTGCTATTAGTTTATATTCTACGGGAATAGTTATTATCCTGGGATTTATAT
CTGGACCAACGTCCGTAGGGAATTTTAATGCGGCCAATACTATAAGAAACGCGCTTCAAGGGCTATTAAAT
CCTATCACCCAAGCAATATACCCAAGAATATCAAGTACGCTTGTTCTTAATCGTGTGAAGGGTGTGATTTT
AATTAAAAAATCATTGACCTGCTTGAGTTTGATTGGTGGTGCTTTTTCATTAATTCTGCTCTTGGGTGCAT
CTATACTAGTAAAAATAAGTATAGGGCCGGGATATGATAATGCAGTGATTGTGCTAATGATTATATCGCCT
CTGCCTTTTCTTATTTCATTAAGTAATGTCTATGGCATTCAAGTTATGCTGACCCATAATTATAAGAAAGA
ATTCAGTAAGATTTTAATCGCTGCGGGTTTGTTGAGTTTGTTGTTGATTTTTCCGCTAACAACTCTTTTTA
AAGAGATTGGTGCAGCAATAACATTGCTTGCAACAGAGTGCTTAGTTACGTCACTCATGCTGATGTTCGTA
AGAAATAATAAATTACTGGTTTGCTGAGGATTTTATGTACGATTATATCATTGTTGGTTCTGGTTTGTTTG
GTGCCGTTTGTGCGAATGAGTTAAAAAAGCTAAACAAAAAAGTTTTAGTGATTGAGAAAAGAAATCATATC
GGTGGAAATGCGTACACAGAGGACTGTGAGGGTATCCAGATTCATAAATATGGTGCACATATTTTTCATAC
CAATGATAAATATATATGGGATTACGTTAATGATTTAGTAGAATTTAATCGTTTTACTAATTCTCCACTGG
CGATTTATAAAGACAAATTATTCAACCTTCCTTTTAATATGAATACTTTCCACCAAATGTGGGGAGTTAAA
GATCCTCAAGAAGCTCAAAATATCATTAATGCTCAGAAAAAAAAGTATGGTGACAAGGTACCTGAAAATTT
GGAGGAGCAGGCGATTTCATTAGTTGGGGAGGACTTATACCAAGCATTGATAAAGGGTTATACGGAGAAGC
AGTGGGGAAGAAGTGCAAAAGAATTGCCTGCATTTATTATTAAGCGAATCCCAGTGAGATTTACGTTTGAT
AACAATTATTTTTCCGATCGCTATCAAGGTATTCCGGTGGGAGGCTACACTAAGCTTATTGAAAAAATGCT
TGAAGGTGTGGACGTAAAATTAGGCATTGATTTTTTGAAAGACAAAGATTCTCTAGCGAGTAAAGCCCATA
GAATCATCTACACTGGACCCATTGATCAGTACTTCGACTATAGGTTTGGAGCGTTAGAATATCGCTCTTTA
AAATTTGAGACGGAACGCCATGAATTTCCAAACTTCCAAGGGAATGCAGTAATAAATTTCACTGATGCTAA
TGTACCATATACCAGAATAATTGAGCATAAACATTTTGACTATGTTGAGACAAAGCATACGGTTGTTACAA
AAGAATATCCATTAGAGTGGAAAGTTGGCGACGAACCCTACTATCCAGTTAATGATAATAAAAACATGGAG
CTTTTTAAGAAATATAGAGAGTTAGCTAGCAGAGAAGACAAGGTTATATTTGGCGGGCGTTTGGCCGAGTA
TAAATATTATGATATGCATCAAGTGATATCTGCCGCTCTTTATCAAGTGAAAAATATAATGAGTACGGATT
AATGATCTATCTTGTAATTAGTGTCTTTCTCATTACAGCATTTATCTGTTTATATCTTAAGAAGGATATAT
TTTATCCAGCCGTATGCGTTAATATCATCTTCGCACTGGTCTTATTGGGATATGAAATAACGTCAGATATA
TATGCTTTTCAGTTAAATGACGCTACGTTGATTTTTCTACTTTGCAATGTTTTGACATTTACCCTGTCATG
TTTATTGACGGAAAGTGTATTAGATCTAAATATCAGAAAAGTCAATAATGCTATTTATAGCATACCATCGA
AGAAAGTGCATAATGTAGGCTTGTTAGTTATTTCTTTTTCGATGATATATATATGCATGAGGTTAAGTAAC
TACCAGTTCGGGACTAGCTTACTTAGCTATATGAATTTGATAAGAGATGCTGATGTTGAAGACACATCAAG
AAATTTCTCAGCATACATGCAGCCAATCATTCTAACTACTTTTGCTTTATTTATTTGGTCTAAAAAATTTA
CTAATACAAAGGTAAGTAAAACATTTACTTTACTTGTTTTTATTGTATTCATCTTTGCAATTATACTGAAT
ACTGGTAAGCAAATTGTCTTTATGGTTATCATCTCTTATGCATTCATCGTAGGTGTTAATAGAGTAAAACA
TTATGTTTATCTTATTACAGCTGTAGGTGTTCTATTCTCCTTGTATATGCTCTTTTTACGTGGACTGCCTG
GGGGGATGGCATATTATCTATCCATGTATTTGGTCAGCCCTATAATCGCGTTTCAGGAGTTTTATTTTCAG
CAAGTATCTAACTCTGCCAGTTCTCATGTCTTTTGGTTTTTTGAAAGGCTGATGGGGCTATTAACAGGTGG
AGTCTCTATGTCGTTGCATAAAGAATTTGTGTGGGTGGGTTTGCCAACAAATGTTTATACTGCTTTTTCGG
ATTATGTTTATATTTCCGCGGAGCTAAGCTATTTGATGATGGTTATTCATGGCTGTATTTCAGGTGTTTTA
TGGAGATTGTCTCGAAATTACATATCTGTGAAAATATTTTATTCATATTTTATTTATACCTTTTCTTTCAT
TTTTTATCATGAAAGCTTCATGACTAATATTAGCAGTTGGATACAAATAACTCTTTGTATCATAGTATTCT
CTCAATTTCTTAAGGCCCAGAAAATAAAGTGAAAATGTATTTTTTGAATGATTTAAATTTCTCTAGACGCG
ATGCTGGATTTAAAGCAAGAAAAGATGCACTGGACATTGCTTCAGATTATGAAAACATTTCTGTTGTTAAC
ATTCCTCTATGGGGTGGAGTAGTCCAGAGAATTATTAGTTCTGTTAAGCTTAGTACATTTCTCTGCGGTCT
TGAAAATAAAGATGTTTTAATTTTCAATTTCCCGATGGCCAAACCATTTTGGCATATATTGTCATTCTTTC
ACCGCCTTCTAAAATTTAGAATAGTACCTCTGATTCATGATATTGATGAATTAAGAGGAGGAGGGGGTAGT
GATTCTGTGCGGCTTGCTACCTGTGATATGGTCATAAGTCACAATCCACAAATGACAAAGTACCTTAGTAA
ATATATGTCTCAGGATAAAATCAAAGACATAAAAATATTTGATTACCTCGTCTCATCTGATGTGGAGCATC
GAGATGTTACGGATAAGCAACGAGGGGTCATATATGCTGGCAACCTTTCTAGGCATAAATGTTCTTTCATA
TATACTGAAGGATGCGATTTTACTCTCTTTGGTGTCAACTATGAAAATAAAGATAATCCTAAATATCTTGG
AAGTTTTGATGCTCAATCTCCGGAAAAGATTAACCTCCCAGGCATGCAATTTGGACTCATTTGGGATGGAG
ATTCTGTCGAAACCTGTAGTGGTGCCTTTGGCGACTATTTAAAGTTTAATAACCCTCATAAGACATCTCTT
TATCTTTCAATGGAACTTCCAGTATTTATATGGGATAAAGCCGCCCTTGCGGATTTCATTGTAGATAATAG
AATAGGATATGCAGTGGGATCAATCAAAGAAATGCAAGAGATTGTTGACTCCATGACAATAGAAACTTATA
AGCAAATTAGTGAGAATACAAAAATTATTTCTCAGAAAATTCGAACAGGAAGTTACTTCAGGGATGTTCTT
GAAGAGGTGATCGATGATCTTAAAACTCGCTAAACGATATGGTCTCTGTGGTTTTATTCGGCTTGTTAGAG
ATGTCTTATTGACTCGTGTATTTTACCGGAACTGTAGAATTATTCGATTTCCCTGCTATATTCGCAATGAT
GGTAGCATTAATTTTGGTGAAAATTTCACAAGTGGAGTCGGTCTCAGGCTGGATGCATTTGGACGTGGCGT
GATTTTTTTTTCCGATAATGTGCAAGTTAACGACTATGTTCATATCGCCTCAATTGAGAGCGTTACGATAG
GTCGGGATACGCTTATTGCAAGTAAAGTATTTATTACCGATCATAATCACGGTTCCTTTAAGCACTCTGAT
CCAATGAGTTCGCCAAATATACCTCCAGACATGCGCACGTTGGAATCTTCAGCTGTTGTAATTGGCCAGAG
GGTTTGGTTGGGTGAGAATGTGACGGTTTTGCCTGGAACAATTATTGGTAATGGAGTCGTAGTCGGCGCCA
ATTCTGTTGTTAGAGGTTCTATTCCCGAAAATACTGTCATTGCGGGAGTACCAGCAAAAATCATAAAGAAA
TACAATCATGAGACCAAATTATGGGAAAAAGCATAGTCGTTGTTTCTGCGGTCAATTTTACCACTGGCGGT
CCATTTACCATTTTGAAAAAATTTTTGGCAGCAACTAATAATAAAGAAAATGTCAGTTTTATCGCATTAGT
CCATTCTGCTAAAGAGTTAAAAGAAAGTTATCCATGGGTTAAATTCATTGAGTTTCCTGAGGTTAAAGGGT
CGTGGCTAAAACGTTTGCACTTTGAATATGTAGTTTGTAAAAAACTTTCAAAAGAGCTGAATGCTACGCAT
TGGATTTGTCTGCATGATATTACGGCCAATGTCGTCACTAAAAAAAGATATGTGTATTGTCATAACCCTGC
CCCTTTTTATAAAGGAATTTTATTCCGTGAAATTCTTATGGAGCCTAGCTTTTTCTTATTTAAAATGCTAT
ACGGGCTGATATATAAAATAAACATTAAAAAAAATACTGCAGTGTTTGTTCAACAATTCTGGATGAAAGAA
AAATTTATCAAGAAATATTCTATAAATAACATCATTGTCAGTCGGCCAGAAATTAAATTATCTGATAAAAG
CCAACTTACTGATGATGATTCTCAATTTAAGAATAACCCTTCTGAGTTGACAATATTTTACCCTGCTGTTC
CACGAGTATTTAAAAATTACGAGCTTATTATTAGTGCAGCAAGGAAATTGAAAGAACAATCCAATATTAAA
TTTCTGCTTACTATCAGTGGTACAGAAAATGCGTATGCAAAATATATTATCAGTCTTGCAGAAGGACTGGA
TAATGTTCATTTCCTCGGGTACTTGGATAAAGAAAAAATCGATCATTGTTATAATATTTCAGATATAGTTT
GTTTTCCCTCTAGGTTAGAAACATGGGGATTGCCGTTGTCTGAGGCTAAAGAGCGAGGTAAGTGGGTATTA
GCATCAGATTTCCCATTTACTAGAGAAACTCTTGGTAGTTATGAAAAGAAAGCTTTTTTTGATTCTAATAA
CGATGACATGTTAGTTAAACTTATTATTGACTTCAAAAAAGGTAACCTCAAAAAAGATATCTCTGATGCAA
ATTTCATTTATCGTAATGAAAATGTATTAGTTGGGTTTGATGAACTAGTTAATTTTATTACTGAAGAACAT
TGAAATGGTATATATAATAATCGTTTCCCACGGACATGAAGACTACATCAAAAAATTACTCGAAAATCTTA
ATGCTGACGATGAGCACTACAAGATTATCGTACGCGACAACAAAGACTCTCTATTATTGAAACAAATATGC
CAGCATTATGCAGGCCTGGACTATATTAGTGGAGGTGTATACGGCTTTGGTCATAATAATAATATTGCGGT
GGCGTATGTAAAGGAAAAATATAGACCCGCAGATGATGATTACATTTTGTTTTTGAATCCCGATATCATCA
TGAAGCATGATGATTTGCTGACATATATTAAATATGTCGAAAGTAAGCGTTATGCTTTTAGTACATTATGC
CTGTTCCGAGATGAAGCGAAATCTTTACATGATTATTCCGTAAGAAAATTTCCTGTGCTTTCTGATTTTAT
TGTGTCATTTATGTTAGGGATTAATAAAACAAAAATTCCTAAAGAAAGTATCTATTCTGATACGGTTGTTG
ATTGGTGCGCAGGATCATTTATGCTGGTACGTTTTTCAGATTTTGTGCGTGTAAATGGCTTCGATCAAGGT
TACTTTATGTACTGTGAAGATATTGACCTGTGCTTGAGGCTTAGCCTGGCTGGTGTCAGACTTCATTATGT
TCCCGCTTTTCATGCGATACATTATGCTCATCATGACAATCGAAGTTTTTTTTCAAAAGCCTTCAGATGGC
ACTTAAAAAGTACTTTTAGATATTTAGCCAGAAAACGTATTTTATCAAATCGCAACTTTGATCGAATTTCA
TCAGTTTTTCACCCGTAAGAGCTCGGTACCCGGGCCTAGGGTGTAGGCTGGAGCTGCTTCGAAGTTCCTAT
ACTTTCTAGAGAATAGGAACTTCGGAATAGGAACTAAGGAGGATATTCATATCCGTCGACGGCGGCCGCCC
TGCAGGCATGCAAGCTTGATCCATATGGATCGCTAGCTTAATTAAATAAAGCCGTAAGCATATAAGCATGG
ATAAGCTATTTATACTTTAATAAGTACTTTGTATACTTATTTGCGAACATTCCAGGCCGCGAGCATTCAGC
GCGGTGATCACACCTGACAGGAGTATGTAATGTCCAAGCAACAGATCGGCGTAGTCGGTATGGCAGTGATG
GGACGCAACCTTGCGCTCAACATCGAAAGCCGTGGTTATACCGTCTCTATTTTCAACCGTTCCCGTGAGAA
GACGGAAGAAGTGATTGCCGAAAATCCAGGCAAGAAACTGGTTCCTTACTATACGGTGAAAGAGTTTGTCG
AATCTCTGGAAACGCCTCGTCGCATCCTGTTAATGGTGAAAGCAGGTGCAGGCACGGATGCTGCTATTGAT
TCCCTCAAACCATATCTCGATAAAGGAGACATCATCATTGATGGTGGTAACACCTTCTTCCAGGACACTAT
TCGTCGTAATCGTGAGCTTTCAGCAGAGGGCTTTAACTTCATCGGTACGGGTGTTTCTGGCGGTGAAGAGG
GGGCGCTGAAAGGTCCTTCTATTATGCCTGGTGGCCAGAAAGAAGCCTATGAATTGGTAGCACCGATCCTG
ACCAAAATCGCCGCCGTAGCTGAAGACGGTGAACCATGCGTTACCTATATTGGTGCCGATGGCGCAGGTCA
CTATGTGAAGATGGTTCACAACGGTATTGAATACGGCGATATGCAGCTGATTGCTGAAGCCTATTCTCTGC
TTAAAGGTGGCCTGAACCTCACCAACGAAGAACTGGCGCAGACCTTTACCGAGTGGAATAACGGTGAACTG
AGCAGTTACCTGATCGACATCACCAAAGATATCTTCACCAAAAAAGATGAAGACGGTAACTACCTGGTTGA
TGTGATCCTGGATGAAGCGGCTAACAAAGGTACGGGTAAATGGACCAGCCAGAGCGCGCTGGATCTCGGCG
AACCGCTGTCGCTGATTACCGAGTCTGTGTTTGCACGTTATATCTCTTCTCTGAAAGATCAGCGTGTTGCC
GCATCTAAAGTTCTCTCTGGTCCGCAAGCACAGCCAGCAGGCGACAAGGCTGAGTTCATCGAAAAAGTTCG
TCGTGCGCTGTATCTGGGCAAAATCGTTTCTTACGCCCAGGGCTTCTCTCAGCTGCGTGCTGCGTCTGAAG
AGTACAACTGGGATCTGAACTACGGCGAAATCGCGAAGATTTTCCGTGCTGGCTGCATCATCCGTGCGCAG
TTCCTGCAAAAAATCACCGATGCTTATGCCGAAAATCCACAGATCGCTAACCTGTTGCTGGCTCCGTACTT
CAAGCAAATTGCCGATGACTACCAGCAGGCGCTGCGTGATGTCGTTGCTTATGCAGTACAGAACGGTATTC
CGGTTCCGACCTTCTCCGCAGCGGTTGCCTATTACGACAGCTACCGTGCTGCTGTTCTGCCTGCGAACCTG
ATCCAGGCACAGCGTGACTATTTTGGTGCGCATACTTATAAGCGTATTGATAAAGAAGGTGTGTTCCATAC
CGAATGGCTGGATTAA
SEQ ID NO: 17 (example O18A rfb locus nucleotide seauence - O18A-EPA production strain BVEC-
L-00559)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGTGAAAATACTT
GTTACTGGTGGCGCAGGATTTATTGGTTCAGCTGTAGTTCGTCACATTATAAATAATACGCAGGATAGTGT
TGTTAATGTCGATAAATTAACGTACGCCGGAAACCGGGAATCACTTGCTGATGTTTCTGATTCTGAACGCT
ATGTTTTTGAACATGCGGATATTTGCGATGCACCTGCAATGGCACGGATTTTTGCTCAGCATCAGCCGGAT
GCAGTGATGCACCTGGCTGCTGAAAGCCATGTTGACCGTTCAATTACAGGCCCTGCGGCATTTATTGAAAC
CAATATTGTTGGTACTTATGTCCTTTTGGAAGCCGCTCGCAATTACTGGTCTGCTCTTGATAGCGACAAGA
AAAATAGCTTCCGTTTTCATCATATTTCTACTGACGAAGTCTATGGTGATTTGCCTCATCCAGATGAAGTA
AATAATACAGAAGAATTACCCTTATTTACTGAGACGACAGCTTACGCGCCAAGCAGCCCTTATTCCGCATC
CAAAGCATCCAGCGATCATTTAGTCCGCGCGTGGAAACGTACATATGGTTTACCGACAATTGTGACTAATT
GCTCGAACAACTATGGTCCTTATCATTTCCCGGAAAAGCTTATTCCACTGGTTATTCTTAATGCACTGGAA
GGTAAGGCATTACCTATTTATGGCAAAGGAGATCAGATCCGCGACTGGTTGTATGTTGAAGATCATGCGCG
TGCGTTATATACCGTCGTAACCGAAGGTAAAGCGGGTGAAACTTATAACATTGGTGGGCACAACGAAAAGA
AAAACATCGATGTAGTGCTCACTATTTGTGATTTGCTGGATGAGATTGTACCGAAAGAGAAATCTTATCGT
GAGCAAATCACTTATGTTGCTGATCGTCCGGGACACGATCGCCGCTATGCTATTGATGCTGAGAAGATTGG
TCGCGCATTGGGATGGAAACCACAGGAAACGTTTGAGAGCGGGATTCGTAAAACGGTGGAATGGTACCTGT
CCAATACAAAATGGGTTGATAATGTGAAAAGTGGTGCCTATCAATCGTGGATTGAACAGAACTATGAGGGC
CGCCAGTAATGAATATCCTCCTTTTTGGCAAAACAGGGCAGGTAGGTTGGGAACTACAGCGTGCTCTGGCA
CCTTTGGGTAATTTGATTGCTTTTGATGTTCACTCTACTGATTATTGCGGTGATTTTAGTAATCCTGAAGG
TGTAGCTGAAACCGTAAGAAGCATTCGGCCGGATATTATTGTCAATGCAGCCGCTCACACCGCAGTAGACA
AAGCAGAATCAGAACCGGAGTTTGCACAATTAATTAACGCAACAAGTGTCGAAGCGATTGCGAAAGCAGCA
AATGAAGTTGGAGCCTGGGTTATCCATTACTCGACTGATTACGTCTTCCCTGGAAATGGCGATATGCCATG
GCTGGAGACGGATGCAACCGCACCACTAAATGTTTACGGTGAAACCAAGTTAGCCGGAGAAAAAGCGTTAC
AGGAATATTGCGCGAAGCATCTTATTTTCCGGACCAGCTGGGTCTATGCAGGAAAAGGAAATAACTTCGCC
AAAACGATGTTACGTCTGGCAAAAGAGCGTGAAGAATTAGCGGTTATTAACGATCAGTTTGGTGCGCCAAC
AGGTGCTGAACTGCTGGCTGATTGTACAGCACATGCCATTCGTGTCGCACTGAATAAACCGGATGTCGCAG
GCTTGTACCATTTGGTAGCCAGTGGTACCACAACCTGGTACGATTATGCTGCGCTGGTTTTTGAAGAGGCG
CGCAAAGCAGGCATTCCCCTTGCACTCAACAAGCTCAACGCAGTACCAACAACAGCCTATCCTACACCAGC
TCGTCGTCCACATAACTCTCGCCTTAATACAGAAAAATTTCAGCAGAACTTTGCGCTTGTCTTGCCTGACT
GGCAGGTTGGCGTGAAACGAATGCTCAATGAATTATTTACGACTACAGCAATTTAATAGTTTTTGCATCTT
GTTCGTGATGGTGGAGCAAGATGAATTAAAAGGAATGATGAAATGAAAATGCGTAAAGGTATTATTTTAGC
GGGTGGTTCTGGTACACGTCTTTATCCTGTGACTATGGCTGTCAGTAAACAGCTATTACCTATTTATGATA
AACCGATGATCTATTACCCGCTCTCTACACTGATGTTGGCGGGTATTCGCGATATTTTGATTATCAGTACA
CCTCAGGATACTCCTCGTTTTCAACAATTGCTGGGTGACGGTAGCCAGTGGGGCCTGAATCTTCAGTACAA
AGTGCAACCTAGCCCAGATGGCCTCGCGCAGGCATTTATCATCGGTGAAGAGTTTATTGGTGGTGATGATT
GTGCTTTGGTTCTTGGTGATAATATCTTTTACGGTCACGATCTGCCGAAGCTAATGGAGGCCGCTGTTAAC
AAAGAAAGTGGTGCAACGGTATTTGCCTATCACGTTAATGATCCAGAACGCTATGGTGTCGTTGAGTTTGA
TAAAAACGGTACGGCAATCAGTCTGGAAGAAAAACCGTTAGAACCAAAGAGTAATTACGCCGTTACAGGTC
TGTACTTTTATGATAACGACGTGGTTCAGATGGCGAAAAACTTGAAGCCGTCTGCACGTGGTGAGTTAGAA
ATTACAGATATTAACCGTATTTATCTTGAGCAGGGACGTCTGTCTGTCGCGATGATGGGGCGTGGCTACGC
GTGGCTGGACACGGGGACTCATCAGAGTCTGATAGAAGCAAGTAATTTTATTGCGACAATTGAAGAGCGCC
AGGGATTGAAGGTTTCCTGTCCTGAAGAGATTGCATTTCGTAAAGGTTTTATTGATGTTGAGCAAGTAAGA
AAATTAGCTGTACCACTAATAAAGAATAATTATGGGCAGTATCTTTATAAAATGACGAAGGATTCAAATTA
ATGAATGTGATTAGAACTGAAATTGAAGATGTGCTAATTCTGGAGCCAAGAGTATTTGGTGATGATAGAGG
TTTCTTTTATGAGAGCTTTAATCAATCAGCATTTGAACATATTCTAGGCTATCCGGTCAGCTTTGTTCAAG
ACAATCACTCACGTTCATCAAAAAATGTACTCAGAGGCCTTCACTTTCAACGCGGCGAGTACGCACAAGAT
AAACTTGTACGCTGCACTCATGGAGCAGTTTTTGATGTTGCTGTTGATATTCGACCCAATTCGGTATCCTT
TGGTAAATGGGTTGGTGTTCTGCTTTCAGCTGATAATAAGCAGCAGTTGTGGATACCAAAAGGGTTTGCTC
ATGGCTTTTTGGTTCTGTCTGATATCGCTGAATTTCAATATAAAACTACAAACTATTATCATCCTGAAAGC
GATTGTGGAATATGTTGGAATGATGAACGCATTGCAATTGATTGGCCCCAAACATCAGGGTTAATCCTTTC
GCCAAAAGATGAAAGGCTCTTTACGTTAGATGAGCTTATCAGATTAAAATTAATTGCATGAGGCCGGCCTT
AAGGAGGACTAGTCCCGGCGCGCCATGAGTTTAATCAAAAACAGTTTTTGGAACCTTTGCGGGTATGTACT
TCCAGCTATTGTGACACTACCAGCTTTGGGTATTATGGGGCGAAAATTAGGCCCAGAATTATTTGGTGTAT
TCACTTTGGCATTAGCTGTTGTGGGTTATGCAAGCATTTTTGATGCAGGCCTTACTCGCGCAGTGATACGA
GAAGTCGCAATTGAAAAAGATAATGAAGAAAATAAGTTGAAAATTATTTCTTCAGCGACAGTTGTAATTAT
TTATTTGAGTTTGGCCGCCTCACTCTTATTATTTTTTTTTAGTGGTCATATCGCATTGCTACTGAACATTA
GTGAGACTTTTTTTCATAATGTAAGTGTCTCGCTTAAAATTCTCGCAGCATCCATACCATTATTTTTGATT
ACTCAAATATGGTTGTCAATTTTAGAAGGTGAAGAAAGATTTGGTTTACTTAATATCTACAAATCAATTAC
GGGAGTGATATTAGCAATCTCACCGGCATTATTTATACTTATTAAACCCTCTTTGATGTATGCGATAATAG
GCTTAGTTCTAGCAAGGTTTTTATGTTTTATTTTGGCTTTTATAATTTGTCACGATAAAGTGCTTAAAGCT
AAACTAACAATCGATATACCAACAATTAAAAGATTGTTTATGTTCGGTGGTTGGATTACAGTAAGTAATAT
CATCAGCCCTGTGCTATCATATTTTGATAGGTTTATTGTTTCAAATCAACTTGGGGCTGCTAATGTTGCTT
TTTATACTGCACCATCAGAAATTATTTCTCGGCTTAGTATAATTCCAGGTGCGTTTTCAAGAGCCTTATTT
CCAAGATTAGCTAATGCAAATAATTCCGCTGAAAGATATAAAACGAAAAGATTAATTACAATTTCACTTTT
AATAATCATCACCCCTATTTTTTGTATTGGCGTGTTATTTTCAGAGAAGATAATGGTTTTATGGATGGGGG
CATCATTTTTTGGTGAGCCTGGTTTGGTATTATCAATATTACTGATTGGCTTTATTTTTAATGGATTGGCA
CAAGTACCATTTGCCAGTATTCAATCCCGAGGTCATGCTAAGATAACTGCATTTGTTCATCTCTTAGAGTT
GTTTCCTTATTTATTACTTTTATTTTACCTCATAAAAGCACATGGGGTTGTTGGCGCGGGTATTGCGTGGT
CAGTGAGGATGATAGTAGATTATATAGCATTAAGTCTTTTGGACGGTAAGTATATTAATAAATAAAATTCA
AAATGCAAGTTAATAACTCATGGCTTTATTTGGGTAGGTGACAATTTATAATGATATATATATTAACTTTA
ACTCTTCTTCTAGTTATAGCCATAATGTTTTCTCTTCTCGGCACAAAAAGTAGGATCACATCTCCATTACC
TTTGCATTTTTTACCATGGTTACTAACTTTAATTGTCGGGATAAGTAATTACGATCAATTTTACGAGTTTA
ATGAAAGAAGCTTTTACTCTTTGTTGATTTGGTTTACAGTTATTTTTATATTTTATTTCATAGGGGAACTG
GTTAATTATAAACGTGAAAATATAAATGTTTATTATGGTCTTTCACATATTAAATATGAATGTAAAAAATA
TTGGATCATTGTCATCCCAATTTCATTATATACCATTTTCGAAATATATATGGTTGGTATGGGGGGAGCAG
ATGGATTCTTTCTCAATTTACGTCTTGCAAATACATTGGAGGGCTATACGGGTAAAAAATTTATCTTAATG
CCTGCTGTATATCCTCTAATGATGGCTATGTTCGCAATTGTTTGTCTAACAAAAACTTCCAAATTAAATAA
ATACTCCATTTATTTCTGGATGTTTTTGTATTGTATTGGCACAATGGGAAAATTTTCAATATTAACGCCAA
TATTGACATATTTAATTATTTATGACTTCAAACATAGATTAAAAGTAAAAAAAACAATAAAGTTTACATTG
TTGATAATTATATTAGCTTTAACTTTGCATTTTACACGTATGGCTGAGAATGACCACTCAACATTTTTATC
TATTTTAGGGCTCTATATTTATTCACCAATAATTGCTTTAGGCCAGTTGAATGAAGTAAATAGTAGTCATT
TTGGTGAGTATACGTTTAGATTCATATATGCTATAACTAATAAAATTGGCCTTATTAAAGAATTGCCAGTA
AATACTATTCTTGACTATTCATACGTTCCTGTACCAACAAATGTATATACTGCACTTCAACCATTTTACCA
GGATTTTGGTTATACTGGCATCATATTTGGAGCAGTATTATACGGACTAATATATGTGAGTTTATACACGG
CCGGTGTTCGTGGAAATAATACACAGGCATTACTGATTTACGCATTGTTTTCAGTTAGCAGTGCAACGGCT
TTCTTCGCTGAAACGCTAGTAACGAATTTAGCTGGAAATGTGATGTTAGTATTATGTACCATCTTACTATG
GCGATTTACAGTAATATGCAAACCAGTACAGTAACCATTCTAATGGCCACCTACAATGGCGAGGCCTTCAT
CAAAAATCAGATTTTGTCACTACAACAACAAACATTTTCTAACTGGCGGTTATTTATTCAGGATGATGGGT
CTACAGACAATACTATATCTATAATAAAAAACTTCCAAAAATCTGACTCCAGAATTCGGCTAGTTGATGAT
AATTTGAAAGGTCAAGGTGCAGGAAAAAATTTTTTATCGCTGATAAAGTACAGCGAGACAGATTATACAAT
TTATTGTGACCAAGATGATATTTGGTTAGAAAACAAAATATTTGAATTAGTAAAGTATGCAAATGAAATTA
AATTGAATGTATCAGATGCGCCTTCGCTAGTTTATGCTGATGGCTATGCTTATATGGATGGTGAGGGTACA
ATCGATTTTTCTGGGATATCTAACAATCATGCTGATCAATTAAAGGATTTTCTTTTTTTTAATGGTGGATA
CCAAGGATGTTCTATTATGTTCAATCGTGCAATGACCAAATTTCTTCTGAATTATCGAGGATTTGTATATC
TACATGACGATATCACAACATTAGCTGCATACGCTCTTGGTAAAGTTTATTTTCTCCCGAAATACCTTATG
TTATATAGACAGCACACGAATGCGGTAACTGGTATCAAAACATTCCGCAATGGATTGACTTCTAAATTTAA
ATCACCAGTAAACTATCTTTTATCACGAAAACATTATCAGGTAAAAAAATCTTTTTTTGAATGTAACAGCT
CTATCTTATCAGAGACGAATAAAAAAGTTTTTTTGGATTTTATTTCATTTTGTGAATCAAATAATAAATTT
ACAGATTTTTTTAAGTTATGGCGAGGTGGGTTTAGATTAAATAACAGTAGAACTAAATTATTATTAAAATT
CTTAATACGGAGAAAATTTAGCGAATGATTTCAATACTTACACCTACTTTTAATCGGCAACATACTTTATC
AAGGCTATTCAATTCTCTTATATTACAAACTGATAAAGATTTTGAGTGGATAATAATTGATGATGGTAGTA
TAGATGCAACAGCGGTACTTGTAGAAGATTTTAGAAAAAAATGTGATTTTGACTTGATTTATTGCTATCAG
GAAAATAATGGTAAGCCCATGGCTTTAAACGCTGGTGTTAAAGCTTGTAGAGGCGATTATATCTTTATTGT
TGACAGTGATGATGCACTAACTCCCGATGCCATAAAATTAATTAAAGAATCAATACATGATTGCTTATCTG
AGAAGGAAAGTTTCAGCGGAGTCGGTTTTAGAAAAGCATATATAAAAGGGGGGATTATTGGTAATGATTTA
AATAATTCTTCAGAACATATATACTATTTAAATGCGACTGAGATTAGCAATTTAATAAATGGTGATGTTGC
ATATTGTTTTAAAAAAGAAAGTTTGGTAAAAAATCCATTCCCCCGTATAGAAGATGAAAAATTTGTTCCAG
AATTATATATTTGGAATAAAATAACTGACAAGGCGAAGATTCGATTTAACATAAGCAAAGTTATATATCTT
TGTGAGTATCTTGATGATGGTCTTTCTAAAAATTTCCATAACCAGCTTAAAAAATACCCAAAGGGGTTTAA
GATTTATTACAAAGATCAAAGAAAACGAGAGAAAACTTATATAAAAAAAACAAAGATGCTAATTAGATATT
TGCAATGTTGTTATTATGAGAAAATAAAATGAAAATACTATTTGTCATTACAGGTTTAGGCCTTGGAGGTG
CTGAGAAGCAGGTTTGTCTTTTAGCTGATAAATTAAGTTTAAGCGGGCACCATGTAAAGATTATTTCACTT
GGACATATGTCTAATAATAAAGTCTTTCCTAGCGAAAATAATGTTAATGTCATTAATGTAAATATGTCAAA
AAACATTTCTGGAGTTATAAAAGGTTGTGTCAGAATTAGAGATGTTATAGCTAATTTCAAACCAGACATTG
TACACAGTCATATGTTTCATGCAAACATTATCACTAGATTGTCTGTAATTGGAATCAAAAACAGACCTGGT
ATTATATCAACTGCACATAATAAAAATGAAGGTGGGTATTTCAGAATGCTCACATATAGAATAACCGATTG
TTTAAGTGATTGTTGTACAAATGTTAGCAAAGAAGCAGTGGATGAGTTTTTACGGATAAAAGCCTTTAATC
CCGCTAAAGCAATTACTATGTATAATGGGATAGATACCAATAAATTTAAATTTGATTTATTGGCAAGGAGG
GAAATTCGAGACGGTATTAATATAAAAAATGATGATATATTATTACTTGCTGCAGGTCGTTTAACGTTAGC
TAAAGATTATCCTAATTTATTGAATGCAATGACTCTGCTTCCTGAACACTTTAAACTTATTATTATTGGTG
ATGGTGAATTGCGTGACGAAATTAATATGCTTATAAAAAAATTGCAATTATCTAATAGGGTGTCCTTGTTG
GGAGTTAAAAAAAATATTGCTCCCTATTTTTCTGCATGTGATATTTTTGTTCTCTCTTCTCGTTGGGAAGG
ATTTGGATTAGTCGTGGCAGAAGCTATGTCATGTGAGCGAATTGTTGTTGGCACGGATTCAGGGGGAGTAA
GAGAAGTTATTGGTGACGATGATTTTCTTGTACCCATATCTGATTCAACACAACTTGCAAGCAAAATTGAA
AAATTGTCTTTGAGCCAGATACGTGATCACATTGGTTTTCGGAATCGTGAGCGTATTTTAAAAAATTTCTC
AATAGATACTATTATTATGCAGTGGCAAGAACTCTATGGAACTATAATTTGCTCAAAACATGAAAGGTAGA
TTTATATTTGGAACGTGTCTTTTGTTTGAATTTAATTCAATCTCAATTGAGATTTTTGTATTTCAAAAATA
CCATCATAGCTAACGATGATTGGTATTTATTTTAAGATGCTTTCTATAAATATATTGACGTTTTTAATGCG
CCGAAACGATTGGGCTGGGAACAGAGAAGTAAAACTGTTTTGAGAATGAAGAGTTTTTGAGATGTTTATGG
ATATTAAAAATTGATCCAGTGAATTAATTATTTATAATAAATCAAGATTTAATGTTAATAAATGATAATCT
TTTCTGACACTCATATTAATTATGAGTGGTACGTTTGGTAAACGGTAAACTATTATATGACAGCTAGAACA
ACTAAAGTTTTGCACTTACAATTACTCCCACTCTTAAGTGGCGTTCAAAGGGTAACATTAAACGAAATTAG
TGCGTTATATACTGATTATGATTATACACTAGTTTGCTCAAAAAAAGGTCCACTAACAAAAGCATTGCTGG
AATATGATGTCGATTGTCATTGTATCCCCGAACTTACGAGAGAAATTACCGTAAAGAATGATTTTAAAGCA
TTGTTCAAGCTTTATAAGTTCATAAAAAAAGAAAAATTTGACATTGTGCATACACATTCTTCAAAAACAGG
TATTTTGGGGCGAGTTGCTGCCAAATTAGCACGTGTTGGAAAGGTGATCCACACTGTACATGGTTTTTCTT
TTCCAGCCGCATCTAGTAAAAAAAGTTATTACCTTTATTTTTTCATGGAATGGATAGCAAAGTTCTTTACG
GATAAGTTAATCGTCTTGAATGTAGATGATGAATATATAGCAATAAACAAATTAAAATTCAAGCGGGATAA
AGTTTTTTTAATTCCTAATGGAGTAGACACTGATAAGTTTTCTCCTTTAGAAAATAAAATTTATAGTAGCA
CCTTGAATCTAGTAATGGTTGGTAGATTATCCAAGCAAAAAGATCCTGAGACATTATTGCTTGCTGTTGAA
AAACTGCTGAATGAAAATGTTAATGTTAAGCTGACACTTGTAGGAGATGGTGAACTAAAAGAACAGTTAGA
AAGCAGGTTCAAACGGCAAGATGGACGTATAATTTTTCATGGATGGTCAGATAACATTGTTAATATTTTAA
AAGTTAATGATCTTTTTATATTACCTTCTCTTTGGGAGGGTATGCCATTAGCAATTTTAGAAGCATTGAGC
TGTGGACTTCCATGTATAGTCACTAATATTCCAGGTAATAATAGCTTAATAGAAGATGGCTATAATGGTTG
TTTGTTTGAAATTAGAGATTGTCAGTTATTATCTCAAAAAATCATGTCATATGTTGGTAAGCCAGAACTGA
TTGCACAGCAATCTACCAATGCACGATCATTTATTCTGAAAAATTATGGATTAGTTAAAAGAAATAATAAG
GTCAGACAGCTATATGATAATTAAGAGCTCGGTACCCGGGCCTAGGGTGTAGGCTGGAGCTGCTTCGAAGT
TCCTATACTTTCTAGAGAATAGGAACTTCGGAATAGGAACTAAGGAGGATATTCATATCCGTCGACGGCGG
CCGCCCTGCAGGCATGCAAGCTTGATCCATATGGATCGCTAGCTTAATTAAATAAAGCCGTAAGCATATAA
GCATGGATAAGCTATTTATACTTTAATAAGTACTTTGTATACTTATTTGCGAACATTCCAGGCCGCGAGCA
TTCAGCGCGGTGATCACACCTGACAGGAGTATGTAATGTCCAAGCAACAGATCGGCGTAGTCGGTATGGCA
GTGATGGGACGCAACCTTGCGCTCAACATCGAAAGCCGTGGTTATACCGTCTCTATTTTCAACCGTTCCCG
TGAGAAGACGGAAGAAGTGATTGCCGAAAATCCAGGCAAGAAACTGGTTCCTTACTATACGGTGAAAGAGT
TTGTCGAATCTCTGGAAACGCCTCGTCGCATCCTGTTAATGGTGAAAGCAGGTGCAGGCACGGATGCTGCT
ATTGATTCCCTCAAACCATATCTCGATAAAGGAGACATCATCATTGATGGTGGTAACACCTTCTTCCAGGA
CACTATTCGTCGTAATCGTGAGCTTTCAGCAGAGGGCTTTAACTTCATCGGTACCGGTGTTTCTGGCGGTG
AAGAGGGGGCGCTGAAAGGTCCTTCTATTATGCCTGGTGGCCAGAAAGAAGCCTATGAATTGGTAGCACCG
ATCCTGACCAAAATCGCCGCCGTAGCTGAAGACGGTGAACCATGCGTTACCTATATTGGTGCCGATGGCGC
AGGTCACTATGTGAAGATGGTTCACAACGGTATTGAATACGGCGATATGCAGCTGATTGCTGAAGCCTATT
CTCTGCTTAAAGGTGGCCTGAACCTCACCAACGAAGAACTGGCGCAGACCTTTACCGAGTGGAATAACGGT
GAACTGAGCAGTTACCTGATCGACATCACCAAAGATATCTTCACCAAAAAAGATGAAGACGGTAACTACCT
GGTTGATGTGATCCTGGATGAAGCGGCTAACAAAGGTACGGGTAAATGGACCAGCCAGAGCGCGCTGGATC
TCGGCGAACCGCTGTCGCTGATTACCGAGTCTGTGTTTGCACGTTATATCTCTTCTCTGAAAGATCAGCGT
GTTGCCGCATCTAAAGTTCTCTCTGGTCCGCAAGCACAGCCAGCAGGCGACAAGGCTGAGTTCATCGAAAA
AGTTCGTCGTGCGCTGTATCTGGGCAAAATCGTTTCTTACGCCCAGGGCTTCTCTCAGCTGCGTGCTGCGT
CTGAAGAGTACAACTGGGATCTGAACTACGGCGAAATCGCGAAGATTTTCCGTGCTGGCTGCATCATCCGT
GCGCAGTTCCTGCAAAAAATCACCGATGCTTATGCCGAAAATCCACAGATCGCTAACCTGTTGCTGGCTCC
GTACTTCAAGCAAATTGCCGATGACTACCAGCAGGCGCTGCGTGATGTCGTTGCTTATGCAGTACAGAACG
GTATTCCGGTTCCGACCTTCTCCGCAGCGGTTGCCTATTACGACAGCTACCGTGCTGCTGTTCTGCCTGCG
AACCTGATCCAGGCACAGCGTGACTATTTTGGTGCGCATACTTATAAGCGTATTGATAAAGAAGGTGTGTT
CCATACCGAATGGCTGGATTAA
SEQ ID NO: 18 (example O25B rfb locus nucleotide sequence - O25B-EPA production strain
stGVXN4459)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGCTAGCAGTGAA
GATACTTGTTACTGGTGGCGCAGGATTTATTGGTTCTGCTGTTGTTCGTCACATAATAAATAATACGCAAG
ATAGTGTTGTTAATGTCGATAAATTAACATACGCCGGAAACCTGGAATCACTTGCAGATGTTTCTGATTCT
GAACGCTATTTCTTTGAACATGCGGATATTTGTGATGCAGCTGCAATGGCACGGATTTTTGCTCAGCATCA
GCCGGATGCAGTGATGCACCTGGCAGCTGAAAGCCATGTTGACCGTTCAATTACAGGCCCTGCGGCATTTA
TTGAAACCAATATTGTGGGTACTTATGTCCTTTTAGAAGCGGCTCGGAATTATTGGTCTGGTCTGGATGAT
GAAAAGAAAAAAAACTTCCGTTTTCATCATATTTCTACTGATGAGGTGTATGGTGACTTACCCCATCCGGA
TGAAGTAAATAGCAATGAAACGTTGCCGCTATTTACGGAAACGACAGCATACGCGCCAAGTAGTCCATATT
CTGCTTCTAAAGCTTCCAGCGATCATTTGGTTCGCGCATGGAAACGTACTTATGGTTTACCGACCATTGTG
ACTAATTGCTCGAACAACTATGGTCCTTATCATTTCCCGGAAAAGCTTATTCCACTGGTTATTCTTAATTC
ACTGGAAGGTAAGGCATTACCTATTTATGGCAAAGGAGATCAGATCCGCGACTGGTTGTATGTAGAGGATC
ATGCTCGAGCGTTATATACCGTCGTAACCGAAGGTAAAGCGGGCGAAACTTATAACATTGGTGGACACAAC
GAAAAGAAAAACATCGACGTAGTGTTCACTATTTGTGATTTGTTGGATGAGATAGTCCCGAAAGAGAAATC
TTACCGCGAGCAAATTACTTATGTTACCGATCGTCCGGGACACGATCGCCGTTATGCGATTGATGCTGAGA
AGATTGGTCGCGAATTGGGATGGAAACCACAGGAAACGTTTGAGAGTGGGATTCGTAAAACGGTGGAATGG
TACCTGTCCAATACAAAATGGGTTGATAATGTGAAAAGTGGTGCCTATCAATCGTGGATTGAACAGAACTA
TGAGGGCCGCCAGTAATGAATATCCTCCTTTTTGGCAAAACAGGGCAGGTAGGTTGGGAACTACAGCGTGC
TCTGGCACCTCTGGGTAATTTGATTGCTCTTGATGTTCACTCCACTGATTACTGTGGTGATTTTAGTAATC
CTGAAGGTGTAGCTGAAACCGTAAGAAGCATTCGGCCTGATATTATTGTCAACGCAGCCGCTCACACCGCA
GTAGACAAAGCAGAATCAGAACCGAAGTTTGCACAATTACTGAACGCGACGAGTGTCGAAGCGATCGCGAA
AGCAGCCAATGAAGTCGGCGCCTGGGTTATTCACTACTCTACTGACTACGTATTTCCGGGGACCGGTGAAA
TACCATGGCAGGAGGAGGATGCAACCGCACCGCTAAATGTTTACGGTGAAACCAAGTTAGCGGGAGAAAAA
GCATTACAAGAGCATTGTGCGAAGCACCTTATTTTCCGGACCAGCTGGGTCTATGCAGGTAAAGGAAATAA
CTTCGCCAAAACAATGTTGCGTCTGGCAAAAGAGCGTGAAGAATTAGCCGTTATTAATGATCAGTTTGGTG
CGCCAACTGGCGCAGAGTTACTGGCTGATTGTACGGCACATGCTATTCGTGTGGCACTGAATAAACCGGAA
GTCGCAGGCTTGTACCATCTGGTAGCTAGTGGTACCACAACGTGGCACGATTATGCTGCGCTGGTTTTTGA
AGAGGCGCGCAAAGCAGGCATTCCCCTTGCACTCAACAAGCTCAACGCAGTACCAACAACAGCCTATCCTA
CACCAGCTCGTCGTCCACATAACTCTCGCCTTAATACAGAAAAATTTCAGCAGAACTTTGCGCTTGTCTTG
CCTGACTGGCAGGTTGGCGTGAAACGAATGCTTAACGAATTATTTACGACTACAGCAATTTAATAGTTTTT
GCATCTTGTTCGTAATGGTGGAGCAAGATGTATTAAAAGGAATGATGAAATGAAAACGCGTAAAGGTATTA
TTTTGGCGGGTGGTTCTGGTACTCGTCTTTATCCTGTGACGATGGCCGTCAGTAAACAGCTGTTACCGATT
TATGATAAACCGATGATCTATTACCCGCTCTCTACACTGATGTTAGCGGGTATTCGCGATATTCTGATTAT
CAGTACACCACAGGATACTCCTCGTTTTCAACAACTGCTGGGTGACGGGAGCCAGTGGGGCCTGAATCTTC
AGTACAAAGTGCAACCGAGTCCGGATGGTCTTGCGCAGGCGTTTATTATCGGTGAAGAGTTTATTGGTGGT
GATGATTGTGCTTTGGTACTTGGTGATAATATCTTCTACGGCCACGACCTGCCGAAGTTAATGGACGTAGC
TGTTAACAAAGAAAGTGGTGCAACGGTATTTGCCTATCACGTTAATGATCCTGAACGTTATGGTGTCGTGG
AGTTTGATAATAACGGTACTGCAATTAGCCTGGAAGAAAAACCGCTGGAACCAAAAAGTAACTATGCGGTT
ACTGGGCTTTATTTCTATGACAATGACGTTGTGGAAATGGCGAAAAACCTTAAGCCTTCTGCCCGAGGTGA
ACTGGAAATTACCGATATTAACCGTATTTATATGGAACAAGGACGTTTGTCTGTCGCTATGATGGGGCGTG
GCTATGCATGGCTGGATACAGGGACGCATCAAAGTCTTATTGAAGCAAGCAACTTCATTGCCACCATTGAA
GAGCGCCAGGGACTAAAGGTTTCCTGTCCGGAAGAAATTGCTTATCGTAAAGGGTTTATTGATGCTGAGCA
GGTAAAAGTATTAGCCGAACCGTTGAAGAAAAATGCTTATGGTCAGTATCTGCTCAAAATGATTAAAGGTT
ATTAATAAGATGAACGTAATTAAAACTGAAATTCCTGATGTGCTGATTTTTGAACCAAAAGTTTTTGGGGA
TGAACGTGGCTTCTTTTTTGAGAGTTTTAATCAGAGGATTTTTGAAGAAGCAGTAGGTCGTAAGGTTGAGT
TTGTTCAGGATAACCATTCTAAGTCCAGTAAAGGTGTTTTACGTGGTCTTCATTATCAGTTAGAACCTTAT
GCTCAAGGAAAACTGGTGCGCTGTGTTGTTGGCGAGGTTTTTGATGTTGCGGTTGATATTCGTAAATCGTC
ACCTACATTTGGGAAATGGGTTGGGGTGAATTTGTCTGCTGAGAATAAGCGTCAGTTGTGGATTCCTGAGG
GATTTGCACATGGTTTTTTGGTGCTGAGTGATTTAGCAGAAGTTTTATATAAAACGAATCAATATTATGCT
CCATCACATGAAAAAAATATTATATGGAATGACCTCTTGCTTAATATTAAATGGCCGAGCACAGCACTGAT
CACTCTGTCTGATAAGGATGCAAATGGGGAAAGATTTGAACTAAGTGAGTTTTGAAATGTCTCTCTTAAAA
CATAGTATATGGAATGTTGCGGGCTACTTTATACCAACATTAATTGCAATTCCCGCCTTTGGATTAATTGC
GAGGAAAATTGGTGTAGAACTATTTGGTTTGTATACGTTAGCAATGATTTTTATAGGGTATGCAAGTATAT
TTGATGCTGGGTTAACAAGAGCTGTTGTGCGTGAAATAGCATTACTAAAAAACAGAGTGGACGATTGTAAT
ACGATAATAGTAACTTCTATTATCGCTGTGATATTTTTAGGGTTTATCGGAGGCGGGGGAGTGTTTCTGCT
TAAAGGCGATATTATTGAACTGTTAAATATCTCACCAATATATTACGCCGATTCGATAAAGTCTCTAGTAT
TATTATCATCTCTGATACCTGTATTCTTAGTCACGCAAATACTATTAGCAGAGCTTGAGGGTCGGGAATAT
TTTGGGATTCTAAATATACAAAAAAGTGTAGGGAATTCTTTAATTGCAGGGTTACCTGCATTATTTGTTTT
AATTAATCAAACGCTTTTTTCTGCAATTATTGGTGTAGCGATTGCAAGAGTTATATGCTTGTGGTTAAGCT
ACATTATGAGCAGGGAAAGAATAACTATCGATATCTCATTTTTTTCAATAACTGTTTTAAAGCGGTTATTT
AGATATGGCGGGTGGGTAACTATAAGTAACATAATATCTCCTATATTAGCGAGTATGGATAGATTTATTCT
ATCCCATATCCAGGGAGCATCAAAAATATCATTCTATACAGTCCCTAATGAGCTGGTAACTAGGCTTGGAA
TAGTTCCAGGCTCTCTTGGGAAAGCTGTTTTTCCAAAATTAAGTCATGCAAGGAATTTTACAGCGTCATAT
GCAGAGCAAAAAAAAGCTTATATATTAATGACTGTCATTGTAATGCCTTTGGTTTTATTTGTATATTATTA
CGCAAAGTTTATTTTAACATTGTGGATGGGGGCTGAGTATGCAGGGATTTCGGTCGAAATATTACGGATTA
TGCTTATAGGGTATATTTTTAACTGTTATTCACAAATCTCTTTTGCCAACATACAGGCCTTTGGAAAAGCA
AAATACACTGCATACATCCATATGATGGAATTTATTCCTTATTTGATAATGTTATATATAATTTCAAAGGA
ATATGGGGTTATTGGTGTTGCGTGGTTATGGACAATTCGAGTAATAATTGATTTTTTGATGCTTTTATATA
TGAGTTATCGTTGTAATAATCTTATGAAAAAAGGGTAGCCTGATGATATATATTGTGGTATTAAATTGGAA
TGGGGCTATAGATACCATTAATTGTGTTAAAAGTTTAATGGATTTAAATGTTAGCGATTATAAAATTATCA
TTGTTGATAACTGTTCTATGGATAACTCATATGATACTATAAAAGAAAATCTTAATTCATTATATATTGCT
GATAAAAGTATCATTGAGGTGAAGTATGAGGATAGAAATAAATATAAAACCTTAGAAAACGATAAAATCAT
ATTAATACAATCTCCGCAAAATAATGGGTACGCAAGTGGTAATAATATTGGCATAGAGTTCGCTCTTAATC
AGGAGAATATGAAATACGTCTGGGTTCTGAATAATGATACTGAAGTGGATAAAGAGGCTTTAACTCATTTA
ATTAGTAAATGTGATTCAGATAAAAGTATAGGGATTTGCGGTTCTCGTTTAGTCTATTTTGCCGACAGAGA
GATGCAGCAAGGACTAGGTGGGGTGCATAACAAATGGTTATGCACTACAAAAAATTATGAAATGGGAAGAT
TAGTTTCCAAAAAATATGATGATGAAGTCATTAGTAATGATATAGATTATATAATTGGCGCATCGATGTTT
TTCTCTAGAGAATGTTTGGAAACAGTTGGATTGATGAATGAAGAATATTTTTTATACTATGAAGAGTTAGA
TATTTGCCTCAGAGCAAAAGCAAAGAACTTTAAATTAGGTATTTGCTCAGAAAGTTTGGTTTATCATAAAA
TAGGTGCAAGTACTGATGGGGGAAAGAGCATGATGGCTGATCTTTGCTCAATAAAAAATAGGCTGGTCATT
ACAGAAAGGTTTTATCCCCAATATTATTGGACGGTATGGTTGTCACTTTTTGTTGTAGCATTTAACCGTGC
TAGAAGAGGTGAGTTTAATAAGATGAAAAGATGTTTGAATGTTATGTTTAACTTCAAACGAAACAAAGGTA
GCAAATGCCATTAGAATATGCACTTAATCATGGTGTTAATAAATCTATAGTTTGATATGTTATTAAAGGGT
ATTTAATGAAAGTGGCTTTTTTATCTGCTTATGATCCACTATCTACATCCAGTTGGTCTGGCACACCTTAT
TATATGCTAAAGGCATTATCGAAGAGAAATATTTCCATTGAAATATTAGGACCGGTAAATAGCTATATGAT
ATACATGTTAAAAGTATATAAATTAATATTAAGGTGTTTCGGAAAAGAATATGATTATAGTCATTCGAAGT
TGCTTTCCAGGTATTACGGTAGAATATTCGGTAGGAAATTAAAAAAAATTGATGGTTTGGATTTTATTATC
GCACCTGCAGGTTCCTCACAAATTGCTTTTTTAAAAACAACCATACCAATAATATATCTATCGGATACAAC
ATATGATCAATTAAAAAGCTATTATCCGAATTTAAATAAAAAAACAATTATAAATGATGAGGATGCAAGTT
TAATCGAACGCAAGGCTATTGAAAAAGCAACAGTAGTATCTTTCCCATCTAAATGGGCAATGGATTTTTGC
AGGAATTATTACAGATTAGATTTTGATAAATTAGTTGAAATACCATGGGGGGCTAATTTATTTGATGATAT
TCACTTTGCTAATAAAAATATAATTCAAAAGAATAGTTATACTTGTCTTTTCTTGGGAGTTGATTGGGAAA
GAAAAGGTGGGAAAACAGCCTTGAAAGCAATTGAATATGTAAGGCAGTTATATGGGATCGATGTTAGACTA
AAAATTTGTGGATGTACTCCGAATCAAAAGATTTTACCTACTTGGGTTGAATTAATTGATAAAGTAGATAA
AAATAACGTTGACGAATATCAGAAATTCATCGATGTGTTATCTAACGCTGATATACTTCTTTTACCAACCA
TTGCTGAATGTTATGGAATGGTATTTTGTGAAGCTGCTGCTTTTGGATTGCCTGTTGTCGCTACAGATACA
GGTGGAGTCAGTTCTATAGTTATCAACGAAAGGACGGGGATATTAATTAAAGACCCGTTAGACTATAAGCA
CTTTGGAAATGCAATTCATAAAATAATTAGTTCCGTAGAGACTTATCAAAACTACTCCCAAAACGCAAGAA
TTAGATATAATAATATATTGCATTGGGACAATTGGGCTAAAAAGATAATTGAGATTATGTATGAGCATAAG
AATAGAAGAATCAAATAGCACAAAAAGAATTATATGTTTATTTATACTTTTTCTTGTTTTCCCTGATTTTT
TGTTTTATACATTAGGGGTTGATAATTTTAGCATTTCAACGATAATCTCAATTACATTGCTTTTTGTTTTT
TTAAGAGCTAAAAATATTTGCAAAGATAATTTTCTAATAATAGTAGCGTTATTCATATTGTTGTGTTTTAA
CTGTTTGTTAAGTATGCTATTTAATATTGAACAGGCTTTAACATTTAAAGTTGTACTTTCAATATATAGCA
TCTTAATAATGGCATACGTCTCCTCTTGTTATGCACAGACGTTGTGGTTATGTTCTGAAGAAATACTTAAG
AGATCCGTCTTTTATTTGTTCGCATTTCTTTGCCTTATTGGCATTATAAGTATTCTTTTACAGAAGACTGA
GATTATACATGATAAAAGTATGATTCTTTTTCCTGAACCATCAGCATTTGCATTGGTTTTTATACCTATCT
TTTCATTTTGTTTATACTATACAAGAGGGGGGGGGCTACTATTGCTCTATATATTATCTTTGGGTATTGCG
TTAGGTATCCAGAATTTAACAATGTTGGTAGGCATTGTGATTAGTGTTTTTGTGATGAAAAAAATAACTAT
AAGGCAAACTATTGTTATACTTTTGGGGGCATGGATTTTTTCCATGATATTAAGTGATTTAGACATTTCTT
ACTATACATCGCGGCTTGATTTTAAAAATACTACGAACCTATCAGTGCTTGTATATCTTTCAGGAATTGAA
AGAGCTTTCTTGAATTTTATTACAAGTTATGGTCTTGGTATTGGTTTTCAACAAATGGGAGTGAATGGGGA
GATAGGAATATATCAACAAATTTTAGCTGAACTTGATGCCCCTATGTTAAATATATACGATGGCTCATTTA
TTTCTTCTAAGTTAATATCTGAGTTTGGGGTTATTGGTGCATTAATGTGTATTTTCTATTTTTTTTATTTT
TCCCGATTTTATCTGCGTTTCAAAAAAAGTAAGAGATATTCACCGCAGTATATTTTAGCATATAGCTTCTA
CATGTGTTTCTTCATCCCTCTTTTTATACGTGGTGCTGGTTATATAAACCCCTATGTGTTTATGTTATTTT
CATCAATATTTTTGTGCAAATATCACGCTAAAAATATCTTGATGAAATCTAATGTCCAGATAGCTATATAA
TAGTAGATTATATTATCATTATCACGTAAATTACATATTAATAGCATATATGATAACTAGGACATAAATAA
TGTGCATTAAAAAAAAACTTAAGTTAATTAAACGATATGGCCTTTATGGTGGTCTTAGGCTTCTTAAAGAT
ATATTCTTAACAAAATTTTTATTTTGTTCAAATGTTAGGATTATTAGATTTCCATGTTATATTAGAAAAGA
TGGAAGTGTTAGTTTTGGAAAAGGTTTTACATCAGGTGTAGGATTACGAGTTGATGCATTTATGGATGCCG
TAGTTTCCATTGGAGAAAATGTTCAAATTAATGACTATGTTCACATCGCGGCTATTAATAATGTCATTATT
GGTAGAGATACATTAATAGCAAGTAAAGTATTTATTAGTGATCATAATCATGGTATTTTTTCTAAATCCGA
TATCCATAGTTCACCAACTATTATTCCTTCGTCTAGGCCCCTTGAATCTGCACCTGTGTATATTGGAGAGC
GTGTGTGGATTGGCGAAAATGTGACAATATTACCAGGTGCGTGTATAGGTAATGGTGTAGTTATTGGCGCA
AACAGTGTTGTTCGTGGTGAGATTCCTAATAATGTGATCATTGCTGGTGTTCCAGCTAAAATTGTTAAAAA
ATATAACTATGAGCGTATGCAATGGGAAAGAATATAGTTGTAATATCGGCTGTTAATTTTACAACCGGAGG
CCCCTTTACCGTACTAAAAAATGTGCTTACAGCAACTAAAGATAGAGCCGAATGTAAATTTATTGCACTGG
TTCATAGCTCTGCTGAACTAATGGAATTATTTCCGTGGGTTGAATTTATAGAGTATCCAGAAGTCAAGTCT
TCGTGGGTTAAAAGATTATATTTCGAATATATAACTTGCAATAGATTATCTAAGGTGATTAAGGCAACTCA
TTGGGTATGCTTACATGATATTACAGCAAATGTTAGTGTACCCTATAGATTTGTTTATTGCCACAATCCTG
CACCGTTCTATAAATATTTAAGCTATCGAGATATTATAGGAGAACCTAAATTTTATCTTTTTTATCTTTTT
TATGGGCTTTTATACAATATCAATATAAAAAAGAACACAGCAGTTTTTGTTCAGCAGCAGTGGCTAAAAAA
AGAATTCGAAAAAAAATATAAGTTAAAGAATGTTGTTGTTAGTCGCCCTGAAGATATTTGCCCTTTTGAAA
GTGATGGTTTGGTAAGAAATAATAATAAAAAGGATGTGAGGATATTTTACCCAGCAGTGCCCCGTATATTT
AAAAACTTTGAAGTTATCATACGTGCTGCACAAATATTACAAGATAAAAATATTCATTTTTATCTTACTTT
TGATGGTACTGAAAATAAGTATGCAAAAAGAATATATAAATTAGCTTCCGAACTGAAAAATGTACATTTCC
TCGGTTACCTTAATGCAACCGAGATGGTTAACTTTTATCAAGATTCAGATATTATTTGTTTCCCATCGAAA
CTAGAAACGTGGGGATTACCATTATCAGAAGCTAAAACATACAAAAAATGGATATTTGCGGCAGACTTACC
TTATGCTCATGAAGTTTTATATAACTATTCAAAAACTAGATATTTTCCATTTGACGATGAGAAAATACTTG
TTCGCTACATATTAGAGTACACAAGTAAAAATATGCATGAAGATATAAAAAATAGTAGGGTGAATTTTAAT
AATGATGCATTGACTGGTTTTGAACAGTTTATTGAATATATCCTCAAGGGGAACTGACGTGGTTTATATTA
TAATCGTTTCACATGGCCATGATGACTATATAGAAAATCTTTTATTAAATTTAAAGTTGCCCTCTGGAAGA
TTTAAAATAATAGTTCGTGATAACAAAAGTTCAATGGTTTTAAAAAAAACATGCGAAAAAAATTGCGTAAC
CTATTTGCATGGAGGGCAATATGGATTTGGACATAATAATAACATAGCAGTGTCATATATAATTAATAACT
TCATGATTATGAATAATGATTATTTTCTCTTTCTTAACCCCGATGTATTCATAACCAGTGAAAGTTTGATT
AATTATGTTGATTATATAATTAGTAATGATTATAAGTTTAGCACATTATGTCTTTATCGAGATTTTACTAA
AAGCAAACATGATTATTCAATACGGAGTTTTCCAACTTTATATGATTTTCTTTGTTCTTTTTTATTGGGGG
TGAATAAAAGTAAAATTAAGAAGGAAAATATACTTTCTGATACTGTAGTTGATTGGTGTGCTGGCTCATTT
ATGCTTATTCATGCTTTAAGTTTCTTAAATGTGAATGGTTTTGATCAAAAATATTTTATGTATTGTGAAGA
TATTGACCTTTGTATGCGTTTAAAATTAAGTGGAGTAGATCTTTACTATACTCCCCATTTTGATGCTATTC
ATTATGCGCAGCATGAAAATAGAAGAATATTTACTAAAGCATTTCGATGGCATATAAGGAGTATTACGCGC
TACATATTACGGAAACCAATTCTTTCTTATAAAAACTATAGAAAAATTACATCCGAACTGGTAAAGTGATT
AAGGATCCGTGTAGGCTGGAGCTGCTTCGAAGTTCCTATACTTTCTAGAGAATAGGAACTTCGGAATAGGA
ACTAAGGAGGATATTCATATGGATAAAGCCGTAAGCATATAAGCATGGATAAGCTATTTATACTTTAATAA
GTACTTTGTATACTTATTTGCGAACATTCCAGGCCGCGAGCATTCAGCGCGGTGATCACACCTGACAGGAG
TATGTAATGTCCAAGCAACAGATCGGCGTAGTCGGTATGGCAGTGATGGGACGCAACCTTGCGCTCAACAT
CGAAAGCCGTGGTTATACCGTCTCTATTTTCAACCGTTCCCGTGAGAAGACGGAAGAAGTGATTGCCGAAA
ATCCAGGCAAGAAACTGGTTCCTTACTATACGGTGAAAGAGTTTGTCGAATCTCTGGAAACGCCTCGTCGC
ATCCTGTTAATGGTGAAAGCAGGTGCAGGCACGGATGCTGCTATTGATTCCCTCAAACCATATCTCGATAA
AGGAGACATCATCATTGATGGTGGTAACACCTTCTTCCAGGACACTATTCGTCGTAATCGTGAGCTTTCAG
CAGAGGGCTTTAACTTCATCGGTACCGGTGTTTCTGGCGGTGAAGAGGGGGCGCTGAAAGGTCCTTCTATT
ATGCCTGGTGGCCAGAAAGAAGCCTATGAATTGGTAGCACCGATCCTGACCAAAATCGCCGCCGTAGCTGA
AGACGGTGAACCATGCGTTACCTATATTGGTGCCGATGGCGCAGGTCACTATGTGAAGATGGTTCACAACG
GTATTGAATACGGCGATATGCAGCTGATTGCTGAAGCCTATTCTCTGCTTAAAGGTGGCCTGAACCTCACC
AACGAAGAACTGGCGCAGACCTTTACCGAGTGGAATAACGGTGAACTGAGCAGTTACCTGATCGACATCAC
CAAAGATATCTTCACCAAAAAAGATGAAGACGGTAACTACCTGGTTGATGTGATCCTGGATGAAGCGGCTA
ACAAAGGTACCGGTAAATGGACCAGCCAGAGCGCGCTGGATCTCGGCGAACCGCTGTCGCTGATTACCGAG
TCTGTGTTTGCACGTTATATCTCTTCTCTGAAAGATCAGCGTGTTGCCGCATCTAAAGTTCTCTCTGGTCC
GCAAGCACAGCCAGCAGGCGACAAGGCTGAGTTCATCGAAAAAGTTCGTCGTGCGCTGTATCTGGGCAAAA
TCGTTTCTTACGCCCAGGGCTTCTCTCAGCTGCGTGCTGCGTCTGAAGAGTACAACTGGGATCTGAACTAC
GGCGAAATCGCGAAGATTTTCCGTGCTGGCTGCATCATCCGTGCGCAGTTCCTGCAGAAAATCACCGATGC
TTATGCCGAAAATCCACAGATCGCTAACCTGTTGCTGGCTCCGTACTTCAAGCAAATTGCCGATGACTACC
AGCAGGCGCTGCGTGATGTCGTTGCTTATGCAGTACAGAACGGTATTCCGGTTCCGACCTTCTCCGCAGCG
GTTGCCTATTACGACAGCTACCGTGCTGCTGTTCTGCCTGCGAACCTGATCCAGGCACAGCGTGACTATTT
TGGTGCGCATACTTATAAGCGTATTGATAAAGAAGGTGTGTTCCATACCGAATGGCTGGATTAA
SEQ ID NO: 19 (example O75 rfb locus nucleotide sequence - O75-EPA production strain
stLMTB11737)
ATGACGAATTTAAAAGCAGTTATTCCTGTAGCGGGTCTCGGGATGCATATGTTGCCTGCCACTAAGGCGAT
ACCCAAAGAGATGCTACCAATCGTCGACAAGCCAATGATTCAGTACATTGTTGACGAGATTGTGGCTGCAG
GGATCAAAGAAATCCTCCTGGTAACTCACGCGTCCAAGAACGCGGTCGAAAACCACTTCGACACCTCTTAT
GAGTTAGAATCACTCCTTGAGCAGCGCGTGAAGCGTCAACTGCTGGCGGAAGTACAGTCCATCTGTCCGCC
GGGCGTGACCATTATGAACGTGCGTCAGGGCGAACCTTTAGGTTTAGGCCACTCCATTTTGTGTGCGCGAC
CTGCCATTGGTGACAACCCATTTGTCGTGGTACTGCCAGACGTTGTGATCGACGATGCCAGCGCCGACCCG
CTACGTTACAACCTTGCTGCCATGATTGCACGTTTCAACGAAACGGGCCGCAGCCAGGTGCTGGCAAAACG
TATGCCGGGTGACCTCTCTGAATACTCCGTCATCCAGACTAAAGAGCCGCTGGACCGTGAGGGTAAAGTCA
GCCGCATTGTTGAATTTATCGAAAAACCGGATCAGCCGCAGACGCTGGACTCAGACATCATGGCCGTAGGT
CGCTATGTGCTTTCTGCCGATATTTGGCCGGAACTGGAACGTACTCAGCCTGGTGCATGGGGACGTATTCA
GCTGACTGATGCTATTGCCGAGCTGGCGAAAAAACAATCCGTTGATGCAATGCTGATGACCGGCGACAGTT
ACGACTGCGGCAAAAAAATGGGCTATATGCAGGCGTTTGTGAAGTATGGCCTACGCAACCTGAAAGAAGGG
GCGAAGTTCCGTAAAGGTATTGAGAAGCTGTTAAGCGAATAATGAAAATCTGACCGGATGTAACGGTTGAT
AAGAAAATTATAACGGCAGTGAAAATTCGCAGCAAAAGTAATTTGTTGCGAATCTTCCTGCCGTTGTTTTA
TATAAACCATCAGAATAACAACGAGTTAGCAGTAGGGTTTTATTCAAAGTTTTCCAGGATTTTCCTTGTTT
CCAGAGCGGATTGGTAAGACAATTAGCGTTTGAATTTTTCGGGTTTAGCGCGAGTGGGTAACGCTCGTCAC
ATCATAGGCATGCATGCAGTGCTCTGGTAGCTGTAAAGCCAGGGGCGGTAGCGTGCATTAATACCTCTATT
AATCAAACTGAGAGCCGCTTATTTCACAGCATGCTCTGAAGTAATATGGAATAAATTAAGCTAGCAGTGAA
GATACTTGTTACTGGTGGCGCAGGATTTATTGGTTCTGCTGTTGTTCGTCACATAATAAATAATACGCAAG
ATAGTGTTGTTAATGTCGATAAATTAACATACGCCGGAAACCTGGAATCGCTCGCTGAAATTTCTGATTCT
GAACGTTATTCATTTGAGCATGCAGATATCTGCGATGCCGAAGCGATGGCTCGTATTTTCGCACAGCACCA
GCCAGACGCGGTGATGCACCTGGCAGCAGAGAGCCACGTTGACCGCTCAATAACTGGCCCTGCGGCATTTA
TTGAAACCAATATTGTGGGTACTTATGTTCTTTTAGAAGCGGCGCGCAATTATTGGTCTGGTCTGGATGAT
GAAAAGAAAAAAAACTTCCGCTTTCATCATATTTCTACTGATGAGGTGTATGGTGACTTACCCCATCCGGA
TGAAGTAAATAGCAATGAAACGTTGCCGCTATTTACGGAAATGACAGCATACGCGCCAAGTAGTCCATATT
CTGCTTCTAAAGCTTCCAGCGATCATTTGGTTCGCGCATGGAAACGTACTTATGGTTTACCGACCATTGTG
ACTAATTGCTCGAACAACTATGGTCCTTATCATTTCCCGGAAAAGCTTATTCCACTGGTTATTCTTAATGC
ACTGGAAGGTAAGGCATTACCTATTTATGGCAAAGGAGATCAGATCCGCGACTGGTTGTATGTAGAGGATC
ATGCTCGAGCGTTATATACCGTCGTAACCGAAGGTAAAGCGGGCGAAACTTATAACATTGGTGGACACAAC
GAAAAGAAAAACATCGACGTAGTGTTCACTATTTGTGATTTGTTGGATGAGATAGTCCCGAAAGAGAAATC
TTATCGTGAGCAAATTACCTATGTTGCTGATCGCCCAGGGCATGATCGCCGTTATGCAATTGATGCCGATA
AAATTAGCCGCGAATTGGGCTGGAAACCACAGGAAACGTTTGAGAGCGGGATTCGTAAAACTGTGGAATGG
TATCTGTCCAATACAAAATGGGTTGATAATGTGAAAAGTGGTGCCTATCAATCGTGGATTGAACAGAACTA
TGGGGGCCGCCACTAATGAATATCCTCCTTTTTGGCAAAACAGGGCAGGTTGGTTGGGAACTACAGCGTGC
TCTGGCACCTCTGGGTAATTTGATTGCTCTTGATGTTCACTCCACTGATTACTGTGGTGATTTTAGTAACC
CTGAAGGTGTGGCTGAAACCGTTAGAAGCATTCGGCCTGATATTATTGTCAACGCAGCCGCTCACACCGCA
GTAGACAAAGCAGAATCAGAACCGGAGTTTGCACAATTACTGAACGCGACGAGTGTCGAAGCGATCGCGAA
AGCAGCCAATGAAGTCGGCGCTTGGGTTATTCACTACTCTACTGACTACGTATTTCCGGGGACCGGTGAAA
TACCATGGCAGGAGGAGGATGCAACCGCACCGCTAAATGTTTACGGTGAAACCAAGTTAGCAGGAGAAAAA
GCATTACAAGAGCATTGTGCGAAGCACCTTATTTTCCGGACCAGCTGGGTCTATGCAGGTAAAGGAAATAA
CTTCGCCAAAACGATGTTGCGTCTGGCAAAAGAGCGTGAAGAATTAGCCGTTATTAATGATCAGTTTGGTG
CGCCAACTGGCGCAGAGTTGCTGGCTGATTGTACGGCACATGCCATTCGTGTGGCACTGAATAAACCGGAA
GTCGCAGGTTTGTACCATCTGGTAGCCAGTGGTACCACAACCTGGCACGATTATGCTGCGCTGGTTTTTGA
AGAGGCGCGCAAAGCAGGCATTCCCCTTGCACTCAACAAGCTCAACGCAGTACCAACAACAGTCTATCCTA
CACCAGCTCGTCGTCCACATAACTCTCGCCTTAATACAGAAAAATTTCAGCAGAACTTTGCGCTTGTCTTG
CCTGACTGGCAGGTTGGTGTGAAACGCATGCTCAACGAATTATTTACGACTACAGCAATTTAATAGTTTTT
GCATCTTGTTCGTGATGGTGGAACAAGATGAATTAAAAGGAATGATGGAATGAATACGCGTAAAGGTATTA
TTTTAGCGGGTGGTTCTGGTACACGTCTTTATCCTGTGACTATGGCTGTCAGTAAACAGCTGTTACCGATT
TATGATAAACCGATGATCTATTACCCGCTCTCTACACTGATGTTGGCGGGTATTCGCGATATTTTGATTAT
CAGCACGCCACAGGATACTCCTCGTTTTCAACAACTGCTGGGTGATGGGAGCCAGTGGGGGCTAAATCTTC
ACTACAAAGTGCAACCGAGTCCGGATGGTCTTGCGCAGGCATTTATCATCGGTGAAGAGTTTATCGGTGGT
GATGATTGTGCTTTGGTACTTGGTGATAATATCTTCTACGGTCACGACCTGCCTAAGTTAATGGATGCCGC
TGTTAACAAAGAAAGTGGTGCAACGGTATTTGCCTATCACGTTAATGATCCTGAACGCTATGGTGTCGTTG
AGTTTGATAAAAACGGTACTGCAATCAGCCTGGAAGAAAAACCGTTACAACCAAAAAGTAATTATGCGGTA
ACCGGGCTTTATTTCTATGATAACTACGTTGTGGAAATGGCGAAAAATCTTAAGCCTTCTGCCCGCGGTGA
ACTGGAAATTACCGATATTAACCGTATCTATATGGAACAGGGGCATTTATCTGTTGCCATGATGGGACGTG
GATATGCCTGGCTGGACACGGGGACACATCAAAGTCTTATTGAAGCAAGCAACTTCATTGCCACCATTGAA
GAGCGCCAGGGCTTGAAAGTTTCCTGCCCGGAAGAAATTGCTTACCGTAAAGGGTTTATTGATGCTGAGCA
GGTGAAAGTATTAGCTAAACCGCTGAAAAAAAATGCTTATGGTCAGTATCTGCTAAAAATGATTAAAGGTT
ATTAATAAAATGAATGTTATTAAAACAGAAATTCCAGATGTACTGATTTTTGAACCGAAAGTTTTTGGTGA
TGAGCGTGGTTTCTTTATGGAAAGCTTTAATCAGAAAGTTTTCGAAGAGGCTGTAGGGCGGAAGGTTGAAT
TTGTTCAGGATAATCATTCTAAATCGTGTAAAGGTGTACTTAGAGGTTTACACTTTCAGCTTCCTCCCTTT
GAGCAGGCAAAATTAGTAAGGTGTATAGTTGGCGAGGTATTTGATGTTGCAGTAGACATTAGACCTAATTC
TGAAACATTTGGTTCATGGGTTGGAGTAACTCTTTCGTCAGAAAATAAAAGGCAGCTATGGATTCCAGAAG
GATTCGCCCATGGTTTTTTAACTTTAAGTGATATTGCAGAGTTTGTTTATAAAACTAACAACTATTATTCT
TTAAATCATGAAAGGGGAGTCATTTGGAACGATGAGGAAATTAACATTGCCTGGCCCTCTCAATCAGAGAA
GATTCTGTCACAGAAAGATATTAATTTACCATCATTTAGATTTGTTCAAATGTTTAGCAAGTAGTGTTATC
TTTACACTGCACATAGTCATCATTTTTTATGCTTTAAGTAAATTATATTGCACATCTATAACACAAAGCGC
AATAATATTTCGACCTGATGAAGGTTTGTGGTTATTTATCTTTCTAGGCGTTTTTTATGACTAAAATAGTT
GTGGTTTCTACAGCTCCAATATTCCCGACAAATAATGGGTACAAAAGTTCTGTATTAGGAAGAATTGATGA
GTTATTAAATGAGGATAATGAGGTCGTTTTGATTGAAATAAACCTTGAAAATGTTACGGAAAAGAAAGATG
AATTAATACCAACAAGATTTAATAATATTCAAAGATATGAAGTAAAAAAAATATCTAGATCATTTATTGCC
GAGTTACAAATATTATTTGATATCAGAACTCGGTATGAACAATTATTTTCTTCTGCTGACATTAGAGATAA
CATAAAAAAGATAATTGATTTAGAAAAACCTTCTATTATTATTGCTGAGTCTATATGGGCGTTGCAAGCAT
TGCCTATTGAAATTAGTGCGAGAATACACTGTGTTATTCATGATGTGGCAACTGATTTCTTTAAAGAAATG
TTTGTATCTCATAATGAGGTTGTACGAAAAATTTTGTTTTTTAATGATTACCTAAAGTTGAAAATTACTGA
AGAAAATATTATCAAACGTTTGAGAGTTGAGCAATTTATCTTTCTGACAGAAGAAGATAAATGTTGGTATA
AAACAAGATACAATATTGATGAGGGTTGTTGTTCCTTAGCGAGCAATCATCTTTATGTAGAAAAGATTAAG
AGAACTATCAATTTCCAAACCCCTTTCCTGCTTATTCCCGGTAGCATTGAATTTTCACAAAATTTTTACGG
CTTAAATTGGTTTATAAAAAATATATATCCTGGATTAAATAGGAAAATAAGAATAGTTGTAACAGGAAAGG
CATCAGATAAAAAAATAAAGATGTTAAACTGTGGAGAGGAAATTACCTTTACGGGAGAGCTTGACTTTTCC
ACATATAATAAACTTAGCTCAACATGCTTGTGTGTTATTGCACCGATTACAACGGGCACTGGAATTAAAAT
AAAAATATTAGAAGCTGTACAAAAAGGTATTCCTGTACTTACAACAAAATTTGCTTCAAAAGGAATATGTT
CCGATTTATGTTTTTATTGCGAGGAGGATACTGACACAAACTTTGTCAATTTAATTAACAGTTTTCTTGAA
ACGACATTAAGAGTCCAAGAATGAATTTATTGCTTTTTTCAGTCCTTGCGTTTGGTTTAATATTGGCTTTG
GCCCATAATAATAAAAGTGGAGATATTAACGCATACTTAATGTTTTTTCTCGTGGTCCTAATGGTATTAAT
ATCAGGGCTGCGTATGAATGATAGTGATTATATCGAATACAGGAAAATGTATAATGAAGTGCCTATTTTAT
GTGACTTTAGTCTCGCATCTATAAGAGATATACATGGGGAGGTAGGCTATCTATTCTTATCATCAATCTTT
AAAACTTTATGCTTGCCATTTCAATTATTTCTTTTTTTTATTGCTTTTTTATCACTCCTGCTTACATATTT
TTCATTCAGAAAAATAAGTTTAATACCGATACTATCGTTAGTTTTTTATTTAAGCCATGCTTTTATAGTTA
GAGATTTGATTCAAATTAGGGCAGGATTAGCTGTTAGCATATCATTATATTCAATAATTAAATTTAAAGGA
AATAAAAGTATAATTACAGGAGTTTTATTTGCTTCTTTGATTCATTCTGGGGCGCTTATTATTGCTCTTTG
TTATCCTTTTTTCAAAAAAAAATACATAACATTAAAAATGATGTTGTTTTTATTTTTAGTGTCAATTATTT
TTTCTTATTTGAATGGGCTTAATTTATCGATACAACTCTTATCTCAATATAGTTTGCTTCCAACTGCAATT
TCGAATTATGTTGGTTGGGAAGAATATGATTATCGGGTGAGTATATTTACTAATCCGGTTTTTATTAAAGG
TGTTTTTTTAATTGTCTTAATGCACAAATATGTACTTTCAGATATTAAAAATGAGAAAATTATAGTGCTTT
ATAACTTATATGTTTTAGGTGTATTAGCTATGGTTGCATTGAGTGGGATGGCTATTCTTTCAGGCCGTCTT
TCATCCTTTCTGACACTAGGTGAAAGCATTTTAATTGTATATGCTCTGTTCTACAAAAGAAATACACCTCT
GGCGTTTCTAATTTTTTCTTTTTTAACAATTGTGCAATTAGGATATGATCTATTTATTTCTAATGTGCATC
CTGAGCTTACTCTGATTATATTTGGGTGAATCTAAGTGAAAAATAATAAAATAGGCATACTTATCTCTAAA
ATACAAAATCTTGGACCTGTGAATGTAGTACGAGGATTGATAAAAGAAAATAAAAAATATGCTTTTACTGT
TTTTTGTTTAACAAATAGCGTAGATAAAAATATATATGATGAGTTATGCTGTTTAGGAGCCAAGGTTATAT
TAATACCAGATGGTACTTGGTTCAGCAAAATTTTATTTGTGAGAAGTTTTTTAAAGGAACATCCACATAAT
ATCTTACATTCACATGGGATCACGGCCGATATGTTTTCTTACTTTCTGAATGGCGTGAAAATATCTACTAT
TCACAATAGACTAGATGAGGATTATATCCCATTATTTGGCGCGGTTAAAGGGAATGCTATATATTATCTTC
ATCGTTTTATATTACGAAGATTTAATCATATCGTTGCTTGCTCAGCAGCGGTCCAATCAAAACTGAAACAA
TCGAAAGTAAAAACTAAAATAACCACCATCCAGAATGGGATTGATATAACTAGGTTTAAGACACTTGAGTC
TGATAAAAAAAAATTATTGAGGGAAAAACACGGATTTGATAGTGAAAAAAGAATATTTATATATTGTGGCT
CGTTATCATTAAGGAAAAATATTGCTTACCTCTTGGAACACTTAGCCATCGAAGAAAATGATATATTTTTA
ATTCTAGGTGATGGTGAACTTTTTAGATATTGTAAGGATAAATATTCTAAAGATTTACGGTATATATTTAT
GGGGAAAGTTGAATGCCCTCTTGAATATTATCAATTATCAGATATTTTTGTTTCCGCTTCTTTATCGGAAG
GGCTCCCCTTGGCACTATTAGAAGCTGCCTCTACTGGGTGCTATTTATATGTTAGCGATATAGAGCCCCAT
AGAGAAATTGCATCTCTATTAGGAGAGGAAAATATTTCTATGTTTAAAATTAAGGATGGATCATATAATTA
TTTGCAACCTAAAATAAAAAAAGCTGACTATAACGCTCTTTCTGACGATAAACTTTACAATATATCCGATA
AAAAAATGTCAAATCTTTATGACAAACTTTTTGTTTCTTTATTAGAGCAGAGGCACTAATATAATGATTTA
TGTTTCGGTAATTTCTCATGGTCATTTCAAAACTCTTAAGGAATTAGGAGCAGTATCAAAATTAAATAATC
ACAGCAGAATTAAAGTTATCATCAAAGATAATTTAGGAGAGAGCGAGCTTTTGGATTTTTGTCAGGAAAAC
AAAATAACTTATTTAAGGTCTAAAGAGAAAAAAGGATTTGGAGAGAATAATAATGAAGTTTTTTCCTCTAT
ATCCTCCTTAATTACTAAGGAAGATTTTTTTGTGGTTATGAATCCTGATATATATATTGAGTGCTCTGATC
TATTAGATGTCGTAGATGAGTGTGGTTCAGCGAATGTTAATCTAGCAACGATAAATTTATACAGGGATTTT
GATAAAAAAACATATGATAACTCAGTAAGGAAATTTCCCTCGGCAATTGATTTTTTTATGTCATTTTTATT
TAAGAAAAATGACTGTGTAGTAAATAAGAACAAAATAACGAAACCAACATATGTTGATTGGGCTGCAGGTT
CTTTTCTAATATTTAATGCCTTCTTTTATTCAAAACTCAACGGATTCAACGAAAAGTATTTTATGTATTGC
GAAGATATTGATATATGTTGGCGAGCTAAAAAACACTTCAATACTTCAGTTTTATACTATCCATGCTATGC
AGCAATTCATTTGGCACAATTTAACAATCGTAGGATTTTTAGTAGACATTTCATTTGGCATATAAAAAGTA
TTATCCTTTTTTTATTATATAAAAATGGTATGCTGCGTTCTAGTAAGTTGCTTTAATGCTAATATTCTTTT
AAGAGGTGAGAATGATACCTGTTATTTTGGCTGGTGGTTCGGGAAGTCGCTTGTGGCCACTTTCACGAGAA
AAGTTCCCCAAGCAGTTTTTAAAGTTGACTGGCAGTTTGACAATGTTGCAGTCAACATTGTCACGTCTTAA
TAATTTAAATGCTGATGATTCAATAGTTATATGCAACGAAGAGCATAGATTTATTGTTGCAGAACAATTAA
GAGAGTTAGGCAAACTTTCAAATAACATTATTCTTGAACCCAAAGGTCGTAATACAGCCCCTGCTATAACA
CTCGCAGCATTAGCAGCAAAAAGAAAATTCGCTGATGAAGATCCATTGATTCTTATTTTAGCTGCAGATCA
CAACATCCAAGACGAACATGTTTTCTGTGAGGCAATTAATAAGGCGTCATCTTTAGCTAGTTATGGAAAAC
TAGTGACTTTTGGTATCGTTCCATTCAAACCTGAAACTGGGTATGGCTATATTCGTCGCGGTGATGAAGTG
CCTGTAGATGAGCAGCATGCGGTGGCCTTTGAAGTGGCGCAGTTTGTCGAAAAACCGAATCTGGAAACCGC
GCAGGCCTATGTGGCAAGCGGCGAATATTACTGGAACAGCGGTATGTTCCTGTTCCGTGCCGGACGCTATC
TCGAAGAACTGAAAAAGTATCGTCCGGATATTCTCGATGCCTGTGAAAAAGCGATGAGCGCCGTCGATCCG
GATCTCGATTTTATTCGTGTGGATGAAGAGGCGTTTCTCGCTTGTCCGGAAGAGTCGGTGGATTACGCGGT
CATGGAATGCACGGCAGATGCCGTTGTGGTGCCGATGGATGCGGGCTGGAGCGATGTCGGTTCCTGGTCTT
CATTATGGGAGATCAGCGCCCACACCGCCGAGGGCAACGTTTGCCACGGCGATGTGATTAATCACAAAACT
GAAAACAGCTATGTGTACGCCGAATCTGGCCTGGTCACCACCGTCGGGGTGAAAGATTTGGTGGTAGTGCA
GACCAAAGATGCAGTGCTGATTGCCGACCGTAATGCGGTGCAGGATGTGAAGAAAGTGGTCGAGCAGATCA
AAGCTGATGGTCGCCATGAGCATCGGGTGCATCGCGAAGTGTATCGTCCGTGGGGCAAATATGACTCTATC
GACGCGGGCGACCGCTACCAGGTGAAACGCATCACCGTGAAACCGGGCGAAGGTTTGTCGGTACAGATGCA
TTATCATCGCGCGGAACACTGGGTGGTTGTCGCGGGAACGGCAAAAGTCACTATCAACGGTGATATCAAAC
TGCTTGGTGAAAACGAGTCCATTTATATTCCGCTGGGGGCGATGCACTGCCTGGAAAACCCGGGGAAAATA
GATTTAGAATTAATTGAAGTTCGCTCTGGTGCATATCTTGAAGAAGATGATGTTATTAGATGTTATGATCG
CTATGGACGAAAGTAATATATAATAATTATTTCAGAATTAGAAATGATAATTATAAGTTTTCGTCTGGATA
AACAATAGATAGTATGGGTTGGAAAATATGAGTTCTTTAACTTGTTTTAAAGCTTACGACATTCGCGGGAA
ATTAGGTGAAGAACTGAATGAAGATATCGCCTGGCGCATTGGTCGCGCCTATGGCGAATTTCTCAAACCGA
AAACCATTGTGTTAGGCGGTGATGTCCGTCTCACCAGCGAAACCTTAAAACTGGCGCTGGCAAAAGGTTTA
CAGGATGCGGGCGTCGATGTGCTGGATATTGGCATGTCCGGCACCGAAGAGATTTATTTCGCCACGTTCCA
TCTCGGCGTGGATGGCGGCATTGAAGTTACCGCCAGCCATAATCCGATGGATTACAACGGCATGAAGCTGG
TGCGCGAAGGGGCTCGCCCGATCAGCGGTGATACCGGACTGCGCGACGTCCAGCGTCTGGCAGAAGCTAAC
GACTTTCCTCCCGTCGATGAAACCAAACGCGGTCGCTATCAGCAAATCAATCTGCGTGACGCTTACGTTGA
TCACCTGTTCGGTTATATCAATGTCAAAAACCTTACGCCGCTCAAGCTGGTGATCAACTCCGGGAATGGCG
CAGCGGGTCCGGTGGTGGACGCTATCGAAGCCCGCTTTAAAGCCCTCGGCGCACCGGTGGAGTTAATCAAA
GTGCATAACACGCCGGACGGCAATTTCCCCAACGGTATTCCTAACCCGTTGCTGCCGGAATGTCGCGACGA
CACCCGCAATGCGGTCATCAAACACGGCGCGGATATGGGCATTGCCTTTGATGGCGATTTTGACCGCTGTT
TCCTGTTTGACGAAAAAGGGCAGTTTATTGAGGGCTACTACATTGTCGGCCTGCTGGCAGAAGCGTTCCTC
GAAAAAAATCCCGGCGCGAAGATCATCCACGATCCACGTCTCTCCTGGAACACCATTGATGTGGTGACGGC
CGCGGGCGGCACGCCGGTGATGTCGAAAACAGGACACGCCTTTATTAAAGAACGTATGCGCAAGGAAGACG
CCATCTACGGTGGCGAAATGAGCGCTCACCATTACTTCCGCGATTTCGCTTACTGTGACAGCGGCATGATC
CCGTGGCTGCTGGTCGCCGAACTGGTGTGCCTGAAAGGAAAAACGCTGGGCGAACTGGTGCGCGACCGGAT
GGCGGCGTTTCCGGCAAGCGGTGAGATCAACAGAAAACTGGCGCACCCTGTTGAGGCGATTAACCGCGTGG
AACAGCATTTTAGCCGTGAGGTGCTGGCGGTGGATCGCACCGATGGCATCAGCATGACCTTTGCCGACTGG
CGCTTTAACCTGCGCTCTTCCAACACCGAACCGGTGGTGCGCCTGAATGTGGAATCTCGCGGTGATGTTCA
GGTTATGGTAATCCATACTCAAGAAATATTATCAATTTTGACGTCATAAAGAATAAGCCCTGACAAGTTAG
GGCTTAATTAATATATATTTTTTTTGAATTGGGGATTTGTGGTAAGATTTTTAATATGTTATTTAATGTGG
TTGAATTAATGTTGACTGGAAAATAATAATGAGAACGAAAAAAGCATTACACAACTTTAAAGTTGATTTAT
TAATTACTTTTTTATTGGTTTTGCTAGGGTTTTATATTCGAACTGTTTTTGTTTCAAAAATGGGAAGTGAT
ATTACTGGAGTGATGTTACTATTCACACAGTTGACAGCATATCTCAATTTGGCAGAATTAGGTATTGGAAT
TGCAGCTGCCAGCGTATTATATAAACCGCTCAGCGAGAATGAATACAATAAAATAACTTACATAATATCTT
TGCTCTCAGTCATATACAAATATATATTTGTGTTTGTTTTGATTCTTGGCGTTGTTATAGGTATCTGTATT
TATTACTTTATTGATTCTGTAAAGGTTGTAAATGGCGTTTTTTTATATTGGGCTTTGTTCGTTTTTAATAC
ATCGTTGACATATAGTTATGCTAAATACTCCACATTATTAACTGCTAATCAGCGGTACTCAGCAGTAAGAA
AAATTCAAGGTGGCGGAAAAGTTATAATAATTGTATTTCAGATATTAATTTTGTGCTTTACGCAAAGTTTC
ATACTTTATTTGTTAGTTGAGACTTTAGGTATTTTTTCTCAATATTTGATTTTTAAAAAAATAATTGGGAA
CGGAAATCAATATCTCAGTAATGAGGTTTTACTTATTGAAAGCGATAAACTTTTGATAAAAAAAGAATTAA
AAATAAGAATAAAAAATATGTTCTTCCATAAAATAGGTGCTGTGCTTGTCCTTAATACAGACTACCTGCTT
GTATCAAAGTTTCTGACATTAAGTTATGTGACAATTTTTGGCAGCTATATGATGGTATTTCAGATAGTAAC
TGTTTTGATGTCAAGTTTTGTTAATGCTATTACTGCAGGAATGGGTAATTACTTAATTAATAAAAGTAATT
TAGAAATTAAGGAAATTACACGTCAATTTTATGTGATATTTATCGCCTTTGCAACATTCATATCACTAAAT
ATGTTTTTTCTTGTTAATGATTTTATCGCAAAATGGATAGGTGTTAATTATACATTAAGTAACACCCTAGT
TGCATTAATGATTGTTAACGTATTCATTAGTGTTGTCAGGGTACCTTCTGATATATTAAAAAACGCAAGTG
GACATTTTGGTGATATTTATTATCCATTATTAGAAGGTGTGCTGAATATTACGATATCCATCATTTTGGCT
ATCATTATTGGATTACCTGGCATTATTATAGGGACAATAGTATCTAACTTAATAGTAATAATGCTTGCGAA
ACCATTATATCTTTACTCTAAGTTATTTAATCTTAGAAATCCGACGAGGGTTTATTTTGAATTTATTTCTC
GGCCTATGTTATATTCATTATGTGTGATTGGGGTGAGCTATTTATTGCGCGATGAAATATATTCATTTAAA
GTAAGTACATGGTTGGATTTTATTAACAAGCTACTCTTAGTCTCTACTCCTAGCATATTGGTAATATGTGC
TATTTTCTCTACGGATAGTGACTTTAGATTATTTTTCAGAAAAATTATATATGTGATTATGAAGAAATAAA
AATTTCGAAAATGTATTAATCGAAATTATGCAACGAGCTTTATTTTTATAAATGATATGTGATCTTTTCGC
GAATAGGAGTAAGGATCCGTGTAGGCTGGAGCTGCTTCGAAGTTCCTATACTTTCTAGAGAATAGGAACTT
CGGAATAGGAACTAAGGAGGATATTCATATGGATAAAGCCGTAAGCATATAAGCATGGATAAGCTATTTAT
ACTTTAATAAGTACTTTGTATACTTATTTGCGAACATTCCAGGCCGCGAGCATTCAGCGCGGTGATCACAC
CTGACAGGAGTATGTAATGTCCAAGCAACAGATCGGCGTAGTCGGTATGGCAGTGATGGGACGCAACCTTG
CGCTCAACATCGAAAGCCGTGGTTATACCGTCTCTATTTTCAACCGTTCCCGTGAGAAGACGGAAGAAGTG
ATTGCCGAAAATCCAGGCAAGAAACTGGTTCCTTACTATACGGTGAAAGAGTTTGTCGAATCTCTGGAAAC
GCCTCGTCGCATCCTGTTAATGGTGAAAGCAGGTGCAGGCACGGATGCTGCTATTGATTCCCTCAAACCAT
ATCTCGATAAAGGAGACATCATCATTGATGGTGGTAACACCTTCTTCCAGGACACTATTCGTCGTAATCGT
GAGCTTTCAGCAGAGGGCTTTAACTTCATCGGTACCGGTGTTTCTGGCGGTGAAGAGGGGGCGCTGAAAGG
TCCTTCTATTATGCCTGGTGGCCAGAAAGAAGCCTATGAATTGGTAGCACCGATCCTGACCAAAATCGCCG
CCGTAGCTGAAGACGGTGAACCATGCGTTACCTATATTGGTGCCGATGGCGCAGGTCACTATGTGAAGATG
GTTCACAACGGTATTGAATACGGCGATATGCAGCTGATTGCTGAAGCCTATTCTCTGCTTAAAGGTGGCCT
GAACCTCACCAACGAAGAACTGGCGCAGACCTTTACCGAGTGGAATAACGGTGAACTGAGCAGTTACCTGA
TCGACATCACCAAAGATATCTTCACCAAAAAAGATGAAGACGGTAACTACCTGGTTGATGTGATCCTGGAT
GAAGCGGCTAACAAAGGTACCGGTAAATGGACCAGCCAGAGCGCGCTGGATCTCGGCGAACCGCTGTCGCT
GATTACCGAGTCTGTGTTTGCACGTTATATCTCTTCTCTGAAAGATCAGCGTGTTGCCGCATCTAAAGTTC
TCTCTGGTCCGCAAGCACAGCCAGCAGGCGACAAGGCTGAGTTCATCGAAAAAGTTCGTCGTGCGCTGTAT
CTGGGCAAAATCGTTTCTTACGCCCAGGGCTTCTCTCAGCTGCGTGCTGCGTCTGAAGAGTACAACTGGGA
TCTGAACTACGGCGAAATCGCGAAGATTTTCCGTGCTGGCTGCATCATCCGTGCGCAGTTCCTGCAGAAAA
TCACCGATGCTTATGCCGAAAATCCACAGATCGCTAACCTGTTGCTGGCTCCGTACTTCAAGCAAATTGCC
GATGACTACCAGCAGGCGCTGCGTGATGTCGTTGCTTATGCAGTACAGAACGGTATTCCGGTTCCGACCTT
CTCCGCAGCGGTTGCCTATTACGACAGCTACCGTGCTGCTGTTCTGCCTGCGAACCTGATCCAGGCACAGC
GTGACTATTTTGGTGCGCATACTTATAAGCGTATTGATAAAGAAGGTGTGTTCCATACCGAATGGCTGGAT
TAA

1-7. (canceled)

8. A bioconjugate comprising a polysaccharide covalently linked to a carrier protein produced by a process comprising culturing a recombinant prokaryotic host cell under conditions for production of the bioconjugate, wherein said host cell comprises:
a) a nucleotide sequence of a rfb gene cluster for a O4 polysaccharide;
b) a nucleotide sequence encoding the carrier protein, wherein the carrier protein comprises the amino acid sequence of SEQ ID NO: 3;
c) a nucleotide sequence encoding an oligosaccharyl transferase PglBy; wherein and the PglBy comprises the amino acid mutation N311V or the amino acid mutations Y77H and N311V relative to wild-type PglB having the amino acid sequence of SEQ ID NO: 6:
d) a nucleotide sequence encoding a glucosyltransferase GtrS having at least 95% identity to SEQ ID NO: 4:
e) a nucleotide sequence encoding a glycosyltransferase GtrB having at least 95% sequence identity to SEQ ID NO: 7; and
f) a nucleotide encoding a translocase having at least 95% sequence identity to SEQ ID NO: 8.

9. The bioconjugate of claim 8, wherein the polysaccharide is a glucosylated O4 antigen polysaccharide having the structure: 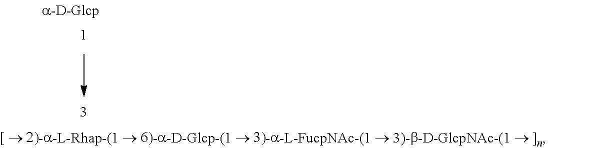
and n is an integer of 5 to 40.

10. A composition comprising a bioconjugate of claim 8 further comprising one or more conjugates each comprising an E. coli antigen polysaccharide covalently coupled to a carrier protein.

11-15. (canceled)

16. The bioconjugate of claim 8, wherein said rfb gene cluster comprises the sequence of SEQ ID NO: 9, the nucleotide sequence encoding said glucosyltransferase GtrS comprises the sequence of SEQ ID NO: 4, the nucleotide sequence encoding said glycosyltransferase GtrB comprises the sequence of SEQ ID NOs 7, and the nucleotide encoding said translocase comprises the sequence of SEQ ID NO: 8.

17. The bioconjugate of claim 16, wherein said prokaryotic host is E. coli.

18. The bioconjugate of claim 16, wherein said prokaryotic host is E. coli strain K-12.

19. The bioconjugate of claim 16, wherein said prokaryotic host is E. coli strain W3110.

20. The bioconjugate of claim 17, wherein said PglBy comprises the amino acid sequence of SEQ ID NO: 6 with said N311V mutation.

21. The bioconjugate of claim 17, wherein said PglBy comprises the amino acid sequence of SEQ ID NO: 6 with said Y77H mutation and said N311V mutation.

22. The bioconjugate of claim 18, wherein said PglBy comprises the amino acid sequence of SEQ ID NO: 6 with said N311V mutation.

23. The bioconjugate of claim 18, wherein said PglBy comprises the amino acid sequence of SEQ ID NO: 6 with said Y77H mutation and said N311V mutation.

24. The bioconjugate of claim 19, wherein said PglBy comprises the amino acid sequence of SEQ ID NO: 6 with said N311V mutation.

25. The bioconjugate of claim 19, wherein said PglBy comprises the amino acid sequence of SEQ ID NO: 6 with said Y77H mutation and said N311V mutation.

26. The bioconjugate of claim 20, wherein said process further comprises isolating said bioconjugate.

27. The bioconjugate of claim 21, wherein said process further comprises isolating said bioconjugate.

28. The bioconjugate of claim 22, wherein said process further comprises isolating said bioconjugate.

29. The bioconjugate of claim 23, wherein said process further comprises isolating said bioconjugate.

30. The bioconjugate of claim 24, wherein said process further comprises isolating said bioconjugate.

31. The bioconjugate of claim 25, wherein said process further comprises isolating said bioconjugate.

32. The bioconjugate of claim 9, wherein said prokaryotic host is E. coli, said rfb gene cluster comprises the sequence of SEQ ID NO: 9, the nucleotide sequence encoding said glucosyltransferase GtrS comprises the sequence of SEQ ID NO: 4, the nucleotide sequence encoding said glycosyltransferase GtrB comprises the sequence of SEQ ID NOs 7, and the nucleotide encoding said translocase comprises the sequence of SEQ ID NO: 8.


SEQUENCE LISTING (19 sequences)

SEQ ID NO: 1-2 - skipped (2 entries)

SEQ ID NO: 3 - protein, 652 aa, synthetic construct
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 4 - protein, 421 aa, synthetic construct
MNNLIMNNWCKLSIFIIAFILLWLRRPDILTNAQFWAEDSVFWYKDAYENGFLSSLTTPRNGYFQTVSTFIVGLTALLNPDYAPFVSNFFGIMIRSVIIWFLFTERFNFLTLTTRIFLSIYFLCMPGLDEVHANITNAHWYLSLYVSMILIARNPSSKSWRFHDIFFILLSGLSGPFIIFILAASCFKFINNCKDHISVRSFINFYLRQPYALMIVCALIQGTSIILTFNGTRSSAPLGFSFDVISSIISSNIFLFTFVPWDIAKAGWDNLLLSYFLSVSILSCAAFVFVKGTWRMKVFATLPLLIIIFSMAKPQLTDSAPQLPTLINGQGSRYFVNIHIAIFSLLCVYLLECVRGKVATLFSKIYLTILLFVMGCLNFVITPLPNMNWREGATLINNAKTGDVISIQVLPPGLTLELRKK

SEQ ID NO: 5 - DNA, 1266 bp, synthetic construct
  source 1..1266: mol_type = other DNA
atgaataatttaattatgaataactggtgtaaattatctatatttattattgcatttattttgctatggcttagaaggccggatatactcacaaacgcacaattttgggcagaagattccgttttctggtataaggacgcctatgagaacggattcttaagttcactaacaacgcctaggaatgggtatttccagactgtttctacatttatagttggtctgactgctttattaaatccagattatgcaccttttgtttctaatttttttggcataatgattcgctcagtaattatatggtttttatttacagaaagattcaacttcctcacattgactactaggattttcttatctatttattttctatgcatgcctggattggatgaagttcatgcaaatataacaaatgcacattggtatttgtcattatatgtatcaatgatcctgatagctcgcaatccaagttcaaaatcatggaggtttcatgatatattctttatcttgctatccgggctcagtggcccatttataattttcattttagcagcttcatgctttaaatttataaataattgtaaagatcatattagtgtaagatctttcataaatttctacttgcgtcagccatacgcattaatgattgtttgcgctttaattcaaggaacttctataattctaactttcaatggcacacgttcctcagcaccgctaggattcagttttgatgtgatttcgtctattatatcatcgaatatttttttatttacatttgtcccatgggatattgcaaaggctgggtgggataatttactgttatcttattttttgtctgtttcgattttgtcgtgtgcggcctttgtttttgttaaaggtacgtggcgaatgaaagtatttgcaactttaccattgctaattataatattttcaatggcaaaaccacaattgacagactcggcacctcaattgccaacacttattaatgggcaaggttcaagatacttcgtaaatatacatattgcgatattctctttgctatgtgtttacttacttgagtgcgtcagggggaaagtggcaactttattttccaaaatatacttaacaattttgctattcgtgatgggatgtttgaattttgttatcaccccactcccaaacatgaactggagggaaggtgctactttgattaataatgcaaaaactggtgatgtcatttcgattcaagtgctaccacctggcctaacacttgaactaaggaaaaaataa

SEQ ID NO: 6 - protein, 713 aa, synthetic construct
MLKKEYLKNPYLVLFAMIILAYVFSVFCRFYWVWWASEFNEYFFNNQLMIISNDGYAFAEGARDMIAGFHQPNDLSYYGSSLSALTYWLYKITPFSFESIILYMSTFLSSLVVIPTILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAIMVKKYSQLTSNVCIVFATILTLAPVFIHIYNYKAPTVFSQNEASLLNQLKNIANREDYVVTWWDYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 7 - protein, 120 aa, Escherichia coli
MLKLFAKYTSIGVLNTLIHWVVFGVCIYVAHTNQALANFAGFVVAVSFSFFANAKFTFKASTTTMRYMLYVGFMGTLSATVGWAADRCALPPMITLVTFSAISLVCGFVYSKFIVFRDAK

SEQ ID NO: 8 - protein, 306 aa, Escherichia coli
MKISLVVPVFNEEEAIPIFYKTVREFEELKSYEVEIVFINDGSKDATESIINALAVSDPLVVPLSFTRNFGKEPALFAGLDHATGDAIIPIDVDLQDPIEVIPHLIEKWQAGADMVLAKRSDRSTDGRLKRKTAEWFYKLHNKISNPKIEENVGDFRLMSRDVVENIKLMPERNLFMKGILSWVGGKTDIVEYVRAERIAGDTKFNGWKLWNLALEGITSFSTFPLRIWTYIGLVVASVAFIYGAWMILDTIIFGNAVRGYPSLLVSILFLGGIQMIGIGVLGEYIGRTYIETKKRPKYIIKRVKK

SEQ ID NO: 9 - DNA, 14440 bp, synthetic construct
  source 1..14440: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtatatggtgatttgcctcatcctgacgaggtaaataatacagaagaattacccttatttactgagacaacagcttacgcgccaagcagcccttattccgcatccaaagcatccagcgatcatttagtccgcgcgtggaaacgtacctatggtttaccgaccattgtgactaattgctctaacaattatggtccttatcatttcccggaaaaattgattccattggttattctcaatgctctggaaggtaaagcattacctatttatggtaaaggggatcaaattcgcgactggctgtatgttgaagatcatgcgcgtgcgttatataccgtcgtaaccgaaggtaaagcgggtgaaacttataacattggtgggcacaacgaaaagaaaaacatagatgtagtgctcactatttgtgatttgctggatgagattgtaccgaaagagaaatcttatcgtgagcaaatcacttatgttgccgatcgtccgggacacgatcgccgttatgcgattgatgctgagaatattggtcgcgaattgggatggaaaccacaggaaacgtttgagagcgggattcggaagacagtggaatggtatctgtccaatacaaaatgggttgataatgtgaaaagtggtgcctatcaatcgtggattgaagagaactatgagggccgccagtaatgaatatcctcctttttggcaaaacagggcaggtaggttgggaactacagcgtgctctggcacctctgggtaacttgattgctcttgatgttcattccactgattattgtggcgatttcagtaaccccgaaggtgtggctgaaaccgtcaaaaaaattcgcccagatgttattgttaatgctgctgctcataccgcggtagataaggctgagtcagaaccagaatttgcacaattactcaatgcgaccagcgttgaagcaattgcaaaagcggctaatgaagttggggcttgggtaattcattactcaactgactacgtcttccctggaaatggcgacatgccatggctcgagactgatgtaaccgctccgctcaatgtttatggcaaaaccaaattggctggagaaagagcattacaagaacattgcgcaaagcatcttattttccgtaccagctgggtatatgcaggtaaaggaaataactttgccaaaacaatgttacgtctggcaaaagagcgcgaagaactggctgtgataaacgatcagtttggcgcaccaacaggtgctgaattgctggctgattgcaccgctcatgccattcgcgtggcattaaaaaaaccagaagttgctggcttgtaccatctggtagcaaatggcacaacaacctggcacgattacgccgcgctagtattcgaagaagcccgtaaagcagggattgaccttgcacttaacaaactcaacgccgtaccaacaacggcttatcctactccagcccgccgtcctcataattctcgcctcaataccgaaaagtttcagcagaactttgcgcttgtcttgcctgactggcaggtgggcgtgaaacgtatgctcaacgaattatttacgactacggcaatttaacaaatttttgcatctcgctcatgatgccagagcgggatgaattaaaaggaatggtgaaatgaaaacgcgtaaaggtattattctggctggtggttccggcactcgtctttatcctgtgacgatggcagtgagtaaacaactgctgccgatttatgataagccgatgatttattatccgctttcaacgcttatgttagcgggtattcgcgatattcttattatcagtacgccacaggatacaccgcgtttccaacaattgttgggggacgggagtcagtgggggcttaatctacagtataaagtacaaccgagtccggatggcctggcgcaagcgtttattattggtgaagactttattggtggtgatgattgtgcactcgtacttggcgataatatcttctatggacacgacttgccgaaattaatggaagctgctgttaacaaagaaatcggtgcaacggtatttgcttatcacgtcaatgatcctgaacgttatggtgtcgtggagtttgataataacggtactgcaattagcctggaagaaaaaccgctggaaccaaaaagtaactatgcggttactgggctttatttctatgacaatgatgttgtagaaatggcgaaaaaccttaagccttctgcccgtggcgaactggaaattaccgatattaaccgtatttatatggagcagggacgtttgtctgtcgctatgatggggcgtggttatgcctggttggatactggtacacatcaaagtcttattgaagcaagtaacttcattgccaccattgaagagcgtcagggattaaaggtatcttgcccggaagagattgcttaccgtaaagggtttattgatgctgagcaggtgaaagtattagccgaaccgctgaagaaaaatgattatggtcagtatctgctaaaaatgattaaaggttattaataaaatgaacgtaattaaaactgaaattcctgatgtgctgatttttgaaccaaaagtttttggtgatgaacgtggcttcttttttgagagttttaaccagaaagtatttgaagaagctgtaggacggaaggttgaatttgttcaggataaccattctaagtctaaaataaatgtattgcgtgggatgcattatcaaacacaaaatactcaaggaaaactggttcgggtaatttctggttcagtatatgatgttgccgtagatttaagagaaaaatcaaagacatttggcaaatgggtgggtgtagaattatctgggaataataaaagacaattgtggatccccgaaggttttgcccatggtttttatgtgttggaggagaataccgaatttgtttataaatgtaccgatacttataaccctgctcatgaacacacattgctatggaatgatccaactatcaatataagttggccaatcatacaaaactgcaagccaattatttctgaaaaagatgctaatggacatcttttttcacataaaacctatttctgaaatgcaatattatgagtttaattagaaacagtttctataatattgctggttttgctgtgccgacattagttgcagtccctgctttggggattcttgccaggctgcttggaccggagaattttggacttttcacactagcattcgctttgataggatatgcaagtattttcgacgccgggattagtcgagctgtaatcagagaaatcgctctttatcgagaaagtgaaaaagagcaaatacaaattatttcgacagcaagtgtaatcgtactattcttaggggtggttgcagctttgttactttattttagtagtaataaagttgttgagttattgaatgttagttccgtttatattgaaacagcagtgcgtgcattctctgttatttcatttataatacctgtgtatctgattaaccagatttggcttggttatctggaagggctagaaaaatttgcaaatataaatgttcagagaatgatttctagcacaagcttggctatattaccagtgatattttgttattacaatccctcgttgctttatgctatgtatgggttggtggttgggcgtgtgatttcatttttgattagcgcaataatttgtcgagatattattcttaaaagtaaactttactttaatgtggcaacttgcaatcgtcttatctcttttggtggatggataacagttagtaatatcataagcccaatcatggcatatttcgaccgctttatcatctctcatattatgggggcttcgagaattgcattttatacagcgccctcagagggtgtatcaaggttaattaatatcccatatgctttggcaagagctctatttcctaaattggcatatagcaataatgatgatgaacgaaaaaaattacaactacagagctacgcaattataagcattgtatgtctacccatagttgttattggtgtcatttttgcctcattcataatgacaacatggatgggacctgattatgccttagaagcagcaactatcatgaaaatacttcttgctggttttttctttaactctttagcgcaaataccttatgcatacttgcaatctatcggaaagtcaaaaattaccgcatttgtgcatctcatagaacttgcgccatacttattattattgtattacttcacaatgcatttcggcataattggcacggcaatcgcttggtcacttagaacattttgtgattttgttatactactttcgatatcgagaagaaaatgattgcggttgatattgcgcttgcaacctacaatggtgctaattttattcggcaacagattgaatctatccagaaacaaacttatagaaattggcgtcttataataagtgatgataactcgagtgatgatactgttgatattattaaggatatgatgtctaacgacagtcgtatctatttggtaggaaataaaagacaaggaggggttattcagaactttaattatgctctttcacaaactacatctgaaattgtgttactatgtgaccaggatgacatttggccggaggagcgtctggaaattcttatagataaatttaaggccttgcagcgtaatgattttgttccggcaatgatgtttactgatttgaaattagtagacgaaaataattgtttgattgcagaaagtttttatcgaacgaataatattaatccacaagataatctgaaaaataataatcttctctggcgttcaacggtatatggctgtacttgcatcatgaataagaaacttgttgatattgcattgcctatacctacatatgcacatatgcatgatcaatggttggcattattagcgaagcaatatggtaacattttttatttcgactatgcgtctgttcgttataggcaacattctacaaatgttgttggtggtagaaataaaacgccatttcaaaaatttaattccatacaaaaaaacctaaaaaggattaatttgctagtggatagaactgttgctttaattaaatcaaataacgatttctatccagggaataaaatggaaaataaaattgattacttaaaatttggagtgaatgaagtattaccttatctttttaaaggaaacaagaaagttttttcactttgtgtattaattagtttggcattacaaaaatgatatatttattatttttttttgcactgtttatgatctgtacgtttttaacacacaggcgacaggcattatatgttgtatctgcgttagtatttctttttttggctttaacctatccatcaggaggggactggataggttattttctccattatgactgcatggttaatgagcagtgtaataatggttttataatgtttgaacctggatatgaattaattgtttccttatttggatatttgggatttcagacaattattatttttatagccgctgtaaatgtaattctaatattaaattttgcaaagcattttgaaaacggaagttttgttattgttgcgataatgtgcatgttcctttggagtgtttatgttgaggcgattagacaggctctggccttatctatagttatatttgggattcattctctttttttgggtagaaaaaggaaatttataacattagtattatttgcgtcaactttccatataactgctttgatttgttttcttctaatgactcctctattttcaaagaaattaagcaagataataagttatagcctattaattttcagtagcttctttttcgctttttctgaaaccatattaagtgcactccttgcaattttgccagaaggatccattgccagtgaaaaattaagtttttacttagcaaccgagcaatacaggccacagttatctattgggagtggcactattcttgacattatacttatttttctgatatgtgtaagttttaaacgaataaagaaatatatgctcgctaattataatgctgcaaatgagatattgcttattggttgctgtctttatatttctttcggtatttttatcgggaaaatgatgccagttatgactcgcattggttggtatggttttccatttgttatagtacttctttatattaacttgggttattcagaatattttaagaggtatataaataaaagagggtgtgggtatagcaaattattaattgctttttattttttgctacaaattttgcgaccattaacatatgattatagctattataatataatgcaccaggatactttgctgaataggtttgatgcattagatgatgcatcattaagacaatcagcgaagagaaaatgtttcgatttgggaaagataggatatggtttcttatgtagtatataatatcctgcattcattcggataatttcctatggaagtgtcctttgctctgtctgtcctcatttgttgaaattttatgttaataagaagctttagataaccacttaggaactgtatgtttgatctgtccaaaaattatattattgtaagtgcgacggcgctggcttccggaggtgcattaactatattaaagcaatttataaaacatgcatcacaaaattcaaatgactatattatgtttgtatctgcgggattggagttgccggtctgtgataacatcatttacatagaaaacacaccaaaaggatggttgaaaagaatatattgggattggttcggttgtcggaagtttatctcggaacataagattaacgttaagaaagtaatttctctacaaaattccagtttgaatgttccttacgaacagattatttacttgcaccagccaattccttttagtaaagttgattcttttttaaaaaatatcacatccgataacgtaaagctttttttatataaaaagttttattcctattttatatttaaatatgtgaatgccaatacaaccatcgtagtgcaaacgaattggatgaaaaaaggagtgctggagcaatgtgataaaattagtaccgaaagggtccttgttataaaacctgatatcaaagcatttaataatactaattttgatgtagatatggatgtatctgcaaaaacactcttatatccagcgacaccacttacctataaaaatcatttggtcattctgaaggcgttggttattttaaagaaaaagtattttatagatgatctgaaattccaagtgacttttgaaaagaataggtacaaaaattttgataagtttgtgcaattaaataacttaagcaaaaacgttgattatctcggcgttctttcatactcgaacttgcaaaaaaaatatatggcggcatctttaatcgtttttcctagctatatcgaatcatatgggttaccactcatcgaagctgctagtttaggaaaaaaaatcattagtagtgatcttccttatgcccgggatgttttaaaggattatagcggcgtagattttgtaatttacaataatgaagatggctgggctaaggcgttgtttaatgttttaaatggcaattcgaagctcaattttaggccttatgaaaaagatagtcgttcatcttggccacagttcttctctattttgaaataaggtgtattatgtttaatggtaaaatattgttaattactggtggtacggggtctttcggtaatgctgttctaagacgttttcttgacactgatatcaaagaaatacgtattttttcccgggatgaaaaaaaacaagatgacatgaggaaaaaatataataatccgaaacttaagttctatataggtgatgttcgcgactattcgagtatcctcaatgcttctcgaggtgttgattttatttatcatgctgcagctctgaagcaagtaccttcctgcgaattccacccaatggaagctgtaaaaacgaatgttttaggtacggaaaacgtactggaagcggcaatagctaatggagttaggcgaattgtatgtttgagtacagataaagctgtatatcctatcaatgcaatgggtatttccaaagcgatgatggaaaaagtaatggtagcaaaatcgcgcaatgttgactgctctaaaacggttatttgcggtacacgttatggcaatgtaatggcatctcgtggttcagttatcccattatttgtcgatctgattaaatcaggtagaccaatgacgataacagaccctaatatgactcgtttcatgatgactctcgaagacgctgttgatttggttctttacgcatttgaacatggcaataatggtgatatttttgtccaaaaggcacctgcggctaccatcgaaacgttggctattgcactcaaagaattacttaatgtaaaccaacaccctgtaaatataatcggcacccgacacggggaaaaactgtacgaagcgttattgagccgagaggaaatgattgcagcggaggatatgggtgattattatcgtgttccaccagatctccgcgatttgaactatggaaaatatgtggaacatggtgaccgtcgtatctcggaagtggaagattataactctcataatactgataggttagatgttgagggaatgaaaaaattactgctaaaacttccttttatccgggcacttcggtctggtgaagattatgagttggattcataatatgaaaattttagttactggcgctgcagggtttatcggtcgaaatttggtattccggcttaaggaagctggatataacgaactcattacgatagatcgtaactcttctttggcggatttagagcagggacttaagcaggcagattttatttttcaccttgctggggtaaatcgtcccgtgaaggagtgtgaatttgaagagggaaatagtaatctaactcaacagattgttgatatcctgaaaaaaaacaataaaaatactcctatcatgctgagttcttccatccaggctgaatgtgataacgcttatggaaagagtaaagcagctgcggaaaaaatcattcagcagtatggggaaacgacaaacgctaaatattatatttatcgcttgccgaatgtattcggtaagtggtgtcgaccaaattataactcctttatagcaactttctgccatcgcattgcaaatgatgaagctattacaattaatgatccttcagcagttgtaaatctggtgtatatagatgacttttgttctgacatattaaagctattagaaggagcgaacgaaactggttacaggacatttggtccaatttattctgttactgttggtgaagtggcacaattaatttaccggtttaaagaaagtcgccaaacattaatcaccgaagatgtaggtaatggatttacacgtgcattgtactcaacatggttaagttacctgtctcctgaacagtttgcgtatacggttccttcttatagtgatgacagaggggtattctgtgaagtattgaaaacgaaaaacgcgggccagttttcgttctttactgcgcatccaggaattactcggggtggtcattatcatcattccaaaaatgagaaatttattgtcatccgaggaagtgcttgtttcaaatttgaaaatattgtcacgagtgaacgatatgaacttaatgtttcctctgatgattttaaaattgttgaaacagttccgggatggacgcataacattactaataatggctcggatgagctagttgttatgctttgggcaaatgaaatatttaatcgttctgaaccagatactatagcgagagttttatcgtgaaaaaattgaaagtcatgtcggttgttgggactcgtccagaaattattcgactctcgcgtgtccttgcaaaattagatgaatattgtgaccaccttattgttcataccgggcaaaactacgattatgaactgaatgaagtttttttcaaagatttgggtgttcgcaaacctgattattttcttaatgccgcaggtaaaaatgcagcagagactattggacaagttatcattaaagttgatgaggtccttgaacaggaaaaaccagaagccatgttagtacttggcgatactaactcctgtatttcagcaataccagcaaagcgtcgaagaattccgatcttccatatggaggctgggaatcgttgttttgaccaacgcgtaccggaagaaactaacagaaaaatagttgatcataccgctgatatcaatatgacatatagtgatatcgcgcgtgaatatcttctggctgaaggtgtaccagccgatagaattattaaaaccggtagcccaatgtttgaagtactcactcattatatgccgcagattgatggttccgatgtactttctcgcctgaatttaacacctgggaatttctttgtggtaagtgcccacagagaagaaaatgttgatacccctaaacaacttgtgaaactggcgaatatacttaataccgtggctgaaaaatatgatgtcccggtagttgtttctactcatcctcgcactcgtaaccgcatcaacgaaaacggtattcaattccataaaaatatcttgcttcttaagccattaggatttcacgattacaaccatctgcaaaaaaatgcacgtgctgttttatcggatagtgggactattacagaagagtcctccattatgaacttccctgcactcaatatacgagaagcgcacgaacgcccggaaggcttcgaagaaggggcagtaatgatggtcggtcttgaatctgatcgcgttttacaggcattagaaattattgcaacacagcctcgtggagaagtacgcttacttcgtcaggttagtgactatagcatgccaaatgtttcagataaagttctgcgtattatccattcatatactgactacgttaaacgggttgtctggaagcaatactaatgaaacttgcattaatcattgatgattatttgccccatagcacacgcgttggggctaaaatgtttcatgagttaggccttgaattactgagcagaggccatgatgtaactgtaattacgcctgacatctcattacaagcaatttattctattagtatgattgatggtataaaggtttggcgtttcaaaagtggacctttaaaggatgtaggtaaggctaaacgtgccataaatgaaactcttttatcttttcgcgcatggcgcgcatttaagcacctcattcaacatgatacatttgatggtatcgtttattattccccctctattttttggggcgacttggttaaaaaaataaaacaacgatgccagtgcccaagctatctgatcctaagggatatgtttccacagtgggtcattgatgcaggtatgttgaaagccggttcaccaattgaaaaatattttaggtattttgaaaaaaagtcatatcagcaggctggccggataggggtaatgtctgataagaatcttgagatatttcgccagaccaataaaggttatccgtgtgaagttttacgtaattgggcctcaatgactcctgtgtctgccagcgatgattatcattcacttcgtcaaaaatacgatctaaaagataaagtcatttttttctatggcggtaatattgggcatgctcaggatatggcaaacttaatgcgccttgcgcgtaatatgatgcgttatcatgatgctcatttcctgtttatagggcagggtgatgaagttgagctgataaaatctcttgctgcagaatggaatttaactaatttcactcatctaccttcagtgaaccaggaagagtttaaattaattttatctgaagttgatgtcggcctgttctccctttcatctcgccattcttcacataatttccccggaaaattactagggtatatggttcaatcaatcccgatccttgggagtgtgaatggcggcaatgatttaatggatgtaattaataagcacagagccggtttcattcatgttaatggtgaagatgataaactgtttgaatctgcacaattgcttcttagtgattcagttttaagaaaacagctaggtcagaacgctaatgtgttgttaaagtctcaattttcggttgaatcggcggcacatactatcgaagtccgactggaggctggagaatgcgtttagttgatgacaatattctggatgaactttttcgcactgcagcaaattctgaacgtttgcgcgctcattatttattgcacgcatctcatcaggagaaggttcaacgtttacttattgcatttgtacgcgacagctatgttgaaccccattggcatgagttaccgcatcagtgggaaatgtttgtcgtcatgcaagggcaattagaagtttgtttgtatgagcaaaatggtgagatccaaaaacagtttgttgttggagacggtacgggaataagcgtcgtggaattttccccaggagatatacatagtgtcaaatgcctgtcaccaaaagcccttatgttggagataaaggaggggccatttgacccactcaaagctaaggctttttctaagtggttatagggcgatacaccaccgtttattcttctatcttattctatacatgctgggttaccatcttagcttcttcaagccgcgcaaccccgcggtgaccacccctgacaggagtagctagcatttgaccacccctgacaggattagctagcatatgagctcgaggatatctactgtgggtacccgggatccgtgtaggctggagctgcttcgaagttcctatactttctagagaataggaacttcggaataggaactaaggaggatattcatat

SEQ ID NO: 10 - protein, 19 aa, synthetic construct
MKKIWLALAGLVLAFSASA

SEQ ID NO: 11 - DNA, 13043 bp, synthetic construct
  source 1..13043: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcgtgaagatacttgttactaggggcgcaggatttattggttctgctgtagttcgtcacattataaataatacgcaggatagtgttgttaatgtcgataaattaacgtacgccggaaacctggaatcacttgctgatgtttctgactctgaacgctatgtttttgaacatgcggatatttgcgatgctgctgcaatggcgcggatttttgctcagcatcagccggatgcagtgatgcacctggctgctgaaagccatgtggatcgttcaattacaggccctgcggcatttattgaaaccaatattgttggtacttatgtccttttggaagcggctcgcaattactggtctgctcttgatggcgacaagaaaaatagcttccgttttcatcatatttctactgacgaagtctatggtgatttgcctcatcctgacgaagtaaataataaagaacaattacccctctttactgagacgacagcttacgcgcctagtagtccttattccgcatcaaaagcatccagcgatcatttagtccgcgcgtggaaacgtacctatggtttaccgactattgtgactaactgttcgaataactacggtccttatcactttccggaaaaattgattccactagtaattcttaatgctctggaaggtaaggcattacctatttatggcaaaggggatcaaattcgtgactggctgtatgttgaagatcatgcgcgtgcgttatataccgtagttactgaaggtcaagcgggtgaaacctataacattggcggacacaacgaaaagaaaaacatcgatgttgtgctgactatttgtgatttgttggacgagatagtcccgaaagagaaatcttatcgtgagcaaattacttatgttgctgatcgcccagggcatgatcgccgttatgcgattgatgctgagaagattggtcgcgaattgggatggaaaccacaggaaacgtttgagagtgggattcgtaaaacggtggaatggtatttggctaatgcaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctctgggtaatttgattgctcttgatgttcactccactgattactgtggtgattttagtaaccctgaaggtgtggctgaaacagtcaaaagaattcgacctgatgttattgttaatgctgcggctcacaccgcagtagataaggctgagtcagaacccgaatttgcacaattactcaatgcgactagcgttgaatcaattgcaaaagcggcaaatgaagttggggcttgggtaattcattactcaactgactacgtattccctggaaatggcgacacgccatggctggagatggatgcaaccgcaccgctaaatgtttacggtgaaaccaagttagctggagaaaaagcattacaagagcattgtgcgaagcacctaattttccgtaccagctgggtctatgcaggtaaaggaaataatttcgccaaaacgatgttgcgtctggcaaaagagcgtgaagaactagccgttattaatgatcagtttggtgcgccaacaggtgctgaactgctggctgattgtacggcacatgccattcgtgtcgcactgaataaaccggatgtcgcaggcttgtaccatttggtagccagtggtaccacaacctggtacgattatgctgcgctggtttttgaagaggcgcgcaatgcaggcattcctcttgcactcaacaagctcaacgcagtaccaacaactgcctatcctacaccagctcgtcgtccacataactctcgccttaatacagaaaaatttcagcagaattttgcgcttgtattgcctgactggcaggttggtgtgaaacgcatgctcaacgaattatttacgactacagcaatttaatagtttttgcatcttgttcgtgatggtggagcaagatgaattaaaaggaatgatgaaatgaaaacgcgtaaaggtattattttagcgggtggttctggtactcgtctttatcctgtgactatggtcgtcagtaaacagctattacctatatatgataaaccgatgatctattatccgctttctacactgatgttagcgggtattcgcgatattctgattattagtacgccacaggatactcctcgttttcaacaactgctgggtgacggtagccagtggggcctgaatcttcagtacaaagtgcaaccgagtccggatggtcttgcgcaggcatttattatcggtgaagagtttattggtggtgatgattgtgctttggtacttggtgataatatcttctacggtcacgacctgcctaagttaatggatgccgctgttaacaaagaaagtggtgcaacggtatttgcctatcacgttaatgatcctgaacgctatggtgtcgttgagtttgataaaaacggtacggcgatcagcctggaagaaaaaccgctacaaccaaaaagtaattatgcggtaaccgggctttatttttatgataacgacgttgtcgaaatggcgaaaaatcttaagccttctgcccgcggtgaactggaaattaccgatattaaccgtatctatatggaacaagggcgtttatctgttgccatgatggggcgtggttatgcgtggttagacacggggacacatcagagcctgattgaggcaagcaactttattgcaacaattgaagagcgtcaggggctgaaagtttcctgcccggaagaaattgcttaccgtaaagggtttgttgatgctgagcaggtgaaagtattagctgaacctctgaaaaaaaatgcttatggtcagtatctgctgaaaatgattaaaggttattaataaaatgaacgtaattaaaacagaaattcctgatgtactgatttttgaaccgaaagtttttggtgatgagcgtggtttcttttttgagagctttaaccagaaggtttttgaggaagctgtaggccgcaaagttgaatttgttcaggataaccattcgaagtctagtaaaggtgttttacgcgggctgcattatcagttggaaccttatgcacaaggaaaattggtgcgttgcgttgtcggtgaagtttttgacgtagctgttgatattcgtaaatcgtcatcgacttttggcaaatgggttggggtgaatttatctgctgagaataagcggcaattgtggattcctgagggatttgcacatggttttttagtgctgagtgagacggcggagtttttgtataagacgacaaattattatcatcctcagagtgatagaggaataaaatgggatgatccaagcatcaatatttcatggccagtcgattcacaagtgctgctatcagctaaagataataagcatcctccattaacaaagattgaaatgtatagttaagatcacgataaatcttggaagggttgcaaaattgaataaaatagtgagcaaaagtgaaataaggaacgtaatccacaatgctggctatatgatgattactcagatagctttatatgttgcaccattatttatactgagttatctgttaaaaacactgggggttgcacagtttggtaattatgccttaatactatcaatcgttgcatatttacagattataacggattatggtttttcttttagtgcaagtcgtgcgatctcacagaatagagaggacaaagaatatatatcaaaaatttatctgtcaactatgactatcaagttggcgatatgcgctttcttattcttattgctcatgctatttttaaatcttttgcctgtgcaagctgaattaaaacaaggaatattatatggatatcttcttgtaataggaaatactttccaaccacaatggtttttccaaggtatcgaaaaattaaaaatcatagccctttctaatgttatatcaagatgcgccgcgtgtttacttgtatttatctatgtgaggaatagcgaggatttacaaaaagcacttttagtacagtcacttccattagtaatttctgcgattggattaaatatatttatattgaaatatatcaatattatttttccggaaaaaaaattatttaaggtaattttaaaagaaggtaaggatttttttcttgcatcactttattctgttattctcaataatagtggcatttttctattagggatttttactaatcctgttattgttggtgtatatgccgccgctgaaaagatagtcaaggccgtattgtcgctatttacaccactgacgcaagctatatatccttataattgtcgtaagttttcactatccgtatttgacggcattgaggcagcaaaaaaaactggtataccaattataattttagcatttatagctgctgttatcgttgcaattaccttacctgttgcaatcgactatcttaattttccaaaagaaacaatttttgtaggtcaaatattaagtgcatggatcttttttggtgttcttaataatgtattcggcattcagatattgagtgcatcaggaagaagtaaaatatatagtaggatggtattcgtatcagcgcttataacattacttttgattactctattattgcagttttgtaacgccactggagtggcatgtgcaatattattgggtgaaatgttcttatcaatattgttacttaagcgatataaaaaaataatttaaggaatagttatgaagaagttattattagtgttcggtactaggcctgaagcaataaagatggcctctatcattgaattattaaaaaaagattgtagattcgaatataaaatatgtgtgacaggccaacataaagagatgcttgatcaagttatgcaagtatttgatgttaaacctgattataatttacggattatgcagcctgggcaaacattagtatctatagcaacaaatatactctcacggttaagtgaagttttaattatagaaaagccagatattatacttgtgcatggggatacaacgactacccttgctgctactttagctgggtattaccaccaaataaaagtttgtcatgtggaagcaggattaagaacaggggatatttactctccttggcctgaagagggcaatcgtaaagttacaggggcattagcatgtattcatttcgccccaacagagagatcaaaagataatctcctgagggagggggtcaaagtaaataatatatttgtaacgggtaataccgtcatcgactctttatttattgcaaaagatatcatagataatgaccctaatataaagaacgctttacataataaatttaattttcttgataaaagccgacgagtagtacttataacaggtcatcgaagagaaaatttcgggaaaggttttgaagatatatgctttgcaataaaggaattagctttcatttatcctaatgtagattttatttatccggtgcatcttaatcccaatgtaatggaaccagtacatcgtatattagataatatatgtaatatttaccttattgagcccttggattatttgccttttgtttatttaatgaatgagtcatatttaatattgactgattcaggggggatacaagaagaagcgccttcgttaggtaaaccggttttggttatgcgtgatactactgaacgccctgaggcggttgaggctggtactgttgtattagtggggacttctaagataaaaatagtaaataaagtaacggagctattaaacaatgctgatatctacaatgctatgtctctgttacataatccatatggcgatggaacagctgctcaaaaaattcttaatgtgctcgcccaagagctaatttaatttaagctaaaaatatgttattaattattgctgattatccaaacgaaatgaatatgcgcgagggagctatgcaacgaatagatgcgatagactctctcattcgagatcgcaagcgagtgtatttgaatatttcattcaaaaagcatctagttcgctcaaatagttcctttaataatgttatagttgaaaatctaaatgcaattattcacagaaacatcataaaacagtacatgcaaaaatcaacaactatatatgttcattctgtttataatttattaaaggttataacgctcattgatctaaaaaaaacaattcttgatatacatggtgttgtaccggaagaacttttggcagataataaaaaattacttagtaaagtatataacatggtggaaaaaaaaggtgtccttggatgcaaaaaattaatacacgtcagtacagaaatgcaaaaacactatgaagcaaaatatggagtaaacttggctgaaaggtcaatagtgctcccgatttttgaatataaaaatataacccaatcgcaaaacaaatggacagaaaataaaatacgaagtatctatcttggaggattacaaacatggcaaaatattgataaaatgattcaagtttgtgatgacacagtgataaacaatgaagcaggtaagtatgaattcaactttttcatcccacagagtaacttggaagggtttatagataaatattcgttaaaattacataatatcaatgctaatgcatctacgctatcacgtgatgaagtaattccctttctaaaagaatgtcatattggttttgtattgcgcgatgatataatagtaaacagagttgcgtgccctacaaaattggttgaatatttagagtgtggtgtcgttccagttgtgctctccccacttataggtgatttttattcgatgggatatcaatacattactacagaggaaatggctaacagaagtataagtttgttggatcttgaaaaaatggctgcacataatttacaaattttgacttcttatcagaagagaacctacaaggcacagaaagaacttattgctcaactgtgctgaattttttacatatataaaattatgtaagcatatcgcgggtcaggtaattgtatgcgtatcaaatataaagataacggttatatattatgttttctattatgtttcattttgagctacttagttttactcaaatctgactactttcctgctgattttctgccatatacagaaatatacgatgggacatacggagaaatcaataatattgagcctgcctttttatatttaacacggttgtttcattatttaaatttcccctatatattttttgcaatgttagtttgtgccttatgtttaagttggaaaataaaatatgcaagaaaaataattaaagatagttatatatatttgttcttgtatgtatatgtatcattttatgtgtttttgcatgaaatgactcaattgcgcatagcaattgcagtcactatgtgctatgtgtcggtttattattacttttataaaaattgtattaaacatgcactgccatggatggtgttggctattttgtttcattacagcgccttgcttttatttatgtcattatttatatacagttataggaggttattaatagtaattatagggtttgtaatatgtatgagctttttaaacgtgtatgcagatacaattgcactatatttgccaaatgaaaaaatagtaaattatttatatagtatttcatcatcattagacaatagaaatgatttggcaatattcaacctgaataatataatatttttatcaatatttattttgatcttttatcttagccgatatataaaattaaatgataatgaggcgaagtttattaagtatgtgcaatgttcaggaatattagccttttgtattttctttctggctagtggagtcccggtcattgcttatcgaactgcagagttgctgcgaatattttatccgatggctttagtattaatcctttcgcatataaaaaataataatatgcgttattttattgcagtcattatagttatcctttcaggcttaatgttgtttataacactaagggctgtatcaatagttggtcaaggattataaaatgaatgttgctattttgttgtctacgtataatggcgaaaaatatttagaggaacaactggattcattgctgcttcaaagttatcaggattttgtagtgtatatccgtgatgacggatcatctgatagaactgtaaatataataaaccaatacgtaatgaaagataacagatttattaacgtgggtaattcagaaaatcttggttgtgctgcttcgtttattaatttattaagaaatgcttcagccgatatttatatgttttgtgaccaagatgattattggcttccgaataaattacagcgtgctgtggattatttttcggctattgatcctttacaacctaccttgtatcattgcgatctaagcgttgttgatgaaaaacttaatattatacaaaattcatttttgcagcatcagaaaatgtcagcgtatgattcaatgagaaaaaataatcttttcatacaaaattttgttgttggttgttcatgtgctgttaatgcttcacttgcggaatttgttctttcgcgaattggagagcagcatgtaaaaatgatagctatgcatgactggtggttagccgtgactgcaaaactttttggtcgaatccattttgataatactcaaacgattctttatcgacaacatcagggcaatgtattaggtgcaaaatcatcaggtatgatgcgttttattcgattaggattaaatgggcaagggatttcgcgagtagtatcttttagaaaaaaagtttgtgcgcaaaataagcttcttttagatgtctatgataaagatttaaatcttgagcaaaaaaaatctatcaggcttgtaattgagggccttaaagagaactcttcaattgctgaccttttaaaatgtttctatcatggtagctatatgcaaggttttaaacgtaatcttgccttaatatattcagttctttacacaaaaaaaagaagatagtgtatccttatgaaaaaaattgctattatcggtactgttggcataccagcatcatatggcggatttgaaacattagttgaaaatttaacaagatacaattcctcgggagttgaatataatgttttttgttcatcgtttcactacaaatcccaccaaaaaaaacataatggggcccgtttaatttatattccgcttaaagccaatggatggcagagcattgcgtatgacataatttcgttagcatattctatttttttgaagcctgatgtgattctgattttaggggtttctggttgttcatttttgcctttcttcaaactcttaacacgcgctaagtttattactaatattgatggcctggaatggcgaagagataaatggaattcaaaagtgaaacgtttcttaaaattttcagaaaaaatcgcagttcaatattcggatgtcgttattacggataatgaggcaatttctgagtacgtttttaacgagtataataaagatagccgagttattgcctatggaggggatcatgcatggttaaatactgaggatgtatttacaacaagaaattataaaagcgattactacctttctgtatgtcgtatcgaacccgaaaacaatgtagaattaattttaaaaacattttcaaagctaaaatataaaataaaatttattggaaattggaatggcagcgagtttggaaagaaacttaggctgcattattctaactatccaaatattgaaatgattgatccgatttatgatcttcaacaattatttcacttacgaaataattgcataggatatatacatggtcattcggctggaggaacaaacccttctttagtcgaggcaatgcattttagtaaacctatatttgcatatgattgtaagtttaataggtacactactgaaaatgaagcatgttatttttctaatgaatctgacctcgcagagaaaatcataatgcattgtgagctatcattaggtgtctctggcacgaaaatgaaagaaattgctaaccagaaatacacttggagacgaatagcagaaatgtatgaggattgctattaactctgttaaacttcaaatcttttacaatatatggcatgactataagcgcattaattgtttttcaagccgctctcgcggtgaccaccccctgacaggggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtatcgataaagaaggtgtgttccataccgaatggctggattaa

SEQ ID NO: 12 - DNA, 13790 bp, synthetic construct
  source 1..13790: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatccagatgaagtaaataatacagaagaattacccttatttactgagacgacagcttacgcgccaagcagcccttattccgcatccaaagcatccagcgatcatttagtccgcgcatggaaacgtacgtatggtttaccgaccattgtgactaattgctcgaacaactatggtccgtatcacttcccggaaaagcttattccattggttattcttaatgcactggaaggtaaggcattacctatttatggcaaaggggatcaaattcgcgactggttgtatgtagaggatcatgctcgtgcgttatataccgtcgtaaccgaaggtaaagcgggtgaaacttataacattggcggacacaacgaaaagaaaaacatcgatgttgtgctgactatttgtgatttgttggatgagattgtaccgaaagagaaatcttatcgtgagcaaattacttatgttgctgatcgcccagggcatgatcgccgttatgcaattgatgccgataaaattagccgcgaattgggctggaaaccacaggaaacgtttgagagcgggattcgcaaaacggtggaatggtatctggctaatacaaattgggttgagaatgtgaaaagcggtgcttatcagtcatggatcgaacaaaactatgagggccgtcagtaatgaatatcctgcttttcggcaaaacagggcaggtgggttgggaactgcagcgtgctctggcgccgctgggtaatctgatcgctcttgatgttcactccactaattattgtggagatttcagcaaccccgaaggtgtggcagaaaccgtcaaaaaaattcgtcctgacgttattgttaatgctgctgctcacactgcagtagataaagcagaatcagaaccggatttcgcacaattacttaacgcgacaagcgtcgaagcgattgcaaaagctgctaatgaagtcggggcctgggttatacactactctactgattatgttttcccaggcagtggtgacgcgccatggctggaaacggatgcaacagcaccgctaaatgtttacggtgaaacaaaattagctggggaaaaggcattacaagaacattgcgcaaagcatcttattttccgtaccagctgggtatacgctggtaaaggaaataactttgctaaaacgatgttgcgtttggcaaaagaacgcgaagaactggctgtgataaacgatcagtttggcgcaccaacaggtgctgaattgctggctgattgcaccgctcatgccattcgcgtggcattaaaaaaaccagaagtcgctggcttgtaccatctggtagcaagtggcacaacaacctggcacgattatgctgcgctggtttttgaagaggcgcgcaaagcagggattaatcttgcacttaacaaacttaacgccgtgccaacaacggcctatcccacaccagcccgtcgaccccataactctcgcctcaatacagaaaagtttcagcagaactttgcgcttgtcttgcctgactggcaggtgggcgtgaaacgtatgctcaacgaattatttacgactacggcaatttaacaaatttttgcatctcgctcatgatgccagagcgggatgaattaaaaggaatggtgaaatgaaaacgcgtaaaggtattattctggctggtggttccggcactcgtctttatcctgtgacgatggcagtgagtaaacaattgctgccgatttatgataagccgatgatttattatccgctttcaacgcttatgttagcgggtattcgcgatattcttattattagtacgccacaggatacaccgcgtttccaacaattattgggggacgggagccagtggggtcttaatctacagtataaagtacaaccgagtccggatggcctggcgcaagcgtttattattggcgaagactttattggtggtgatgattgtgcactcgtacttggcgataatatcttctatggacacgacttgccgaaattgatggaagctgctgttaacaaagaaagcggtgcaacggtatttgcttatcacgttaatgatcctgaacgctatggtgtcgtggagtttgataataacggtacggcaattagcctggaagaaaaaccgctggagccaaaaagcaactatgcggttactgggctttatttctatgacaatgacgttgtggaaatggctaaaaaccttaagccttctgcccgtggcgaactggaaattaccgatattaaccgtatttatatggaacaaggacgtttgtctgtagccatgatggggcgtggctatgcatggttggatacagggacgcatcaaagccttattgaagcaagtaacttcattgcaacaattgaagagcgtcagggattaaaggtatcttgcccggaagagattgcttaccgtaaagggtttattgatgccgagcaggtgaaagtattagccgaaccgcttatcaagaatcaatatggtcaatatttgctgaaaatgatcagcgaatagtatatgggaactcaatgatggatattaaattaatctctttgcaaaaacatggggatgagcgcggtgcattaattgctcttgaagagcaacgaaatatacctttcgaagtcaaaagaatatattacatacttgagactcttaatggagtaagacgcggatttcatgcgcacaaggttactcgtcagttagctattgtagtcaagggagcttgtaaatttcatctggataatggtaaagaaacaaagcaggtggaacttaatgatccaacaattgcgttgctgatagaaccctatatatggcatgaaatgtatgattttagtgatgattgtgtgctgcttgtaattgcggatgatttctataaagagtctgattatatccgcaattatgatgattttattagaagagtaaattcaattgagaattcataagctaagtgacgtccagacaacatcaattggtgatggaacaactatctggcagtttgttgtgatactaaaaggtgctgtaattggtaataattgcaacatctgtgcaaataccttaattgaaaataacgttgtaattggtaacaatgtcacagtcaaaagcggtgtgtatatttgggatggcgttaaaatagaggataatgtttttattggtccttgtgtagcatttacaaatgataagtatcctcgctctaaagtctatcctgatgaatttttgcaaacaataatacgcaaaggagcatcaataggtgctaacgcaaccatcctgccaggaattgaaattggtgaaaaagcaatcgttggtgcggggagtgttgtaaccaaaaatgtaccgccatgcgcaatagtagtaggtaatccagctcgatttattaaatgggtagaggataatgaataaaattgattttttagatctttttgcaattaaccagcgacagcacaaagaattagtctctgcgtttagtagggtgctagattctggttggtatatcatgggcgaagaacttgagcagttcgagaaagagttcgcagaatactgtggagttaagtattgcattggtgtagcaaatggccttgatgcgttgatactagtattgagggcatggaaagaacttggctatcttgaagacggtgacgaggtattagtaccggcaaatacatatattgcttctattcttgctataacagagaacaaacttgttcctgttcttgttgaaccagatatagaaacttataatattaatcctgctttaattgaaaattacattacggaaaaaactaaagcaatattaccggttcacttatatggtctattgtgcaatatgccagaaattagtgcaatcgccagaaaatataatctgttgattcttgaagattgtgcacaagcacatggtgcaatacgtgatggtcgcaaagctggagcttggggggatgctgcaggatttagtttttatccaggaaaaaaccttggagctttgggggatgcgggagctgttactacaaataatgcagaattatcctcaactataaaagctttgcgaaattatgggtcacataagaaatatgaaaatatttatcagggattgaatagtcgattggatgaactgcaagcagccttattgcgtgtaaaaatccatacattaccggaagatactgcgattcggcaaaggattgctgaaaaatatattcgtgaaataaaaaaccctgcgattacgttaccagtgtacgaaggccaaggtgcgcatgtttggcatttatttgtagtaagaatcgctaatcgtgaaaaattccagtcatacttattagagaagggtatcaaaaccttaattcactatccattaccaccccataagcagcaagcatatcaaaatatgtctagccttagccttccaattactgagcaaattcatgatgaagtcatttctttacctataagtccggtaatgagtgaagatgatgtcaattatgtaatcaaaatggtcaatgattacaagtaatgaaaaaatttcttcaggtaactatattatccgctatctatacattcattaaaatgattgcgggttttatcatcggtaaggtagtagcaatttatacagggccatcaggggtagcaatgcttggccaagtgcaaagtttaatcacaatagttgcaggtactacctctgcacctgtaagcacaggccttgttcgatatactgcggaaaattggcaagaaggacaagaagcatgcgcgccatggtggcgcgcatgcttaagggttactctgtttttattcttgcttattattcccgttgttattatattgtcgaaaaatattagtgagttactttttagcgatggacaatacacatggttaatcattttcgcatgttgtatattgccattctccattataaatacattgatcgcttcagttttaaatggtcaacaattttataagcaatatatattggttgggatgttttctgtattcatttctactatgtttatgattttgttgattgtagcttataatcttaaaggtgcattgattgccacagctataaatagtgctattgctggtcttgtattggttttattttgtctcaataaatcttggtttagatttaaatattggtggggtaaaacggataaagacaaaattataaaaattattcattatactctgatggctctggtttctgttatctccatgcctacagcattgatgtgtattagaaaaatattgattgctaaaactggttgggaggatgcagggcaatggcaggccgtatggaagatatctgaggtttatcttggtgttgtgacaattgctttgtcaacatatttcttaccaagattgacaattataaaaacaagtttccttataaaaaaagaagtaaatagtactatattatacataatatctattacttcattcatggcgttgagtatctatttattccgcgatttggtaataacagttttatttactgaacagtttcgctcagctcgtgaattatttttattacaacttataggggatgtaataaaaattgctgggtttctttatgcataccctcttcaaagtcaggggcatactaaactattcatcagttcagaagtgattttttctatgctctttatcattaccacctatatttttgttgtaaattatggagtacatggtgctaacataagttatgtcattacatatagtttatattttgtgtttgcatttgtgtttactaattttattaatgttagaagaaataattaaaaacagaggttgaattttgaaaataattatacctgtcttaggatttggcagggctggtggtgaaagagttctttctaagctggcaactgaattgatgaattatggacatgatgtaagttttgttgttccagataatagaactaatccatattatgctaccacagcaaaaattgtcacgagtaaatctagtcaaaaccgtgtaaaaatattgagaatcattaaaaattactataatctgtggcgtaaatgcatagagttaaatcctgatgctgtagttgctagttttcatttgactgcctatcttgtcgcattattaccaatcacccgtcgtaagaaatattattatattcaggcgtatgaagttaatttttttgataatataatatggaaattaatagcgggtttaacatattatttaccgcttaaaaaaatactaaatagtcctaatttgcttcctcataaacatgatgattttataggagtagttcctgcaggagtagatttaaacgttttctatccgaaaccatcaaataggttattaaatggtcacacatcaatagggattattggtagaaaagagaagcacaaaggaactagcgaaattatttcagtattgtgttcactggaaaataaagctggaattataatcaatattgcgatctatcttgaagaagttgataagcagcgtttaatcgctgccgggtttcaggttaatttttttccgattacttctgatttagaattggcatccttttatcgaagcaatgacatcatgattgctgttgggttaattgaagatggcgctttccattatccttgtgctgaatcaatggcttgtggttgtcttgttatttcaaattatgcgccacttactgaaactaacagtgtacttaaattagtcaagtttgatgcttgcaaacttggtgaagcaattaatctttgtctcaatcttgacctagaagaaaaaagcaaagaaatccaatctaatatttctgtgttgaataaatatgactggaaaattgttggtgaaactttcaatagtttattgttagatgcaaataaatagtatacgttgatggggaaaatatgaatattgttaaaactgatattccagatctgatcgttcttgaaccaaaagtgtttagtgatgaacgcggcttttttatggagagttataatcagattgaatttgagaaggcaataggaaggcacgtaaattttgttcaggataatcattcaaaatctagtaaaggcgtactacgtgggttgcattatcaattagcaccgtatgcacaggctaaattagttcgatgtgttgtaggtcaggtatttgatgttgctgttgatcttagaaaaaattcaccaacgttcaaaaaatggtttggaataaccctttccgcagaaaataaacgacaattatggatacccgaaggatttgctcatggtttcttggtgaccagtgatgaagctgagttcatttataagacaactaactactatgctcctggtcatcagcaagcaattatttacaatgatcctattttaaacatcgattggcctttctgcagtagtgctctgtcattatcacaaaaagatcaagaagcaaaattattttcagaattattggacagtgaactgttctaataaagtgtgccaccttatccgtctgaaggataggtggttgcttatatttttttgagtatgtttgtataatgacagaaaatagtccgaaatataaacacgataaaagcttaataagttttatctacttattttttatatttacacttattgtaggctttattatcgcaaatacccagtttttggggcgaagtagagactatgataattatatacagatcttttctggtaaagaaggggagggggttcttgaattattttatcgcggattgatgttaataacgaccagctatgaaactatcatttttataattttaacatgttctttttttataaaggcaaggtttctcgctaactattcgcgtaatttttcaggcttgaccttattctttatttattatgcaagcgttgcactttgggttttagattatactcaattcagaaatggtctatgtatttccattttaatgttttccgtatactatttatttataaataaaccgacttatttttatttctcggtattatgtgcaattgcaactcattggtctgctttgccttttttgcttttatatccttttgtctattcaacaaaaataagacgccttggttatttttgtttcagtattcttgttttgattgcgatctcaggagaaggaaaagagatcatatcttttataagaaattttggagtgggacaaaaaataggaaatgaagctggtgtaaatttaataaattcattatcccttaccgctatttcctggtttattattagttacatatcaagcattggaaatgaaaggagaaatttaaggcttttcttttgttatggtgtcatgcaatacgtgacttttagccttttctctctacctgttatggctttccgtattttggaaatgtattttttccttatgctaaccattggggtgtttattaagcaaaaaaagaattattattttattttttgcaaagtgttaattttattgtatctaacatactattatcatatggtctttggagtgattaatgtgtaaggctaaggtgttggctataattgttacttacaacccggaaattattcgattgacggaatgtattaactctttagccccacaagttgagagaataattcttgtagataatggctcaaataatagtgatttgataaaaaatatcagtattaataaccttgaaattattttactttcggaaaacaaaggcattgcatttgctcagaaccatggtgttaagaagggcctggaagcaaaagagtttgactatttatttttctcagatcaggatacttgctttcctagcgatgttattgaaaaacttaagagtacatttacgaaaaataataaaaaaggtaaaaatgttgcttgtgcttctcctttttttaaagaccatcgttcaaattatatgcatccgtcagtcagcctaaatatttttacgagtacaaaagttatatgtagtgaagtagacgatgatctttatccctcgcatgttattgcttctgggatgttaatgtctcgtgaagcatggcgcgtcgtcggaccattttgtgaaaaactctttatagactgggttgatacagaatggtgttggcgtgcattagctaataatatgattattgttcagacaccatcagtcatcatttctcatgaacttgggtatgggcagaaaatttttgctggtcgatctgttacaatacataattctttcagaaatttttataaaatacgcaatgcaatatacttaatgctgcattcaaattatagcttcaagtatcgttatcatgctttttttcatgcgacaaagaatgttgtatttgaaattttatattcgaaagaaaaattaaattcactgaaggtttgttttaaagctgtacgtgatggtatgttcaataatttttaatacgaaaatagttaggctcaaggtgtttaaatggaagaaaataatatgaagacggtcgctgtagttggcacagtgggtgttcctgcttgttatggtgggttcgaatcacttgttcagaatctaattgattatcaatctgatggtatacaatatcagatattttgctcttcaaaaaaatatgataaaaaatttaaaaattataaaaatgcagaattaatctatttgccgataaatgccaatggcgtctctagcataatttatgatattatgtgtttaattatttgtttattcaaaaggccagatgttgttttaatattgggggtgtctggttgtttatttctaccaatttataaactattttcaaaatcaaagattattgtcaatattgatgggcttgaatggcgtagaaataaatggggaacgtttgctaagaaatttcttaaaatatctgaggcgatatctattagaatagctgatattatcatttcagataatcaagcaatagctgattatgtggaaaataagtacaagaaaaaaagtgtagttatagcttatggcggagatcatgccactaatcttagtacaccgatagacaatgatcaaaaaaaagaaggttattatttggggctttgtaggatagagcctgagaataatatagaaatgattctgaatgccttcattaatacagataaaaaaattaaatttatgggtaattgggataacagcgagtatggacgccagctaaaaaaatattattcaaactatccaaatatcaccctactagaacctaactataatattgaagagctttataaactaagaaaaaattgtcttgcatacattcatggacactcggctggtggaacaaacccttctttagttgaagcgatgcattttaatattcctatttttgctttcgattgtgactttaatcgttacacaactaacaatttagctcattactttaatgattctgaacaacttagcttattagcagaaagtttgtcttttggaaatcttaaatgtcgagtattagatttaaaaaattatgctgaagatatgtataactggaggcatatagctgctatgtatgaatctatttattaaacgcattaacaataatataattgaccttatatagcagggaaagatcacgtaacgctgcggcgcgccgatccccatatgaatatcctccttagttcctattccgaagttcctattctttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtatcgataaagaaggtgtgttccataccgaatggctggattaa

SEQ ID NO: 13 - DNA, 13777 bp, synthetic construct
  source 1..13777: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatttgcccgccgggcgtgacaattatgaacgtgcgtcagggcgaacctttaggtttgggccactccattttatgtgcacgacctgccattggtgacaatccatttgtcgtggtgctgccagacgttgtgatcgacgacgccagcgccgacccgctgcgctacaaccttgctgccatgattgcgcgcttcaacgaaacgggccgcagccaggtgctggcaaaacgtatgccgggtgacctctctgaatactctgtcatccagaccaaagagccgctggaccgcgaaggtaaagtcagccgcattgttgaattcatcgaaaaaccggatcagccgcagacgctggactcagacatcatggccgttggtcgctatgtgctttctgccgatatttggccggaacttgaacgcactcagcctggtgcatgggggcgtattcagctgactgatgccattgccgaactggcgaaaaaacagtccgttgatgccatgctgatgaccggcgacagctacgactgcggtaaaaaaatgggttatatgcaagcgttcgtgaagtatggactacgcaacctcaaagaaggggcgaagttccgtaaagggattgagaagctgttaagcgaataatgaaaatctgaccggatgtaacggttgataagaaaattataacggcagtgaagattagcggcgaaagtaatttgttgcgaattttcctgccgttgttttatataaacaatcagaataacaacgacttagcaataggattttcgtcaaagttttccaggattttccttgtttccagagcggattggtaagacaattagcatttgaattttacgggtttagcgcgagtgggtaacgctcgtcacatcgtagacatgcatgcagtgctctggtagctgtaaagccaggggcggtagcgtgctgaaattataaagtcattcttatagaacatcgcatttcaataatataattacacctaaatgaataggatacaacgtgtgcacaattatttaaggcttaaagataaaataaaaaacgtatttttagggttgtatatattgcagttatttaattatatcgcgccattggtaattatccctatcctgataaaatatattgggttgggggaatatggggaattggtctatattacatctatttatcaaatagtggctttgattattgattttggctttacttacacaggacctgtggttgctgcgagacatagatgtgagacccaaaatttacagcgctattactcaatagttgttcttttaaaatcattgctttttataattgcattaacatgtgtatttttattgtgcagattaaatatagtccacttgtcattttttgggtttttgtcaatttttctatgcactattggtaatatattatcgcccaattggtttttgcaggggattggtgattttaaaaaactttcatactcacaagtaatagtgagaataacattgtttatcatacttcttgtttatgtctgtagtggcggagataatgtttttatcctaagttttttgcaaaatgcaacattactcatatgctgtatatacttatggccaaatattcatattagccatgttgttcatcttaaacctaatgaatgcattgtggaatttaagaaggcaggaaatgtttttattggcgtaataggtacgattggttacaatggtctaattcctgtgttaattggaaacctttgcggtaatacgagtcttggtgttttttcaatcgttcaaaaaatgacaacagcatgtcaaagtctaattaatccaatatcacagtatatgttatctcaagtttcagaaattaaacctcaagataaactgttttattatagaattaaaaaaagtttttttgtgcatttaacaattagcataattgcatgtttatgttatatggggttagggcaatatgtggcgacttttataggtaaagttgacgtttcatttgttattattttatttgcgtcaataattaccattttttcatctttaaataatgtccttggtatacagtttcttataccgacagataatgtaaaaatactacgaagtataaatgttatggcgggaattattgttgttagtttgtcctggctgttaatatcacgctttgacattctggggggggttttattaaacctaattggtgagtttcttgtattcagtatgctagcttttattgcccatcgaaagtggggagcgagagtataatgaaagtgaaggcggttcctgctattacattctatttaagtttaatgctgacaattttagtgttactgtttggtaatgaaccaaataaatcacaatatatccttgttatagcaacgataacagttttttatatcgcatatatcactaataaaataacttctccggccagccttctcgttatatcatcttttgtgtttttaggttgtcgccctttattatctttgtttgcaaactatgattataggattgccgattggtttattgaaggatatatggatgacgatgtgattttggctaactatgctataacactaatgtattatggttatacattgggactaattctatgcaaaaatactgaaaaattttatccgcatggtccttatcctgaaaaacaattgctaaaaataaagtttcttttgactttattttttctgggttcgataggtatggttgtaaaagggatattcttttttaactttatagaatctaatagttatgttgatatttatcaatcaaatataacaacgccaataggttatgattttctatcttatttattttattgttcttttttccttatatgtgcgtttcatatacagttcagaacaaataaaaaatttctttttattgcgatatgcattgctgcatttagcaccttgaagggtagtcgtagtgaagctataacgtttcttttaacggttacatgtatatattttaatgaagtaaagacaagaaacttacgtctgctgattacaatgatttttgtttttagcgtcatttttgtgattagtgaatttatctcaatgtggcgcactggagggagtttttttcaattaatgcagggtaataatcctgttataaactttgtatacggcatgggagtatcatatctttccatttatcaatcagtaaaactacaactattgtcagggggatataatgttacctatctattcagccagttaataataacttgctcgtcaatatttaatgtcaaattgagcttgccggaaataagctatagccatttggcctcatacacagcaaacccagaactatataatcttgggttcggacttggggggagttatttagcagaatcgtttttagcatttggtctgattggatgtttcattataccctttttacttttacttaatttaaatgtattggaaaaatatacaaaaaacaaaccaattatatattttgtttattatagtgtgttgccacctatattattcacaccaagagagactttgttctatttcttcccctatcttgtcaaaagtatatttgttgcttttttagttacattatacatccagtataaaaaggattgaccaaaatgtcagaaaaaaatgtcagcataataatcccaagttataacagggctcatattcttaaggaggtcataccaagttattttcaggatgagactttagaggttatagttatcaatgatggatcaacagataatacaaatagtgtattagctgaactgaaggaaaaatattctcagttagttattttagaaaatgaaacgaataaaaaacagatgtattctaaaaaccgagggattgaaatagccaaagggaaatatattttttttggtgatgatgactcttacctcttacccggtgttatatctcggttattggctacaaaatatgagacaggcgctgatgtaatcggcgcaagaatactttatatgaataataacgagaaaacaattgaagattgcataaatcgacataaaaaagaggggcgttttgttagtgatctaaatagattggattttagttatacatgtgatttggaccatccgattgaatgtttttatgcacagccttttgttctagctgaaagggaactaatatcgaaatatcgatttgatatatcttatacgggaaactgctatcgtgaggaaactgatttcatgctatctctatttattaaaaataaaaaatttatatatgattcaaaggctttgttaataaatttacctccaagaaaagcgacgggaggggcaagaacagctaatcgattaaaatatcattacgaaagttgcataaataattatagatttttaaaaaaatataatgataatttgaatcttctttcaggacaaaagcatgctatattttaccgacagtgtcaattcgttctgctaaaaatgaagtcgtttatcgggaagtttttaaaatgattatatatatcgccgcgtataatggttcaggagggcaaggtggggtggaaagggttgttgcccaacaatgtaacattcttaaaaatttgggggttaaagtcattatacttgataaaacatacttcaaaatttctaacaaaattcgtaacaaaaaaatacaagtagcactttatccaatattagtttctctttatttaaccttacaaaaattacgtggcgtgacgtttaaagttattgcacatggctattgttctcctttttataggaatgacatcttaatagctcatggcaatatgaaatgttattttcaaacagtcatgaataaaaaacctaatcggttgtctggcagtggtcttttatctttctatgagcgttgggctggagcattttcaaaaaatatctgggctgtttcaaataaggttaaaagtgaatggaatgagctttacaatattaattcacataaaatcaaagttgttcgaaattttataaatcttgcacaatttgattacactgatgttaatgaagcagaatatgtgacatttgtcgggcgattggaaaaaggaaaaggaatagatgatctgtattacatatgtaaaaatctgccagatacttccttccatttagtttcaagtattcccgccccacaaaattttgcttcgctaaataatgttctgaccagcattgctgtcccctatgcgaaaatgccagaaatatttaagaaatccagagtacttattttaccgtcctattatgaaggatatgagctggttactattgaagcgctatgctgtggttgccctgtgataggctataatgttggtgcaattagagagttgtatgcagaaagttttcctggcgtatttattgccaataataaagaagatttagcacaagtagcctacaaattaattagtcttgataatgaaaaatattatcatttgagacaaactatttatagcaagcgtgagcttttttctgaagagagatatgcggaaattttaacggcggcatttaatgaaaaaaaataagaaactctgtctcatttcaattaactcatataatgaacttaccggaggaggagtatatttacgtacgcttgttagttttctacaaaaacagaatgttaatttaacacttattgataaaaaatcctcaggtaaactattcgaagacaatacttttcaacatatatcatttattaaaggtaaacgtcaggatataatatccaggcttttttttataccatcattttatgtcccttatattttctcaataattaaaattttacggaagcaagatattcttgcttttcacaactctcggcttggattgttatgtctgctttttagaatactcatgccccacaaaaagatcatattgtttacggataacttcgaatatgacttaataagacaaaaagataaaaacataactacttttattgaaaaattaattgtttatctcaatgaatttatcgggcttaagaattcagatttagttagctatattacccggcaagataaaaatgcaatggataaattttatgggattaaaaaaagcagaaatttaattctccctgtgatatttagtagagaaaaaccaactgatgtattgtcagctcactttattaatgagtataatcgattgaataatgataataggaaaaaagtagtatttactgcatcttttgatttttttccaaatatagatgctgccaactatgttttaaatgcagcaaagtctaataatgattattgctatattttggcaggtaggaaaagtactactttgaatcttcctgatttggataatttattttttttcgataatctatctaatagtgaaatgtcatatttattatctgcttgtgatgttttttattctcctatagttttaggaagtggaatgaaaacaaaaattgcagaagcactatcatatggattatatatttatgcgacagagcattccttaatcggctatgatgaaattatacacaataaggagtgtgttaaaaaaatctcacatttggatgaggaatttcctaaagatttcaagatgaaaagtatcaataaacagctaataatgtcttatcagcaaaaatattattcacattatcggtttaatggccatgaacttgatataataaattttgacgattagttagtggagatataatatgaacatattagtaactggtggtgctggatatatcggatctcatacggctattgaattactgaatgcaggtcatgagattatcgttctggacaatttcagtaatgcttcatacaagtgtatcgaaaaaataaaagaaattactcgacgtgattttataacaattactggagatgctgggtgtaggaagacactctccgctattttcgagaaacacgccatagatatagttattcattttgctggctttaaatctgtttcagagtctaaaagtgaacccttaaagtattaccagaataatgttggagtgaccattactttattacaggtaatggaagagtacagaattaaaaaatttatctttagttcatctgcgacagtctatggtgaaccagagataattccaattccagaaacagctaaaattggaggaactacgaatccatatggcacatcgaagtattttgttgaaaaaattctagaggatgttagttccacgggaaaactggatataatttgcttgagatattttaatcctgtcggtgctcattctagtggtaaaataggtgaggctccatctggtatccctaataatcttgttccttatttattggatgttgcgagtggtaaacgtgataaattatttatttatggcaatgattaccctactaatgatggaacaggtgtaagggattttattcatgttgttgacttagcgaaaggtcatttggctgcaatgaattatttaagtatcaattcgggatataatatctttaatcttggtacaggaaaaggttattcggtacttgaattaatcactacatttgaaaaattaacaaacattaaggtcaataaatcttttatagagagaagggcaggggatgttgcgtcttgttgggctgatgcagataaagctaattctttattggactggcaagccgaacaaactctagaacagatgttattggactcgtggcgttggaaaaaaaattatccagacggattctgaatataaaaggtttcagttttatgaatcaatcagagcagagaaaaaaaatactggttcttacacctcgctttccctaccctgtcattggaggggatagattaagagtctatatgttatgtaaagaactttccaaaaaatatgatcttattcttctgagcttatgtgatcaaccactagaacttgaaataaatataaatgactcggtcttcaaagaaattcatcgtgtctatctaccaaaatataaatcatattataatgtattaaaagctttggttacgcaaaaaccgttgcaaattgcttattatcaatcggacacatttaagaataaatacaataaattaattaaacaatgcgatgcagtattttgtcatctgataagagttgctgattatgttaaggatacagacaagttcaaaattcttgatatgacagatgcaatatctttgaattacagtcgcgttaaaaaattagcaagtaaaaaaagtttgcgtgcaattatttattctctggaacaaaaaagattagaatcatatgaacgttctgtggcgaatctttttgatttgaccacttttatttcatccgtagaccgtgactatctctaccctaatctgggcagtaatatccatatagtcaataatggggttgatacatcagccttgagatatataaaaagagaaataaaaatcgataagcctgtggaacttatatttatcggaaatatgtattctttacaaaatatggatgctgcaaaacattttgctaagaatattttaccttgcttgtatgatgagtttaatattatttttaaagtgattggtaagatctcagaaactaataaaaatatattaaattcatttaaaaatacaattgctttaggtactgttgatgatatcaattcttccgcttctacagggcatataggtatatgtcctgttcgtcttggagcaggcgtacaaaataaaattcttgaatacatggctttaggtttaccatgtattacatctagcattggttatgaaggtattaatgcaaaatcaggtagcgaaatttttgttgcagatacagtagagcaatataaaaacgtactaagagaaataatttacgattataatcgttatactgaagtggctgaaaatgcccgtagttttgtagaaaataatttttcttgggaatcaaaagttgccaatttaatgaatacattagatgagaaattatatgaacaataataaaattattacacctatcattatggctggtggttcaggcagtcggttgtggccactatcaagaattctctatccgaaacaatttcttagcctaatcggtagtcataccatgcttcaaacaacggctaatcgtctggatggtttggattgtaccaacccttatgtcatttgtaatgaacaataccgctttatagttgctgaacagcttagaaaaatcgatagattgacttcaaagaatatcatccttgagcctgttgggcgtaacactgcccctgcaattgcattagcggcgttgctgatgtctaagtctgataaaagtgcagatgatcttatgctcgtactggctgcagatcacgttatacacgatgaagaaaaattttgtaacgctgttagatcggcaattccatacgctgctgatgggaaattggtaacatttggtataattccagacaaagcagaaactggttatggttatatacatcgaggacaatatattaatcaggaagattcggatgcatttatagtgtcatcatttgttgaaaagccaaatcatgagacagccactaaatatcttgcttccggtgagtattattggaatagcggtatgtttttgtttagtgcaaatcgttatatagaggaacttaaacaatttcggcctgatattttatccgcttgtgaaaaagcaattgcttcagcgaactttgaccttgattttgtgcgtttagatgaaagttctttctctaagtgccctgaagaatcaattgattacgctgtaatggaaaaaacaaaagacgcaattgttattccaatggatgctggctggagtgatgtcggttcatggtcttctctttgggaaattaatgataaagactcagacggcaacgtaatagttggggatattttctctcatgaaacaaagaattctttcatatatgccgaatcgggaattgttgctacagttggagtggaaaatttagttgttgtccaaacaaaggatgctgttcttgtctcagagagaaataaagttcaggatgtaaagaaaatagtagaacaaattaaaaattcaggtcgtagcgagcattatgttcatcgcgaagtatatcgtccttggggtaaatatgattccattgacacaggggagcgttatcaggtcaaacgtataacagtaaatcctggtgaaggactttctttacaaatgcaccatcatagggcagaacattggatcatagtttctggaactgcaagggtgactataggttctgaaactaagattcttagcgaaaatgaatctgtttacatacctcttggtgtaatacactgcttggaaaatccagggaaaattcctcttgatttaattgaagttcgttctggatcttatttagaagaagacgatgttatccgttttcaggaccgatatggtcgtagctaaatttttgataatgtaacgttagtagaagagcgctaatatttttagttaatctgtaataagtattatttgtttaaggtatatcatgtcgagtttaccctgctttaaagcctatgatattcgcgggaaattaggcgaagaactgaatgaagatattgcctggcgcattggtcgcgcttatggcgaatttctcaaaccgaaaaccattgtgttaggcggtgacgtccgactcaccagcgaaaccttaaaactggcgctggcgaaggggttacaggatgcgggcgtcgatgtgctggatattggcatgtccggcaccgaagagatctatttcgccacgttccatctcggcgtggatggcggcatcgaagttaccgccagccataacccgatggattacaacggcatgaaactggtgcgcgaaggggctcgcccgatcagcggtgataccggactgcgcgacatccagcgtctggcagaagccaacgactttcctcccgttgatgaaaccaaacgcggtcgctatcagcaaatcaatctgcgtgacgcttacgttgatcacctgttcggttatatcaacgtcaaaaacctcacgccgctcaagctggtgattaactccgggaacggcgcggcgggtccggtggtggacgccattgaagcccgctttaaagccctcggcgcacccgtggaattaatcaaagtgcacaacacgccggacggcaatttccccaacggtattcctaacccgctactgccggaatgtcgcgacgacacccgcaatgcggtcatcaaacacggcgcggatatgggcattgcctttgatggcgattttgaccgctgtttcctgtttgacgaaaaagggcagtttattgagggctactacattgtcggcctgctggcagaagcgttcctcgaaaaaaatcccggcgcgaagatcatccacgatccacgtctctcctggaacaccgttgatgtggtgactgccgcaggcggcaccccggtaatgtcgaaaaccggacacgcctttattaaagaacgtatgcgcaaggaagacgctatctacggtggcgaaatgagcgcccaccattacttccgtgatttcgcttactgcgacagcggcatgatcccgtggctgctggtcgccgaactggtgtgcctgaaaggaaaaacgctgggcgaactggtgcgcgaccggatggcagcgtttccggcaagcggtgagatcaacagcaaactggcacaccccgttgaggcgattaaccgcgtggaacagcactttagccgcgaggcgctggcggtggatcgcaccgatggcatcagcatgacctttgccgactggcgctttaacctgcgctcctctaacaccgaaccggtggtgcggttgaatgtggaatcgcgcggcgatgtaccgctgatggaagaaaagacaaaacttatccttgagttactgaacaagtaattcagtaatttcatataaatgggttttaaaaaacggaaaagatgagatatccggtgtggtatatccaaggtaatgctattcagtatctctatgagtgagttaacatctataccacatttaagccgcacacttcgggatccccatatgaatatcctccttagttcctattccgaagttcctattctttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtatcgataaagaaggtgtgttccataccgaatggctggattaa

SEQ ID NO: 14 - DNA, 15027 bp, synthetic construct
  source 1..15027: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcgatcgcttaagatctaggatttcattatgttacttcctgtaattatggctggtggtaccggcagtcgtctctggccgatgtcacgcgagctttatccgaaacagttcctccgcctgttcgggcagaactccatgctgcaggaaaccatcacccgactctcgggccttgaaatccatgaaccgatggtcatctgtaacgaagagcaccgcttcctggtggctgaacagctacgccagctcaataagctgtcgaataatattattcttgagccggtcgggcgcaacaccgccccggccatcgccctggcagcccttcaggccacccgcgacggcgacgacccgctgatgctggttctcgccgctgaccatatcatcaataaccagtcggccttccacgacgccatccgggtcgccgagcagtatgctgatgaaggtcatctggtcaccttcggtatcgtgccgaatgccccggaaactggctacggttacattcagcgcggcgtggcgctcaccgatagtgcccattccgcgtaccaggtggcccgctttgtggagaagccggatcgcgagcgcgccgaggcttacctcgcctccggggagtactactggaacagcggcatgtttatgttccgcgccaagaaatacctcatcgagctggccaaataccgtccggatatcctggaagcctgccaggctgcggtgaatgccgccgataatggcagcgatttcatcaatatcccgcatgatattttctgcgagtgcccggatgagtccgtggactatgccgttatggagaaaaccgccgatgcggtggtggtcggtctcgatgctgactggagcgacgtcggctcctggtccgcactatgggaggtcagcccgaaagacgagcagggcaatgtcctcagcggtgacgcgtgggtacacaacagcgaaaactgctacatcaacagcgacgagaagctagtggcggccattggcgtagagaatctggtgattgtcagcactaaggacgccgtgctggtgatgaatcgcgagcgttcccaggacgtgaagaaggcggtcgagttcctcaagcagaaccagcgcagcgagtacaagcgccaccgtgagatttaccgcccctggggccgttgcgacgtagtggtccagaccccgcgcttcaacgtcaaccgcatcacggtgaaaccaggcggtgccttctcgatgcagatgcaccaccatcgcgccgagcattgggttattctcgccggcaccggtcaggtgactgtcaacggtaagcagttcctgttgtccgagaaccagtccacctttattccgattggcgccgagcactgcctggaaaaccctggctgtattccgctggaagtgctggagatccagtcgggggcgtaccttggcgaggacgacattattcgtattaaagaccagtatggtcgttgctaattattttcgggacaagacgcagaatgacacagttaacttgttttaaagcttatgacatccgtggtgaactgggtgaggaactgaacgaggacatcgcctaccgtatcggtcgcgcctacggcgaatttctgaaacccgggaagatagtggtggggggcgatgtgcgcctcacaagcgagtcgctgaagctggcgctggcccgcgggttaatggacgccggtaccgacgtgctggacatcggcctgagcggtaccgaagagatttactttgccaccttccaccttggggtagatggtggcatcgaggtgaccgcgagccacaatcctatgaactacaacggcatgaagctggtgcgcgagaatgcgaagcccatcagcggcgacaccggcctgcgggatatccagcgcctggcggaggaaaaccagttcccgccagtggacccggcgcgtcgcgggaccctgagcaagatatcggtactgaaggagtatgttgaccatctgatgagctacgtggacttctcgaacttcacccgtccactgaagttggtggtgaactccggaaacggggctgcggggcacgtgattgatgaggtggagaaacgcttcgcggcggctggggtgccggtaacctttatcaaggtgcatcaccagccggatggccatttccctaacggtatcccgaatccgctgctgccggagtgccgccaggataccgccgacgcggtgcgcgagcatcaggccgacatggggattgcctttgacggcgacttcgatcgctgcttcctgttcgatgacgaagcttcgtttatcgaggggtattacattgtcggcctgctggctgaggcgttcctgcagaagcagccgggagcgaaaatcattcacgacccgcgcttgacgtggaacacggtagacatcgtgacccgcaacggcggccagccggtgatgtcgaagacggggcatgcgttcatcaaggagcggatgcgtcaggaagacgctatctacggcggggagatgagtgcgcaccattacttccgcgatttcgcctactgcgatagcgggatgatcccgtggctgctggtggcggagctgctgtgtctgaagaacagctcgctgaaatcgctggtggcggaccgccagaaggcgttccctgcgtcgggagagatcaaccgcaagctaagtaatgctgctgaggcgatcgcccgcatccgggcgcagtatgagccggcggctgcacacatcgacacaacggacgggatcagtattgaataccctgaatggcgctttaacctgcgcacgtctaacaccgagccggtggtgcgtctgaacgttgagtccagagctgatgtggcgcttatgaatgaaaaaacgaccgagctgttacacctgttaagcggggaataaggtgagagatttactaacgacgatttatcgttatcggggatttatctggagcagtgttaaacgtgattttcaggcacgctatcaaactagtatgctgggcgcactatggctcgttttacaaccgctctctatgattctggtctataccctggttttttccgaggtgatgaaggcaagaatgcccgataataccgggtcgtttgcctatagtatttatctctgttccggggtactgacctggggattatttactgagatgctggataaaggtcagagcgtatttattaacaatgctaatctgatcaagaaactcagttttccgaaaatctgtctgccgatcatcgtgacgttatcggcggtgctaaatttcgcgattattttcagtctgtttctaatttttatcattgtcaccggtaacttccccggctggctctttctctcggtgataccggtcctgcttttgcagatcctgtttgccggtgggctggggatgatccttggtgtcatgaacgtctttttcagggatgtggggcaactggttggcgttgcgctgcaattctggttttggttcacacccattgtttatgtactgaattcattacctgcatgggcaaaaaatctgatgatgtataacccgatgactcggatcatgcaatcttatcagtccatcttcgcctatcatctggcccccaactggtattcgctatggccagtattggctctcgccattattttctgcgtcatcggtttcaggatgttccgcaagcatgcggcggatatggtggatgaattataatgagttatatcagagtaaataatgtcggtaaggcgtatcgccagtatcactcaaagaccgggagactgatcgaatggttatcccctctgaataccaaacgccataatttgaaatggatcctccgcgatattaatttcgaagtcgctccgggcgaggctgtcggtattatcggtatcaacggtgcaggcaagagtaccctgcttaaactcataaccgggacgtccaggccgacgactggagaaattgaaatctccggacgtgtcgctgcattactcgaattggggatggggtttcattctgatttcactggtcggcagaatgtttatatgtctgggcaactgttggggttatcgtcagagaaaataactgaactgatgccgcaaattgaagagtttgctgagattggggactatatcgatcaacctgtgcgcgtctactccagtgggatgcaagttcgattagcttttagtgtagcgacggctatccgtcctgatgtgctaattatcgatgaggcattatctgttggggatgcatatttccagcataaaagctttgagcgtattcgaaaatttcgtcaggaagggaccacgctgttgctggtatcccatgataaacaagcgatccaaagcatttgcgaccgggccattttattgaataaaggccaaattgaaatggaaggtgaacctgaagcagtgatggattattacaatgctcttctggccgataaacaaaatcagtccattaaacaagttgagcataatggtaaaacgcaaactgtttcaggcactggtgaggtgactatctctgaggttcatcttctcgatgaacagggcaatgtgactgaatttgtttcggtagggcatcgtgtcagcttgcaggtcaacgttgaggtcaaggacgatattcctgagcttgttgtcggatatatgattaaggatcgacttgggcagccgattttcgggaccaatacgtaccatctcaatcagacactcacctccctgaaaaaaggagaaaagcgttcgttcttattttctttcgatgcgagattgggggttggctcctattctgtcgctgtcgcgttgcatacttccagtacgcacctcggcaaaaactatgaatggcgcgatctggccgtggtattcaacgtcgttaacacggaacaacaagagtttgtcggcgtgtcctggttgccgcctgaactggagatttcttaatgggttcgtcgttttatcgttcatttgaagaacgacacagaggttcggttgaagaaatcaagcgccgcttgagtttttatttaccttttcttgcaggtctgaaggacatttatcctgatggcgtgattgcggatattggttgcggacgtggcgaatggttggagatcctgactgaaaatggcattgcgaacatcggcgtcgatctcgatgatggcatgctggcgcgcgccagggaggccggactgaatgtgcagaaaatggattgtctgcagtttttgcaaagtcaggcggatcagagcctgatagcgttgaccggttttcatattgctgagcatttgccgtttgaggtcctgcagcaactcgccatgcataccctacgggtgctgaaaccaggtggtttgctgatcctcgaaacgccgaacccggagaatgtaagcgtcggcacctgttcattttatatggatccaacgcataatcatcctctgccaccgccactgcttgagtttttacctattcattatggttttacccgagcaattaccgttcgtctgcaggaaaaagaggttcttcaatctccggatgcagccgttaatttggtcgatgtactcaaaggggtgagccccgactacagcatcattgctcagaaagcagcgccaacagatattcttgaacgctttgacaccctgtttacccagcagtacggtctgacgctggatgctctgagcaaccgttacgatgcgattttgcgccaacagttttcgtccgttgtctcacggctggagacgttgaaccaaacctatatgcaacagataagccaaatgtcagagactattcagacgttgcaaggtgaggttgacgatctgagtcatgtcatcgatcagaaccatcagcttcatcagcaaatggcggatttacataacagtcgttcatggcgtattactcaaccactacgctggttgtctttgcaacgtcaattattacgtcaggaaggggctaaagtgcgagcccgtagggctgggaaaaaaatattgcgcaaagggatggcgctctcgctggtctttttccatcgttaccctaagtctaaggtttatctgtttaaggttctgagaaaaactggctgctatacattgctacaacgtttgttccaacgcgtaatgctggtgcaatctgacacgatgatgatgcagtccagaagatatgatgtgggtactgaagaaatgacaagtcgcgcgatgagtatttataacgaattaaaaaataaaaatacggagaaataacgatgcgtattgtcatagatttacaaggcgcacagacggaaagccgctttcgtggcatcggtcgttatagtatcgcaatcgccagaggcataatcagaaataacagccggcatgagattttcatcgcgctatccgccatgctggatgagtcgattgcaaatattaaggcgcaatttgccgatctcctgccggcagaaaatatagtcgtatggcatgccgtaggccctgttcgtgcgatggaccaaggtaatgaatggcgtcgggagagcgcagaactgattcgggaagcgtttcttgaatcattgtgtccagatgtcgttttcattacgagtttgtttgaaggtcatgtcgacgatgcggctacatcggtacacaaatttagtcgtcagtataaagtagccgtactgcaccacgatcttatccccctcgtgcaggcggaaacctatctgcaggacgatgtatacaaaccctactatttacagaaagttgagtggttaaaaaacgctgaccttttgttgactaactctgcttataccgcacaggaagcgatcgagcatctgcatttacagggcgatcatgtgcagaatattgcagccgcagtcgattctcagttttgtatggcggaggtggcagcgagcgaaaaagagaccgtccttggccattacggtattcagcgcgagttcatgttgtatgcgcccggaggatttgactcaaggaaaaactttaaacggttgattgaggcctatgccgggctcagtgatgccttacgtcgcagtcatcaactggtcatcgtcagtaagctttccatcggtgatcgtcagtatctggaatcccttgcgtcaggtaatggtttacagcagggcgaactggtactcactggttatgtgccggaagatgagctgatccagctctatcgcctatgtaagctgttcatctttgcttcactacatgaaggttttgggttgccggttctggaagcaatgtcgtgcggtgcgccggtgattggctcaaatgtcaccagtattcctgaagtcatcggtaatcctgaggcattattcgacccgtattctgtctcttccatgagggataagatcgcgcaatgtttgactgatgataccttcctcgcgcgtctgaaagaaatggcgcagcagcaagcgcgtaatttctcttgggataaagctgcggtgactgctctggaagctttcgaaaagatcgcggtagaagacaccggtactgcgcaggttttgcctgaagctttgattcagaagatccttgctatctcacaagggcagccagatgaccgcgatctgcgcttgtgcgcaacggccattgattacaatctgaaaacggcagaactttatcaaatcgacgataaatcgctgaactggcgtgtggaaggcccattcgatagctcatatagtctggcgttggtcaaccgcgaatttgcccgggcactctcagccgatggtgtagaggttttattgcattccactgaaggaccaggtgattttgccccagatgcctcgtttatggcacagtcggaaaatagtgatcttctggcattttataatcaatgtcagacccgcaagagtaacgaaaagatagatattattagcagaaatatctatccaccgcgggttaccaaaatggatgccaaagtaaaattccttcattgttatgcttgggaagaaacgggctttccgcaaccgtggatcaatgaatttaatcgggaacttgacggagtgctgtgtacttcggaacatgttcgtaaaatactgattgataacggactgaatgtgcccgcatttgttgttggcaatggctgtgaccattggctcaatatcccagccgagacgacaaaagatgtggatcacggaacattccgtttcctgcacgtctcttcttgtttcccacgcaaagggatacaggcaatgcttcaggcttgggggaaggcgttcactcgtcgtgacaatgttatcttaatcattaagacttttaacaatccgcacaatgaaattgacgcatggctggctcaggcccaggctcaattcatagactatcccaaagttgaagtgatcaaagaggatatgtcagccaccgagcttaaagggctttatgaaagctgtgatgttttggttgctccaggttgcgctgaaggctttggtttacctattgctgaagcaatgctgagtgggctaccggctatcgtcaccaattggagcgggcaacttgattttgttaattcacaaaattcatggctggttgactatcagttcactcgggtaaaaacgcactttggtctgttttcctcagcctgggccagtgtggatattgacaacttaacagatgcattaaaagcggcagcctcaaccgataaatcagtgctgcgtgacatggccaatgctggtcgcgagcttcttctgcagcagtttacctggaaagcggtggctgatcgttcttgccaggcggtcaagactctgcgtgcgcatattgatattgcacagcatcgggcgcgcattggctgggtgacgacctggaacacgaaatgtgggatcgcaacctattcccagcatctggtggaaagcgcacctcatggcgcggatgttgtttttgctccccaggtcagcgctggcgatcttgtgtgtgcagacgaagagtttgtacttcgcaactggattgtaggtaaagagagcaactatctggaaaacctccagccacacattgatgctctgagactcgatgtcattgtgatccaattcaactatggattctttaatcatcgagaactgtcggcgtttattcgtcgccagcatgacgccggtcgttcagttgttatgacgatgcactcaactgtggatccgctggaaaaagagccgagctggaatttccgtcttgctgaaatgaaagaggcgctggcactttgcgaccggttgttggtgcattcgattgccgatatgaaccgccttaaagatttaggcttaactgcgaatgttgctttattcccgcacggtgttatcaactactccgcagcgagcgtcacacgtcaacagcagtctttaccgctaattgcgagctatggcttctgcttaccgcataagggcctgatggaactagtagaatccgtccatagactcaagcaagccggtaaaccggttcgtttacgactggtgaacgcagagtatcctgttggggagtcacgcgatctggtggcagagcttaaagctgctgctcagcggttaggtgttaccgatctgattgagatgcataatgatttcctacctgatgcggagagtctgcggttgctttcagaagccgatcttctgatttttgcttatcagaatactggggagtctgctagcggggcggtacgttatggtatggcgactcaaaaacctgttgcggtaacgcccctggcgatatttgatgatttggacgatgccgtctttaaatttgatggatgcagcgtcgatgatatcagtcaggggattgaccggatcctgaattccatccgtgaacagaactcttgggcaaccaggactcaacaacgtgccgatgcatggcgggaacaacatgattatcaagctgtttcacgccgtctggttaatatgtgtcaaggcttagctaaagctaaatattttaaataaaaatatctctcttgtattttttgcctttgaatacaagaggggttagataatgtgtcatttattatgaaaattatttttgctactgagccaattaaatacccattaacgggcatcggtcggtattccctggagctggttaagcggctggcggtcgcccgcgaaattgaagaattaaagctatttcacggtgcgtcgtttatagaacagatccctttggtggagaataaaagcgataccaaagccagcaatcatggtcgtctgtcggcgtttctacgccgacagacgctgttgattgaggcttatcgcttgctgcatccgcggcgccaggcgtgggcattgcgcgactataaggattatatctaccatggccccaatttttatctgccgcataaactggaacgcgccgtgaccacgtttcatgacatatccatttttacctgcccggaatatcatccaaaagatcgggttcgctatatggagaagtccctgcatgagagtctggattcggcaaagctgatcctgaccgtttctgatttctcgcgcagtgaaattatccgcttgttcaactatccggcggagcggatcgtaaccaccaagctagcctgcagcagtgactatatcccacgcagcccggcagagtgtctgccggtactgcagaaatatcagctggcgtggcaggcctacgcgctatatatcggcactatggagccacgtaaaaatatccgaggcctgctgcatgcctatcagctgctaccgatggagatccgcatgcgctatccgctaatccttagcggctatcgcggctgggaagacgatgtgctgtggcagttagtcgagcgcggtactcgggaaggctggatccgttacctcggatatgttccggatgaagacctgccgtatctgtacgcagcggccagagtctttgtttatccctccttctacgagggattcggtttacctattcttgaagcgatgtcttgcggtgtgccggtagtatgctccaatgtcacctctttgcctgaggttgttggcgatgccggcctcgttgccgatcctaatgatatagacgcgattagcgcgcaaattttgcagagcctgcaagatgatagctggcgggaaatcgccaccgcgcgcggtcttgctcaggcgaaacagttttcgtgggagaactgtgcgacacagaccattaacgcctataaattactctaagggtgtcagttgagagttctacacgtctataagacttactatcccgatacctacggcggtattgagcaggtcatttatcagctaagtcagggctgcgcccgccggggaatcgcagccgatgttttcacttttagcccggacaaagatacaggtcctgtcgcttacgaagatcatcgggtcatttataataaacagctttttgaaattgcctccacgccgttttcgctgaaagcgttaaagcgttttaagctgattaaagatgactacgatatcatcaactaccattttccgtttccctttatggatatgctgcatctttcggcgcggcctgacgccaggactgtggtgacctatcactctgatatagtgaaacaaaaacggttaatgaagctgtaccagccgctgcaggagcgatttctcagcggcgtagattgcatcgttgcctcgtcgcccaattacgtggcttccagccagaccctgaaaaaatatctggataaaacggtggtgatcccgtttggtctggagcagcaggacgtgcagcacgatccgcagagggtcgcgcactggcgggaaactgtcggcgataagttctttctcttcgtcggcactttccgctactacaaagggctgcatattctgatggatgccgctgagcgtagccgactgccagtggtggttgtagggggcgggccgctggaatcggaagtgcggcgtgaagcgcagcagcgcgggctgagcaatgtgatgtttaccggcatgctcaacgacgaagataagtacattctcttccagctctgccggggcgtggtattcccctcgcatctgcgctctgaggcgtttggcattacgttattggaaggcgcacgctttgcaaggccgctgatctcttgcgagatcggtacaggtacctctttcattaaccaggacaaagtgagtggttgcgtgattccgccgaatgatagccaggcgctggtggaggcgatgaatgagctctggaataacgaggaaacctccaaccgctatggcgaaaactcgcgtcgtcgttttgaagagatgtttactgccgaccatatgattgacgcctatgtcaatctctacactacattgctggaaagcaaatcctgagcggccgcgagctcgtcgactcgaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 15 - DNA, 11283 bp, synthetic construct
  source 1..11283: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcatgagcaaaactaaactaaatgttctttaccttgcaataagtcagggtgccaattacctactgccattattaatttttccttatcttgttagagtcattggtgtatcgaattttggtgatctgagtttttcattgataactatacaagtgttgttaatggttgttgaatatggttttggatatagtgggacaagagaaatagcactaaataacgataaaaaataccattctgaatttttttgcggtgtggtgcttgctcgttttatattaatgctaattgcagctataatactcataatactctgttttttttatgtttttaacgacgttaagtctttgttatgtgttggttttctgtccgtaattgcaggtgttttcaatccaaattggtttttgcaaggtaaggaaatgatgagtgtgatggctgtgctgtcactattttcacgaggcatagcagtcgttgcagtttatctaattataaaacccgcaacgccgatgtacatcagtgccttattattgagcatgccatatattttgtattcattctgtggcgttgcctacttacttattatcaaggagatttttttatgtaggccaccgataaagaaaattcaagtaattttaaaaaatggatttcattttttttgttcaacacttgcgactagtgcatacacaatgttgacccctcttgtattgggtggcgtatctggaaagtttgatgtaggcatctttaactcagctaacatgatcaaacaaggtttggctggacttgcatcaccattagtccaagctttttatccaagaattaacattttgcaaagagagaatccatatattgcaaacttaaaatctagaatgattcttaaatacttgcttgttttttacatggctttagcaataccatttttactttttgccaaccaattatcattattaatattcggcatgaaaggtgaagtaattgcaggtgcaatgcaattaatgacattgcttcctatattcataggttttaatacagttgtcgggttacttgtattagtacctaatgggatgcaaaaacagtatttcaaatctattttcctaggaactattacttgtttaagcatagtttatccagcatgtaaatattatggagcaacgggtgcgattgtgagtcttattgtagctgaaattttcgttggcatgggaatgcttaaacaattcattaaagtaaataaaaccgtatgtaggcctcataaattatgaatatctcggtaataatatctgtttggaaacgcccagttcaattagaattgattctctctgagctcgattctcaggctaaagacaatagtctacacctagaagtaattgtttccgatagtcatagtggtaaagaaattgatgatgtagttgctgataatattcataaaaagaaaaatattaatattatccatcaacatactaaaaatatactctccgctaagcgcaatttcggagcatccctagcccatggggattatttaatatttcttgatgatgattgtatacccgcaagtggatatatatcatcgttgctgaactatttaaaaaaaatgaatagtaaaagcgttttatgtggggaagttagattcgaaaatgaactcattgagaccagcaattactatcgctacaggaactctttacaccctaagtttagtgatagtcctgatatctctatgaatgcctggacttttgtcgcaatgaattgtgttcttgatagaaaggcattttcatcaggtatagtttcatataatgaaaattttattggttatggttgtgaagatcatgagtttgggtggcaacttgaaaaaaatgacttcaaaattatttttgctgattttaaaatattacatcacgaatacagtggcgatatagaaggatatacaaaaaaaattcgtgctacagcacgtgatggtatgaatgtattaagcaaagtaaggcctgaaatgttttctactaataaaaaattattcctagttgagaaaatatttagtaaacacaaaacgtttagtaaaatatgccaatcaatatttttcaataaatttatttttaaaaaaataatacaatttttaaaaaaaacagatgcaaataaaaaactctatttcccaattctttacagatatgtgttgatttcggcatatatacatggtattggagagcgtggcacctcaaaaacagatgatttgcttaagaactggtatatatagatgatgctatcttcatttattaagacatttgtatggaaggtaaaaaacaatgaagtataatgcattgatggcttttttattattttttgttgttttttttagattgtcgctgataatacctttcttatatttggcatttattcctgcattttttggtattatgtatttagtgcgtaattttatgattactatgggcaatggattggtatctatagatcgtaaaaatttgttgctgttatctatattcataattatttttttattttgtttggttttcgatttgtttcaaaaaagccattcttttcaaagttattttaccgttagattatttatgttgtttttattttcatttgttcctgcgtattatttagtaaatagattcataaagggtgacttgaaattaatggagcgaatattagtgtattctctctgggttcaaatagttattttttttggtatgtatataagtccagagttaaaaagattgttatatactttctttggtatgtctgactctgttaatctttgggaacaaaatgctaaagtaagaggatttgggttgtcgggtgaaataaatttcatgacaccatttttgatgatctatatgtcattttttatgatgaaaaggcgttatgctttaattactttaatttgtctgactcaaatcgtaaattctaacatggctgtgattgcagccattattggtatcggttgctctagacttaatattaatataaaaattgcaacagtattgattttgggagttttagtttatagcttaggagcggtgttctttcctcgattttatgatgagttcgtttctggagatggcacaagaactctggatatcttattacagcaacatgtgtttgttgtaggtaatttagatttttttaatattatatttggattacagcaaaacatatcttcatcaatccccgatattaaacaaagttcggatatgggctgggttatactgtttaattacggtgggttaacatttattacactctttttatttttaatctttactatttctattgcgacatttggaatgacatatcaagcaattatatggatgttaattgggataattttcaataccaaaggtttagttttaggatctaacggctatttctttctatcttttatatatatgtttttgaatagagtaacacttagtggacagagttcaattactaataagttaggtcaagtaagtaaatagcttccagagtatatttgtcaatgatttgaggttcggttattatgttttcatctaaaacactgttaattactggtggtactggctctttcgggaatgctgtattaaatagatttcttgatacagatattgcagaaatccgtatatttagtcgtgatgaaaaaaaacaagatgatatgcggaaaaaatacaataatcaaaaattaaagttctatattggtgatgtcagagattaccgtagtattttgaatgcgactcgcggtgttgattttatatatcatgcagcggcacttaagcaagttccatcatgtgaatttcatcctatggaagccgttaaaactaatatccttggtacggaaaatgttcttgaagcagctatagcgaatgaagtgaagagggttgtatgcctaagtactgataaagctgtatacccgattaacgcaatgggtatttcaaaagctatgatggaaaaggtcatggtcgcgaaatcccgtaatgttgatcgcaataaaacagtaatatgtggtacccgttatgggaatgttatggcatctcgcggttcagttattccattatttgttgatcttattagagcgggcaagccactcacaataactgatcctaatatgacccgctttatgatgactcttgaggatgcggtagatttagttctttatgcgtttgaacatggtaataatggtgatatctttgtgcaaaaagcacctgcagcaactattgacacattagctattgctttaaaggaattactaaatgttcctgaccatccggtaaatgtcattggaacgcgtcatggcgagaaattatatgaagctctacttagtcgtgaggaaatgatcgctgctatagatatgggcgattattaccgtgtcccgccagatcttcgtgaccttaattatggcaaatatgttgagcaaggtgatagccgaatatctgaaatagaagattataactctcataatactcaacggttagatgttgaaggcatgaaagagctcttgctaaaattagcctttattcgagcaattcgtgctggtgaaaaatataatctggattcatgatatgaaaatattagttactggtgcaaatggttttattggtcgtaatttatgtttgaggcttgaggaacttggttataaagatcttattagaattgatcgagaatcaacgaagcaagatcttgaacaaggcttacaggatgccgattttatttatcacttagctggtatcaatagacctaagactgatgatgagtttatttctggaaacagtgatttaacaaagcatatagttgagtatctcctttctattggtaagaatacaccaattatgctaagttcttcgatacaagctgaacttaataatgcttatggggttagcaaagctgtagctgaaagctatgtcgaaaaatatgctgctgctagtggttcttcgtattatattttcagatatccaaacgtttttggtaaatggtgtaagccaaactataattcttttatagcaactttttgctacaatatttccaatgatattgagattactatcaatgatgcagcagcgccagtcaatctggtctatattgatgatgtttgtactgatgctatagctcttctctctgggacggttgaaagtggatataaagttgttgcaccaatttattcaacaacagttggtgaagttgcagaattaatttatagcttcaaaaatagccgttccaccctgatcacagaggctgtcggggcgggatttacccgtgcattgtattctacatggctgagttatttaccagcagagaagtttgcgtacaaggtacctttttatggggatgcccgcggagtcttttgtgagatgttgaaaacgccttcagcggggcagttttcattttttactgctcaccctggtattacgcgtggcggacattaccatcacagtaaaaatgagaagtttttggtcattcgaggtcaggcatgctttaaatttgaacatgtgattaccggtgagcgatatgaactgaaagtttcatcgggtgagtttaagattgttgaaacagttcctggttggacacatgacattacaaatattggaactgatgaattaatagtcatgctctgggcaaatgaaattttcaaccgtgatgagcccgatactattgcgagacctctataatgaaaaaattaaaagttatgtctgttgttggaacccgtcctgagattatccgtttgtcgagggttcttgctaagtttgatgaatactgcgagcatattattgtccatactggtcaaaattatgattacgaattaaatgaagtgttcttcaatgacttgggtgttcgaaaacctgattattttttaaatgcagcgggtaaaaatgcggcggaaaccattggtcaggttattattaaggtagatgaagtattagaaatcgaaaaacctgaagcaatactggtattgggcgatacgaattcatgtatttctgccattccggccaaacgccgtaaagtgcctatatttcatatggaagcaggtaaccgttgtttcgatcaacgcgtgcctgaagaaaccaacagacgtattgttgaccatacggctgatatcaatatgacctacagtgatattgctcgtgaatatctcttggctgaaggtatcccagctgatcggatcataaaaactggtagccctatgtttgaggttctttcatattatatgccccaaattgatggttcagatgtgctatcgcgtttgaatctacagtctggtgagttttttgtagtaagtgcgcatcgtgaagagaatgttgattctccaaaacagctcgtaaagcttgcgaacattctaaatactgttgctgaaaaatataatcttccagttattgtctccacacacccaaggacacgtaaccgaatccgtgagcaaggaattgaatttcattcaaatataaatctactgaaaccattgggtttccatgattataaccacttgcagaagaactcacgagctgtgctttcagatagcggtactatcactgaagagtcatccatcatgaatttcccagcggtaaacatccgggaagcgcatgagcgtccggaaggctttgaggaagcatccgtcatgatggtggggttagagtgtgaacgcgtattacaagcgctggatattctggcaacacaaccgcgaggtgaagtccgtcttttacgtcaggttagtgattacagcatgccaaatgtgtcggataaagttgtcagaattgttcactcttacacagattatgttaagagagtcgtctggaaagaatattgatgaaacttgctttaatcatagatgattacctgcccaacagtactcgtgttggtgcaaaaatgtttcatgaacttgctcaagaatttatccagcgtgggcacgatgttacggtaattactcctggtacgggcatgcaagaagagatttcttttgatacctttcagggggtaaaaacatggcgttttaaaagcgggccgctcaaggatgtaagtaaaattcagcgagcggtcaatgaaacgcttttgtcctatcgggcgtggaaagccatcaaaaaatgggtaaaaaaagagacctttgagggggtgatttattattcaccttccatattctgggggcctttagttaaaaaaattaaagctcgttgccaatgtcctgcttatcttattttaagagatatgtttccacaatgggtaattgatgcaggaatgcttaatgctggttccccaatagaacgctactttcgtctttttgaaaaaatatcttatcgtcaggcaaatcgtattggacttatgtctgataagaatcttgatgtttttcggaaagataataaaggctatccgtgcgaagttttgcgtaattgggcatccctaacaccaacgatcatacccaaggattatataccactacgtaagcgacttggcctagaggataaaaccattttcttctatggtggaaacataggtcatgcacaggacatgacaaacttgatgcgacttgtgagaaacatggcagcatatcctcaagctcatttcctatttattggccagggggatgaagttgaattaattaattcattagcatctgagtgggcattgacgaatttcacctatttgccctcggttaaccaagatgaatttaagttcattttgtcggaaatggatatcggcttgttttctctttccgctagacactcttcccataattttcctggtaagttattaggctatatggttcagtcgctacctattttaggtagcgtaaatgccggaaatgatttgctcgacattgtcaatcaaaataatgcgggattaatccatgtcaatggtgaggacgataaattatgtcaatctgcgctattaatgttgcatgatattgatgtgcgccggcaacttggttcgggggcgaatatattgttgaaagaacaattctccgttgagtctgcggcacagacgatagaaatgaggttggaggcatgcaatgcgattaattgataatgaccaactcgacgaattatatgatcaagccgggcaatcggaacgtttacgttcccaccttatgatgcacggctcgcatcaagaaaaggtacagcgtttacttattgcattagtaaagggcagctatgttgaaccgcattatcacgaacttcctcatcagtgggaaatgttcattgttatggaggggcaacttcaggtttgtttgtatggtagaaatggtgaggttataaagcaatttatagcaggagataatactggaatgagcattgtggagttttctccgggcgatatacacagtgtcgaatgcctatctccgcgtgctcttatggtggaagttaaggaggggccatttgacccttcttttgcaaaatcgttcgtgtgagcggccgcgagctcgtcgactcgaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 16 - DNA, 13435 bp, synthetic construct
  source 1..13435: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatccagatgaagtaaataatacagaagaattacccttatttactgagacgacagcttacgcgccaagcagcccttattccgcatccaaagcatccagcgatcatttagtccgcgcgtggaaacgtacatatggtttaccgacaattgtgactaattgctcgaacaactatggtccttatcatttcccggaaaagcttattccactggttattcttaatgcactggaaggtaaggcattacctatttatggcaaaggagatcagatccgcgactggttgtatgttgaagatcatgcgcgtgcgttatataccgtcgtaaccgaaggtaaagcgggtgaaacttataacattggtgggcacaacgaaaagaaaaacatcgatgtagtgctcactatttgtgatttgctggatgagattgtaccgaaagagaaatcttatcgtgagcaaatcacttatgttgctgatcgtccgggacacgatcgccgctatgctattgatgctgagaagattggtcgcgcattgggatggaaaccacaggaaacgtttgagagcgggattcgtaaaacggtggaatggtacctgtccaatacaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctttgggtaatttgattgcttttgatgttcactctactgattattgcggtgattttagtaatcctgaaggtgtagctgaaaccgtaagaagcattcggccggatattattgtcaatgcagccgctcacaccgcagtagacaaagcagaatcagaaccggagtttgcacaattaattaacgcaacaagtgtcgaagcgattgcgaaagcagcaaatgaagttggagcctgggttatccattactcgactgattacgtcttccctggaaatggcgatatgccatggctggagacggatgcaaccgcaccactaaatgtttacggtgaaaccaagttagccggagaaaaagcgttacaggaatattgcgcgaagcatcttattttccggaccagctgggtctatgcaggaaaaggaaataacttcgccaaaacgatgttacgtctggcaaaagagcgtgaagaattagcggttattaacgatcagtttggtgcgccaacaggtgctgaactgctggctgattgtacagcacatgccattcgtgtcgcactgaataaaccggatgtcgcaggcttgtaccatttggtagccagtggtaccacaacctggtacgattatgctgcgctggtttttgaagaggcgcgcaaagcaggcattccccttgcactcaacaagctcaacgcagtaccaacaacagcctatcctacaccagctcgtcgtccacataactctcgccttaatacagaaaaatttcagcagaactttgcgcttgtcttgcctgactggcaggttggcgtgaaacgaatgctcaatgaattatttacgactacagcaatttaatagtttttgcatcttgttcgtgatggtggagcaagatgaattaaaaggaatgatgaaatgaaaatgcgtaaaggtattattttagcgggtggttctggtacacgtctttatcctgtgactatggctgtcagtaaacagctattacctatttatgataaaccgatgatctattacccgctctctacactgatgttggcgggtattcgcgatattttgattatcagtacacctcaggatactcctcgttttcaacaattgctgggtgacggtagccagtggggcctgaatcttcagtacaaagtgcaacctagcccagatggcctcgcgcaggcatttatcatcggtgaagagtttattggtggtgatgattgtgctttggttcttggtgataatatcttttacggtcacgatctgccgaagctaatggaggccgctgttaacaaagaaagtggtgcaacggtatttgcctatcacgttaatgatccagaacgctatggtgtcgttgagtttgataaaaacggtacggcaatcagtctggaagaaaaaccgttagaaccaaagagtaattacgccgttacaggtctgtacttttatgataacgacgtggttcagatggcgaaaaacttgaagccgtctgcacgtggtgagttagaaattacagatattaaccgtatttatcttgagcagggacgtctgtctgtcgcgatgatggggcgtggctacgcgtggctggacacggggactcatcagagtctgatagaagcaagtaattttattgcgacaattgaagagcgccagggattgaaggtttcctgtcctgaagagattgcatttcgtaaaggttttattgatgttgagcaagtaagaaaattagctgtaccactaataaagaataattatgggcagtatctttataaaatgacgaaggattcaaattaatgaatgtgattagaactgaaattgaagatgtgctaattctggagccaagagtatttggtgatgatagaggtttcttttatgagagctttaatcaatcagcatttgaacatattctaggctatccggtcagctttgttcaagacaatcactcacgttcatcaaaaaatgtactcagaggccttcactttcaacgcggcgagtacgcacaagataaacttgtacgctgcactcatggagcagtttttgatgttgctgttgatattcgacccaattcggtatcctttggtaaatgggttggtgttctgctttcagctgataataagcagcagttgtggataccaaaagggtttgctcatggctttttggttctgtctgatatcgctgaatttcaatataaaactacaaactattatcatcctgaaagcgattgtggaatatgttggaatgatgaacgcattgcaattgattggccccaaacatcagggttaatcctttcgccaaaagatgaaaggctctttacgttagatgagcttatcagattaaaattaattgcatgaatacgaataaattatctttaagaagaaacgttatatatctggctgtcgttcaaggtagcaattatcttttaccattgcttacatttccatatcttgtaagaacacttggtcctgaaaatttcggtatattcggtttttgccaagcgactatgctatatatgataatgtttgttgaatatggtttcaatctcacagcaactcagagtattgccaaagcagcagatagtaaagataaagtaacgtctattttttgggcggtgatattttcaaaaatagttcttatcgtcattacattgattttcttaacgtcgatgaccttgcttgttcctgaatataacaagcatgccgtaattatatggtcgtttgttcctgcattagtcgggaatttaatctaccctatctggctgtttcagggaaaagaaaaaatgaaatggctgactttaagtagtattttatcccgcttggctattatccctctaacatttatttttgtgaacacaaagtcagatatagcaattgccggttttattcagtcaagtgcaaatctggttgctggaattattgcactagctatcgttgttcatgaaggttggattggtaaagttacgctatcattacataatgtgcgtcgatctttagcagacggttttcatgtttttatttccacatctgctattagtttatattctacgggaatagttattatcctgggatttatatctggaccaacgtccgtagggaattttaatgcggccaatactataagaaacgcgcttcaagggctattaaatcctatcacccaagcaatatacccaagaatatcaagtacgcttgttcttaatcgtgtgaagggtgtgattttaattaaaaaatcattgacctgcttgagtttgattggtggtgctttttcattaattctgctcttgggtgcatctatactagtaaaaataagtatagggccgggatatgataatgcagtgattgtgctaatgattatatcgcctctgccttttcttatttcattaagtaatgtctatggcattcaagttatgctgacccataattataagaaagaattcagtaagattttaatcgctgcgggtttgttgagtttgttgttgatttttccgctaacaactctttttaaagagattggtgcagcaataacattgcttgcaacagagtgcttagttacgtcactcatgctgatgttcgtaagaaataataaattactggtttgctgaggattttatgtacgattatatcattgttggttctggtttgtttggtgccgtttgtgcgaatgagttaaaaaagctaaacaaaaaagttttagtgattgagaaaagaaatcatatcggtggaaatgcgtacacagaggactgtgagggtatccagattcataaatatggtgcacatatttttcataccaatgataaatatatatgggattacgttaatgatttagtagaatttaatcgttttactaattctccactggcgatttataaagacaaattattcaaccttccttttaatatgaatactttccaccaaatgtggggagttaaagatcctcaagaagctcaaaatatcattaatgctcagaaaaaaaagtatggtgacaaggtacctgaaaatttggaggagcaggcgatttcattagttggggaggacttataccaagcattgataaagggttatacggagaagcagtggggaagaagtgcaaaagaattgcctgcatttattattaagcgaatcccagtgagatttacgtttgataacaattatttttccgatcgctatcaaggtattccggtgggaggctacactaagcttattgaaaaaatgcttgaaggtgtggacgtaaaattaggcattgattttttgaaagacaaagattctctagcgagtaaagcccatagaatcatctacactggacccattgatcagtacttcgactataggtttggagcgttagaatatcgctctttaaaatttgagacggaacgccatgaatttccaaacttccaagggaatgcagtaataaatttcactgatgctaatgtaccatataccagaataattgagcataaacattttgactatgttgagacaaagcatacggttgttacaaaagaatatccattagagtggaaagttggcgacgaaccctactatccagttaatgataataaaaacatggagctttttaagaaatatagagagttagctagcagagaagacaaggttatatttggcgggcgtttggccgagtataaatattatgatatgcatcaagtgatatctgccgctctttatcaagtgaaaaatataatgagtacggattaatgatctatcttgtaattagtgtctttctcattacagcatttatctgtttatatcttaagaaggatatattttatccagccgtatgcgttaatatcatcttcgcactggtcttattgggatatgaaataacgtcagatatatatgcttttcagttaaatgacgctacgttgatttttctactttgcaatgttttgacatttaccctgtcatgtttattgacggaaagtgtattagatctaaatatcagaaaagtcaataatgctatttatagcataccatcgaagaaagtgcataatgtaggcttgttagttatttctttttcgatgatatatatatgcatgaggttaagtaactaccagttcgggactagcttacttagctatatgaatttgataagagatgctgatgttgaagacacatcaagaaatttctcagcatacatgcagccaatcattctaactacttttgctttatttatttggtctaaaaaatttactaatacaaaggtaagtaaaacatttactttacttgtttttattgtattcatctttgcaattatactgaatactggtaagcaaattgtctttatggttatcatctcttatgcattcatcgtaggtgttaatagagtaaaacattatgtttatcttattacagctgtaggtgttctattctccttgtatatgctctttttacgtggactgcctggggggatggcatattatctatccatgtatttggtcagccctataatcgcgtttcaggagttttattttcagcaagtatctaactctgccagttctcatgtcttttggttttttgaaaggctgatggggctattaacaggtggagtctctatgtcgttgcataaagaatttgtgtgggtgggtttgccaacaaatgtttatactgctttttcggattatgtttatatttccgcggagctaagctatttgatgatggttattcatggctgtatttcaggtgttttatggagattgtctcgaaattacatatctgtgaaaatattttattcatattttatttataccttttctttcattttttatcatgaaagcttcatgactaatattagcagttggatacaaataactctttgtatcatagtattctctcaatttcttaaggcccagaaaataaagtgaaaatgtattttttgaatgatttaaatttctctagacgcgatgctggatttaaagcaagaaaagatgcactggacattgcttcagattatgaaaacatttctgttgttaacattcctctatggggtggagtagtccagagaattattagttctgttaagcttagtacatttctctgcggtcttgaaaataaagatgttttaattttcaatttcccgatggccaaaccattttggcatatattgtcattctttcaccgccttctaaaatttagaatagtacctctgattcatgatattgatgaattaagaggaggagggggtagtgattctgtgcggcttgctacctgtgatatggtcataagtcacaatccacaaatgacaaagtaccttagtaaatatatgtctcaggataaaatcaaagacataaaaatatttgattacctcgtctcatctgatgtggagcatcgagatgttacggataagcaacgaggggtcatatatgctggcaacctttctaggcataaatgttctttcatatatactgaaggatgcgattttactctctttggtgtcaactatgaaaataaagataatcctaaatatcttggaagttttgatgctcaatctccggaaaagattaacctcccaggcatgcaatttggactcatttgggatggagattctgtcgaaacctgtagtggtgcctttggcgactatttaaagtttaataaccctcataagacatctctttatctttcaatggaacttccagtatttatatgggataaagccgcccttgcggatttcattgtagataatagaataggatatgcagtgggatcaatcaaagaaatgcaagagattgttgactccatgacaatagaaacttataagcaaattagtgagaatacaaaaattatttctcagaaaattcgaacaggaagttacttcagggatgttcttgaagaggtgatcgatgatcttaaaactcgctaaacgatatggtctctgtggttttattcggcttgttagagatgtcttattgactcgtgtattttaccggaactgtagaattattcgatttccctgctatattcgcaatgatggtagcattaattttggtgaaaatttcacaagtggagtcggtctcaggctggatgcatttggacgtggcgtgatttttttttccgataatgtgcaagttaacgactatgttcatatcgcctcaattgagagcgttacgataggtcgggatacgcttattgcaagtaaagtatttattaccgatcataatcacggttcctttaagcactctgatccaatgagttcgccaaatatacctccagacatgcgcacgttggaatcttcagctgttgtaattggccagagggtttggttgggtgagaatgtgacggttttgcctggaacaattattggtaatggagtcgtagtcggcgccaattctgttgttagaggttctattcccgaaaatactgtcattgcgggagtaccagcaaaaatcataaagaaatacaatcatgagaccaaattatgggaaaaagcatagtcgttgtttctgcggtcaattttaccactggcggtccatttaccattttgaaaaaatttttggcagcaactaataataaagaaaatgtcagttttatcgcattagtccattctgctaaagagttaaaagaaagttatccatgggttaaattcattgagtttcctgaggttaaagggtcgtggctaaaacgtttgcactttgaatatgtagtttgtaaaaaactttcaaaagagctgaatgctacgcattggatttgtctgcatgatattacggccaatgtcgtcactaaaaaaagatatgtgtattgtcataaccctgcccctttttataaaggaattttattccgtgaaattcttatggagcctagctttttcttatttaaaatgctatacgggctgatatataaaataaacattaaaaaaaatactgcagtgtttgttcaacaattctggatgaaagaaaaatttatcaagaaatattctataaataacatcattgtcagtcggccagaaattaaattatctgataaaagccaacttactgatgatgattctcaatttaagaataacccttctgagttgacaatattttaccctgctgttccacgagtatttaaaaattacgagcttattattagtgcagcaaggaaattgaaagaacaatccaatattaaatttctgcttactatcagtggtacagaaaatgcgtatgcaaaatatattatcagtcttgcagaaggactggataatgttcatttcctcgggtacttggataaagaaaaaatcgatcattgttataatatttcagatatagtttgttttccctctaggttagaaacatggggattgccgttgtctgaggctaaagagcgaggtaagtgggtattagcatcagatttcccatttactagagaaactcttggtagttatgaaaagaaagctttttttgattctaataacgatgacatgttagttaaacttattattgacttcaaaaaaggtaacctcaaaaaagatatctctgatgcaaatttcatttatcgtaatgaaaatgtattagttgggtttgatgaactagttaattttattactgaagaacattgaaatggtatatataataatcgtttcccacggacatgaagactacatcaaaaaattactcgaaaatcttaatgctgacgatgagcactacaagattatcgtacgcgacaacaaagactctctattattgaaacaaatatgccagcattatgcaggcctggactatattagtggaggtgtatacggctttggtcataataataatattgcggtggcgtatgtaaaggaaaaatatagacccgcagatgatgattacattttgtttttgaatcccgatatcatcatgaagcatgatgatttgctgacatatattaaatatgtcgaaagtaagcgttatgcttttagtacattatgcctgttccgagatgaagcgaaatctttacatgattattccgtaagaaaatttcctgtgctttctgattttattgtgtcatttatgttagggattaataaaacaaaaattcctaaagaaagtatctattctgatacggttgttgattggtgcgcaggatcatttatgctggtacgtttttcagattttgtgcgtgtaaatggcttcgatcaaggttactttatgtactgtgaagatattgacctgtgcttgaggcttagcctggctggtgtcagacttcattatgttcccgcttttcatgcgatacattatgctcatcatgacaatcgaagttttttttcaaaagccttcagatggcacttaaaaagtacttttagatatttagccagaaaacgtattttatcaaatcgcaactttgatcgaatttcatcagtttttcacccgtaagagctcggtacccgggcctagggtgtaggctggagctgcttcgaagttcctatactttctagagaataggaacttcggaataggaactaaggaggatattcatatccgtcgacggcggccgccctgcaggcatgcaagcttgatccatatggatcgctagcttaattaaataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtacgggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtacgggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcaaaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 17 - DNA, 13228 bp, synthetic construct
  source 1..13228: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatccagatgaagtaaataatacagaagaattacccttatttactgagacgacagcttacgcgccaagcagcccttattccgcatccaaagcatccagcgatcatttagtccgcgcgtggaaacgtacatatggtttaccgacaattgtgactaattgctcgaacaactatggtccttatcatttcccggaaaagcttattccactggttattcttaatgcactggaaggtaaggcattacctatttatggcaaaggagatcagatccgcgactggttgtatgttgaagatcatgcgcgtgcgttatataccgtcgtaaccgaaggtaaagcgggtgaaacttataacattggtgggcacaacgaaaagaaaaacatcgatgtagtgctcactatttgtgatttgctggatgagattgtaccgaaagagaaatcttatcgtgagcaaatcacttatgttgctgatcgtccgggacacgatcgccgctatgctattgatgctgagaagattggtcgcgcattgggatggaaaccacaggaaacgtttgagagcgggattcgtaaaacggtggaatggtacctgtccaatacaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctttgggtaatttgattgcttttgatgttcactctactgattattgcggtgattttagtaatcctgaaggtgtagctgaaaccgtaagaagcattcggccggatattattgtcaatgcagccgctcacaccgcagtagacaaagcagaatcagaaccggagtttgcacaattaattaacgcaacaagtgtcgaagcgattgcgaaagcagcaaatgaagttggagcctgggttatccattactcgactgattacgtcttccctggaaatggcgatatgccatggctggagacggatgcaaccgcaccactaaatgtttacggtgaaaccaagttagccggagaaaaagcgttacaggaatattgcgcgaagcatcttattttccggaccagctgggtctatgcaggaaaaggaaataacttcgccaaaacgatgttacgtctggcaaaagagcgtgaagaattagcggttattaacgatcagtttggtgcgccaacaggtgctgaactgctggctgattgtacagcacatgccattcgtgtcgcactgaataaaccggatgtcgcaggcttgtaccatttggtagccagtggtaccacaacctggtacgattatgctgcgctggtttttgaagaggcgcgcaaagcaggcattccccttgcactcaacaagctcaacgcagtaccaacaacagcctatcctacaccagctcgtcgtccacataactctcgccttaatacagaaaaatttcagcagaactttgcgcttgtcttgcctgactggcaggttggcgtgaaacgaatgctcaatgaattatttacgactacagcaatttaatagtttttgcatcttgttcgtgatggtggagcaagatgaattaaaaggaatgatgaaatgaaaatgcgtaaaggtattattttagcgggtggttctggtacacgtctttatcctgtgactatggctgtcagtaaacagctattacctatttatgataaaccgatgatctattacccgctctctacactgatgttggcgggtattcgcgatattttgattatcagtacacctcaggatactcctcgttttcaacaattgctgggtgacggtagccagtggggcctgaatcttcagtacaaagtgcaacctagcccagatggcctcgcgcaggcatttatcatcggtgaagagtttattggtggtgatgattgtgctttggttcttggtgataatatcttttacggtcacgatctgccgaagctaatggaggccgctgttaacaaagaaagtggtgcaacggtatttgcctatcacgttaatgatccagaacgctatggtgtcgttgagtttgataaaaacggtacggcaatcagtctggaagaaaaaccgttagaaccaaagagtaattacgccgttacaggtctgtacttttatgataacgacgtggttcagatggcgaaaaacttgaagccgtctgcacgtggtgagttagaaattacagatattaaccgtatttatcttgagcagggacgtctgtctgtcgcgatgatggggcgtggctacgcgtggctggacacggggactcatcagagtctgatagaagcaagtaattttattgcgacaattgaagagcgccagggattgaaggtttcctgtcctgaagagattgcatttcgtaaaggttttattgatgttgagcaagtaagaaaattagctgtaccactaataaagaataattatgggcagtatctttataaaatgacgaaggattcaaattaatgaatgtgattagaactgaaattgaagatgtgctaattctggagccaagagtatttggtgatgatagaggtttcttttatgagagctttaatcaatcagcatttgaacatattctaggctatccggtcagctttgttcaagacaatcactcacgttcatcaaaaaatgtactcagaggccttcactttcaacgcggcgagtacgcacaagataaacttgtacgctgcactcatggagcagtttttgatgttgctgttgatattcgacccaattcggtatcctttggtaaatgggttggtgttctgctttcagctgataataagcagcagttgtggataccaaaagggtttgctcatggctttttggttctgtctgatatcgctgaatttcaatataaaactacaaactattatcatcctgaaagcgattgtggaatatgttggaatgatgaacgcattgcaattgattggccccaaacatcagggttaatcctttcgccaaaagatgaaaggctctttacgttagatgagcttatcagattaaaattaattgcatgaggccggccttaaggaggactagtcccggcgcgccatgagtttaatcaaaaacagtttttggaacctttgcgggtatgtacttccagctattgtgacactaccagctttgggtattatggggcgaaaattaggcccagaattatttggtgtattcactttggcattagctgttgtgggttatgcaagcatttttgatgcaggccttactcgcgcagtgatacgagaagtcgcaattgaaaaagataatgaagaaaataagttgaaaattatttcttcagcgacagttgtaattatttatttgagtttggccgcctcactcttattatttttttttagtggtcatatcgcattgctactgaacattagtgagactttttttcataatgtaagtgtctcgcttaaaattctcgcagcatccataccattatttttgattactcaaatatggttgtcaattttagaaggtgaagaaagatttggtttacttaatatctacaaatcaattacgggagtgatattagcaatctcaccggcattatttatacttattaaaccctctttgatgtatgcgataataggcttagttctagcaaggtttttatgttttattttggcttttataatttgtcacgataaagtgcttaaagctaaactaacaatcgatataccaacaattaaaagattgtttatgttcggtggttggattacagtaagtaatatcatcagccctgtgctatcatattttgataggtttattgtttcaaatcaacttggggctgctaatgttgctttttatactgcaccatcagaaattatttctcggcttagtataattccaggtgcgttttcaagagccttatttccaagattagctaatgcaaataattccgctgaaagatataaaacgaaaagattaattacaatttcacttttaataatcatcacccctattttttgtattggcgtgttattttcagagaagataatggttttatggatgggggcatcattttttggtgagcctggtttggtattatcaatattactgattggctttatttttaatggattggcacaagtaccatttgccagtattcaatcccgaggtcatgctaagataactgcatttgttcatctcttagagttgtttccttatttattacttttattttacctcataaaagcacatggggttgttggcgcgggtattgcgtggtcagtgaggatgatagtagattatatagcattaagtcttttggacggtaagtatattaataaataaaattcaaaatgcaagttaataactcatggctttatttgggtaggtgacaatttataatgatatatatattaactttaactcttcttctagttatagccataatgttttctcttctcggcacaaaaagtaggatcacatctccattacctttgcattttttaccatggttactaactttaattgtcgggataagtaattacgatcaattttacgagtttaatgaaagaagcttttactctttgttgatttggtttacagttatttttatattttatttcataggggaactggttaattataaacgtgaaaatataaatgtttattatggtctttcacatattaaatatgaatgtaaaaaatattggatcattgtcatcccaatttcattatataccattttcgaaatatatatggttggtatggggggagcagatggattctttctcaatttacgtcttgcaaatacattggagggctatacgggtaaaaaatttatcttaatgcctgctgtatatcctctaatgatggctatgttcgcaattgtttgtctaacaaaaacttccaaattaaataaatactccatttatttctggatgtttttgtattgtattggcacaatgggaaaattttcaatattaacgccaatattgacatatttaattatttatgacttcaaacatagattaaaagtaaaaaaaacaataaagtttacattgttgataattatattagctttaactttgcattttacacgtatggctgagaatgaccactcaacatttttatctattttagggctctatatttattcaccaataattgctttaggccagttgaatgaagtaaatagtagtcattttggtgagtatacgtttagattcatatatgctataactaataaaattggccttattaaagaattgccagtaaatactattcttgactattcatacgttcctgtaccaacaaatgtatatactgcacttcaaccattttaccaggattttggttatactggcatcatatttggagcagtattatacggactaatatatgtgagtttatacacggccggtgttcgtggaaataatacacaggcattactgatttacgcattgttttcagttagcagtgcaacggctttcttcgctgaaacgctagtaacgaatttagctggaaatgtgatgttagtattatgtaccatcttactatggcgatttacagtaatatgcaaaccagtacagtaaccattctaatggccacctacaatggcgaggccttcatcaaaaatcagattttgtcactacaacaacaaacattttctaactggcggttatttattcaggatgatgggtctacagacaatactatatctataataaaaaacttccaaaaatctgactccagaattcggctagttgatgataatttgaaaggtcaaggtgcaggaaaaaattttttatcgctgataaagtacagcgagacagattatacaatttattgtgaccaagatgatatttggttagaaaacaaaatatttgaattagtaaagtatgcaaatgaaattaaattgaatgtatcagatgcgccttcgctagtttatgctgatggctatgcttatatggatggtgagggtacaatcgatttttctgggatatctaacaatcatgctgatcaattaaaggattttcttttttttaatggtggataccaaggatgttctattatgttcaatcgtgcaatgaccaaatttcttctgaattatcgaggatttgtatatctacatgacgatatcacaacattagctgcatacgctcttggtaaagtttattttctcccgaaataccttatgttatatagacagcacacgaatgcggtaactggtatcaaaacattccgcaatggattgacttctaaatttaaatcaccagtaaactatcttttatcacgaaaacattatcaggtaaaaaaatctttttttgaatgtaacagctctatcttatcagagacgaataaaaaagtttttttggattttatttcattttgtgaatcaaataataaatttacagatttttttaagttatggcgaggtgggtttagattaaataacagtagaactaaattattattaaaattcttaatacggagaaaatttagcgaatgatttcaatacttacacctacttttaatcggcaacatactttatcaaggctattcaattctcttatattacaaactgataaagattttgagtggataataattgatgatggtagtatagatgcaacagcggtacttgtagaagattttagaaaaaaatgtgattttgacttgatttattgctatcaggaaaataatggtaagcccatggctttaaacgctggtgttaaagcttgtagaggcgattatatctttattgttgacagtgatgatgcactaactcccgatgccataaaattaattaaagaatcaatacatgattgcttatctgagaaggaaagtttcagcggagtcggttttagaaaagcatatataaaaggggggattattggtaatgatttaaataattcttcagaacatatatactatttaaatgcgactgagattagcaatttaataaatggtgatgttgcatattgttttaaaaaagaaagtttggtaaaaaatccattcccccgtatagaagatgaaaaatttgttccagaattatatatttggaataaaataactgacaaggcgaagattcgatttaacataagcaaagttatatatctttgtgagtatcttgatgatggtctttctaaaaatttccataaccagcttaaaaaatacccaaaggggtttaagatttattacaaagatcaaagaaaacgagagaaaacttatataaaaaaaacaaagatgctaattagatatttgcaatgttgttattatgagaaaataaaatgaaaatactatttgtcattacaggtttaggccttggaggtgctgagaagcaggtttgtcttttagctgataaattaagtttaagcgggcaccatgtaaagattatttcacttggacatatgtctaataataaagtctttcctagcgaaaataatgttaatgtcattaatgtaaatatgtcaaaaaacatttctggagttataaaaggttgtgtcagaattagagatgttatagctaatttcaaaccagacattgtacacagtcatatgtttcatgcaaacattatcactagattgtctgtaattggaatcaaaaacagacctggtattatatcaactgcacataataaaaatgaaggtgggtatttcagaatgctcacatatagaataaccgattgtttaagtgattgttgtacaaatgttagcaaagaagcagtggatgagtttttacggataaaagcctttaatcccgctaaagcaattactatgtataatgggatagataccaataaatttaaatttgatttattggcaaggagggaaattcgagacggtattaatataaaaaatgatgatatattattacttgctgcaggtcgtttaacgttagctaaagattatcctaatttattgaatgcaatgactctgcttcctgaacactttaaacttattattattggtgatggtgaattgcgtgacgaaattaatatgcttataaaaaaattgcaattatctaatagggtgtccttgttgggagttaaaaaaaatattgctccctatttttctgcatgtgatatttttgttctctcttctcgttgggaaggatttggattagtcgtggcagaagctatgtcatgtgagcgaattgttgttggcacggattcagggggagtaagagaagttattggtgacgatgattttcttgtacccatatctgattcaacacaacttgcaagcaaaattgaaaaattgtctttgagccagatacgtgatcacattggttttcggaatcgtgagcgtattttaaaaaatttctcaatagatactattattatgcagtggcaagaactctatggaactataatttgctcaaaacatgaaaggtagatttatatttggaacgtgtcttttgtttgaatttaattcaatctcaattgagatttttgtatttcaaaaataccatcatagctaacgatgattggtatttattttaagatgctttctataaatatattgacgtttttaatgcgccgaaacgattgggctgggaacagagaagtaaaactgttttgagaatgaagagtttttgagatgtttatggatattaaaaattgatccagtgaattaattatttataataaatcaagatttaatgttaataaatgataatcttttctgacactcatattaattatgagtggtacgtttggtaaacggtaaactattatatgacagctagaacaactaaagttttgcacttacaattactcccactcttaagtggcgttcaaagggtaacattaaacgaaattagtgcgttatatactgattatgattatacactagtttgctcaaaaaaaggtccactaacaaaagcattgctggaatatgatgtcgattgtcattgtatccccgaacttacgagagaaattaccgtaaagaatgattttaaagcattgttcaagctttataagttcataaaaaaagaaaaatttgacattgtgcatacacattcttcaaaaacaggtattttggggcgagttgctgccaaattagcacgtgttggaaaggtgatccacactgtacatggtttttcttttccagccgcatctagtaaaaaaagttattacctttattttttcatggaatggatagcaaagttctttacggataagttaatcgtcttgaatgtagatgatgaatatatagcaataaacaaattaaaattcaagcgggataaagtttttttaattcctaatggagtagacactgataagttttctcctttagaaaataaaatttatagtagcaccttgaatctagtaatggttggtagattatccaagcaaaaagatcctgagacattattgcttgctgttgaaaaactgctgaatgaaaatgttaatgttaagctgacacttgtaggagatggtgaactaaaagaacagttagaaagcaggttcaaacggcaagatggacgtataatttttcatggatggtcagataacattgttaatattttaaaagttaatgatctttttatattaccttctctttgggagggtatgccattagcaattttagaagcattgagctgtggacttccatgtatagtcactaatattccaggtaataatagcttaatagaagatggctataatggttgtttgtttgaaattagagattgtcagttattatctcaaaaaatcatgtcatatgttggtaagccagaactgattgcacagcaatctaccaatgcacgatcatttattctgaaaaattatggattagttaaaagaaataataaggtcagacagctatatgataattaagagctcggtacccgggcctagggtgtaggctggagctgcttcgaagttcctatactttctagagaataggaacttcggaataggaactaaggaggatattcatatccgtcgacggcggccgccctgcaggcatgcaagcttgatccatatggatcgctagcttaattaaataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtacgggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcaaaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 18 - DNA, 13554 bp, synthetic construct
  source 1..13554: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcagtgaagatacttgttactggtggcgcaggatttattggttctgctgttgttcgtcacataataaataatacgcaagatagtgttgttaatgtcgataaattaacatacgccggaaacctggaatcacttgcagatgtttctgattctgaacgctatttctttgaacatgcggatatttgtgatgcagctgcaatggcacggatttttgctcagcatcagccggatgcagtgatgcacctggcagctgaaagccatgttgaccgttcaattacaggccctgcggcatttattgaaaccaatattgtgggtacttatgtccttttagaagcggctcggaattattggtctggtctggatgatgaaaagaaaaaaaacttccgttttcatcatatttctactgatgaggtgtatggtgacttaccccatccggatgaagtaaatagcaatgaaacgttgccgctatttacggaaacgacagcatacgcgccaagtagtccatattctgcttctaaagcttccagcgatcatttggttcgcgcatggaaacgtacttatggtttaccgaccattgtgactaattgctcgaacaactatggtccttatcatttcccggaaaagcttattccactggttattcttaattcactggaaggtaaggcattacctatttatggcaaaggagatcagatccgcgactggttgtatgtagaggatcatgctcgagcgttatataccgtcgtaaccgaaggtaaagcgggcgaaacttataacattggtggacacaacgaaaagaaaaacatcgacgtagtgttcactatttgtgatttgttggatgagatagtcccgaaagagaaatcttaccgcgagcaaattacttatgttaccgatcgtccgggacacgatcgccgttatgcgattgatgctgagaagattggtcgcgaattgggatggaaaccacaggaaacgtttgagagtgggattcgtaaaacggtggaatggtacctgtccaatacaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctctgggtaatttgattgctcttgatgttcactccactgattactgtggtgattttagtaatcctgaaggtgtagctgaaaccgtaagaagcattcggcctgatattattgtcaacgcagccgctcacaccgcagtagacaaagcagaatcagaaccgaagtttgcacaattactgaacgcgacgagtgtcgaagcgatcgcgaaagcagccaatgaagtcggcgcctgggttattcactactctactgactacgtatttccggggaccggtgaaataccatggcaggaggaggatgcaaccgcaccgctaaatgtttacggtgaaaccaagttagcgggagaaaaagcattacaagagcattgtgcgaagcaccttattttccggaccagctgggtctatgcaggtaaaggaaataacttcgccaaaacaatgttgcgtctggcaaaagagcgtgaagaattagccgttattaatgatcagtttggtgcgccaactggcgcagagttactggctgattgtacggcacatgctattcgtgtggcactgaataaaccggaagtcgcaggcttgtaccatctggtagctagtggtaccacaacgtggcacgattatgctgcgctggtttttgaagaggcgcgcaaagcaggcattccccttgcactcaacaagctcaacgcagtaccaacaacagcctatcctacaccagctcgtcgtccacataactctcgccttaatacagaaaaatttcagcagaactttgcgcttgtcttgcctgactggcaggttggcgtgaaacgaatgcttaacgaattatttacgactacagcaatttaatagtttttgcatcttgttcgtaatggtggagcaagatgtattaaaaggaatgatgaaatgaaaacgcgtaaaggtattattttggcgggtggttctggtactcgtctttatcctgtgacgatggccgtcagtaaacagctgttaccgatttatgataaaccgatgatctattacccgctctctacactgatgttagcgggtattcgcgatattctgattatcagtacaccacaggatactcctcgttttcaacaactgctgggtgacgggagccagtggggcctgaatcttcagtacaaagtgcaaccgagtccggatggtcttgcgcaggcgtttattatcggtgaagagtttattggtggtgatgattgtgctttggtacttggtgataatatcttctacggccacgacctgccgaagttaatggacgtagctgttaacaaagaaagtggtgcaacggtatttgcctatcacgttaatgatcctgaacgttatggtgtcgtggagtttgataataacggtactgcaattagcctggaagaaaaaccgctggaaccaaaaagtaactatgcggttactgggctttatttctatgacaatgacgttgtggaaatggcgaaaaaccttaagccttctgcccgaggtgaactggaaattaccgatattaaccgtatttatatggaacaaggacgtttgtctgtcgctatgatggggcgtggctatgcatggctggatacagggacgcatcaaagtcttattgaagcaagcaacttcattgccaccattgaagagcgccagggactaaaggtttcctgtccggaagaaattgcttatcgtaaagggtttattgatgctgagcaggtaaaagtattagccgaaccgttgaagaaaaatgcttatggtcagtatctgctcaaaatgattaaaggttattaataagatgaacgtaattaaaactgaaattcctgatgtgctgatttttgaaccaaaagtttttggggatgaacgtggcttcttttttgagagttttaatcagaggatttttgaagaagcagtaggtcgtaaggttgagtttgttcaggataaccattctaagtccagtaaaggtgttttacgtggtcttcattatcagttagaaccttatgctcaaggaaaactggtgcgctgtgttgttggcgaggtttttgatgttgcggttgatattcgtaaatcgtcacctacatttgggaaatgggttggggtgaatttgtctgctgagaataagcgtcagttgtggattcctgagggatttgcacatggttttttggtgctgagtgatttagcagaagttttatataaaacgaatcaatattatgctccatcacatgaaaaaaatattatatggaatgacctcttgcttaatattaaatggccgagcacagcactgatcactctgtctgataaggatgcaaatggggaaagatttgaactaagtgagttttgaaatgtctctcttaaaacatagtatatggaatgttgcgggctactttataccaacattaattgcaattcccgcctttggattaattgcgaggaaaattggtgtagaactatttggtttgtatacgttagcaatgatttttatagggtatgcaagtatatttgatgctgggttaacaagagctgttgtgcgtgaaatagcattactaaaaaacagagtggacgattgtaatacgataatagtaacttctattatcgctgtgatatttttagggtttatcggaggcgggggagtgtttctgcttaaaggcgatattattgaactgttaaatatctcaccaatatattacgccgattcgataaagtctctagtattattatcatctctgatacctgtattcttagtcacgcaaatactattagcagagcttgagggtcgggaatattttgggattctaaatatacaaaaaagtgtagggaattctttaattgcagggttacctgcattatttgttttaattaatcaaacgcttttttctgcaattattggtgtagcgattgcaagagttatatgcttgtggttaagctacattatgagcagggaaagaataactatcgatatctcatttttttcaataactgttttaaagcggttatttagatatggcgggtgggtaactataagtaacataatatctcctatattagcgagtatggatagatttattctatcccatatccagggagcatcaaaaatatcattctatacagtccctaatgagctggtaactaggcttggaatagttccaggctctcttgggaaagctgtttttccaaaattaagtcatgcaaggaattttacagcgtcatatgcagagcaaaaaaaagcttatatattaatgactgtcattgtaatgcctttggttttatttgtatattattacgcaaagtttattttaacattgtggatgggggctgagtatgcagggatttcggtcgaaatattacggattatgcttatagggtatatttttaactgttattcacaaatctcttttgccaacatacaggcctttggaaaagcaaaatacactgcatacatccatatgatggaatttattccttatttgataatgttatatataatttcaaaggaatatggggttattggtgttgcgtggttatggacaattcgagtaataattgattttttgatgcttttatatatgagttatcgttgtaataatcttatgaaaaaagggtagcctgatgatatatattgtggtattaaattggaatggggctatagataccattaattgtgttaaaagtttaatggatttaaatgttagcgattataaaattatcattgttgataactgttctatggataactcatatgatactataaaagaaaatcttaattcattatatattgctgataaaagtatcattgaggtgaagtatgaggatagaaataaatataaaaccttagaaaacgataaaatcatattaatacaatctccgcaaaataatgggtacgcaagtggtaataatattggcatagagttcgctcttaatcaggagaatatgaaatacgtctgggttctgaataatgatactgaagtggataaagaggctttaactcatttaattagtaaatgtgattcagataaaagtatagggatttgcggttctcgtttagtctattttgccgacagagagatgcagcaaggactaggtggggtgcataacaaatggttatgcactacaaaaaattatgaaatgggaagattagtttccaaaaaatatgatgatgaagtcattagtaatgatatagattatataattggcgcatcgatgtttttctctagagaatgtttggaaacagttggattgatgaatgaagaatattttttatactatgaagagttagatatttgcctcagagcaaaagcaaagaactttaaattaggtatttgctcagaaagtttggtttatcataaaataggtgcaagtactgatgggggaaagagcatgatggctgatctttgctcaataaaaaataggctggtcattacagaaaggttttatccccaatattattggacggtatggttgtcactttttgttgtagcatttaaccgtgctagaagaggtgagtttaataagatgaaaagatgtttgaatgttatgtttaacttcaaacgaaacaaaggtagcaaatgccattagaatatgcacttaatcatggtgttaataaatctatagtttgatatgttattaaagggtatttaatgaaagtggcttttttatctgcttatgatccactatctacatccagttggtctggcacaccttattatatgctaaaggcattatcgaagagaaatatttccattgaaatattaggaccggtaaatagctatatgatatacatgttaaaagtatataaattaatattaaggtgtttcggaaaagaatatgattatagtcattcgaagttgctttccaggtattacggtagaatattcggtaggaaattaaaaaaaattgatggtttggattttattatcgcacctgcaggttcctcacaaattgcttttttaaaaacaaccataccaataatatatctatcggatacaacatatgatcaattaaaaagctattatccgaatttaaataaaaaaacaattataaatgatgaggatgcaagtttaatcgaacgcaaggctattgaaaaagcaacagtagtatctttcccatctaaatgggcaatggatttttgcaggaattattacagattagattttgataaattagttgaaataccatggggggctaatttatttgatgatattcactttgctaataaaaatataattcaaaagaatagttatacttgtcttttcttgggagttgattgggaaagaaaaggtgggaaaacagccttgaaagcaattgaatatgtaaggcagttatatgggatcgatgttagactaaaaatttgtggatgtactccgaatcaaaagattttacctacttgggttgaattaattgataaagtagataaaaataacgttgacgaatatcagaaattcatcgatgtgttatctaacgctgatatacttcttttaccaaccattgctgaatgttatggaatggtattttgtgaagctgctgcttttggattgcctgttgtcgctacagatacaggtggagtcagttctatagttatcaacgaaaggacggggatattaattaaagacccgttagactataagcactttggaaatgcaattcataaaataattagttccgtagagacttatcaaaactactcccaaaacgcaagaattagatataataatatattgcattgggacaattgggctaaaaagataattgagattatgtatgagcataagaatagaagaatcaaatagcacaaaaagaattatatgtttatttatactttttcttgttttccctgattttttgttttatacattaggggttgataattttagcatttcaacgataatctcaattacattgctttttgtttttttaagagctaaaaatatttgcaaagataattttctaataatagtagcgttattcatattgttgtgttttaactgtttgttaagtatgctatttaatattgaacaggctttaacatttaaagttgtactttcaatatatagcatcttaataatggcatacgtctcctcttgttatgcacagacgttgtggttatgttctgaagaaatacttaagagatccgtcttttatttgttcgcatttctttgccttattggcattataagtattcttttacagaagactgagattatacatgataaaagtatgattctttttcctgaaccatcagcatttgcattggtttttatacctatcttttcattttgtttatactatacaagagggggggggctactattgctctatatattatctttgggtattgcgttaggtatccagaatttaacaatgttggtaggcattgtgattagtgtttttgtgatgaaaaaaataactataaggcaaactattgttatacttttgggggcatggattttttccatgatattaagtgatttagacatttcttactatacatcgcggcttgattttaaaaatactacgaacctatcagtgcttgtatatctttcaggaattgaaagagctttcttgaattttattacaagttatggtcttggtattggttttcaacaaatgggagtgaatggggagataggaatatatcaacaaattttagctgaacttgatgcccctatgttaaatatatacgatggctcatttatttcttctaagttaatatctgagtttggggttattggtgcattaatgtgtattttctattttttttatttttcccgattttatctgcgtttcaaaaaaagtaagagatattcaccgcagtatattttagcatatagcttctacatgtgtttcttcatccctctttttatacgtggtgctggttatataaacccctatgtgtttatgttattttcatcaatatttttgtgcaaatatcacgctaaaaatatcttgatgaaatctaatgtccagatagctatataatagtagattatattatcattatcacgtaaattacatattaatagcatatatgataactaggacataaataatgtgcattaaaaaaaaacttaagttaattaaacgatatggcctttatggtggtcttaggcttcttaaagatatattcttaacaaaatttttattttgttcaaatgttaggattattagatttccatgttatattagaaaagatggaagtgttagttttggaaaaggttttacatcaggtgtaggattacgagttgatgcatttatggatgccgtagtttccattggagaaaatgttcaaattaatgactatgttcacatcgcggctattaataatgtcattattggtagagatacattaatagcaagtaaagtatttattagtgatcataatcatggtattttttctaaatccgatatccatagttcaccaactattattccttcgtctaggccccttgaatctgcacctgtgtatattggagagcgtgtgtggattggcgaaaatgtgacaatattaccaggtgcgtgtataggtaatggtgtagttattggcgcaaacagtgttgttcgtggtgagattcctaataatgtgatcattgctggtgttccagctaaaattgttaaaaaatataactatgagcgtatgcaatgggaaagaatatagttgtaatatcggctgttaattttacaaccggaggcccctttaccgtactaaaaaatgtgcttacagcaactaaagatagagccgaatgtaaatttattgcactggttcatagctctgctgaactaatggaattatttccgtgggttgaatttatagagtatccagaagtcaagtcttcgtgggttaaaagattatatttcgaatatataacttgcaatagattatctaaggtgattaaggcaactcattgggtatgcttacatgatattacagcaaatgttagtgtaccctatagatttgtttattgccacaatcctgcaccgttctataaatatttaagctatcgagatattataggagaacctaaattttatcttttttatcttttttatgggcttttatacaatatcaatataaaaaagaacacagcagtttttgttcagcagcagtggctaaaaaaagaattcgaaaaaaaatataagttaaagaatgttgttgttagtcgccctgaagatatttgcccttttgaaagtgatggtttggtaagaaataataataaaaaggatgtgaggatattttacccagcagtgccccgtatatttaaaaactttgaagttatcatacgtgctgcacaaatattacaagataaaaatattcatttttatcttacttttgatggtactgaaaataagtatgcaaaaagaatatataaattagcttccgaactgaaaaatgtacatttcctcggttaccttaatgcaaccgagatggttaacttttatcaagattcagatattatttgtttcccatcgaaactagaaacgtggggattaccattatcagaagctaaaacatacaaaaaatggatatttgcggcagacttaccttatgctcatgaagttttatataactattcaaaaactagatattttccatttgacgatgagaaaatacttgttcgctacatattagagtacacaagtaaaaatatgcatgaagatataaaaaatagtagggtgaattttaataatgatgcattgactggttttgaacagtttattgaatatatcctcaaggggaactgacgtggtttatattataatcgtttcacatggccatgatgactatatagaaaatcttttattaaatttaaagttgccctctggaagatttaaaataatagttcgtgataacaaaagttcaatggttttaaaaaaaacatgcgaaaaaaattgcgtaacctatttgcatggagggcaatatggatttggacataataataacatagcagtgtcatatataattaataacttcatgattatgaataatgattattttctctttcttaaccccgatgtattcataaccagtgaaagtttgattaattatgttgattatataattagtaatgattataagtttagcacattatgtctttatcgagattttactaaaagcaaacatgattattcaatacggagttttccaactttatatgattttctttgttcttttttattgggggtgaataaaagtaaaattaagaaggaaaatatactttctgatactgtagttgattggtgtgctggctcatttatgcttattcatgctttaagtttcttaaatgtgaatggttttgatcaaaaatattttatgtattgtgaagatattgacctttgtatgcgtttaaaattaagtggagtagatctttactatactccccattttgatgctattcattatgcgcagcatgaaaatagaagaatatttactaaagcatttcgatggcatataaggagtattacgcgctacatattacggaaaccaattctttcttataaaaactatagaaaaattacatccgaactggtaaagtgattaaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 19 - DNA, 15197 bp, synthetic construct
  source 1..15197: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcagtgaagatacttgttactggtggcgcaggatttattggttctgctgttgttcgtcacataataaataatacgcaagatagtgttgttaatgtcgataaattaacatacgccggaaacctggaatcgctcgctgaaatttctgattctgaacgttattcatttgagcatgcagatatctgcgatgccgaagcgatggctcgtattttcgcacagcaccagccagacgcggtgatgcacctggcagcagagagccacgttgaccgctcaataactggccctgcggcatttattgaaaccaatattgtgggtacttatgttcttttagaagcggcgcgcaattattggtctggtctggatgatgaaaagaaaaaaaacttccgctttcatcatatttctactgatgaggtgtatggtgacttaccccatccggatgaagtaaatagcaatgaaacgttgccgctatttacggaaatgacagcatacgcgccaagtagtccatattctgcttctaaagcttccagcgatcatttggttcgcgcatggaaacgtacttatggtttaccgaccattgtgactaattgctcgaacaactatggtccttatcatttcccggaaaagcttattccactggttattcttaatgcactggaaggtaaggcattacctatttatggcaaaggagatcagatccgcgactggttgtatgtagaggatcatgctcgagcgttatataccgtcgtaaccgaaggtaaagcgggcgaaacttataacattggtggacacaacgaaaagaaaaacatcgacgtagtgttcactatttgtgatttgttggatgagatagtcccgaaagagaaatcttatcgtgagcaaattacctatgttgctgatcgcccagggcatgatcgccgttatgcaattgatgccgataaaattagccgcgaattgggctggaaaccacaggaaacgtttgagagcgggattcgtaaaactgtggaatggtatctgtccaatacaaaatgggttgataatgtgaaaagtggtgcctatcaatcgtggattgaacagaactatgggggccgccactaatgaatatcctcctttttggcaaaacagggcaggttggttgggaactacagcgtgctctggcacctctgggtaatttgattgctcttgatgttcactccactgattactgtggtgattttagtaaccctgaaggtgtggctgaaaccgttagaagcattcggcctgatattattgtcaacgcagccgctcacaccgcagtagacaaagcagaatcagaaccggagtttgcacaattactgaacgcgacgagtgtcgaagcgatcgcgaaagcagccaatgaagtcggcgcttgggttattcactactctactgactacgtatttccggggaccggtgaaataccatggcaggaggaggatgcaaccgcaccgctaaatgtttacggtgaaaccaagttagcaggagaaaaagcattacaagagcattgtgcgaagcaccttattttccggaccagctgggtctatgcaggtaaaggaaataacttcgccaaaacgatgttgcgtctggcaaaagagcgtgaagaattagccgttattaatgatcagtttggtgcgccaactggcgcagagttgctggctgattgtacggcacatgccattcgtgtggcactgaataaaccggaagtcgcaggtttgtaccatctggtagccagtggtaccacaacctggcacgattatgctgcgctggtttttgaagaggcgcgcaaagcaggcattccccttgcactcaacaagctcaacgcagtaccaacaacagtctatcctacaccagctcgtcgtccacataactctcgccttaatacagaaaaatttcagcagaactttgcgcttgtcttgcctgactggcaggttggtgtgaaacgcatgctcaacgaattatttacgactacagcaatttaatagtttttgcatcttgttcgtgatggtggaacaagatgaattaaaaggaatgatggaatgaatacgcgtaaaggtattattttagcgggtggttctggtacacgtctttatcctgtgactatggctgtcagtaaacagctgttaccgatttatgataaaccgatgatctattacccgctctctacactgatgttggcgggtattcgcgatattttgattatcagcacgccacaggatactcctcgttttcaacaactgctgggtgatgggagccagtgggggctaaatcttcactacaaagtgcaaccgagtccggatggtcttgcgcaggcatttatcatcggtgaagagtttatcggtggtgatgattgtgctttggtacttggtgataatatcttctacggtcacgacctgcctaagttaatggatgccgctgttaacaaagaaagtggtgcaacggtatttgcctatcacgttaatgatcctgaacgctatggtgtcgttgagtttgataaaaacggtactgcaatcagcctggaagaaaaaccgttacaaccaaaaagtaattatgcggtaaccgggctttatttctatgataactacgttgtggaaatggcgaaaaatcttaagccttctgcccgcggtgaactggaaattaccgatattaaccgtatctatatggaacaggggcatttatctgttgccatgatgggacgtggatatgcctggctggacacggggacacatcaaagtcttattgaagcaagcaacttcattgccaccattgaagagcgccagggcttgaaagtttcctgcccggaagaaattgcttaccgtaaagggtttattgatgctgagcaggtgaaagtattagctaaaccgctgaaaaaaaatgcttatggtcagtatctgctaaaaatgattaaaggttattaataaaatgaatgttattaaaacagaaattccagatgtactgatttttgaaccgaaagtttttggtgatgagcgtggtttctttatggaaagctttaatcagaaagttttcgaagaggctgtagggcggaaggttgaatttgttcaggataatcattctaaatcgtgtaaaggtgtacttagaggtttacactttcagcttcctccctttgagcaggcaaaattagtaaggtgtatagttggcgaggtatttgatgttgcagtagacattagacctaattctgaaacatttggttcatgggttggagtaactctttcgtcagaaaataaaaggcagctatggattccagaaggattcgcccatggttttttaactttaagtgatattgcagagtttgtttataaaactaacaactattattctttaaatcatgaaaggggagtcatttggaacgatgaggaaattaacattgcctggccctctcaatcagagaagattctgtcacagaaagatattaatttaccatcatttagatttgttcaaatgtttagcaagtagtgttatctttacactgcacatagtcatcattttttatgctttaagtaaattatattgcacatctataacacaaagcgcaataatatttcgacctgatgaaggtttgtggttatttatctttctaggcgttttttatgactaaaatagttgtggtttctacagctccaatattcccgacaaataatgggtacaaaagttctgtattaggaagaattgatgagttattaaatgaggataatgaggtcgttttgattgaaataaaccttgaaaatgttacggaaaagaaagatgaattaataccaacaagatttaataatattcaaagatatgaagtaaaaaaaatatctagatcatttattgccgagttacaaatattatttgatatcagaactcggtatgaacaattattttcttctgctgacattagagataacataaaaaagataattgatttagaaaaaccttctattattattgctgagtctatatgggcgttgcaagcattgcctattgaaattagtgcgagaatacactgtgttattcatgatgtggcaactgatttctttaaagaaatgtttgtatctcataatgaggttgtacgaaaaattttgttttttaatgattacctaaagttgaaaattactgaagaaaatattatcaaacgtttgagagttgagcaatttatctttctgacagaagaagataaatgttggtataaaacaagatacaatattgatgagggttgttgttccttagcgagcaatcatctttatgtagaaaagattaagagaactatcaatttccaaacccctttcctgcttattcccggtagcattgaattttcacaaaatttttacggcttaaattggtttataaaaaatatatatcctggattaaataggaaaataagaatagttgtaacaggaaaggcatcagataaaaaaataaagatgttaaactgtggagaggaaattacctttacgggagagcttgacttttccacatataataaacttagctcaacatgcttgtgtgttattgcaccgattacaacgggcactggaattaaaataaaaatattagaagctgtacaaaaaggtattcctgtacttacaacaaaatttgcttcaaaaggaatatgttccgatttatgtttttattgcgaggaggatactgacacaaactttgtcaatttaattaacagttttcttgaaacgacattaagagtccaagaatgaatttattgcttttttcagtccttgcgtttggtttaatattggctttggcccataataataaaagtggagatattaacgcatacttaatgttttttctcgtggtcctaatggtattaatatcagggctgcgtatgaatgatagtgattatatcgaatacaggaaaatgtataatgaagtgcctattttatgtgactttagtctcgcatctataagagatatacatggggaggtaggctatctattcttatcatcaatctttaaaactttatgcttgccatttcaattatttcttttttttattgcttttttatcactcctgcttacatatttttcattcagaaaaataagtttaataccgatactatcgttagttttttatttaagccatgcttttatagttagagatttgattcaaattagggcaggattagctgttagcatatcattatattcaataattaaatttaaaggaaataaaagtataattacaggagttttatttgcttctttgattcattctggggcgcttattattgctctttgttatccttttttcaaaaaaaaatacataacattaaaaatgatgttgtttttatttttagtgtcaattattttttcttatttgaatgggcttaatttatcgatacaactcttatctcaatatagtttgcttccaactgcaatttcgaattatgttggttgggaagaatatgattatcgggtgagtatatttactaatccggtttttattaaaggtgtttttttaattgtcttaatgcacaaatatgtactttcagatattaaaaatgagaaaattatagtgctttataacttatatgttttaggtgtattagctatggttgcattgagtgggatggctattctttcaggccgtctttcatcctttctgacactaggtgaaagcattttaattgtatatgctctgttctacaaaagaaatacacctctggcgtttctaattttttcttttttaacaattgtgcaattaggatatgatctatttatttctaatgtgcatcctgagcttactctgattatatttgggtgaatctaagtgaaaaataataaaataggcatacttatctctaaaatacaaaatcttggacctgtgaatgtagtacgaggattgataaaagaaaataaaaaatatgcttttactgttttttgtttaacaaatagcgtagataaaaatatatatgatgagttatgctgtttaggagccaaggttatattaataccagatggtacttggttcagcaaaattttatttgtgagaagttttttaaaggaacatccacataatatcttacattcacatgggatcacggccgatatgttttcttactttctgaatggcgtgaaaatatctactattcacaatagactagatgaggattatatcccattatttggcgcggttaaagggaatgctatatattatcttcatcgttttatattacgaagatttaatcatatcgttgcttgctcagcagcggtccaatcaaaactgaaacaatcgaaagtaaaaactaaaataaccaccatccagaatgggattgatataactaggtttaagacacttgagtctgataaaaaaaaattattgagggaaaaacacggatttgatagtgaaaaaagaatatttatatattgtggctcgttatcattaaggaaaaatattgcttacctcttggaacacttagccatcgaagaaaatgatatatttttaattctaggtgatggtgaactttttagatattgtaaggataaatattctaaagatttacggtatatatttatggggaaagttgaatgccctcttgaatattatcaattatcagatatttttgtttccgcttctttatcggaagggctccccttggcactattagaagctgcctctactgggtgctatttatatgttagcgatatagagccccatagagaaattgcatctctattaggagaggaaaatatttctatgtttaaaattaaggatggatcatataattatttgcaacctaaaataaaaaaagctgactataacgctctttctgacgataaactttacaatatatccgataaaaaaatgtcaaatctttatgacaaactttttgtttctttattagagcagaggcactaatataatgatttatgtttcggtaatttctcatggtcatttcaaaactcttaaggaattaggagcagtatcaaaattaaataatcacagcagaattaaagttatcatcaaagataatttaggagagagcgagcttttggatttttgtcaggaaaacaaaataacttatttaaggtctaaagagaaaaaaggatttggagagaataataatgaagttttttcctctatatcctccttaattactaaggaagatttttttgtggttatgaatcctgatatatatattgagtgctctgatctattagatgtcgtagatgagtgtggttcagcgaatgttaatctagcaacgataaatttatacagggattttgataaaaaaacatatgataactcagtaaggaaatttccctcggcaattgatttttttatgtcatttttatttaagaaaaatgactgtgtagtaaataagaacaaaataacgaaaccaacatatgttgattgggctgcaggttcttttctaatatttaatgccttcttttattcaaaactcaacggattcaacgaaaagtattttatgtattgcgaagatattgatatatgttggcgagctaaaaaacacttcaatacttcagttttatactatccatgctatgcagcaattcatttggcacaatttaacaatcgtaggatttttagtagacatttcatttggcatataaaaagtattatcctttttttattatataaaaatggtatgctgcgttctagtaagttgctttaatgctaatattcttttaagaggtgagaatgatacctgttattttggctggtggttcgggaagtcgcttgtggccactttcacgagaaaagttccccaagcagtttttaaagttgactggcagtttgacaatgttgcagtcaacattgtcacgtcttaataatttaaatgctgatgattcaatagttatatgcaacgaagagcatagatttattgttgcagaacaattaagagagttaggcaaactttcaaataacattattcttgaacccaaaggtcgtaatacagcccctgctataacactcgcagcattagcagcaaaaagaaaattcgctgatgaagatccattgattcttattttagctgcagatcacaacatccaagacgaacatgttttctgtgaggcaattaataaggcgtcatctttagctagttatggaaaactagtgacttttggtatcgttccattcaaacctgaaactgggtatggctatattcgtcgcggtgatgaagtgcctgtagatgagcagcatgcggtggcctttgaagtggcgcagtttgtcgaaaaaccgaatctggaaaccgcgcaggcctatgtggcaagcggcgaatattactggaacagcggtatgttcctgttccgtgccggacgctatctcgaagaactgaaaaagtatcgtccggatattctcgatgcctgtgaaaaagcgatgagcgccgtcgatccggatctcgattttattcgtgtggatgaagaggcgtttctcgcttgtccggaagagtcggtggattacgcggtcatggaatgcacggcagatgccgttgtggtgccgatggatgcgggctggagcgatgtcggttcctggtcttcattatgggagatcagcgcccacaccgccgagggcaacgtttgccacggcgatgtgattaatcacaaaactgaaaacagctatgtgtacgccgaatctggcctggtcaccaccgtcggggtgaaagatttggtggtagtgcagaccaaagatgcagtgctgattgccgaccgtaatgcggtgcaggatgtgaagaaagtggtcgagcagatcaaagctgatggtcgccatgagcatcgggtgcatcgcgaagtgtatcgtccgtggggcaaatatgactctatcgacgcgggcgaccgctaccaggtgaaacgcatcaccgtgaaaccgggcgaaggtttgtcggtacagatgcattatcatcgcgcggaacactgggtggttgtcgcgggaacggcaaaagtcactatcaacggtgatatcaaactgcttggtgaaaacgagtccatttatattccgctgggggcgatgcactgcctggaaaacccggggaaaatagatttagaattaattgaagttcgctctggtgcatatcttgaagaagatgatgttattagatgttatgatcgctatggacgaaagtaatatataataattatttcagaattagaaatgataattataagttttcgtctggataaacaatagatagtatgggttggaaaatatgagttctttaacttgttttaaagcttacgacattcgcgggaaattaggtgaagaactgaatgaagatatcgcctggcgcattggtcgcgcctatggcgaatttctcaaaccgaaaaccattgtgttaggcggtgatgtccgtctcaccagcgaaaccttaaaactggcgctggcaaaaggtttacaggatgcgggcgtcgatgtgctggatattggcatgtccggcaccgaagagatttatttcgccacgttccatctcggcgtggatggcggcattgaagttaccgccagccataatccgatggattacaacggcatgaagctggtgcgcgaaggggctcgcccgatcagcggtgataccggactgcgcgacgtccagcgtctggcagaagctaacgactttcctcccgtcgatgaaaccaaacgcggtcgctatcagcaaatcaatctgcgtgacgcttacgttgatcacctgttcggttatatcaatgtcaaaaaccttacgccgctcaagctggtgatcaactccgggaatggcgcagcgggtccggtggtggacgctatcgaagcccgctttaaagccctcggcgcaccggtggagttaatcaaagtgcataacacgccggacggcaatttccccaacggtattcctaacccgttgctgccggaatgtcgcgacgacacccgcaatgcggtcatcaaacacggcgcggatatgggcattgcctttgatggcgattttgaccgctgtttcctgtttgacgaaaaagggcagtttattgagggctactacattgtcggcctgctggcagaagcgttcctcgaaaaaaatcccggcgcgaagatcatccacgatccacgtctctcctggaacaccattgatgtggtgacggccgcgggcggcacgccggtgatgtcgaaaacaggacacgcctttattaaagaacgtatgcgcaaggaagacgccatctacggtggcgaaatgagcgctcaccattacttccgcgatttcgcttactgtgacagcggcatgatcccgtggctgctggtcgccgaactggtgtgcctgaaaggaaaaacgctgggcgaactggtgcgcgaccggatggcggcgtttccggcaagcggtgagatcaacagaaaactggcgcaccctgttgaggcgattaaccgcgtggaacagcattttagccgtgaggtgctggcggtggatcgcaccgatggcatcagcatgacctttgccgactggcgctttaacctgcgctcttccaacaccgaaccggtggtgcgcctgaatgtggaatctcgcggtgatgttcaggttatggtaatccatactcaagaaatattatcaattttgacgtcataaagaataagccctgacaagttagggcttaattaatatatattttttttgaattggggatttgtggtaagatttttaatatgttatttaatgtggttgaattaatgttgactggaaaataataatgagaacgaaaaaagcattacacaactttaaagttgatttattaattacttttttattggttttgctagggttttatattcgaactgtttttgtttcaaaaatgggaagtgatattactggagtgatgttactattcacacagttgacagcatatctcaatttggcagaattaggtattggaattgcagctgccagcgtattatataaaccgctcagcgagaatgaatacaataaaataacttacataatatctttgctctcagtcatatacaaatatatatttgtgtttgttttgattcttggcgttgttataggtatctgtatttattactttattgattctgtaaaggttgtaaatggcgtttttttatattgggctttgttcgtttttaatacatcgttgacatatagttatgctaaatactccacattattaactgctaatcagcggtactcagcagtaagaaaaattcaaggtggcggaaaagttataataattgtatttcagatattaattttgtgctttacgcaaagtttcatactttatttgttagttgagactttaggtattttttctcaatatttgatttttaaaaaaataattgggaacggaaatcaatatctcagtaatgaggttttacttattgaaagcgataaacttttgataaaaaaagaattaaaaataagaataaaaaatatgttcttccataaaataggtgctgtgcttgtccttaatacagactacctgcttgtatcaaagtttctgacattaagttatgtgacaatttttggcagctatatgatggtatttcagatagtaactgttttgatgtcaagttttgttaatgctattactgcaggaatgggtaattacttaattaataaaagtaatttagaaattaaggaaattacacgtcaattttatgtgatatttatcgcctttgcaacattcatatcactaaatatgttttttcttgttaatgattttatcgcaaaatggataggtgttaattatacattaagtaacaccctagttgcattaatgattgttaacgtattcattagtgttgtcagggtaccttctgatatattaaaaaacgcaagtggacattttggtgatatttattatccattattagaaggtgtgctgaatattacgatatccatcattttggctatcattattggattacctggcattattatagggacaatagtatctaacttaatagtaataatgcttgcgaaaccattatatctttactctaagttatttaatcttagaaatccgacgagggtttattttgaatttatttctcggcctatgttatattcattatgtgtgattggggtgagctatttattgcgcgatgaaatatattcatttaaagtaagtacatggttggattttattaacaagctactcttagtctctactcctagcatattggtaatatgtgctattttctctacggatagtgactttagattatttttcagaaaaattatatatgtgattatgaagaaataaaaatttcgaaaatgtattaatcgaaattatgcaacgagctttatttttataaatgatatgtgatcttttcgcgaataggagtaaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa